(19) United States
(12) Patent Application Publication
(10) Pub. No.: US 2022/0119477 A1
(43) Pub. Date: Apr. 21, 2022

(54) TCR AND PEPTIDES

(71) Applicant: Ospedale San Raffaele S.R.L., Milan (IT)

(72) Inventors: Maria Chiara Bonini, Milan (IT); Eliana Eliana, Milan (IT); Zulma Irene Magnani, Milan (IT); Fabio Ciceri, Milan (IT); Erica Carnevale, Milan (IT)

(21) Appl. No.: 17/288,406

(22) PCT Filed: Oct. 31, 2019
(86) PCT No.: PCT/EP2019/079916

§ 371 Date: Apr. 23, 2021

(30) Foreign Application Priority Data
Oct. 31, 2018 (GB) 1817821.0

(51) Int. Cl. C07K 14/725; C12N 15/90; A61K 35/17; A61K 38/00

(52) U.S. Cl. CPC C07K 14/7051; A61K 38/00; A61K 35/17; C12N 15/907

(57) ABSTRACT

A T-cell receptor (TCR), which binds to a Wilms tumour 1 protein (WT1) peptide when presented by a major histocompatibility complex (MHC).

### CROSS-REFERENCE TO RELATED APPLICATIONS

This application is a U.S. National Phase of International Patent Application No. PCT/EP2019/079916, filed on Oct. 31, 2019, which claims priority to United Kingdom Patent Application No. 1817821.0 filed on Oct. 31, 2018.

### INCORPORATION BY REFERENCE OF MATERIAL SUBMITTED ELECTRONICALLY

A Sequence Listing, submitted as part of the specification as a text file, is incorporated herein by reference. The file containing the Sequence Listing is named 56627_Seqlisting, created on Apr. 23, 2021, and is 297,949 bytes in size.

### FIELD OF THE INVENTION

The present invention relates to T-cell receptors (TCRs) which bind to peptides derived from Wilms tumour 1 protein (WT1) when presented by a major histocompatibility complex. In this regard, the present invention relates to complementarity determining regions (CDRs) which specifically recognise WT1 peptides. The present invention further relates to immunogenic peptides derived from WT1.

### BACKGROUND TO THE INVENTION

T cell receptor (TCR) gene therapy is based on the genetic transfer of high-avidity tumour-specific TCR genes into T lymphocytes, thus enabling the specific targeting of the desired tumour-associated antigens and leading to a less toxic and more specific and effective therapy. This approach has shown promise in clinical trials. One of the main barriers limiting the exploitation of TCR gene therapy for clinical treatment of cancers is the lack of tumour-specific T-cells and corresponding TCRs. Thus, the low availability of tumour-specific TCRs still remains an open issue limiting the broad exploitation of TCR-based immunotherapeutic approaches.

The majority of tumour-associated antigens (TAAs) are self antigens, thus T-cells specific for such molecules are either destroyed or anergized due to central and peripheral tolerance.

Despite this, naturally occurring tumour-specific T-cells have been observed in healthy donors and patients, particularly in patients affected by hematological malignancies, after allogeneic hematopoietic stem cell transplantation (allo-HSCT) where frequencies of tumor-specific lymphocytes have been correlated with disease regression (Kapp, M. et al. Bone Marrow Transplantation 43,399-410 (2009); and Tyler, E. M. et al. Blood 121,308-317 (2013)). The choice of a tumor antigen to be targeted by immunotherapeutic approaches is still a matter of debate. Ideal TAAs are highly expressed on tumor cells while being minimally expressed in healthy tissue.

Wilms tumor 1 (WT1) is an intracellular protein encoding a zinc finger transcription factor that plays an important role in cell growth and differentiation (Yang, L. et al. Leukemia 21, 868-876 (2007)). WT1 is widely expressed on a variety of hematological and solid tumors, while showing limited expression on various healthy tissues (e.g. gonads, uterus, kidney, mesothelium, progenitor cells in different tissues). Recent evidence suggests a role for WT1 in leukemogenesis and tumorigenesis.

Several ongoing clinical trials rely on the generation of cytotoxic T lymphocyte (CTL) responses upon vaccination with WT1 peptides. However, despite the recognition that WT1 is useful for immunotherapy, a small number of WT1 epitopes, which are restricted to a limited number of HLA alleles, are presently used for vaccination purposes (Di Stasi, A. et al. Front. Immunol. (2015)). One such epitope is the WT1 126-134 epitope (RMFPNAPYL; SEQ ID NO: 71), which is presented by MHC encoded by the HLA-A*0201 allele (i.e. the epitope is HLA-A*0201 restricted).

HLA-A*0201 restricted epitopes and corresponding TCRs are of interest since major histocompatibility complex (MHC) having the HLA-A*0201 haplotype are expressed in the vast majority (60%) of the Caucasian population. Accordingly, TCRs that target HLA-A*0201-restricted WT1 epitopes are particularly advantageous since an immunotherapy making use of such TCRs may be widely applied.

The WT1 126-134 epitope has been widely studied in several trials, alone or in combination with additional tumor antigens. However, recent reports have highlighted a major concern regarding the processing of this particular epitope, which may impair its use for immunotherapy purposes. Notably, the WT1 126-134 epitope is more efficiently processed by the immunoproteasome compared with standard proteasomes (Jaigirdar, A. et al. J Immunother. 39(3):105-16 (2016)), which leads to poor recognition of many HLA-A*0201 tumour cell lines or primary leukemia cells that endogenously express WT1.

Thus, there remains a need for new WT1 epitopes, particularly those presented by MHC with prevalent HLA haplotypes (e.g. HLA-A*0201).

One naturally processed HLA-A*0201 restricted epitope that has been identified is WT1 37-45, which has the amino acid sequence VLDFAPPGA (SEQ ID NO: 72, see e.g. Smithgall et al 2001; Blood 98 (11 Part 1): 121a). However, few TCR amino acid sequences, particularly CDR sequences, specific for this peptide sequence have been reported (Schmitt, T. M. et al. (2017) Nat Biotechnol 35: 1188-1195).

Accordingly, there remains a need for new WT1 epitopes, particularly those restricted to common HLA alleles and a need for new TCRs capable of binding to WT1 epitopes.

### SUMMARY OF THE INVENTION

We have identified novel TCRs that bind to WT1 peptides when presented by an MHC. Further, we have determined the amino acid sequences of the TCRs, including the amino acid sequences of their CDR regions, which are responsible for binding specificity for WT1. Moreover, we have demonstrated that T-cells expressing TCRs according to the invention specifically target and kill cells that overexpress the WT1 protein. In addition, it has been shown that TCRs of the present invention are restricted to MHC encoded by HLA class I and II alleles common in the Caucasian population, such as HLA-A*0201, HLA-B*38:01, HLA-C*03:03 or HLA-C*07:02.

In one aspect, the invention provides a T-cell receptor (TCR), which binds to a Wilms tumour 1 protein (WT1) peptide when presented by a major histocompatibility complex (MHC), wherein the TCR:
(i) comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions;
(ii) comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions;
(iii) comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions;
(iv) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(v) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vi) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vii) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(viii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(ix) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(x) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xiii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xiv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xvi) comprises a CDR3α comprising the amino acid sequence of CALPDKVIF (SEQ ID NO: 148) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xvii) comprises a CDR3α comprising the amino acid sequence of CAGLYATNKLIF (SEQ ID NO: 153) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xviii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xix) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(xx) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(xxi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions; or
(xxii) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions.

In one embodiment, the TCR comprises the following CDR sequences:

(i)
CDR1α
(SEQ ID NO: 23)
NSAFQY,
CDR2α
(SEQ ID NO: 24)
TYSSGN,
CDR3α
(SEQ ID NO: 25)
CASGGGADGLTF,
CDR1β
(SEQ ID NO: 28)
SGDLS,
CDR2β
(SEQ ID NO: 29)
YYNGEE,
and
CDR3β
(SEQ ID NO: 30)
CASGRGDTEAFF,

or variants thereof each having up to three amino acid substitutions, additions or deletions;

(ii)
CDR1α
(SEQ ID NO: 1)
TSDQSYG,
CDR2α
(SEQ ID NO: 2)
QGSYDEQN,
CDR3α
(SEQ ID NO: 3)
CAMRTGGGADGLTF,
CDR1β
(SEQ ID NO: 6)
SNHLY,
CDR2β
(SEQ ID NO: 7)
FYNNEI,
and
CDR3β
(SEQ ID NO: 8)
CASSEAGLSYEQYF,

or variants thereof each having up to three amino acid substitutions, additions or deletions;

(iii)
CDR1α
(SEQ ID NO: 12)
TISGTDY,
CDR2α
(SEQ ID NO: 13)
GLTSN,
CDR3α
(SEQ ID NO: 14)
CILSTRVWAGSYQLTF,
CDR1β
(SEQ ID NO: 17)
KGHDR,
CDR2β
(SEQ ID NO: 18)
SFDVKD,
and
CDR3β
(SEQ ID NO: 19)
CATGQATQETQYF,

or variants thereof each having up to three amino acid substitutions, additions or deletions;

(iv)
CDR1α
(SEQ ID NO: 34)
DRGSQS,
CDR2α
(SEQ ID NO: 35)
IYSNGD,
CDR3α
(SEQ ID NO: 36)
CAVIGGTDSWGKLQF,
CDR1β
(SEQ ID NO: 39)
LGHNA,
CDR2β
(SEQ ID NO: 40)
YSLEER,
and
CDR3β
(SEQ ID NO: 41)
CASSQEEGAVYGYTF,

or variants thereof each having up to three amino acid substitutions, additions or deletions;

(v)
CDR1α
(SEQ ID NO: 34)
DRGSQS,
CDR2α
(SEQ ID NO: 35)
IYSNGD,
CDR3α
(SEQ ID NO: 36)
CAVIGGTDSWGKLQF,
CDR1β
(SEQ ID NO: 50)
LNHNV,
CDR2β
(SEQ ID NO: 51)
YYDKDF,
and
CDR3β
(SEQ ID NO: 52)
CATSREGLAADTQYF,

or variants thereof each having up to three amino acid substitutions, additions or deletions;

(vi)
CDR1α
(SEQ ID NO: 45)
NSASQS,
CDR2α
(SEQ ID NO: 46)
VYSSGN,
CDR3α
(SEQ ID NO: 47)
CVVPRGLSTDSWGKLQF,
CDR1β
(SEQ ID NO: 50)
LNHNV,
CDR2β
(SEQ ID NO: 51)
YYDKDF,
and
CDR3β
(SEQ ID NO: 52)
CATSREGLAADTQYF,

or variants thereof each having up to three amino acid substitutions, additions or deletions;

(vii)
CDR1α
(SEQ ID NO: 45)
NSASQS,
CDR2α
(SEQ ID NO: 46)
VYSSGN,
CDR3α
(SEQ ID NO: 47)
CVVPRGLSTDSWGKLQF,
CDR1β
(SEQ ID NO: 39)
LGHNA,
CDR2β
(SEQ ID NO: 40)
YSLEER,
and
CDR3β
(SEQ ID NO: 41)
CASSQEEGAVYGYTF,

or variants thereof each having up to three amino acid substitutions, additions or deletions;

(viii)
CDR1α
(SEQ ID NO: 91)
VSNAYN,
CDR2α
(SEQ ID NO: 92)
GSKP,
CDR3α
(SEQ ID NO: 93)
CAAPNDYKLSF,
CDR1β
(SEQ ID NO: 96)
SEHNR,
CDR2β
(SEQ ID NO: 97)
FQNEAQ,
and
CDR3β
(SEQ ID NO: 98)
CASSSGLAFYEQYF,

or variants thereof each having up to three amino acid substitutions, additions or deletions;

(ix)
CDR1α
(SEQ ID NO: 91)
VSNAYN,
CDR2α
(SEQ ID NO: 92)
GSKP,
CDR3α
(SEQ ID NO: 93)
CAAPNDYKLSF,
CDR1β
(SEQ ID NO: 102)
SGHDN,
CDR2β
(SEQ ID NO: 103)
FVKESK,
and
CDR3β
(SEQ ID NO: 104)
CASSQLSGRDSYEQYF,

or variants thereof each having up to three amino acid substitutions, additions or deletions;

(x)
CDR1α
(SEQ ID NO: 108)
VSGNPY,
CDR2α
(SEQ ID NO: 109)
YITGDNLV,
CDR3α
(SEQ ID NO: 110)
CAVRDGGATNKLIF,
CDR1β
(SEQ ID NO: 118)
MNHEY,
CDR2β
(SEQ ID NO: 119)
SMNVEV,
and
CDR3β
(SEQ ID NO: 120)
CASSTLGGELFF,

or variants thereof each having up to three amino acid substitutions, additions or deletions;

(xi)
CDR1α
(SEQ ID NO: 113)
NIATNDY,
CDR2α
(SEQ ID NO: 114)
GYKTK,
CDR3α
(SEQ ID NO: 115)
CLVGGYTGGFKTIF,
CDR1β
(SEQ ID NO: 118)
MNHEY,
CDR2β
(SEQ ID NO: 119)
SMNVEV,
and
CDR3β
(SEQ ID NO: 120)
CASSTLGGELFF,

or variants thereof each having up to three amino acid substitutions, additions or deletions;

(xii)
CDR1α
(SEQ ID NO: 124)
SSVSVY,
CDR2α
(SEQ ID NO: 125)
YLSGSTLV,
CDR3α
(SEQ ID NO: 126)
CAVTLLSIEPSAGGYQKVTF,
CDR1β
(SEQ ID NO: 134)
SEHNR,
CDR2β
(SEQ ID NO: 135)
FQNEAQ,
and
CDR3β
(SEQ ID NO: 136)
CASSLEGRAMPRDSHQETQYF,

or variants thereof each having up to three amino acid substitutions, additions or deletions;

(xiii)
CDR1α
(SEQ ID NO: 124)
SSVSVY,
CDR2α
(SEQ ID NO: 125)
YLSGSTLV,
CDR3α
(SEQ ID NO: 126)
CAVTLLSIEPSAGGYQKVTF,
CDR1β
(SEQ ID NO: 140)
LNHNV,
CDR2β
(SEQ ID NO: 141)
YYDKDF,
and
CDR3β
(SEQ ID NO: 142)
CATSWGLNEQYF,

or variants thereof each having up to three amino acid substitutions, additions or deletions;

(xiv)
CDR1α
(SEQ ID NO: 129)
DSASNY,
CDR2α
(SEQ ID NO: 130)
IRSNVGE,
CDR3α
(SEQ ID NO: 131)
CAATSRDDMRF,
CDR1β
(SEQ ID NO: 134)
SEHNR,
CDR2β
(SEQ ID NO: 135)
FQNEAQ,
and
CDR3β
(SEQ ID NO: 136)
CASSLEGRAMPRDSHQETQYF,

or variants thereof each having up to three amino acid substitutions, additions or deletions;

(xv)
CDR1α
(SEQ ID NO: 129)
DSASNY,
CDR2α
(SEQ ID NO: 130)
IRSNVGE,
CDR3α
(SEQ ID NO: 131)
CAATSRDDMRF,
CDR1β
(SEQ ID NO: 140)
LNHNV,
CDR2β
(SEQ ID NO: 141)
YYDKDF,
and
CDR3β
(SEQ ID NO: 142)
CATSWGLNEQYF,

or variants thereof each having up to three amino acid substitutions, additions or deletions;

(xvi)
(SEQ ID NO: 146)
CDR1α-TRDTTYY,
(SEQ ID NO: 147)
CDR2α-RNSFDEQN,
(SEQ ID NO: 148)
CDR3α-CALPDKVIF,
(SEQ ID NO: 156)
CDR1β-SGDLS,
(SEQ ID NO: 157)
CDR2β-YYNGEE,
and
(SEQ ID NO: 158)
CDR3β-CASSVSAGSTGELFF,

or variants thereof each having up to three amino acid substitutions, additions or deletions;

(xvii)
(SEQ ID NO: 151)
CDR1α-SIFNT,
(SEQ ID NO: 152)
CDR2α-LYKAGEL,
(SEQ ID NO: 153)
CDR3α-CAGLYATNKLIF,
(SEQ ID NO: 156)
CDR1β-SGDLS,
(SEQ ID NO: 157)
CDR2β-YYNGEE,
and
(SEQ ID NO: 158)
CDR3β-CASSVSAGSTGELFF,

or variants thereof each having up to three amino acid substitutions, additions or deletions;

(xviii)
(SEQ ID NO: 91)
CDR1α-VSNAYN,
(SEQ ID NO: 92)
CDR2α-GSKP,
(SEQ ID NO: 93)
CDR3α-CAAPNDYKLSF,
(SEQ ID NO: 118)
CDR1β-MNHEY,
(SEQ ID NO: 119)
CDR2β-SMNVEV,
and
(SEQ ID NO: 120)
CDR3β-CASSTLGGELFF,

or variants thereof each having up to three amino acid substitutions, additions or deletions;

(xix)
(SEQ ID NO: 108)
CDR1α-VSGNPY,
(SEQ ID NO: 109)
CDR2α-YITGDNLV,
(SEQ ID NO: 110)
CDR3α-CAVRDGGATNKLIF,
(SEQ ID NO: 96)
CDR1β-SEHNR,
(SEQ ID NO: 97)
CDR2β-FQNEAQ,
and
(SEQ ID NO: 98)
CDR3β-CASSSGLAFYEQYF,

or variants thereof each having up to three amino acid substitutions, additions or deletions;

(xx)
(SEQ ID NO: 108)
CDR1α-VSGNPY,
(SEQ ID NO: 109)
CDR2α-YITGDNLV,
(SEQ ID NO: 110)
CDR3α-CAVRDGGATNKLIF,
(SEQ ID NO: 102)
CDR1β-SGHDN,
(SEQ ID NO: 103)
CDR2β-FVKESK,
and
(SEQ ID NO: 104)
CDR3β-CASSQLSGRDSYEQYF,

or variants thereof each having up to three amino acid substitutions, additions or deletions;

(xxi)
(SEQ ID NO: 113)
CDR1α-NIATNDY,
(SEQ ID NO: 114)
CDR2α-GYKTK,
(SEQ ID NO: 115)
CDR3α-CLVGGYTGGFKTIF,
(SEQ ID NO: 96)
CDR1β-SEHNR,
(SEQ ID NO: 97)
CDR2β-FQNEAQ,
and
(SEQ ID NO: 98)
CDR3β-CASSSGLAFYEQYF,

or variants thereof each having up to three amino acid substitutions, additions or deletions;

(xxii)
(SEQ ID NO: 113)
CDR1α-NIATNDY,
(SEQ ID NO: 114)
CDR2α-GYKTK,
(SEQ ID NO: 115)
CDR3α-CLVGGYTGGFKTIF,
(SEQ ID NO: 102)
CDR1β-SGHDN,
(SEQ ID NO: 103)
CDR2β-FVKESK,
and
(SEQ ID NO: 104)
CDR3β-CASSQLSGRDSYEQYF,

or variants thereof each having up to three amino acid substitutions, additions or deletions; or

(xxiii)
(SEQ ID NO: 182)
CDR1α-DRGSQS,
(SEQ ID NO: 183)
CDR2α-IYSNGD,
(SEQ ID NO: 25)
CDR3α-CASGGGADGLTF,
(SEQ ID NO: 28)
CDR1β-SGDLS,
(SEQ ID NO: 29)
CDR2β-YYNGEE,
and
(SEQ ID NO: 30)
CDR3β-CASGRGDTEAFF,

or variants thereof each having up to three amino acid substitutions, additions or deletions.

In one embodiment, the TCR comprises:
(i) an α chain variable domain comprising the amino acid sequence of SEQ ID NO: 26 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 31 or a variant thereof having at least 75%, at least 80%, at least 85%, at least 90%, at least 95%, at least 96%, at least 97%, at least 98%, or at least 99%, preferably at least 75%, sequence identity thereto;
(ii) an α chain variable domain comprising the amino acid sequence of SEQ ID NO: 4 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 9 or a variant thereof having at least 75%, at least 80%, at least 85%, at least 90%, at least 95%, at least 96%, at least 97%, at least 98%, or at least 99%, preferably at least 75%, sequence identity thereto;
(iii) an α chain variable domain comprising the amino acid sequence of SEQ ID NO: 15 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 20 or a variant thereof having at least 75%, at least 80%, at least 85%, at least 90%, at least 95%, at least 96%, at least 97%, at least 98%, or at least 99%, preferably at least 75%, sequence identity thereto;
(iv) an α chain variable domain comprising the amino acid sequence of SEQ ID NO: 37 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 42 or a variant thereof having at least 75%, at least 80%, at least 85%, at least 90%, at least 95%, at least 96%, at least 97%, at least 98%, or at least 99%, preferably at least 75%, sequence identity thereto;
(v) an α chain variable domain comprising the amino acid sequence of SEQ ID NO: 37 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 53 or a variant thereof having at least 75%, at least 80%, at least 85%, at least 90%, at least 95%, at least 96%, at least 97%, at least 98%, or at least 99%, preferably at least 75%, sequence identity thereto;
(vi) an α chain variable domain comprising the amino acid sequence of SEQ ID NO: 48 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 53 or a variant thereof having at least 75%, at least 80%, at least 85%, at least 90%, at least 95%, at least 96%, at least 97%, at least 98%, or at least 99%, preferably at least 75%, sequence identity thereto;
(vii) an α chain variable domain comprising the amino acid sequence of SEQ ID NO: 48 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 42 or a variant thereof having at least 75%, at least 80%, at least 85%, at least 90%, at least 95%, at least 96%, at least 97%, at least 98%, or at least 99%, preferably at least 75%, sequence identity thereto;
(viii) an α chain variable domain comprising the amino acid sequence of SEQ ID NO: 94 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 99 or a variant thereof having at least 75%, at least 80%, at least 85%, at least 90%, at least 95%, at least 96%, at least 97%, at least 98%, or at least 99%, preferably at least 75%, sequence identity thereto;
(ix) an α chain variable domain comprising the amino acid sequence of SEQ ID NO: 94 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto;
(x) an α chain variable domain comprising the amino acid sequence of SEQ ID NO: 111 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 121 or a variant thereof having at least 75%, at least 80%, at least 85%, at least 90%, at least 95%, at least 96%, at least 97%, at least 98%, or at least 99%, preferably at least 75%, sequence identity thereto;
(xi) an α chain variable domain comprising the amino acid sequence of SEQ ID NO: 116 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 121 or a variant thereof having at least 75%, at least 80%, at least 85%, at least 90%, at least 95%, at least 96%, at least 97%, at least 98%, or at least 99%, preferably at least 75%, sequence identity thereto;
(xii) an α chain variable domain comprising the amino acid sequence of SEQ ID NO: 127 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 137 or a variant thereof having at least 75%, at least 80%, at least 85%, at least 90%, at least 95%, at least 96%, at least 97%, at least 98%, or at least 99%, preferably at least 75%, sequence identity thereto;
(xiii) an α chain variable domain comprising the amino acid sequence of SEQ ID NO: 127 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 143 or a variant thereof having at least 75%, at least 80%, at least 85%, at least 90%, at least 95%, at least 96%, at least 97%, at least 98%, or at least 99%, preferably at least 75%, sequence identity thereto;
(xiv) an α chain variable domain comprising the amino acid sequence of SEQ ID NO: 132 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 137 or a variant thereof having at least 75%, at least 80%, at least 85%, at least 90%, at least 95%, at least 96%, at least 97%, at least 98%, or at least 99%, preferably at least 75%, sequence identity thereto;
(xv) an α chain variable domain comprising the amino acid sequence of SEQ ID NO: 132 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 143 or a variant thereof having at least 75%, at least 80%, at least 85%, at least 90%, at least 95%, at least 96%, at least 97%, at least 98%, or at least 99%, preferably at least 75%, sequence identity thereto;
(xvi) an α chain variable domain comprising the amino acid sequence of SEQ ID NO: 149 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 159 or a variant thereof having at least 75%, at least 80%, at least 85%, at least 90%, at least 95%, at least 96%, at least 97%, at least 98%, or at least 99%, preferably at least 75%, sequence identity thereto;
(xvii) an α chain variable domain comprising the amino acid sequence of SEQ ID NO: 154 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 159 or a variant thereof having at least 75%, at least 80%, at least 85%, at least 90%, at least 95%, at least 96%, at least 97%, at least 98%, or at least 99%, preferably at least 75%, sequence identity thereto;
(xviii) an α chain variable domain comprising the amino acid sequence of SEQ ID NO: 94 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 121 or a variant thereof having at least 75%, at least 80%, at least 85%, at least 90%, at least 95%, at least 96%, at least 97%, at least 98%, or at least 99%, preferably at least 75%, sequence identity thereto;
(xix) an α chain variable domain comprising the amino acid sequence of SEQ ID NO: 111 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 99 or a variant thereof having at least 75%, at least 80%, at least 85%, at least 90%, at least 95%, at least 96%, at least 97%, at least 98%, or at least 99%, preferably at least 75%, sequence identity thereto;
(xx) an α chain variable domain comprising the amino acid sequence of SEQ ID NO: 111 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto;
(xxi) an α chain variable domain comprising the amino acid sequence of SEQ ID NO: 116 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 99 or a variant thereof having at least 75%, at least 80%, at least 85%, at least 90%, at least 95%, at least 96%, at least 97%, at least 98%, or at least 99%, preferably at least 75%, sequence identity thereto;
(xxii) an α chain variable domain comprising the amino acid sequence of SEQ ID NO: 116 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto; or
(xxiii) an α chain variable domain comprising the amino acid sequence selected from the group consisting of SEQ ID NO: 185, 190 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 31 or a variant thereof having at least 75%, at least 80%, at least 85%, at least 90%, at least 95%, at least 96%, at least 97%, at least 98%, or at least 99%, preferably at least 75%, sequence identity thereto.

In one embodiment, the TCR comprises:
(i) an α chain comprising the amino acid sequence of SEQ ID NO: 27 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 32, SEQ ID NO: 33, SEQ ID NO: 203 and variants of SEQ ID NOs: 32, 33 and 203 having at least 75%, at least 80%, at least 85%, at least 90%, at least 95%, at least 96%, at least 97%, at least 98%, or at least 99%, preferably at least 75%, sequence identity thereto;
(ii) an α chain comprising the amino acid sequence of SEQ ID NO: 5 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 10, SEQ ID NO: 11, SEQ ID NO: 195 and variants of SEQ ID NOs: 10, 11 and 195 having at least 75%, at least 80%, at least 85%, at least 90%, at least 95%, at least 96%, at least 97%, at least 98%, or at least 99%, preferably at least 75%, sequence identity thereto;
(iii) an α chain comprising the amino acid sequence of SEQ ID NO: 16 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 21, SEQ ID NO: 22, SEQ ID NO: 197 and variants of SEQ ID NOs: 21, 22 and 197 having at least 75%, at least 80%, at least 85%, at least 90%, at least 95%, at least 96%, at least 97%, at least 98%, or at least 99%, preferably at least 75%, sequence identity thereto;
(iv) an α chain comprising the amino acid sequence of SEQ ID NO: 38 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75%, at least 80%, at least 85%, at least 90%, at least 95%, at least 96%, at least 97%, at least 98%, or at least 99%, preferably at least 75%, sequence identity thereto;
   (v) an α chain comprising the amino acid sequence of SEQ ID NO: 38 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75%, at least 80%, at least 85%, at least 90%, at least 95%, at least 96%, at least 97%, at least 98%, or at least 99%, preferably at least 75%, sequence identity thereto;
   (vi) an α chain comprising the amino acid sequence of SEQ ID NO: 49 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75%, at least 80%, at least 85%, at least 90%, at least 95%, at least 96%, at least 97%, at least 98%, or at least 99%, preferably at least 75%, sequence identity thereto;
   (vii) an α chain comprising the amino acid sequence of SEQ ID NO: 49 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75%, at least 80%, at least 85%, at least 90%, at least 95%, at least 96%, at least 97%, at least 98%, or at least 99%, preferably at least 75%, sequence identity thereto;
   (viii) an α chain comprising the amino acid sequence of SEQ ID NO: 95 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 100, SEQ ID NO: 101 and variants of SEQ ID NOs: 100 and 101 having at least 75%, at least 80%, at least 85%, at least 90%, at least 95%, at least 96%, at least 97%, at least 98%, or at least 99%, preferably at least 75%, sequence identity thereto;
(ix) an α chain comprising the amino acid sequence of SEQ ID NO: 95 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 106, SEQ ID NO: 107 and variants of SEQ ID NOs: 106 and 107 having at least 75%, at least 80%, at least 85%, at least 90%, at least 95%, at least 96%, at least 97%, at least 98%, or at least 99%, preferably at least 75%, sequence identity thereto;
(x) an α chain comprising the amino acid sequence of SEQ ID NO: 112 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 122, SEQ ID NO: 123 and variants of SEQ ID NOs: 122 and 123 having at least 75%, at least 80%, at least 85%, at least 90%, at least 95%, at least 96%, at least 97%, at least 98%, or at least 99%, preferably at least 75%, sequence identity thereto;
(xi) an α chain comprising the amino acid sequence of SEQ ID NO: 117 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 122, SEQ ID NO: 123 and variants of SEQ ID NOs: 122 and 123 having at least 75%, at least 80%, at least 85%, at least 90%, at least 95%, at least 96%, at least 97%, at least 98%, or at least 99%, preferably at least 75%, sequence identity thereto;
(xii) an α chain comprising the amino acid sequence of SEQ ID NO: 128 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 138, SEQ ID NO: 139 and variants of SEQ ID NOs: 138 and 139 having at least 75%, at least 80%, at least 85%, at least 90%, at least 95%, at least 96%, at least 97%, at least 98%, or at least 99%, preferably at least 75%, sequence identity thereto;
(xiii) an α chain comprising the amino acid sequence of SEQ ID NO: 128 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 144, SEQ ID NO: 145 and variants of SEQ ID NOs: 144 and 145 having at least 75%, at least 80%, at least 85%, at least 90%, at least 95%, at least 96%, at least 97%, at least 98%, or at least 99%, preferably at least 75%, sequence identity thereto;
(xiv) an α chain comprising the amino acid sequence of SEQ ID NO: 133 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 138, SEQ ID NO: 139 and variants of SEQ ID NOs: 138 and 139 having at least 75%, at least 80%, at least 85%, at least 90%, at least 95%, at least 96%, at least 97%, at least 98%, or at least 99%, preferably at least 75%, sequence identity thereto;
(xv) an α chain comprising the amino acid sequence of SEQ ID NO: 133 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 144, SEQ ID NO: 145 and variants of SEQ ID NOs: 144 and 145 having at least 75%, at least 80%, at least 85%, at least 90%, at least 95%, at least 96%, at least 97%, at least 98%, or at least 99%, preferably at least 75%, sequence identity thereto;
(xvi) an α chain comprising the amino acid sequence of SEQ ID NO: 150 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 160, SEQ ID NO: 161 and variants of SEQ ID NOs: 160 and 161 having at least 75%, at least 80%, at least 85%, at least 90%, at least 95%, at least 96%, at least 97%, at least 98%, or at least 99%, preferably at least 75%, sequence identity thereto;
(xvii) an α chain comprising the amino acid sequence of SEQ ID NO: 155 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 160, SEQ ID NO: 161 and variants of SEQ ID NOs: 160 and 161 having at least 75%, at least 80%, at least 85%, at least 90%, at least 95%, at least 96%, at least 97%, at least 98%, or at least 99%, preferably at least 75%, sequence identity thereto;
(xviii) an α chain comprising the amino acid sequence of SEQ ID NO: 95 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 122, SEQ ID NO: 123 and variants of SEQ ID NOs: 122 and 123 having at least 75%, at least 80%, at least 85%, at least 90%, at least 95%, at least 96%, at least 97%, at least 98%, or at least 99%, preferably at least 75%, sequence identity thereto;
(xix) an α chain comprising the amino acid sequence of SEQ ID NO: 112 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 100, SEQ ID NO: 101 and variants of SEQ ID NOs: 100 and 101 having at least 75%, at least 80%, at least 85%, at least 90%, at least 95%, at least 96%, at least 97%, at least 98%, or at least 99%, preferably at least 75%, sequence identity thereto;
(xx) an α chain comprising the amino acid sequence of SEQ ID NO: 112 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 106, SEQ ID NO: 107 and variants of SEQ ID NOs: 106 and 107 having at least 75%, at least 80%, at least 85%, at least 90%, at least 95%, at least 96%, at least 97%, at least 98%, or at least 99%, preferably at least 75%, sequence identity thereto;
(xxi) an α chain comprising the amino acid sequence of SEQ ID NO: 117 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 100, SEQ ID NO: 101 and variants of SEQ ID NOs: 100 and 101 having at least 75%, at least 80%, at least 85%, at least 90%, at least 95%, at least 96%, at least 97%, at least 98%, or at least 99%, preferably at least 75%, sequence identity thereto;
(xxii) an α chain comprising the amino acid sequence of SEQ ID NO: 117 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 106, SEQ ID NO: 107 and variants of SEQ ID NOs: 106 and 107 having at least 75%, at least 80%, at least 85%, at least 90%, at least 95%, at least 96%, at least 97%, at least 98%, or at least 99%, preferably at least 75%, sequence identity thereto; or
(xxiii) (a) an α chain comprising an amino acid sequence selected from the group consisting of SEQ ID NOs: 186, 191, 198, 199, 200, 201, 202 and variants of SEQ ID NOs: 186, 191, 198, 199, 200, 201 and 202 having at least 75%, at least 80%, at least 85%, at least 90%, at least 95%, at least 96%, at least 97%, at least 98%, or at least 99%, preferably at least 75%, sequence identity thereto; and a β chain comprising the amino acid sequence of SEQ ID NO: 32 or a variant thereof having at least 75%, at least 80%, at least 85%, at least 90%, at least 95%, at least 96%, at least 97%, at least 98%, or at least 99%, preferably at least 75%, sequence identity thereto;
   (b) an α chain comprising an amino acid sequence selected from the group consisting of SEQ ID NOs: 186, 191, 198, 199, 200, 201, 202 and variants of SEQ ID NOs: 186, 191, 198, 199, 200, 201 and 202 having at least 75%, at least 80%, at least 85%, at least 90%, at least 95%, at least 96%, at least 97%, at least 98%, or at least 99%, preferably at least 75%, sequence identity thereto; and a β chain comprising the amino acid sequence of SEQ ID NO: 33 or a variant thereof having at least 75%, at least 80%, at least 85%, at least 90%, at least 95%, at least 96%, at least 97%, at least 98%, or at least 99%, preferably at least 75%, sequence identity thereto; or
   (c) an α chain comprising an amino acid sequence selected from the group consisting of SEQ ID NOs: 186, 191, 198, 199, 200, 201, 202 and variants of SEQ ID NOs: 186, 191, 198, 199, 200, 201 and 202 having at least 75%, at least 80%, at least 85%, at least 90%, at least 95%, at least 96%, at least 97%, at least 98%, or at least 99%, preferably at least 75%, sequence identity thereto; and a β chain comprising the amino acid sequence of SEQ ID NO: 203 or a variant thereof having at least 75%, at least 80%, at least 85%, at least 90%, at least 95%, at least 96%, at least 97%, at least 98%, or at least 99%, preferably at least 75%, sequence identity thereto.
(xxiv) an α chain comprising the amino acid sequence of SEQ ID NO: 194 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 10, SEQ ID NO: 11, SEQ ID NO: 195 and variants of SEQ ID NOs: 10, 11 and 195 having at least 75%, at least 80%, at least 85%, at least 90%, at least 95%, at least 96%, at least 97%, at least 98%, or at least 99%, preferably at least 75%, sequence identity thereto;
(xxv) an α chain comprising the amino acid sequence of SEQ ID NO: 196 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 21, SEQ ID NO: 22, SEQ ID NO: 197 and variants of SEQ ID NOs: 21, 22 and 197 having at least 75%, at least 80%, at least 85%, at least 90%, at least 95%, at least 96%, at least 97%, at least 98%, or at least 99%, preferably at least 75%, sequence identity thereto;
(xxvi) an α chain comprising the amino acid sequence of SEQ ID NO: 214 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75%, at least 80%, at least 85%, at least 90%, at least 95%, at least 96%, at least 97%, at least 98%, or at least 99%, preferably at least 75%, sequence identity thereto;
(xxvii) an α chain comprising the amino acid sequence of SEQ ID NO: 214 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75%, at least 80%, at least 85%, at least 90%, at least 95%, at least 96%, at least 97%, at least 98%, or at least 99%, preferably at least 75%, sequence identity thereto;
(xxviii) an α chain comprising the amino acid sequence of SEQ ID NO: 216 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75%, at least 80%, at least 85%, at least 90%, at least 95%, at least 96%, at least 97%, at least 98%, or at least 99%, preferably at least 75%, sequence identity thereto; or
(xxix) an α chain comprising the amino acid sequence of SEQ ID NO: 216 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75%, at least 80%, at least 85%, at least 90%, at least 95%, at least 96%, at least 97%, at least 98%, or at least 99%, preferably at least 75%, sequence identity thereto.

In one embodiment, the TCR of the invention binds to a Wilms tumour 1 protein (WT1) peptide when presented by a major histocompatibility complex (MHC), wherein the WT1 peptide comprises an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87) and variants thereof each having up to three amino acid substitutions, additions or deletions.

In another aspect, the invention provides a T-cell receptor (TCR), which binds to a Wilms tumour 1 protein (WT1) peptide when presented by a major histocompatibility complex (MHC), wherein the WT1 peptide comprises an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87) and variants thereof each having up to three amino acid substitutions, additions or deletions.

In one embodiment, the TCR binds to an MHC I and/or MHC II peptide complex.

In one embodiment, the TCR is restricted to a human leukocyte antigen (HLA) allele. In one embodiment, the TCR is restricted to a HLA-A, HLA-B or a HLA-C allele. In one embodiment, the TCR is restricted to HLA-A*02:01, HLA-B*38:01, HLA-C*03:03 or HLA-C*07:02.

In one embodiment, the TCR is restricted to HLA-A*02:01. In one embodiment, the TCR is restricted to HLA-B*38:01. In one embodiment, the TCR is restricted to HLA-C*03:03. In one embodiment, the TCR is restricted to HLA-C*07:02.

In one embodiment, a TCR of the present invention is restricted to a HLA-C allele. In one embodiment, a TCR of the present invention is restricted to a HLA-C allele selected from the group consisting of HLA-C*07:01, HLA-C*03:04, HLA-C*04:01, HLA-C*05:01, HLA-C*06:02 and HLA-C*07:02.

In one embodiment, the TCR comprises one or more mutations at the α chain/3 chain interface, such that when the α chain and the β chain are expressed in a T-cell, the frequency of mispairing between said chains and endogenous TCR α and β chains is reduced.

In one embodiment, the TCR comprises one or more mutations at the α chain/3 chain interface, such that when the α chain and the β chain are expressed in a T-cell, the level of expression of the TCR α and β chains is increased.

In one embodiment, the one or more mutations introduce a cysteine residue into the constant region domain of each of the α chain and the β chain, wherein the cysteine residues are capable of forming a disulphide bond between the α chain and the β chain.

In one embodiment, the one or more mutations are at amino acid positions selected from those disclosed in Table 1 of Boulter, J. M et al. (2003) Protein Engineering 16: 707-711.

In one embodiment, the TCR comprises one or more mutations to remove one or more N-glycosylation sites (see, for example, Kuball, J et al. (2009) J Exp Med 206: 463-75).

Preferably, the N-glycosylation sites are in the TCR constant domains. In one embodiment, the mutation is a substitution of the amino acid N in an N-X-S/T motif with the amino acid Q.

For example, the substitution may occur at one or more of the positions: TCR alpha constant gene position 36, 90 or 109; and/or TCR beta constant gene position 85.6. Preferably, the substitution is at position 36 of the TCR alpha constant gene.

In one embodiment, the TCR comprises a murinised constant region.

In one embodiment, the TCR is a soluble TCR.

In another aspect, the invention provides an isolated polynucleotide encoding the α chain of a T-cell receptor (TCR) according to the invention, and/or the β chain of a TCR according to the invention.

In one embodiment, the polynucleotide encodes the α chain linked to the β chain.

In one embodiment, the isolated polynucleotide further encodes one or more short interfering RNA (siRNA) or other agents capable of reducing or preventing expression of one or more endogenous TCR genes.

In another aspect, the invention provides a vector comprising a polynucleotide according to the invention. In one embodiment, the vector comprises a polynucleotide, which encodes one or more CD3 chains, CD8, a suicide gene and/or a selectable marker.

In another aspect, the invention provides a cell comprising a TCR of the invention, a polynucleotide of the invention or a vector of the invention.

In one embodiment, the cell further comprises a vector which encodes one or more CD3 chains, CD8, a suicide gene and/or a selectable marker.

In one embodiment, the cell is a T-cell, a lymphocyte, or a stem cell, optionally wherein the T-cell, the lymphocyte, or the stem cell is selected from the group consisting of CD4 cells, CD8 cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CIK) cells, hematopoietic stem cells and pluripotent stem cells.

In one embodiment, the cell is a T-cell which has been isolated from a subject.

In one embodiment, an endogenous gene encoding a TCR α chain and/or an endogenous gene encoding a TCR β chain in the cell is disrupted, preferably such that the endogenous gene encoding a TCR α chain and/or the endogenous gene encoding a TCR β chain is not expressed. In one embodiment, the endogenous gene encoding a TCR α chain and/or the endogenous gene encoding a TCR β chain is disrupted by insertion of an expression cassette comprising a polynucleotide sequence encoding the TCR of the invention. In one embodiment, one or more endogenous genes encoding an MHC is disrupted, preferably wherein the cell is a non-alloreactive universal T-cell. In one embodiment, an endogenous gene involved in persistence, expansion, activity, resistance to exhaustion/senescence/inhibitory signals, homing capacity, or other T-cell functions is disrupted, preferably wherein the endogenous gene involved in persistence, expansion, activity, resistance to exhaustion/senescence/inhibitory signals, homing capacity, or other T-cell functions is selected from the group consisting of PD1, TIM3, LAG3, 2B4, KLRG1, TGFbR, CD160, TIGIT, CTLA4 and CD39. In one embodiment, the endogenous gene involved in persistence, expansion, activity, resistance to exhaustion/senescence/inhibitory signals, homing capacity, or other T-cell functions is disrupted by integration of an expression cassette, wherein the expression cassette comprises a polynucleotide sequence encoding a TCR of the invention.

In another aspect, the invention provides a method of preparing a cell, which comprises the step of introducing the vector of the invention into a cell in vitro, ex vivo or in vivo, for example by transfection or transduction.

In another aspect, the invention provides a method of preparing a cell, which comprises the step of transducing a cell in vitro, ex vivo or in vivo with one or more vectors of the invention.

In one embodiment, the cell to be transduced with the one or more vectors is selected from the group consisting of T-cells, lymphocytes or stem cells, such as hematopoietic stem cells or induced pluripotent stem cells (iPS), optionally the T-cell, the lymphocyte or the stem cell may be selected from the group consisting of CD4 cells, CD8 cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, double negative T-cells, naive T-cells, memory stem T-cells, central memory T-cells, effector memory T-cells, effector T cells, cytokine-induced killer (CIK) cells, hematopoeitic stem cells and pluripotent stem cells.

In one embodiment, the method comprises the step of T-cell editing, which comprises disrupting an endogenous gene encoding a TCR α chain and/or an endogenous gene encoding a TCR β chain with an artificial nuclease, preferably wherein the artificial nuclease is selected from the group consisting of zinc finger nucleases (ZFNs), transcription activator-like effector nucleases (TALENs) and CRISPR/Cas systems.

In one embodiment, the method comprises the step of T-cell editing, which comprises disrupting an endogenous gene encoding a TCR α chain and/or an endogenous gene encoding a TCR β chain with an artificial nuclease, preferably wherein the artificial nuclease is selected from the group consisting of zinc finger nucleases (ZFNs), transcription activator-like effector nucleases (TALENs) and CRISPR/Cas systems.

In one embodiment, the method comprises the step of targeted integration of an expression cassette into the endogenous gene encoding the TCR α chain and/or the endogenous gene encoding the TCR β chain disrupted by the artificial nuclease, wherein the expression cassette comprises a polynucleotide sequence encoding the TCR of the invention or a polynucleotide sequence of the invention.

In one embodiment, the method comprises the step of disrupting one or more endogenous genes encoding an MHC, preferably wherein the cell prepared by the method is a non-alloreactive universal T-cell.

In one embodiment, the method comprises the step of disrupting one or more endogenous MHC genes, preferably wherein the cell prepared by the method is a non-alloreactive universal T-cell.

In one embodiment, the method comprises the step of disrupting one or more endogenous genes to modify the persistence, expansion, activity, resistance to exhaustion/senescence/inhibitory signals, homing capacity, or other T-cell functions, preferably wherein the method comprises the step of targeted integration of an expression cassette into an endogenous gene involved in persistence, expansion, activity, resistance to exhaustion/senescence/inhibitory signals, homing capacity, or other T-cell functions disrupted by an artificial nuclease, wherein the expression cassette comprises a polynucleotide sequence encoding the TCR of the invention, preferably wherein the endogenous gene is selected from the group consisting of PD1, TIM3, LAG3, 2B4, KLRG1, TGFbR, CD160, TIGIT, CTLA4 and CD39.

In another aspect, the invention provides the cell of the invention or a cell prepared by the method of the invention for use in adoptive cell transfer, preferably adoptive T-cell transfer, optionally wherein the adoptive T-cell transfer is allogenic adoptive T-cell transfer, autologous adoptive T-cell transfer, or universal non-alloreactive adoptive T-cell transfer.

In another aspect, the invention provides a chimeric molecule comprising the TCR of the invention, or a portion thereof, conjugated to a non-cellular substrate, a toxin and/or an antibody. In one embodiment, the non-cellular substrate is selected from the group consisting of nanoparticles, exosomes and other non-cellular substrates.

In another aspect, the invention provides the TCR of the invention, the isolated polynucleotide of the invention, the vector of the invention, the cell of the invention, a cell prepared by the method of the invention, or the chimeric molecule of the invention for use in therapy.

In another aspect, the invention provides the TCR of the invention, the isolated polynucleotide of the invention, the vector of the invention, the cell of the invention, a cell prepared by the method of the invention, or the chimeric molecule of the invention for use in treating and/or preventing a disease associated with expression of WT1.

In another aspect, the invention provides a T-cell genetically engineered (e.g. genetically edited) to modify the persistence, expansion, activity, resistance to exaustion/senescence/inhibitory signals, homing capacity or other T cell functions, wherein the T-cell expresses a TCR α chain of the invention and/or a TCR β chain of the invention.

In another aspect, the invention provides a T cell genetically engineered (e.g. genetically edited) by a protocol which comprises the step of targeted integration of an expression cassette into an endogenous gene involved in persistence, expansion, activity, resistance to exhaustion/senescence/inhibitory signals, homing capacity or other T-cell functions disrupted by an artificial nuclease, wherein the expression cassette comprises a polynucleotide sequence encoding TCR α chain of the invention and/or a TCR β chain of the invention.

In another aspect, the invention provides a method for treating and/or preventing a disease associated with expression of WT1, which comprises the step of administering the TCR of the invention, the isolated polynucleotide of the invention, the vector of the invention, the cell of the invention, a cell prepared by the method of the invention, or the chimeric molecule of the invention to a subject in need thereof.

In one embodiment, the disease associated with expression of WT1 is a proliferative disorder. Preferably, the proliferative disorder is a hematological malignancy or a solid tumor. Preferably, the hematological malignancy is selected from the group consisting of acute myeloid leukemia (AML), chronic myeloid leukemia (CML), lymphoblastic leukemia, myelodisplastic syndromes, lymphoma, multiple myeloma, non Hodgkin lymphoma, and Hodgkin lymphoma. Preferably, the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma, liver cancer, melanoma, prostate cancer, renal cancer, soft tissue sarcoma, urothelial cancer, biliary cancer, glioblastoma, cervical cancer, mesothelioma and colorectal cancer.

In a preferred embodiment, the disease associated with expression of WT1 is acute myeloid leukemia (AML).

In another preferred embodiment, the disease associated with expression of WT1 is chronic myeloid leukemia (CML).

In another aspect, the invention provides an isolated immunogenic WT1 peptide comprising an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87) and variants thereof each having up to three amino acid substitutions, additions or deletions.

### DESCRIPTION OF THE DRAWINGS

FIG. 1

Evaluation over time of WT1-specific T cell expansion in 4 healthy donors. Peripheral blood mononuclear cells of four HDs were repetitively stimulated with the WT1 pool-137 (HD12, a) or with the WT1 HLA-A*02:01 pool (HD13-HD15, b-d). Enrichment of antigen-responding T cells was assessed by measuring IFNγ secretion and CD107a production in a 6 hour co-culture with autologous antigen-presenting cells (APCs) pulsed with a pool derived from WT1 protein. In each test, T cells unstimulated, T cells in co-culture with APCs loaded with an unrelated peptide pool and T cells stimulated with Phorbol-12-myristate-13-acetate (PMA) and lonomycin (not shown) were included as controls. Dot plots indicate the results of the intracellular staining for IFNγ production and CD107a exposure on cell surface either at a single time point (a, b, d) or over the culture timeframe (c). HD, healthy donor; WT1, Wilms Tumor 1; PMA, Phorbol 12-myristate 13-acetate; IFNγ, interferon-γ; S, stimulation.

FIG. 2

Identification of the WT1 subpools and peptides recognised by expanded T lymphocytes isolated from each HD. The assessment of the peptides inducing an immune response in T cells isolated from HDs was performed with a co-culture of T cells with APCs loaded with 24 subpools (SPs; HD12) or 11 peptides (HD13 and HD14). Additionally, negative (T-cells unstimulated and T-cells co-cultured with APCs loaded with an unrelated peptide pool or unrelated peptide) and positive (T-cells cultured in the presence of PMA and lonomycin) controls were included in the experimental setting (not shown). Evaluation of IFNγ secretion and CD107a expression was performed by cytofluorimetric analysis. Representative dot plots relative to the co-culture of the T-cells with APCs loaded with the subpools and indicating the expression of IFNγ and CD107a are reported. Dominant responses were observed for subpools 7 and 21 in HD12 (a, b), peptide 13 for HD13 and HD14 (c-e). HD, healthy donor; SP, subpools; WT1, Wilms Tumor 1; APC, antigen-presenting cells; PMA, Phorbol 12-myristate 13-acetate; IFNγ, interferon-γ.

FIG. 3

Epitope specificity of the WT1-reactive T cells generated by sensitisation with the pooled peptides. In order to validate the WT1 immunogenic peptides, T-cells expanded from HD12 were co-cultured for 6 hours in the presence of APCs loaded with the peptide identified after deconvolution of the mapping grid (a) and with at least one unrelated peptide as a negative control. Additionally, negative (T cells unstimulated) and positive (T cells cultured in the presence of PMA and lonomycin) controls were included in the experimental setting (not shown). Dot plots show for each HD the results of the intracellular staining for IFNγ and surface CD107a. Enrichment of CD107a and IFNγ positive cells was observed for T-cells co-cultured with peptide 103 and not for the unrelated peptide (b). WT1, Wilms Tumor 1; APC, antigen-presenting cells; PMA, 2; Phorbol 12-myristate 13-acetate; IFNγ, interferon-γ.

FIG. 4

In silico prediction of HLA-peptide binding for different HDs. HLA typing results for HD12-HD14 (a). Results of the in silico prediction performed with the NetMHC4.0 pan algorithm are reported (b, HD12; c, HD13; d, HD14; e, HD15). In grey are highlighted the peptides identified as strong binders by the algorithm. HD, healthy donor; HLA, human leukocyte antigen.

FIG. 5

HLA-restriction assessment of the WT1 immunogenic epitopes identified. To determine the HLA restriction element for HD12 we co-cultured T cells with different antigen presenting EBV-BLCL cell lines, each one harboring a specific HLA allele of interest shared with HD12 and pulsed with peptide 103 or with an unrelated peptide as a control (a). For HD13 and HD14, WT1-specific T cells were co-cultured with T2 cells harbouring the HLA-A*02:01 restriction element and pulsed with peptide 13 or with an unrelated peptide as a control (b and c, respectively). As readout we determined the expression of the CD107a marker and the secretion of IFNγ. HD, healthy donor; WT1, Wilms Tumor 1; IFNγ, interferon-γ; HLA, human leukocyte antigen; EBV, Epstein-Barr virus; BLCL, B lymphoblastoid cell line.

FIG. 6

Immunogenic peptides are naturally processed. T cells isolated from HD13 (a) and HD14 (b) were co-cultured with primary AML blasts from 3 different patients (indicated as pAML #15, pAML #16.1; pAML #16.2) expressing WT1 at high levels and harboring the HLA-A*02:01 restriction element. As a control, we included co-cultures of the blasts with unrelated T cells. We evaluated the percentage of Caspase 3 (Cas3) expression in target (T) living cells upon 6 hour co-culture with effector (E) T cells at different E:T ratios. Cas3 values obtained in the control conditions were subtracted from the Cas3 values obtained from the co-cultures of primary blasts with HD13 and HD14 T cells. pAML, primary blasts of acute myeloid leukemia patients; HD, healthy donor; HLA, human leukocyte antigen; WT1, Wilms Tumor 1.

FIG. 7

WT1-specific TCR Vs profile characterisation. WT1-specific T cells isolated from HDs were stained in order to quantitatively determine the TCR β-chain variable region (Vβ) repertoire by FACS analysis. Results indicated the expression of a highly dominant Vβ gene in HD12 and HD14, whereas for HD13 a clear enrichment of a defined Vβ was not detected. For HD15 it was not possible to perform the Vβ immunoprofiling analysis due to a reduced cell fitness. TRBV, T cell receptor variable beta chain; HD, healthy donor; FACS, fluorescence activated cell sorter.

FIG. 8

Clonal tracking of WT1-specific TCRs. TCRαβ sequencing was performed on HD RNA at different time points over the culture timeframe. Sequencing results indicated the presence of predominant clonotypes for HD12 (a), HD13 (b), HD14 (c) and HD15 (d). Bar charts depict the ten most predominant CDR3 amino acid sequences identified at each time point (e.g. S4 corresponds to the sequencing results obtained following the 4th round of stimulation). For each bar, starting from the x-axis, the bottom segment represents the most predominant CDR3 sequence. The next nine most predominant sequences are stacked above the bottom segment and are ordered by decreasing frequency going upwards. The remaining sequences are grouped together in the top segment. CDR3, complementarity determining region 3; S, stimulation; HD, healthy donor; S, stimulation; HLA, human leukocyte antigen; P, peptide; RNA, ribonucleic acid.

FIG. 9

Functional avidity of TCR derived from HD12. T cells from 3 different healthy donors were transduced with a lentiviral vector encoding TCR isolated from HD12 upon knock-out of the endogenous a and β chains. a) Expression of the HD12-derived TCR was assessed by Vβ staining before and after Vβ-enrichment. b) Functional avidity of HD12-derived TCR. We co-cultured effector cells with EBV cell line pulsed either with the NYESO-1 peptide as control or with decreasing concentrations of the peptide 103 (40 μg-0.4 μg, as indicated on the x-axis). Upon 6 hours of co-culture, results showed the ability of HD12-edited T cells to recognise target cells in presence of at least 0.4 μg of the cognate peptide. No recognition of the unrelated peptide was measured. As a readout we evaluated by flow cytometry the expression of the CD107a on the CD8 T lymphocytes. TCR, T cell receptor; HD, healthy donor; PBMC, peripheral blood mononuclear cell; NYESO-1, New York esophageal squamous cell carcinoma 1.

FIG. 10

Functional validation of HD13-derived TCR. T cells from one healthy donor were transduced with a lentiviral vector encoding HD13-derived TCR. a) Recognition of WT1 pool by TCR isolated from HD13. We co-cultured effector cells with T2 cell line pulsed either with WT1 pool or an unrelated pool as control. Additionally, negative (T cells unstimulated) and positive (T cells cultured in the presence of PMA and lonomycin) controls were included in the experimental setting. Upon 6 hours of co-culture, T cells transduced with HD13-derived TCR to specifically recognise target cells pulsed with WT1 pool as assessed by measuring IFNγ secretion on CD8 T cells. b) T cells were tested in co-culture with T2 cells pulsed with WT1-derived SPs 1 and 14, both containing peptide 13, or SP 6 as a negative control. Results showed the ability of effector cells to specifically recognise SP1 and 14 as evaluated by measuring IFNγ secretion and the expression of CD107a on CD8 T cells. HD, healthy donor; TCR, T cell receptor; WT1, Wilms tumor 1; SP, subpool; PMA, 2; Phorbol 12-myristate 13-acetate.

FIG. 11

Functional validation of TCR isolated from HD14. T cells isolated from one healthy donor were transduced with a lentiviral vector encoding HD14-derived TCRs (TRAV12-2*01 WT and TRAV12-2*02 WT). a) Transduction efficiency of HD14-transfer T cells expressed as Vβ expression on CD4 and CD8 T lymphocytes. b) Recognition of WT1 pool by HD14-derived TCRs. We co-cultured effector cells with T2 cell line pulsed either with WT1 pool or an unrelated pool as a control. Upon 6 hours of co-culture, results showed the ability of HD14-transfer T cells to specifically recognise target cells pulsed with WT1 pool as measured by evaluating IFNγ secretion on CD8 T cells. c) HD14-derived T cells recognise specific SPs containing peptide 13. T cells were tested in co-culture with T2 cells pulsed with WT1-derived SPs 1 and 14, both containing peptide 13, or SP 6 as a negative control. Results showed the ability of effector cells to specifically recognise SP1 and 14 as evaluated by assessing the expression of CD107a and IFNγ secretion on CD8 T cells. HD, healthy donor; TCR, T cell receptor; WT1, Wilms tumor 1; SP, subpool.

FIG. 12

TCRs derived from HD14 recognise primary AML blasts. TCR-edited T cells from one healthy donor were transduced with a lentiviral vector encoding HD14-derived TCRs (TRAV 12-2*02 WT and TRAV 12-2*02 mut). a) Transduction efficiency of HD14 TCRs was assessed by Vβ expression on CD4 and CD8 T cells. b) Edited T cells transduced with HD14 TCR TRAV 12-2*02 WT, TRAV12-2*02 mut or an unrelated TCR were co-cultured with patient-derived primary AML blasts expressing high levels of WT1 and the HLA-A02*01 restriction element. To assess viability of blasts we included conditions of target cells without T lymphocytes. We evaluated the percentage of Caspase 3 (Cas3) expression in target (T) living cells upon 6 hour co-culture with effector (E) T cells at different E:T ratios (as indicated in the figure). Cas3 values obtained in the control conditions (either T cells transduced with unrelated TCR or blasts alone) were subtracted from the Cas3 values obtained from the co-cultures of primary blasts with T cells harbouring HD14-derived TCRs. pAML, primary blasts of acute myeloid leukemia patients; HD, healthy donor; HLA, human leukocyte antigen; WT1, Wilms Tumor 1.

FIG. 13

Identification of WT1-specific T cells by dextramer staining. Dot plots indicate the results of Dextramer staining at different time points upon T cell sorting (using an APC-labelled dextramer specific for the WT1 VLDFAPPGA peptide) and stimulation (Patient 1, a) or at a single time point (b, Patients 2 and 3). WT1, Wilms Tumor 1.

FIG. 14

Graphs showing results of TCR sequencing of enriched WT1-specific T-cells. T-cells isolated from each patient and sorted based on the positivity for WT1 dextramer staining were characterised by TCR αβ sequencing. Sequencing results indicated the presence of predominant clonotypes for Patient 1 (a, b), Patient 2 (c) and Patient 3 (d). Bar charts depict the ten most predominant CDR3 amino acid sequences identified for each patient and for each TCR chain. For each bar, starting from the x-axis, the bottom segment represents the most predominant CDR sequence. The next nine most predominant sequences are stacked above the bottom segment and are ordered by decreasing frequency going upwards. The remaining sequences are grouped together in the top segment. WT1, Wilms Tumor 1; CDR3, complementarity determining region 3.

### DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

T-Cell Receptor

During antigen processing, antigens are degraded inside cells and then carried to the cell surface by major histocompatibility complex (MHC) molecules. T-cells are able to recognise this peptide:MHC complex at the surface of the antigen presenting cell. There are two different classes of MHC molecules: MHC I and MHC II, each class delivers peptides from different cellular compartments to the cell surface.

A T cell receptor (TCR) is a molecule which can be found on the surface of T-cells that is responsible for recognizing antigens bound to MHC molecules. The naturally-occurring TCR heterodimer consists of an alpha (a) and beta (p) chain in around 95% of T-cells, whereas around 5% of T-cells have TCRs consisting of gamma (γ) and delta (6) chains.

Engagement of a TCR with antigen and MHC results in activation of the T lymphocyte on which the TCR is expressed through a series of biochemical events mediated by associated enzymes, co-receptors, and specialized accessory molecules.

Each chain of a natural TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end.

The variable domain of both the TCR α chain and β chain have three hypervariable or complementarity determining regions (CDRs). A TCR α chain or β chain, for example, comprises a CDR1, a CDR2, and a CDR3 in amino to carboxy terminal order. In general, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule.

A constant domain of a TCR may consist of short connecting sequences in which a cysteine residue forms a disulfide bond, making a link between the two chains.

An α chain of a TCR of the present invention may have a constant domain encoded by a TRAC gene. An example amino acid sequence of an α chain constant domain encoded by a TRAC gene is a shown below:

(SEQ ID NO: 76)
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS

A TCR of the invention may comprise an α chain comprising the amino acid sequence of SEQ ID NO: 76 or a variant thereof having at least 75%, at least 80%, at least 85%, at least 90%, at least 95%, at least 96%, at least 97%, at least 98%, at least 99% sequence identity thereto, preferably at least 75% sequence identity thereto.

A β chain of a TCR of the present invention may have a constant domain encoded by a TRBC1 or a TRBC2 gene. An example amino acid sequence of a β chain constant domain encoded by a TRBC1 gene is a shown below:

(SEQ ID NO: 77)
DLNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSVSYQQGVLSATILYEI
LLGKATLYAVLVSALVLMAMVKRKDF

An example amino acid sequence of a 3 chain constant domain encoded by a TRBC2 gene is a shown below:

(SEQ ID NO: 78)
DLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG

A TCR of the invention may comprise a β chain comprising the amino acid sequence of SEQ ID NO: 77, SEQ ID NO: 78, or variants of SEQ ID NOs: 77 and 78 having at least 75%, at least 80%, at least 85%, at least 90%, at least 95%, at least 96%, at least 97%, at least 98%, at least 99% sequence identity thereto, preferably at least 75% sequence identity thereto.

The TCR of the invention may have one or more additional cysteine residues in each of the α and β chains such that the TCR may comprise two or more disulphide bonds in the constant domains.

Mutations of TCR constant domains disclosed herein may be described based on a numbering convention in which the first amino acid of each of SEQ ID NOs: 76-78 is assigned to be position 2.

The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3- and ζ-chains, together with the TCR, form what is known as the T cell receptor complex.

The signal from the T cell complex is enhanced by simultaneous binding of the MHC molecules by a specific co-receptor. For helper T-cells, this co-receptor is CD4 (specific for class II MHC); whereas for cytotoxic T-cells, this co-receptor is CD8 (specific for class I MHC). The co-receptor allows prolonged engagement between the antigen presenting cell and the T cell and recruits essential molecules (e.g., LCK) inside the cell involved in the signalling of the activated T lymphocyte.

Accordingly, as used herein the term “T-cell receptor” (TCR) refers to molecule capable of recognising a peptide when presented by an MHC molecule. The molecule may be a heterodimer of two chains α and β (or optionally γ and δ) or it may be a single chain TCR construct. A TCR of the invention may be a soluble TCR, e.g. omitting or altering one or more constant domains. A TCR of the invention may comprise a constant domain.

The invention also provides an α chain or a β chain from such a T cell receptor.

The TCR of the invention may be a hybrid TCR comprising sequences derived from more than one species. For example, it has been found that murine TCRs are more efficiently expressed in human T-cells than human TCRs. The TCR may therefore comprise a human variable region and murine sequences within a constant region.

A disadvantage of this approach is that the murine constant sequences may trigger an immune response, leading to rejection of the transferred T-cells. However, the conditioning regimens used to prepare patients for adoptive T-cell therapy may result in sufficient immunosuppression to allow the engraftment of T-cells expressing murine sequences.

In one embodiment, the TCR comprises one or more mutations to remove one or more N-glycosylation sites. Preferably, the N-glycosylation sites are in the TCR constant domains. Deletion of N-glycosylation sites in TCR constant domains is described in Kuball, J et al. (2009) J Exp Med 206: 463-75. In one embodiment, the one or more mutations are substitutions of the amino acid N in an N-X-S/T motif with the amino acid Q. For example, the substitution may at one or more of the positions: TCR alpha constant gene position 36, 90 or 109; and/or TCR beta constant gene position 85.6. Preferably, the substitution is at position 36 of the TCR alpha constant gene.

Complementarity Determining (CDR) Regions

The portion of the TCR that establishes the majority of the contacts with the antigenic peptide bound to the major histocompatibility complex (MHC) is the complementarity determining region 3 (CDR3), which is unique for each T cell clone. The CDR3 region is generated upon somatic rearrangement events occurring in the thymus and involving non-contiguous genes belonging to the variable (V), diversity (D, for p and 5 chains) and joining (J) genes.

Furthermore, random nucleotides inserted/deleted at the rearranging loci of each TCR chain gene greatly increase diversity of the highly variable CDR3 sequence. Thus, the frequency of a specific CDR3 sequence in a biological sample indicates the abundance of a specific T cell population. The great diversity of the TCR repertoire in healthy human beings provides a wide range protection towards a variety of foreign antigens presented by MHC molecules on the surface of antigen presenting cells. In this regard, it is of note that theoretically up to 10¹⁵ different TCRs can be generated in the thymus.

T-cell receptor diversity is focused on CDR3 and this region is primarily responsible for antigen recognition.

The sequences of the CDR3 regions of the TCR of the invention may be selected from those set out in Table 1 below. A TCR may comprise CDRs that comprise or consist of a CDR3α and a CDR3β pair described below.

The CDRs may, for example, comprise one, two, or three substitutions, additions or deletions from the given sequence, provided that the TCR retains the capacity to bind a WT1 peptide when presented by an MHC molecule.

As used herein, the term “protein” includes single-chain polypeptide molecules as well as multiple-polypeptide complexes where individual constituent polypeptides are linked by covalent or non-covalent means. As used herein, the term “polypeptide” refers to a polymer in which the monomers are amino acids and are joined together through peptide or disulphide bonds.

Variants, Derivatives, Analogues, Homologues and Fragments

In addition to the specific proteins and polynucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

A variant amino acid sequence of the invention referred to as having up to three amino acid substitutions, additions or deletions may have, for example, one, two or three amino acid substitutions, additions or deletions.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

A substitution may involve replacement of an amino acid for a similar amino acid (a conservative substitution). A similar amino acid is one which has a side chain moiety with related properties as grouped together, for example as shown below:
(i) basic side chains: lysine (K), arginine (R), histidine (H);
(ii) acidic side chains: aspartic acid (D) and glutamic acid (E);
(iii) uncharged polar side chains: asparagine (N), glutamine (Q), serine (S), threonine (T) and tyrosine (Y); or
(iv) non-polar side chains: glycine (G), alanine (A), valine (V), leucine (L), isoleucine (I), proline (P), phenylalanine (F), methionine (M), tryptophan (W) and cysteine (C).

Any amino acid changes should maintain the capacity of the TCR to bind WT1 peptide presented by MHC molecules.

Variant sequences may comprise amino acid substitutions, additions, deletions and/or insertions. The variation may be concentrated in one or more regions, such as the constant regions, the linker, or the framework regions of the α or β chains, or they may be spread throughout the TCR molecule.

Conservative substitutions, additions or deletions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The invention also encompasses homologous substitution (substitution and replacement are both used herein to mean the interchange of an existing amino acid residue, with an alternative residue), e.g. like-for-like substitution such as basic for basic, acidic for acidic, polar for polar etc. Non-homologous substitution may also occur e.g. from one class of residue to another or alternatively involving the inclusion of unnatural amino acids, such as ornithine.

The term “variant” as used herein may mean an entity having a certain homology with the wild type amino acid sequence or the wild type nucleotide sequence. The term “homology” can be equated with “identity”.

A variant sequence may include an amino acid sequence which may be at least 50%, 55%, 65%, 75%, 85% or 90% identical, preferably at least 95%, at least 97%, or at least 99% identical to the subject sequence. Typically, the variants will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A variant sequence may include a nucleotide sequence which may be at least 40%, 45%, 50%, 55%, 65%, 75%, 85% or 90% identical, preferably at least 95%, at least 97%, or at least 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Identity comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Major Histocompatability Complex (MHC) Molecules

Typically, TCRs bind to peptides as part of peptide:MHC complex.

The MHC molecule may be an MHC class I or II molecule. The complex may be on the surface of an antigen presenting cell, such as a dendritic cell or a B cell, or any other cell, including cancer cells, or it may be immobilised by, for example, coating on to a bead or plate.

The human leukocyte antigen system (HLA) is the name of the gene complex which encodes major histocompatibility complex (MHC) in humans and includes HLA class I antigens (A, B & C) and HLA class II antigens (DP, DQ, & DR). HLA alleles A, B and C present peptides derived mainly from intracellular proteins, e.g. proteins expressed within the cell. This is of particular relevance since WT1 is an intracellular protein.

During T-cell development in vivo, T-cells undergo a positive selection step to ensure recognition of self MHCs followed by a negative step to remove T-cells that bind too strongly to MHC which present self-antigens. As a consequence, certain T-cells and the TCRs they express will only recognise peptides presented by certain types of MHC molecules—i.e. those encoded by particular HLA alleles. This is known as HLA restriction.

One HLA allele of interest is HLA-A*0201, which is expressed in the vast majority (>50%) of the Caucasian population. Accordingly, TCRs which bind WT1 peptides presented by MHC encoded by HLA-A*0201 (i.e. are HLA-A*0201 restricted) are advantageous since an immunotherapy making use of such TCRs will be suitable for treating a large proportion of the Caucasian population.

Other HLA alleles of interest are HLA-B*38:01, HLA-C*03:03 and HLA-C*07:02.

Further alleles of interest are HLA-DPA1, HLA-DPB1, HLA-DQA1, HLA-DQB1, HLA-DRA and HLA-DRB1. These are the six main MHC class II genes in humans.

In one embodiment, the TCR of the invention is HLA-A*0201-, HLA-A*0101-, HLA-A*2402-, HLA-A*0301-, HLA-B*3501- or HLA-B*0702-restricted.

A TCR of the present invention may be HLA-A*02:01-restricted.

In one aspect, where a TCR of the invention comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions, the TCR is HLA-A*02:01 restricted.

In one aspect, where a TCR of the invention comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions, the TCR is HLA-A*02:01 restricted.

In one aspect, where a TCR of the invention comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions, the TCR is HLA-A*02:01 restricted.

In one aspect, where a TCR of the invention comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions, the TCR is HLA-A*02:01 restricted.

In one aspect, where a TCR of the invention comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions, the TCR is HLA-A*02:01 restricted.

In one aspect, where a TCR of the invention comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions, the TCR is HLA-A*02:01 restricted.

In one aspect, where a TCR of the invention comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions, the TCR is HLA-A*02:01 restricted.

In one aspect, where a TCR of the invention comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions, the TCR is HLA-A*02:01 restricted.

In one aspect, where a TCR of the invention comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions, the TCR is HLA-A*02:01 restricted.

In one aspect, where a TCR of the invention comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions, the TCR is HLA-A*02:01 restricted.

In one aspect, where a TCR of the invention comprises a CDR3α comprising the amino acid sequence of CALPDKVIF (SEQ ID NO: 148) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions, the TCR is HLA-A*02:01 restricted.

In one aspect, where a TCR of the invention comprises a CDR3α comprising the amino acid sequence of CAGLYATNKLIF (SEQ ID NO: 153) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions, the TCR is HLA-A*02:01 restricted.

In one aspect, where a TCR of the invention comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions, the TCR is HLA-A*02:01 restricted.

In one aspect, where a TCR of the invention comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions, the TCR is HLA-A*02:01 restricted.

In one aspect, where a TCR of the invention comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions, the TCR is HLA-A*02:01 restricted.

In one aspect, where a TCR of the invention comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions, the TCR is HLA-A*02:01 restricted.

In one aspect, where a TCR of the invention comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions, the TCR is HLA-A*02:01 restricted.

In one embodiment, a TCR of the present invention that is HLA-A*02:01 restricted binds to a WT1 peptide comprising amino acid sequence LLAAILDFLLLQDPA (SEQ ID NO: 82) or a variant thereof having up to three amino acid substitutions, additions or deletions.

In one aspect, the invention provides a TCR which binds a Wilms tumour 1 protein (WT1) peptide when presented by a major histocompatibility complex (MHC), wherein the TCR comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions, wherein the TCR is HLA-A*0201 restricted, and wherein the WT1 peptide comprises the amino acid sequence of LLAAILDFLLLQDPA (SEQ ID NO: 82) or a variant thereof having up to three amino acid substitutions, additions or deletions.

In one aspect, the invention provides a TCR which binds a Wilms tumour 1 protein (WT1) peptide when presented by a major histocompatibility complex (MHC), wherein the TCR comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions, wherein the TCR is HLA-A*0201 restricted, and wherein the WT1 peptide comprises the amino acid sequence of LLAAILDFLLLQDPA (SEQ ID NO: 82) or a variant thereof having up to three amino acid substitutions, additions or deletions.

Another widely expressed HLA allele of interest is HLA-B*38:01. A TCR of the invention may be HLA-B*38:01 restricted.

In one aspect, where a TCR of the invention comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions, the TCR is HLA-B*38:01 restricted.

In one aspect, the invention provides a TCR which binds a Wilms tumour 1 protein (WT1) peptide when presented by a major histocompatibility complex (MHC), wherein the TCR comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions, wherein the TCR is HLA-B*38:01 restricted, and wherein the WT1 peptide comprises the amino acid sequence of GAQYRIHTHGVFRGI (SEQ ID NO: 181) or a variant thereof having up to three amino acid substitutions, additions or deletions.

Another widely expressed HLA allele of interest is HLA-C*07:02. A TCR of the invention may be HLA-C*07:02 restricted.

In one aspect, where a TCR of the invention comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions, the TCR is HLA-C*07:02 restricted.

In one aspect, the invention provides a TCR which binds a Wilms tumour 1 protein (WT1) peptide when presented by a major histocompatibility complex (MHC), wherein the TCR comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions, wherein the TCR is HLA-C*07:02 restricted, and wherein the WT1 peptide comprises the amino acid sequence of GAQYRIHTHGVFRGI (SEQ ID NO: 181) or a variant thereof having up to three amino acid substitutions, additions or deletions.

Another widely expressed HLA allele of interest is HLA-C*03:03. A TCR of the invention may be HLA-C*03:03 restricted.

In one aspect, where a TCR of the invention comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions, the TCR is HLA-C*03:03 restricted.

In one aspect, the invention provides a TCR which binds a Wilms tumour 1 protein (WT1) peptide when presented by a major histocompatibility complex (MHC), wherein the TCR comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions, wherein the TCR is HLA-C*03:03 restricted, and wherein the WT1 peptide comprises the amino acid sequence of LLAAILDFLLLQDPA (SEQ ID NO: 82) or a variant thereof having up to three amino acid substitutions, additions or deletions.

In one embodiment, where a TCR of the invention binds to a WT1 peptide comprising an amino acid sequence of LLAAILDFLLLQDPA (SEQ ID NO: 82) or a variant thereof having up to three amino acid substitutions, additions or deletions, the TCR is HLA-A*02:01 restricted.

In one embodiment, where a TCR of the invention binds to a WT1 peptide comprising an amino acid sequence of GAQYRIHTHGVFRGI (SEQ ID NO: 181) or a variant thereof having up to three amino acid substitutions, additions or deletions, the TCR is HLA-B*38:01 restricted.

In one embodiment, where a TCR of the invention binds to a WT1 peptide comprising an amino acid sequence of GAQYRIHTHGVFRGI (SEQ ID NO: 181) or a variant thereof having up to three amino acid substitutions, additions or deletions, the TCR is HLA-C*07:02 restricted.

In one embodiment, where a TCR of the invention binds to a WT1 peptide comprising an amino acid sequence of LLAAILDFLLLQDPA (SEQ ID NO: 82) or a variant thereof having up to three amino acid substitutions, additions or deletions, the TCR is HLA-C*03:03 restricted.

Wilms Tumor 1 (WT1) Protein

Wilms tumor 1 (WT1) is an intracellular protein encoding a zinc finger transcription factor that 20 plays an important role in cell growth and differentiation (Yang, L. et al. Leukemia 21, 868-876 (2007)). It is widely expressed on a variety of hematological and solid tumors, while showing limited expression on other tissues (gonads, uterus, kidney, mesothelium, progenitor cells in different tissues). Recent evidence suggests that WT1 plays a role in leukemogenesis and tumorigenesis.

WT1 has several isoforms, some of which result from alternative splicing of mRNA transcripts encoding WT1. The complete amino acid sequence of a WT1 isoform was previously published (Gessler, M. et al. Nature; 343(6260):774-778; (1990)). This particular isoform consists of 575 amino acids and includes a first 126 amino acids at the N terminus which are lacking in the exon 5+ and the KTS+ isoforms of WT1.

An example WT1 protein has the amino acid sequence set out in UniProt entry J3KNN9. Another example WT1 protein has the amino acid sequence set out below:

(SEQ ID NO: 79)
SRQRPHPGALRNPTACPLPHFPPSLPPTHSPTHPPRAGTAAQAPGPRRLL
AAILDFLLLQDPASTCVPEPASQHTLRSGPGCLQQPEQQGVRDPGGIWAK
LGAAEASAERLQGRRSRGASGSEPQQMGSDVRDLNALLPAVPSLGGGGGC
ALPVSGAAQWAPVLDFAPPGASAYGSLGGPAPPPAPPPPPPPPPHSFIKQ
EPSWGGAEPHEEQCLSAFTVHFSGQFTGTAGACRYGPFGPPPPSQASSGQ
ARMFPNAPYLPSCLESQPAIRNQGYSTVTFDGTPSYGHTPSHHAAQFPNH
SFKHEDPMGQQGSLGEQQYSVPPPVYGCHTPTDSCTGSQALLLRTPYSSD
NLYQMTSQLECMTWNQMNLGATLKGVAAGSSSSVKWTEGQSNHSTGYESD
NHTTPILCGAQYRIHTHGVFRGIQDVRRVPGVAPTLVRSASETSEKRPFM
CAYPGCNKRYFKLSHLQMHSRKHTGEKPYQCDFKDCERRFSRSDQLKRHQ
RRHTGVKPFQCKTCQRKFSRSDHLKTHTRTHTGKTSEKPFSCRWPSCQKK
FARSDELVRHHNMHQRNMTKLQLAL

WT1 Peptides

As used herein the term peptide refers to a plurality of amino acid residues linked by peptide bonds. As defined herein a peptide may consist of less than about 30, less than about 25, less than about 20, less than 19, less than 18, less than 17, less than 16, less than 15, less than 14, less than 13, less than 12, less than 11, less than 10, less than 9, less than 8, less than 7, less than 6, or less than 5 amino acid residues in length. Preferably, a peptide is about 5 to 20 amino acids in length, more preferably, a peptide is about 8 to 15 amino acid residues in length.

The TCRs of the invention bind to a WT1 peptide when presented by an MHC. As used herein, the term WT1 peptide is understood to mean α peptide comprising an amino acid sequence derived from a WT1 protein.

For example, a WT1 peptide may comprise at least 5, at least 6, at least 7, at least 8, at least 9, at least 10, at least 11, at least 12, at least 13, at least 14, at least 15, at least 16, at least 17, at least 18, at least 19, at least 20, or at least 25 contiguous amino acid residues of a WT1 protein amino acid sequence.

The WT1 peptide may comprise or consist of an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87) or variants thereof each having up to three amino acid substitutions, additions or deletions.

In some embodiments, for WT1 peptides which bind to MHC molecules encoded by HLA-A*0201 allele it may be preferred that the amino acids at position 2 of the peptide (i.e. the second amino acid from the N-terminus) are leucine or methionine, although isoleucine, valine, alanine and threonine may also be preferable. It may also be preferred that the amino acid at position 9 or 10 is valine, leucine or isoleucine, although alanine, methionine and threonine may also be preferable. The preferred MHC binding motifs of other HLA alleles are disclosed in Celis et al (Molecular Immunology, Vol. 31, 8, December 1994, pages 1423 to 1430).

Various uses of the WT1 peptides described herein are contemplated by the invention. For example, the WT1 peptides described herein may be administered to a subject, e.g. a human subject. Administration of the WT1 peptides of the invention may elicit an immune response against cells expressing or overexpressing WT1 protein, i.e. the WT1 peptides are immunogenic WT1 peptides.

Thus in another aspect, the invention provides an isolated immunogenic WT1 peptide comprising an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87), and variants thereof each having up to three amino acid substitutions, additions or deletions.

The WT1 peptides described herein, e.g. WT1 peptides comprising an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87), and variants thereof each having up to three amino acid substitutions, additions or deletions, may be used to screen for and/or identify new TCR sequences which bind to WT1 cells. For example, T2 cells may be pulsed with a WT1 peptide mentioned in the invention and incubated with a T-cell population isolated from a donor. In this approach, expression of cytokines, e.g. CD107a and IFNγ, may be indicative of T-cells which recognise WT1 peptides.

Accordingly, in one aspect, the invention provides a T-cell receptor (TCR), which binds to a Wilms tumour 1 protein (WT1) peptide when presented by a major histocompatibility complex (MHC), wherein the WT1 peptide comprises an amino acid sequence selected from the group consisting of GAQYRIHTHGVFRGI (SEQ ID NO: 181), LLAAILDFLLLQDPA (SEQ ID NO: 82) and CMTWNQMNLGATLKG (SEQ ID NO: 87), and variants thereof each having up to three amino acid substitutions, additions or deletions.

TCR Sequences

We have determined the amino acid sequences for TCRs that bind to WT1 peptides described herein. In particular, we have determined the amino acid sequences of the TCR CDRs, which are important for WT1 peptide recognition and binding.

In one aspect, the invention provides a TCR comprising a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions, which binds to a WT1 peptide comprising the amino acid sequence of LLAAILDFLLLQDPA (SEQ ID NO: 82) or a variant thereof having up to three amino acid substitutions, additions or deletions when presented by an MHC.

In one aspect, the invention provides a TCR comprising a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions, which binds to a WT1 peptide comprising the amino acid sequence of LLAAILDFLLLQDPA (SEQ ID NO: 82) or a variant thereof having up to three amino acid substitutions, additions or deletions when presented by an MHC.

In one aspect, the invention provides a TCR comprising a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions, which binds to a WT1 peptide comprising the amino acid sequence of GAQYRIHTHGVFRGI (SEQ ID NO: 181) or a variant thereof having up to three amino acid substitutions, additions or deletions when presented by an MHC.

In one aspect, the invention provides a TCR comprising a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions, which binds to a WT1 peptide comprising the amino acid sequence of CMTWNQMNLGATLKG (SEQ ID NO: 87) or a variant thereof having up to three amino acid substitutions, additions or deletions when presented by an MHC.

In one aspect, the invention provides a TCR comprising a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions, which binds to a WT1 peptide comprising the amino acid sequence of CMTWNQMNLGATLKG (SEQ ID NO: 87) or a variant thereof having up to three amino acid substitutions, additions or deletions when presented by an MHC.

In one aspect, the invention provides a TCR comprising a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions, which binds to a WT1 peptide comprising the amino acid sequence of CMTWNQMNLGATLKG (SEQ ID NO: 87) or a variant thereof having up to three amino acid substitutions, additions or deletions when presented by an MHC.

In one aspect, the invention provides a TR comprising a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions, which binds to a WT1 peptide comprising the amino acid sequence of CMTWNQMNLGATLKG (SEQ ID NO: 87) or a variant thereof having up to three amino acid substitutions, additions or deletions when presented by an MHC.

Example TCR amino acid sequences of the present invention are provided in Table 1.

TABLE 1

| | | | |
|---|---|---|---|
| Chain | Region | Amino acid sequence | SEQ ID NO |

Donor: HD12

| | | | |
|---|---|---|---|
| Alpha (α) | CDR1α | TSDQSYG | SEQ ID NO: 1 |
| | CDR2α | QGSYDEQN | SEQ ID NO: 2 |
| | CDR3α | CAMRTGGGADGLTF | SEQ ID NO: 3 |
| | Variable | MSLSSLLKVVTASLWLGPGIAQKITQTQPGMFVQEKEAVTLD | SEQ ID NO: 4 |
| | | CTYDTSDQSYGLFWYKQPSSGEMIFLIYQGSYDEQNATEGRY | |
| | | SLNFQKARKSANLVISASQLGDSAMYFCAMRTGGGADGLTFG | |
| | | KGTHLIIQPY | |
| | Full - with | MSLSSLLKVVTASLWLGPGIAQKITQTQPGMFVQEKEAVTLD | SEQ ID NO: 5 |
| | TRAC constant | CTYDTSDQSYGLFWYKQPSSGEMIFLIYQGSYDEQNATEGRY | |
| | domain | SLNFQKARKSANLVISASQLGDSAMYFCAMRTGGGADGLTFG | |
| | | KGTHLIIQPYIQNPDPAVYQLRDSKSSDKSVCLFTDFDSQTN | |
| | | VSQSKDSDVYITDKTVLDMRSMDFKSNSAVAWSNKSDFACAN | |
| | | AFNNSIIPEDTFFPSPESSCDVKLVEKSFETDTNLNFQNLSV | |
| | | IGFRILLLKVAGFNLLMTLRLWSS | |
| | Full - with | MSLSSLLKVVTASLWLGPGIAQKITQTQPGMFVQEKEAVTLD | SEQ ID NO: 194 |
| | TRAC constant | CTYDTSDQSYGLFWYKQPSSGEMIFLIYQGSYDEQNATEGRY | |
| | domain | SLNFQKARKSANLVISASQLGDSAMYFCAMRTGGGADGLTFG | |
| | | KGTHLIIQPYIQNPDPAVYQLRDSKSSDKSVCLFTDFDSQTN | |
| | | VSQSKDSDVYITDKCVLDMRSMDFKSNSAVAWSNKSDFACAN | |
| | | AFNNSIIPEDTFFPSPESSCDVKLVEKSFETDTNLNFQNLSV | |
| | | IGFRILLLKVAGFNLLMTLRLWSS | |
| Beta (β) | CDR1β | SNHLY | SEQ ID NO: 6 |
| | CDR2β | FYNNEI | SEQ ID NO: 7 |
| | CDR3β | CASSEAGLSYEQYF | SEQ ID NO: 8 |
| | Variable | MDTWLVCWAIFSLLKAGLTEPEVTQTPSHQVTQMGQEVILRC | SEQ ID NO: 9 |
| | | VPISNHLYFYWYRQILGQKVEFLVSFYNNEISEKSEIFDDQF | |
| | | SVERPDGSNFTLKIRSTKLEDSAMYFCASSEAGLSYEQYFGP | |
| | | GTRLTVTE | |
| | Full - with | MDTWLVCWAIFSLLKAGLTEPEVTQTPSHQVTQMGQEVILRC | SEQ ID NO: 10 |
| | TRBC1 | VPISNHLYFYWYRQILGQKVEFLVSFYNNEISEKSEIFDDQF | |
| | constant | SVERPDGSNFTLKIRSTKLEDSAMYFCASSEAGLSYEQYFGP | |
| | domain | GTRLTVTEDLNKVFPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |
| | Full - with | MDTWLVCWAIFSLLKAGLTEPEVTQTPSHQVTQMGQEVILRC | SEQ ID NO: 11 |
| | TRBC2 | VPISNHLYFYWYRQILGQKVEFLVSFYNNEISEKSEIFDDQF | |
| | constant | SVERPDGSNFTLKIRSTKLEDSAMYFCASSEAGLSYEQYFGP | |
| | domain | GTRLTVTEDLKNVFPPEVAVFEPSEAEISHTQKATLVCLATG | |
| | | FYPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSESYQQGVLSATILYEILLGKATLYAV | |
| | | LVSALVLMAMVKRKDSRG | |
| | Full - with | MDTWLVCWAIFSLLKAGLTEPEVTQTPSHQVTQMGQEVILRC | SEQ ID NO: 195 |
| | TRBC2 | VPISNHLYFYWYRQILGQKVEFLVSFYNNEISEKSEIFDDQF | |
| | constant | SVERPDGSNFTLKIRSTKLEDSAMYFCASSEAGLSYEQYFGP | |
| | domain | GTRLTVTEDLKNVFPPEVAVFEPSEAEISHTQKATLVCLATG | |
| | | FYPDHVELSWWVNGKEVHSGVCTDPQPLKEQPALNDSRYCLS | |
| | | SRLRVSATFWQNPRNHFRCQVQFYGLSENDEWTQDRAKPVTQ | |
| | | IVSAEAWGRADCGFTSESYQQGVLSATILYEILLGKATLYAV | |
| | | LVSALVLMAMVKRKDSRG | |

Donor: HD13

| | | | |
|---|---|---|---|
| Alpha (α) | CDR1α | TISGTDY | SEQ ID NO: 12 |
| | CDR2α | GLTSN | SEQ ID NO: 13 |
| | CDR3α | CILSTRVWAGSYQLTF | SEQ ID NO: 14 |
| | Variable | MKLVTSITVLLSLGIMGDAKTTQPNSMESNEEEPVHLPCNHS | SEQ ID NO: 15 |
| | | TISGTDYIHWYRQLPSQGPEYVIHGLTSNVNNRMASLAIAED | |
| | | RKSSTLILHRATLRDAAVYYCILSTRVWAGSYQLTFGKGTKL | |
| | | SVIPN | |
| | Full - with | MKLVTSITVLLSLGIMGDAKTTQPNSMESNEEEPVHLPCNHS | SEQ ID NO: 16 |
| | TRAC constant | TISGTDYIHWYRQLPSQGPEYVIHGLTSNVNNRMASLAIAED | |
| | domain | RKSSTLILHRATLRDAAVYYCILSTRVWAGSYQLTFGKGTKL | |
| | | SVIPNIQNPDPAVYQLRDSKSSDKSVCLFTDFDSQTNVSQSK | |
| | | DSDVYITDKTVLDMRSMDFKSNSAVAWSNKSDFACANAFNNS | |
| | | IIPEDTFFPSPESSCDVKLVEKSFETDTNLNFQNLSVIGFRI | |
| | | LLLKVAGFNLLMTLRLWSS | |
| | Full - with | MKLVTSITVLLSLGIMGDAKTTQPNSMESNEEEPVHLPCNHS | SEQ ID NO: 196 |
| | TRAC constant | TISGTDYIHWYRQLPSQGPEYVIHGLTSNVNNRMASLAIAED | |
| | domain | RKSSTLILHRATLRDAAVYYCILSTRVWAGSYQLTFGKGTKL | |
| | | SVIPNIQNPDPAVYQLRDSKSSDKSVCLFTDFDSQTNVSQSK | |
| | | DSDVYITDKCVLDMRSMDFKSNSAVAWSNKSDFACANAFNNS | |
| | | IIPEDTFFPSPESSCDVKLVEKSFETDTNLNFQNLSVIGFRI | |
| | | LLLKVAGFNLLMTLRLWSS | |
| Beta (β) | CDR1β | KGHDR | SEQ ID NO: 17 |
| | CDR2β | SFDVKD | SEQ ID NO: 18 |
| | CDR3β | CATGQATQETQYF | SEQ ID NO: 19 |
| | Variable | MASLLFFCGAFYLLGTGSMDADVTQTPRNRITKTGKRIMLEC | SEQ ID NO: 20 |
| | | SQTKGHDRMYWYRQDPGLGLRLIYYSFDVKDINKGEISDGYS | |
| | | VSRQAQAKFSLSLESAIPNQTALYFCATGQATQETQYFGPGT | |
| | | RLLVLE | |
| | Full - with | MASLLFFCGAFYLLGTGSMDADVTQTPRNRITKTGKRIMLEC | SEQ ID NO: 21 |
| | TRBC1 | SQTKGHDRMYWYRQDPGLGLRLIYYSFDVKDINKGEISDGYS | |
| | constant | VSRQAQAKFSLSLESAIPNQTALYFCATGQATQETQYFGPGT | |
| | domain | RLLVLEDLNKVFPPEVAVFEPSEAEISHTQKATLVCLATGFF | |
| | | PDHVELSWWVNGKEVHSGVSTDPQPLKEQPALNDSRYCLSSR | |
| | | LRVSATFWQNPRNHFRCQVQFYGLSENDEWTQDRAKPVTQIV | |
| | | SAEAWGRADCGFTSVSYQQGVLSATILYEILLGKATLYAVLV | |
| | | SALVLMAMVKRKDF | |
| | Full - with | MASLLFFCGAFYLLGTGSMDADVTQTPRNRITKTGKRIMLEC | SEQ ID NO: 22 |
| | TRBC2 | SQTKGHDRMYWYRQDPGLGLRLIYYSFDVKDINKGEISDGYS | |
| | constant | VSRQAQAKFSLSLESAIPNQTALYFCATGQATQETQYFGPGT | |
| | domain | RLLVLEDLKNVFPPEVAVFEPSEAEISHTQKATLVCLATGFY | |
| | | PDHVELSWWVNGKEVHSGVSTDPQPLKEQPALNDSRYCLSSR | |
| | | LRVSATFWQNPRNHFRCQVQFYGLSENDEWTQDRAKPVTQIV | |
| | | SAEAWGRADCGFTSESYQQGVLSATILYEILLGKATLYAVLV | |
| | | SALVLMAMVKRKDSRG | |
| | Full - with | MASLLFFCGAFYLLGTGSMDADVTQTPRNRITKTGKRIMLEC | SEQ ID NO: 197 |
| | TRBC2 | SQTKGHDRMYWYRQDPGLGLRLIYYSFDVKDINKGEISDGYS | |
| | constant | VSRQAQAKFSLSLESAIPNQTALYFCATGQATQETQYFGPGT | |
| | domain | RLLVLEDLKNVFPPEVAVFEPSEAEISHTQKATLVCLATGFY | |
| | | PDHVELSWWVNGKEVHSGVCTDPQPLKEQPALNDSRYCLSSR | |
| | | LRVSATFWQNPRNHFRCQVQFYGLSENDEWTQDRAKPVTQIV | |
| | | SAEAWGRADCGFTSESYQQGVLSATILYEILLGKATLYAVLV | |
| | | SALVLMAMVKRKDSRG | |

Donor: HD14

| | | | |
|---|---|---|---|
| Alpha (α) | CDR1α | NSAFQY | SEQ ID NO: 23 |
| (with | CDR2α | TYSSGN | SEQ ID NO: 24 |
| TRAV12- | CDR3α | CASGGGADGLTF | SEQ ID NO: 25 |
| 3*01) | Variable | MMKSLRVLLVILWLQLSWVWSQQKEVEQDPGPLSVPEGAIVS | SEQ ID NO: 26 |
| | | LNCTYSNSAFQYFMWYRQYSRKGPELLMYTYSSGNKEDGRFT | |
| | | AQVDKSSKYISLFIRDSQPSDSATYLCASGGGADGLTFGKGT | |
| | | HLIIQPY | |
| | Full - with | MMKSLRVLLVILWLQLSWVWSQQKEVEQDPGPLSVPEGAIVS | SEQ ID NO: 27 |
| | TRAC constant | LNCTYSNSAFQYFMWYRQYSRKGPELLMYTYSSGNKEDGRFT | |
| | domain | AQVDKSSKYISLFIRDSQPSDSATYLCASGGGADGLTFGKGT | |
| | | HLIIQPYIQNPDPAVYQLRDSKSSDKSVCLFTDFDSQTNVSQ | |
| | | SKDSDVYITDKTVLDMRSMDFKSNSAVAWSNKSDFACANAFN | |
| | | NSIIPEDTFFPSPESSCDVKLVEKSFETDTNLNFQNLSVIGF | |
| | | RILLLKVAGFNLLMTLRLWSS | |
| Alpha (α) | CDR1α | DRGSQS | SEQ ID NO: 182 |
| (with | CDR2α | IYSNGD | SEQ ID NO: 183 |
| TRAV12- | CDR3α | CASGGGADGLTF | SEQ ID NO: 25 |
| 2*01) | Variable | MKSLRVLLVILWLQLSWVWSQQKEVEQNSGPLSVPEGAIASL | SEQ ID NO: 185 |
| | | NCTYSDRGSQSFFWYRQYSGKSPELIMFIYSNGDKEDGRFTA | |
| | | QLNKASQYVSLLIRDSQPSDSATYLCASGGGADGLTFGKGTH | |
| | | LIIQPY | |
| | Full - with | MKSLRVLLVILWLQLSWVWSQQKEVEQNSGPLSVPEGAIASL | SEQ ID NO: 186 |
| | TRAC constant | NCTYSDRGSQSFFWYRQYSGKSPELIMFIYSNGDKEDGRFTA | |
| | domain | QLNKASQYVSLLIRDSQPSDSATYLCASGGGADGLTFGKGTH | |
| | | LIIQPYIQNPDPAVYQLRDSKSSDKSVCLFTDFDSQTNVSQS | |
| | | KDSDVYITDKTVLDMRSMDFKSNSAVAWSNKSDFACANAFNN | |
| | | SIIPEDTFFPSPESSCDVKLVEKSFETDTNLNFQNLSVIGFR | |
| | | ILLLKVAGFNLLMTLRLWSS | |
| Alpha (α) | CDR1α | DRGSQS | SEQ ID NO: 182 |
| (with | CDR2α | IYSNGD | SEQ ID NO: 183 |
| TRAV12- | CDR3α | CASGGGADGLTF | SEQ ID NO: 25 |
| 2*02) | Variable | MMKSLRVLLVILWLQLSWVWSQQKEVEQNSGPLSVPEGAIAS | SEQ ID NO: 190 |
| | | LNCTYSDRGSQSFFWYRQYSGKSPELIMSIYSNGDKEDGRFT | |
| | | AQLNKASQYVSLLIRDSQPSDSATYLCASGGGADGLTFGKGT | |
| | | HLIIQPY | |
| | Full - with | MMKSLRVLLVILWLQLSWVWSQQKEVEQNSGPLSVPEGAIAS | SEQ ID NO: 191 |
| | TRAC constant | LNCTYSDRGSQSFFWYRQYSGKSPELIMSIYSNGDKEDGRFT | |
| | domain | AQLNKASQYVSLLIRDSQPSDSATYLCASGGGADGLTFGKGT | |
| | | HLIIQPYIQNPDPAVYQLRDSKSSDKSVCLFTDFDSQTNVSQ | |
| | | SKDSDVYITDKTVLDMRSMDFKSNSAVAWSNKSDFACANAFN | |
| | | NSIIPEDTFFPSPESSCDVKLVEKSFETDTNLNFQNLSVIGF | |
| | | RILLLKVAGFNLLMTLRLWSS | |
| Alpha (α) | Full - with | MMKSLRVLLVILWLQLSWVWSQQKEVEQDPGPLSVPEGAIVS | SEQ ID NO: 198 |
| (TRAV12- | TRAC constant | LNCTYSNSAFQYFMWYRQYSRKGPELLMYTYSSGNKEDGRFT | |
| 3*01 WT) | domain | AQVDKSSKYISLFIRDSQPSDSATYLCASGGGADGLTFGKGT | |
| | | HLIIQPYIQNPDPAVYQLRDSKSSDKSVCLFTDFDSQTNVSQ | |
| | | SKDSDVYITDKCVLDMRSMDFKSNSAVAWSNKSDFACANAFN | |
| | | NSIIPEDTFFPSPESSCDVKLVEKSFETDTNLNFQNLSVIGF | |
| | | RILLLKVAGFNLLMTLRLWSS | |
| Alpha (α) | Full - with | MKSLRVLLVILWLQLSWVWSQQKEVEQNSGPLSVPEGAIASL | SEQ ID NO: 199 |
| (TRAV12- | TRAC constant | NCTYSDRGSQSFFWYRQYSGKSPELIMFIYSNGDKEDGRFTA | |
| 2*01 WT) | domain | QLNKASQYVSLLIRDSQPSDSATYLCASGGGADGLTFGKGTH | |
| | | LIIQPYIQNPDPAVYQLRDSKSSDKSVCLFTDFDSQTNVSQS | |
| | | KDSDVYITDKCVLDMRSMDFKSNSAVAWSNKSDFACANAFNN | |
| | | SIIPEDTFFPSPESSCDVKLVEKSFETDTNLNFQNLSVIGFR | |
| | | ILLLKVAGFNLLMTLRLWSS | |
| Alpha (α) | Full - with | MKSLRVLLVILWLQLSWVWSQQKEVEQNSGPLSVPEGAIASL | SEQ ID NO: 200 |
| (TRAV12- | TRAC constant | NCTYSDRGSQSFFWYRQYSGKSPELIMFIYSNGDKEDGRFTA | |
| 2*01 mut) | domain | QLNKASQYVSLLIRDSQPSDSATYLCASGGGADGLTFGKGTH | |
| | | LIIQPYIQNPDPAVYQLRDSKSSDKSVCLFTDFDSQTQVSQS | |
| | | KDSDVYITDKCVLDMRSMDFKSNSAVAWSNKSDFACANAFNN | |
| | | SIIPEDTFFPSPESSCDVKLVEKSFETDTNLNFQNLSVIGFR | |
| | | ILLLKVAGFNLLMTLRLWSS | |
| Alpha (α) | Full - with | MMKSLRVLLVILWLQLSWVWSQQKEVEQNSGPLSVPEGAIAS | SEQ ID NO: 201 |
| (TRAV12- | TRAC constant | LNCTYSDRGSQSFFWYRQYSGKSPELIMSIYSNGDKEDGRFT | |
| 2*02 WT) | domain | AQLNKASQYVSLLIRDSQPSDSATYLCASGGGADGLTFGKGT | |
| | | HLIIQPYIQNPDPAVYQLRDSKSSDKSVCLFTDFDSQTNVSQ | |
| | | SKDSDVYITDKCVLDMRSMDFKSNSAVAWSNKSDFACANAFN | |
| | | NSIIPEDTFFPSPESSCDVKLVEKSFETDTNLNFQNLSVIGF | |
| | | RILLLKVAGFNLLMTLRLWSS | |
| Alpha (α) | Full - with | MMKSLRVLLVILWLQLSWVWSQQKEVEQNSGPLSVPEGAIAS | SEQ ID NO: 202 |
| (TRAV12- | TRAC constant | LNCTYSDRGSQSFFWYRQYSGKSPELIMSIYSNGDKEDGRFT | |
| 2*02 mut) | domain | AQLNKASQYVSLLIRDSQPSDSATYLCASGGGADGLTFGKGT | |
| | | HLIIQPYIQNPDPAVYQLRDSKSSDKSVCLFTDFDSQTQVSQ | |
| | | SKDSDVYITDKCVLDMRSMDFKSNSAVAWSNKSDFACANAFN | |
| | | NSIIPEDTFFPSPESSCDVKLVEKSFETDTNLNFQNLSVIGF | |
| | | RILLLKVAGFNLLMTLRLWSS | |
| Beta (β) | CDR1β | SGDLS | SEQ ID NO: 28 |
| | CDR2β | YYNGEE | SEQ ID NO: 29 |
| | CDR3β | CASGRGDTEAFF | SEQ ID NO: 30 |
| | Variable | MGFRLLCCVAFCLLGAGPVDSGVTQTPKHLITATGQRVTLRC | SEQ ID NO: 31 |
| | | SPRSGDLSVYWYQQSLDQGLQFLIQYYNGEERAKGNILERFS | |
| | | AQQFPDLHSELNLSSLELGDSALYFCASGRGDTEAFFGQGTR | |
| | | LTVVE | |
| | Full - with | MGFRLLCCVAFCLLGAGPVDSGVTQTPKHLITATGQRVTLRC | SEQ ID NO: 32 |
| | TRBC1 | SPRSGDLSVYWYQQSLDQGLQFLIQYYNGEERAKGNILERFS | |
| | constant | AQQFPDLHSELNLSSLELGDSALYFCASGRGDTEAFFGQGTR | |
| | domain | LTVVEDLNKVFPPEVAVFEPSEAEISHTQKATLVCLATGFFP | |
| | | DHVELSWWVNGKEVHSGVSTDPQPLKEQPALNDSRYCLSSRL | |
| | | RVSATFWQNPRNHFRCQVQFYGLSENDEWTQDRAKPVTQIVS | |
| | | AEAWGRADCGFTSVSYQQGVLSATILYEILLGKATLYAVLVS | |
| | | ALVLMAMVKRKDF | |
| | Full - with | MGFRLLCCVAFCLLGAGPVDSGVTQTPKHLITATGQRVTLRC | SEQ ID NO: 33 |
| | TRBC2 | SPRSGDLSVYWYQQSLDQGLQFLIQYYNGEERAKGNILERFS | |
| | constant | AQQFPDLHSELNLSSLELGDSALYFCASGRGDTEAFFGQGTR | |
| | domain | LTVVEDLKNVFPPEVAVFEPSEAEISHTQKATLVCLATGFYP | |
| | | DHVELSWWVNGKEVHSGVSTDPQPLKEQPALNDSRYCLSSRL | |
| | | RVSATFWQNPRNHFRCQVQFYGLSENDEWTQDRAKPVTQIVS | |
| | | AEAWGRADCGFTSESYQQGVLSATILYEILLGKATLYAVLVS | |
| | | ALVLMAMVKRKDSRG | |
| Beta (β) | Full - with | MGFRLLCCVAFCLLGAGPVDSGVTQTPKHLITATGQRVTLRC | SEQ ID NO: 203 |
| (TRAV12- | TRBC2 | SPRSGDLSVYWYQQSLDQGLQFLIQYYNGEERAKGNILERFS | |
| 3*01 WT) | constant | AQQFPDLHSELNLSSLELGDSALYFCASGRGDTEAFFGQGTR | |
| | domain | LTVVEDLKNVFPPEVAVFEPSEAEISHTQKATLVCLATGFYP | |
| | | DHVELSWWVNGKEVHSGVCTDPQPLKEQPALNDSRYCLSSRL | |
| | | RVSATFWQNPRNHFRCQVQFYGLSENDEWTQDRAKPVTQIVS | |
| | | AEAWGRADCGFTSESYQQGVLSATILYEILLGKATLYAVLVS | |
| | | ALVLMAMVKRKDSRG | |

Donor: HD15

| | | | |
|---|---|---|---|
| Alpha (α) | CDR1α | DRGSQS | SEQ ID NO: 34 |
| S4 | CDR2α | IYSNGD | SEQ ID NO: 35 |
| population | CDR3α | CAVIGGTDSWGKLQF | SEQ ID NO: 36 |
| | Variable | MKSLRVLLVILWLQLSWVWSQQKEVEQNSGPLSVPEGAIASL | SEQ ID NO: 37 |
| | | NCTYSDRGSQSFFWYRQYSGKSPELIMFIYSNGDKEDGRFTA | |
| | | QLNKASQYVSLLIRDSQPSDSATYLCAVIGGTDSWGKLQFGA | |
| | | GTQVVVTPD | |
| | Full - with | MKSLRVLLVILWLQLSWVWSQQKEVEQNSGPLSVPEGAIASL | SEQ ID NO: 38 |
| | TRAC constant | NCTYSDRGSQSFFWYRQYSGKSPELIMFIYSNGDKEDGRFTA | |
| | domain | QLNKASQYVSLLIRDSQPSDSATYLCAVIGGTDSWGKLQFGA | |
| | | GTQVVVTPDIQNPDPAVYQLRDSKSSDKSVCLFTDFDSQTNV | |
| | | SQSKDSDVYITDKTVLDMRSMDFKSNSAVAWSNKSDFACANA | |
| | | FNNSIIPEDTFFPSPESSCDVKLVEKSFETDTNLNFQNLSVI | |
| | | GFRILLLKVAGFNLLMTLRLWSS | |
| | Full - with | MKSLRVLLVILWLQLSWVWSQQKEVEQNSGPLSVPEGAIASL | SEQ ID NO: 214 |
| | TRAC constant | NCTYSDRGSQSFFWYRQYSGKSPELIMFIYSNGDKEDGRFTA | |
| | domain | QLNKASQYVSLLIRDSQPSDSATYLCAVIGGTDSWGKLQFGA | |
| | | GTQVVVTPDIQNPDPAVYQLRDSKSSDKSVCLFTDFDSQTNV | |
| | | SQSKDSDVYITDKCVLDMRSMDFKSNSAVAWSNKSDFACANA | |
| | | FNNSIIPEDTFFPSPESSCDVKLVEKSFETDTNLNFQNLSVI | |
| | | GFRILLLKVAGFNLLMTLRLWSS | |
| Beta (β) | CDR1β | LGHNA | SEQ ID NO: 39 |
| S4 | CDR2β | YSLEER | SEQ ID NO: 40 |
| population | CDR3β | CASSQEEGAVYGYTF | SEQ ID NO: 41 |
| | Variable | MGCRLLCCAVLCLLGAGELVPMETGVTQTPRHLVMGMTNKKS | SEQ ID NO: 42 |
| | | LKCEQHLGHNAMYWYKQSAKKPLELMFVYSLEERVENNSVPS | |
| | | RFSPECPNSSHLFLHLHTLQPEDSALYLCASSQEEGAVYGYT | |
| | | FGSGTRLTVVE | |
| | Full - with | MGCRLLCCAVLCLLGAGELVPMETGVTQTPRHLVMGMTNKKS | SEQ ID NO: 43 |
| | TRBC1 | LKCEQHLGHNAMYWYKQSAKKPLELMFVYSLEERVENNSVPS | |
| | constant | RFSPECPNSSHLFLHLHTLQPEDSALYLCASSQEEGAVYGYT | |
| | domain | FGSGTRLTVVEDLNKVFPPEVAVFEPSEAEISHTQKATLVCL | |
| | | ATGFFPDHVELSWWVNGKEVHSGVSTDPQPLKEQPALNDSRY | |
| | | CLSSRLRVSATFWQNPRNHFRCQVQFYGLSENDEWTQDRAKP | |
| | | VTQIVSAEAWGRADCGFTSVSYQQGVLSATILYEILLGKATL | |
| | | YAVLVSALVLMAMVKRKDF | |
| | Full - with | MGCRLLCCAVLCLLGAGELVPMETGVTQTPRHLVMGMTNKKS | SEQ ID NO: 44 |
| | TRBC2 | LKCEQHLGHNAMYWYKQSAKKPLELMFVYSLEERVENNSVPS | |
| | constant | RFSPECPNSSHLFLHLHTLQPEDSALYLCASSQEEGAVYGYT | |
| | domain | FGSGTRLTVVEDLKNVFPPEVAVFEPSEAEISHTQKATLVCL | |
| | | ATGFYPDHVELSWWVNGKEVHSGVSTDPQPLKEQPALNDSRY | |
| | | CLSSRLRVSATFWQNPRNHFRCQVQFYGLSENDEWTQDRAKP | |
| | | VTQIVSAEAWGRADCGFTSESYQQGVLSATILYEILLGKATL | |
| | | YAVLVSALVLMAMVKRKDSRG | |
| | Full - with | MGCRLLCCAVLCLLGAGELVPMETGVTQTPRHLVMGMTNKKS | SEQ ID NO: 215 |
| | TRBC2 | LKCEQHLGHNAMYWYKQSAKKPLELMFVYSLEERVENNSVPS | |
| | constant | RFSPECPNSSHLFLHLHTLQPEDSALYLCASSQEEGAVYGYT | |
| | domain | FGSGTRLTVVEDLKNVFPPEVAVFEPSEAEISHTQKATLVCL | |
| | | ATGFYPDHVELSWWVNGKEVHSGVCTDPQPLKEQPALNDSRY | |
| | | CLSSRLRVSATFWQNPRNHFRCQVQFYGLSENDEWTQDRAKP | |
| | | VTQIVSAEAWGRADCGFTSESYQQGVLSATILYEILLGKATL | |
| | | YAVLVSALVLMAMVKRKDSRG | |
| Alpha (α) | CDR1α | NSASQS | SEQ ID NO: 45 |
| S1 IFNg | CDR2α | VYSSGN | SEQ ID NO: 46 |
| enriched | CDR3α | CVVPRGLSTDSWGKLQF | SEQ ID NO: 47 |
| population | Variable | MISLRVLLVILWLQLSWVWSQRKEVEQDPGPFNVPEGATVAF | SEQ ID NO: 48 |
| | | NCTYSNSASQSFFWYRQDCRKEPKLLMSVYSSGNEDGRFTAQ | |
| | | LNRASQYISLLIRDSKLSDSATYLCVVPRGLSTDSWGKLQFG | |
| | | AGTQVVVTPD | |
| | Full - with | MISLRVLLVILWLQLSWVWSQRKEVEQDPGPFNVPEGATVAF | SEQ ID NO: 49 |
| | TRAC constant | NCTYSNSASQSFFWYRQDCRKEPKLLMSVYSSGNEDGRFTAQ | |
| | domain | LNRASQYISLLIRDSKLSDSATYLCVVPRGLSTDSWGKLQFG | |
| | | AGTQVVVTPDIQNPDPAVYQLRDSKSSDKSVCLFTDFDSQTN | |
| | | VSQSKDSDVYITDKTVLDMRSMDFKSNSAVAWSNKSDFACAN | |
| | | AFNNSIIPEDTFFPSPESSCDVKLVEKSFETDTNLNFQNLSV | |
| | | IGFRILLLKVAGFNLLMTLRLWSS | |
| | Full - with | MISLRVLLVILWLQLSWVWSQRKEVEQDPGPFNVPEGATVAF | SEQ ID NO: 216 |
| | TRAC constant | NCTYSNSASQSFFWYRQDCRKEPKLLMSVYSSGNEDGRFTAQ | |
| | domain | LNRASQYISLLIRDSKLSDSATYLCVVPRGLSTDSWGKLQFG | |
| | | AGTQVVVTPDIQNPDPAVYQLRDSKSSDKSVCLFTDFDSQTN | |
| | | VSQSKDSDVYITDKCVLDMRSMDFKSNSAVAWSNKSDFACAN | |
| | | AFNNSIIPEDTFFPSPESSCDVKLVEKSFETDTNLNFQNLSV | |
| | | IGFRILLLKVAGFNLLMTLRLWSS | |
| Beta (β) | CDR1β | LNHNV | SEQ ID NO: 50 |
| S1 IFNg | CDR2β | YYDKDF | SEQ ID NO: 51 |
| enriched | CDR3β | CATSREGLAADTQYF | SEQ ID NO: 52 |
| population | Variable | MGPGLLHWMALCLLGTGHGDAMVIQNPRYQVTQFGKPVTLSC | SEQ ID NO: 53 |
| | | SQTLNHNVMYWYQQKSSQAPKLLFHYYDKDFNNEADTPDNFQ | |
| | | SRRPNTSFCFLDIRSPGLGDAAMYLCATSREGLAADTQYFGP | |
| | | GTRLTVLE | |
| | Full - with | MGPGLLHWMALCLLGTGHGDAMVIQNPRYQVTQFGKPVTLSC | SEQ ID NO: 54 |
| | TRBC1 | SQTLNHNVMYWYQQKSSQAPKLLFHYYDKDFNNEADTPDNFQ | |
| | constant | SRRPNTSFCFLDIRSPGLGDAAMYLCATSREGLAADTQYFGP | |
| | domain | GTRLTVLEDLNKVFPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |
| | Full - with | MGPGLLHWMALCLLGTGHGDAMVIQNPRYQVTQFGKPVTLSC | SEQ ID NO: 55 |
| | TRBC2 | SQTLNHNVMYWYQQKSSQAPKLLFHYYDKDFNNEADTPDNFQ | |
| | constant | SRRPNTSFCFLDIRSPGLGDAAMYLCATSREGLAADTQYFGP | |
| | domain | GTRLTVLEDLKNVFPPEVAVFEPSEAEISHTQKATLVCLATG | |
| | | FYPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSESYQQGVLSATILYEILLGKATLYAV | |
| | | LVSALVLMAMVKRKDSRG | |
| | Full - with | MGPGLLHWMALCLLGTGHGDAMVIQNPRYQVTQFGKPVTLSC | SEQ ID NO: 217 |
| | TRBC2 | SQTLNHNVMYWYQQKSSQAPKLLFHYYDKDFNNEADTPDNFQ | |
| | constant | SRRPNTSFCFLDIRSPGLGDAAMYLCATSREGLAADTQYFGP | |
| | domain | GTRLTVLEDLKNVFPPEVAVFEPSEAEISHTQKATLVCLATG | |
| | | FYPDHVELSWWVNGKEVHSGVCTDPQPLKEQPALNDSRYCLS | |
| | | SRLRVSATFWQNPRNHFRCQVQFYGLSENDEWTQDRAKPVTQ | |
| | | IVSAEAWGRADCGFTSESYQQGVLSATILYEILLGKATLYAV | |
| | | LVSALVLMAMVKRKDSRG | |

Patient 1

| | | | |
|---|---|---|---|
| Alpha (α) | CDR1α | VSNAYN | SEQ ID NO: 91 |
| direct | CDR2α | GSKP | SEQ ID NO: 92 |
| sequencing | CDR3α | CAAPNDYKLSF | SEQ ID NO: 93 |
| upon | Variable | MALQSTLGAVWLGLLLNSLWKVAESKDQVFQPSTVASSEGAV | SEQ ID NO: 94 |
| sorting | | VEIFCNHSVSNAYNFFWYLHFPGCAPRLLVKGSKPSQQGRYN | |
| | | MTYERFSSSLLILQVREADAAVYYCAAPNDYKLSFGAGTTVT | |
| | | VRAN | |
| | Full - with | MALQSTLGAVWLGLLLNSLWKVAESKDQVFQPSTVASSEGAV | SEQ ID NO: 95 |
| | TRAC constant | VEIFCNHSVSNAYNFFWYLHFPGCAPRLLVKGSKPSQQGRYN | |
| | domain | MTYERFSSSLLILQVREADAAVYYCAAPNDYKLSFGAGTTVT | |
| | | VRANIQNPDPAVYQLRDSKSSDKSVCLFTDFDSQTNVSQSKD | |
| | | SDVYITDKCVLDMRSMDFKSNSAVAWSNKSDFACANAFNNSI | |
| | | IPEDTFFPSPESSCDVKLVEKSFETDTNLNFQNLSVIGFRIL | |
| | | LLKVAGFNLLMTLRLWSS | |
| Beta (β) 1 | CDR1β | SEHNR | SEQ ID NO: 96 |
| direct | CDR2β | FQNEAQ | SEQ ID NO: 97 |
| sequencing | CDR3β | CASSSGLAFYEQYF | SEQ ID NO: 98 |
| upon | Variable | MGTSLLCWMALCLLGADHADTGVSQNPRHKITKRGQNVTFRC | SEQ ID NO: 99 |
| sorting | | DPISEHNRLYWYRQTLGQGPEFLTYFQNEAQLEKSRLLSDRF | |
| | | SAERPKGSFSTLEIQRTEQGDSAMYLCASSSGLAFYEQYFGP | |
| | | GTRLTVTE | |
| | Full - with | MGTSLLCWMALCLLGADHADTGVSQNPRHKITKRGQNVTFRC | SEQ ID NO: 100 |
| | TRBC1 | DPISEHNRLYWYRQTLGQGPEFLTYFQNEAQLEKSRLLSDRF | |
| | constant | SAERPKGSFSTLEIQRTEQGDSAMYLCASSSGLAFYEQYFGP | |
| | domain | GTRLTVTEDLNKVFPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |
| | Full - with | MGTSLLCWMALCLLGADHADTGVSQNPRHKITKRGQNVTFRC | SEQ ID NO: 101 |
| | TRBC2 | DPISEHNRLYWYRQTLGQGPEFLTYFQNEAQLEKSRLLSDRF | |
| | constant | SAERPKGSFSTLEIQRTEQGDSAMYLCASSSGLAFYEQYFGP | |
| | domain | GTRLTVTEDLKNVFPPEVAVFEPSEAEISHTQKATLVCLATG | |
| | | FYPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSESYQQGVLSATILYEILLGKATLYAV | |
| | | LVSALVLMAMVKRKDSRG | |
| Beta (β) 2 | CDR1β | SGHDN | SEQ ID NO: 102 |
| direct | CDR2β | FVKESK | SEQ ID NO: 103 |
| sequencing | CDR3P | CASSQLSGRDSYEQYF | SEQ ID NO: 104 |
| upon | Variable | MVSRLLSLVSLCLLGAKHIEAGVTQFPSHSVIEKGQTVTLRC | SEQ ID NO: 105 |
| sorting | | DPISGHDNLYWYRRVMGKEIKFLLHFVKESKQDESGMPNNRF | |
| | | LAERTGGTYSTLKVQPAELEDSGVYFCASSQLSGRDSYEQYF | |
| | | GPGTRLTVTE | |
| | Full - with | MVSRLLSLVSLCLLGAKHIEAGVTQFPSHSVIEKGQTVTLRC | SEQ ID NO: 106 |
| | TRBC1 | DPISGHDNLYWYRRVMGKEIKFLLHFVKESKQDESGMPNNRF | |
| | constant | LAERTGGTYSTLKVQPAELEDSGVYFCASSQLSGRDSYEQYF | |
| | domain | GPGTRLTVTEDLNKVFPPEVAVFEPSEAEISHTQKATLVCLA | |
| | | TGFFPDHVELSWWVNGKEVHSGVSTDPQPLKEQPALNDSRYC | |
| | | LSSRLRVSATFWQNPRNHFRCQVQFYGLSENDEWTQDRAKPV | |
| | | TQIVSAEAWGRADCGFTSVSYQQGVLSATILYEILLGKATLY | |
| | | AVLVSALVLMAMVKRKDF | |
| | Full - with | MVSRLLSLVSLCLLGAKHIEAGVTQFPSHSVIEKGQTVTLRC | SEQ ID NO: 107 |
| | TRBC2 | DPISGHDNLYWYRRVMGKEIKFLLHFVKESKQDESGMPNNRF | |
| | constant | LAERTGGTYSTLKVQPAELEDSGVYFCASSQLSGRDSYEQYF | |
| | domain | GPGTRLTVTEDLKNVFPPEVAVFEPSEAEISHTQKATLVCLA | |
| | | TGFYPDHVELSWWVNGKEVHSGVSTDPQPLKEQPALNDSRYC | |
| | | LSSRLRVSATFWQNPRNHFRCQVQFYGLSENDEWTQDRAKPV | |
| | | TQIVSAEAWGRADCGFTSESYQQGVLSATILYEILLGKATLY | |
| | | AVLVSALVLMAMVKRKDSRG | |
| Alpha (α) | CDR1α | VSGNPY | SEQ ID NO: 108 |
| 1 | CDR2α | YITGDNLV | SEQ ID NO: 109 |
| growing | CDR3α | CAVRDGGATNKLIF | SEQ ID NO: 110 |
| colony | Variable | MASAPISMLAMLFTLSGLRAQSVAQPEDQVNVAEGNPLTVKC | SEQ ID NO: 111 |
| | | TYSVSGNPYLFWYVQYPNRGLQFLLKYITGDNLVKGSYGFEA | |
| | | EFNKSQTSFHLKKPSALVSDSALYFCAVRDGGATNKLIFGTG | |
| | | TLLAVQPN | |
| | Full - with | MASAPISMLAMLFTLSGLRAQSVAQPEDQVNVAEGNPLTVKC | SEQ ID NO: 112 |
| | TRAC constant | TYSVSGNPYLFWYVQYPNRGLQFLLKYITGDNLVKGSYGFEA | |
| | domain | EFNKSQTSFHLKKPSALVSDSALYFCAVRDGGATNKLIFGTG | |
| | | TLLAVQPNIQNPDPAVYQLRDSKSSDKSVCLFTDFDSQTNVS | |
| | | QSKDSDVYITDKCVLDMRSMDFKSNSAVAWSNKSDFACANAF | |
| | | NNSIIPEDTFFPSPESSCDVKLVEKSFETDTNLNFQNLSVIG | |
| | | FRILLLKVAGFNLLMTLRLWSS | |
| Alpha (α) | CDR1α | NIATNDY | SEQ ID NO: 113 |
| 2 | CDR2α | GYKTK | SEQ ID NO: 114 |
| growing | CDR3α | CLVGGYTGGFKTIF | SEQ ID NO: 115 |
| colony | Variable | MRQVARVIVFLTLSTLSLAKTTQPISMDSYEGQEVNITCSHN | SEQ ID NO: 116 |
| | | NIATNDYITWYQQFPSQGPRFIIQGYKTKVTNEVASLFIPAD | |
| | | RKSSTLSLPRVSLSDTAVYYCLVGGYTGGFKTIFGAGTRLFV | |
| | | KAN | |
| | Full - with | MRQVARVIVFLTLSTLSLAKTTQPISMDSYEGQEVNITCSHN | SEQ ID NO: 117 |
| | TRAC constant | NIATNDYITWYQQFPSQGPRFIIQGYKTKVTNEVASLFIPAD | |
| | domain | RKSSTLSLPRVSLSDTAVYYCLVGGYTGGFKTIFGAGTRLFV | |
| | | KANTQNPDPAVYQLRDSKSSDKSVCLFTDFDSQTNVSQSKDS | |
| | | DVYITDKTVLDMRSMDFKSNSAVAWSNKSDFACANAFNNSII | |
| | | PEDTFFPSPESSCDVKLVEKSFETDTNLNFQNLSVIGFRILL | |
| | | LKVAGFNLLMTLRLWSS | |
| Beta (β) | CDR1β | MNHEY | SEQ ID NO: 118 |
| growing | CDR2β | SMNVEV | SEQ ID NO: 119 |
| colony | CDR3β | CASSTLGGELFF | SEQ ID NO: 120 |
| | Variable | MGPQLLGYVVLCLLGAGPLEAQVTQNPRYLITVTGKKLTVTC | SEQ ID NO: 121 |
| | | SQNMNHEYMSWYRQDPGLGLRQIYYSMNVEVTDKGDVPEGYK | |
| | | VSRKEKRNFPLILESPSPNQTSLYFCASSTLGGELFFGEGSR | |
| | | LTVLE | |
| | Full - with | MGPQLLGYVVLCLLGAGPLEAQVTQNPRYLITVTGKKLTVTC | SEQ ID NO: 122 |
| | TRBC1 | SQNMNHEYMSWYRQDPGLGLRQIYYSMNVEVTDKGDVPEGYK | |
| | constant | VSRKEKRNFPLILESPSPNQTSLYFCASSTLGGELFFGEGSR | |
| | domain | LTVLEDLNKVFPPEVAVFEPSEAEISHTQKATLVCLATGFFP | |
| | | DHVELSWWVNGKEVHSGVSTDPQPLKEQPALNDSRYCLSSRL | |
| | | RVSATFWQNPRNHFRCQVQFYGLSENDEWTQDRAKPVTQIVS | |
| | | AEAWGRADCGFTSVSYQQGVLSATILYEILLGKATLYAVLVS | |
| | | ALVLMAMVKRKDF | |
| | Full - with | MGPQLLGYVVLCLLGAGPLEAQVTQNPRYLITVTGKKLTVTC | SEQ ID NO: 123 |
| | TRBC2 | SQNMNHEYMSWYRQDPGLGLRQIYYSMNVEVTDKGDVPEGYK | |
| | constant | VSRKEKRNFPLILESPSPNQTSLYFCASSTLGGELFFGEGSR | |
| | domain | LTVLEDLKNVFPPEVAVFEPSEAEISHTQKATLVCLATGFYP | |
| | | DHVELSWWVNGKEVHSGVSTDPQPLKEQPALNDSRYCLSSRL | |
| | | RVSATFWQNPRNHFRCQVQFYGLSENDEWTQDRAKPVTQIVS | |
| | | AEAWGRADCGFTSESYQQGVLSATILYEILLGKATLYAVLVS | |
| | | ALVLMAMVKRKDSRG | |

Patient 2

| | | | |
|---|---|---|---|
| Alpha (α) | CDR1α | SSVSVY | SEQ ID NO: 124 |
| 1 | CDR2α | YLSGSTLV | SEQ ID NO: 125 |
| | CDR3α | CAVTLLSIEPSAGGYQKVTF | SEQ ID NO: 126 |
| | Variable | MLLLLVPAFQVIFTLGGTRAQSVTQLDSQVPVFEEAPVELRC | SEQ ID NO: 127 |
| | | NYSSSVSVYLFWYVQYPNQGLQLLLKYLSGSTLVESINGFEA | |
| | | EFNKSQTSFHLRKPSVHISDTAEYFCAVTLLSIEPSAGGYQK | |
| | | VTFGIGTKLQVIPN | |
| | Full - with | MLLLLVPAFQVIFTLGGTRAQSVTQLDSQVPVFEEAPVELRC | SEQ ID NO: 128 |
| | TRAC constant | NYSSSVSVYLFWYVQYPNQGLQLLLKYLSGSTLVESINGFEA | |
| | domain | EFNKSQTSFHLRKPSVHISDTAEYFCAVTLLSIEPSAGGYQK | |
| | | VTFGIGTKLQVIPNIQNPDPAVYQLRDSKSSDKSVCLFTDFD | |
| | | SQTNVSQSKDSDVYITDKCVLDMRSMDFKSNSAVAWSNKSDF | |
| | | ACANAFNNSIIPEDTFFPSPESSCDVKLVEKSFETDTNLNFQ | |
| | | NLSVIGFRILLLKVAGFNLLMTLRLWSS | |
| Alpha (α) | CDR1α | DSASNY | SEQ ID NO: 129 |
| 2 | CDR2α | IRSNVGE | SEQ ID NO: 130 |
| | CDR3α | CAATSRDDMRF | SEQ ID NO: 131 |
| | Variable | MTSIRAVFIFLWLQLDLVNGENVEQHPSTLSVQEGDSAVIKC | SEQ ID NO: 132 |
| | | TYSDSASNYFPWYKQELGKRPQLIIDIRSNVGEKKDQRIAVT | |
| | | LNKTAKHFSLHITETQPEDSAVYFCAATSRDDMRFGAGTRLT | |
| | | VKPN | |
| | Full - with | MTSIRAVFIFLWLQLDLVNGENVEQHPSTLSVQEGDSAVIKC | SEQ ID NO: 133 |
| | TRAC constant | TYSDSASNYFPWYKQELGKRPQLIIDIRSNVGEKKDQRIAVT | |
| | domain | LNKTAKHFSLHITETQPEDSAVYFCAATSRDDMRFGAGTRLT | |
| | | VKPNIQNPDPAVYQLRDSKSSDKSVCLFTDFDSQTNVSQSKD | |
| | | SDVYITDKCVLDMRSMDFKSNSAVAWSNKSDFACANAFNNSI | |
| | | IPEDTFFPSPESSCDVKLVEKSFETDTNLNFQNLSVIGFRIL | |
| | | LLKVAGFNLLMTLRLWSS | |
| Beta (β) 1 | CDR1β | SEHNR | SEQ ID NO: 134 |
| | CDR2β | FQNEAQ | SEQ ID NO: 135 |
| | CDR3β | CASSLEGRAMPRDSHQETQYF | SEQ ID NO: 136 |
| | Variable | MGTSLLCWMALCLLGADHADTGVSQNPRHKITKRGQNVTFRC | SEQ ID NO: 137 |
| | | DPISEHNRLYWYRQTLGQGPEFLTYFQNEAQLEKSRLLSDRF | |
| | | SAERPKGSFSTLEIQRTEQGDSAMYLCASSLEGRAMPRDSHQ | |
| | | ETQYFGPGTRLLVLE | |
| | Full - with | MGTSLLCWMALCLLGADHADTGVSQNPRHKITKRGQNVTFRC | SEQ ID NO: 138 |
| | TRBC1 | DPISEHNRLYWYRQTLGQGPEFLTYFQNEAQLEKSRLLSDRF | |
| | constant | SAERPKGSFSTLEIQRTEQGDSAMYLCASSLEGRAMPRDSHQ | |
| | domain | ETQYFGPGTRLLVLEDLNKVFPPEVAVFEPSEAEISHTQKAT | |
| | | LVCLATGFFPDHVELSWWVNGKEVHSGVSTDPQPLKEQPALN | |
| | | DSRYCLSSRLRVSATFWQNPRNHFRCQVQFYGLSENDEWTQD | |
| | | RAKPVTQIVSAEAWGRADCGFTSVSYQQGVLSATILYEILLG | |
| | | KATLYAVLVSALVLMAMVKRKDF | |
| | Full - with | MGTSLLCWMALCLLGADHADTGVSQNPRHKITKRGQNVTFRC | SEQ ID NO: 139 |
| | TRBC2 | DPISEHNRLYWYRQTLGQGPEFLTYFQNEAQLEKSRLLSDRF | |
| | constant | SAERPKGSFSTLEIQRTEQGDSAMYLCASSLEGRAMPRDSHQ | |
| | domain | ETQYFGPGTRLLVLEDLKNVFPPEVAVFEPSEAEISHTQKAT | |
| | | LVCLATGFYPDHVELSWWVNGKEVHSGVSTDPQPLKEQPALN | |
| | | DSRYCLSSRLRVSATFWQNPRNHFRCQVQFYGLSENDEWTQD | |
| | | RAKPVTQIVSAEAWGRADCGFTSESYQQGVLSATILYEILLG | |
| | | KATLYAVLVSALVLMAMVKRKDSRG | |
| Beta (β) 2 | CDR1β | LNHNV | SEQ ID NO: 140 |
| | CDR2β | YYDKDF | SEQ ID NO: 141 |
| | CDR3β | CATSWGLNEQYF | SEQ ID NO: 142 |
| | Variable | MGPGLLHWMALCLLGTGHGDAMVIQNPRYQVTQFGKPVTLSC | SEQ ID NO: 143 |
| | | SQTLNHNVMYWYQQKSSQAPKLLFHYYDKDFNNEADTPDNFQ | |
| | | SRRPNTSFCFLDIRSPGLGDAAMYLCATSWGLNEQYFGPGTR | |
| | | LTVTE | |
| | Full - with | MGPGLLHWMALCLLGTGHGDAMVIQNPRYQVTQFGKPVTLSC | SEQ ID NO: 144 |
| | TRBC1 | SQTLNHNVMYWYQQKSSQAPKLLFHYYDKDFNNEADTPDNFQ | |
| | constant | SRRPNTSFCFLDIRSPGLGDAAMYLCATSWGLNEQYFGPGTR | |
| | domain | LTVTEDLNKVFPPEVAVFEPSEAEISHTQKATLVCLATGFFP | |
| | | DHVELSWWVNGKEVHSGVSTDPQPLKEQPALNDSRYCLSSRL | |
| | | RVSATFWQNPRNHFRCQVQFYGLSENDEWTQDRAKPVTQIVS | |
| | | AEAWGRADCGFTSVSYQQGVLSATILYEILLGKATLYAVLVS | |
| | | ALVLMAMVKRKDF | |
| | Full - with | MGPGLLHWMALCLLGTGHGDAMVIQNPRYQVTQFGKPVTLSC | SEQ ID NO: 145 |
| | TRBC2 | SQTLNHNVMYWYQQKSSQAPKLLFHYYDKDFNNEADTPDNFQ | |
| | constant | SRRPNTSFCFLDIRSPGLGDAAMYLCATSWGLNEQYFGPGTR | |
| | domain | LTVTEDLKNVFPPEVAVFEPSEAEISHTQKATLVCLATGFYP | |
| | | DHVELSWWVNGKEVHSGVSTDPQPLKEQPALNDSRYCLSSRL | |
| | | RVSATFWQNPRNHFRCQVQFYGLSENDEWTQDRAKPVTQIVS | |
| | | AEAWGRADCGFTSESYQQGVLSATILYEILLGKATLYAVLVS | |
| | | ALVLMAMVKRKDSRG | |

Patient 3

| | | | |
|---|---|---|---|
| Alpha (α) | CDR1α | TRDTTYY | SEQ ID NO: 146 |
| 1 | CDR2α | RNSFDEQN | SEQ ID NO: 147 |
| | CDR3α | CALPDKVIF | SEQ ID NO: 148 |
| | Variable | MLTASLLRAVIASICVVSSMAQKVTQAQTEISVVEKEDVTLD | SEQ ID NO: 149 |
| | | CVYETRDTTYYLFWYKQPPSGELVFLIRRNSFDEQNEISGRY | |
| | | SWNFQKSTSSFNFTITASQVVDSAVYFCALPDKVIFGPGTSL | |
| | | SVIPN | |
| | Full - with | MLTASLLRAVIASICVVSSMAQKVTQAQTEISVVEKEDVTLD | SEQ ID NO: 150 |
| | TRAC constant | CVYETRDTTYYLFWYKQPPSGELVFLIRRNSFDEQNEISGRY | |
| | domain | SWNFQKSTSSFNFTITASQVVDSAVYFCALPDKVIFGPGTSL | |
| | | SVIPNIQNPDPAVYQLRDSKSSDKSVCLFTDFDSQTNVSQSK | |
| | | DSDVYITDKCVLDMRSMDFKSNSAVAWSNKSDFACANAFNNS | |
| | | IIPEDTFFPSPESSCDVKLVEKSFETDTNLNFQNLSVIGFRI | |
| | | LLLKVAGFNLLMTLRLWSS | |
| Alpha (α) | CDR1α | SIFNT | SEQ ID NO: 151 |
| 2 | CDR2α | LYKAGEL | SEQ ID NO: 152 |
| | CDR3α | CAGLYATNKLIF | SEQ ID NO: 153 |
| | Variable | MLLEHLLIILWMQLTWVSGQQLNQSPQSMFIQEGEDVSMNCT | SEQ ID NO: 154 |
| | | SSSIFNTWLWYKQEPGEGPVLLIALYKAGELTSNGRLTAQFG | |
| | | ITRKDSFLNISASIPSDVGIYFCAGLYATNKLIFGTGTLLAV | |
| | | QPN | |
| | Full - with | MLLEHLLIILWMQLTWVSGQQLNQSPQSMFIQEGEDVSMNCT | SEQ ID NO: 155 |
| | TRAC constant | SSSIFNTWLWYKQEPGEGPVLLIALYKAGELTSNGRLTAQFG | |
| | domain | ITRKDSFLNISASIPSDVGIYFCAGLYATNKLIFGTGTLLAV | |
| | | QPNIQNPDPAVYQLRDSKSSDKSVCLFTDFDSQTNVSQSKDS | |
| | | DVYITDKCVLDMRSMDFKSNSAVAWSNKSDFACANAFNNSII | |
| | | PEDTFFPSPESSCDVKLVEKSFETDTNLNFQNLSVIGFRILL | |
| | | LKVAGFNLLMTLRLWSS | |
| Beta (β) | CDR1β | SGDLS | SEQ ID NO: 156 |
| | CDR2β | YYNGEE | SEQ ID NO: 157 |
| | CDR3β | CASSVSAGSTGELFF | SEQ ID NO: 158 |
| | Variable | MGFRLLCCVAFCLLGAGPVDSGVTQTPKHLITATGQRVTLRC | SEQ ID NO: 159 |
| | | SPRSGDLSVYWYQQSLDQGLQFLIQYYNGEERAKGNILERFS | |
| | | AQQFPDLHSELNLSSLELGDSALYFCASSVSAGSTGELFFGE | |
| | | GSRLTVLE | |
| | Full - with | MGFRLLCCVAFCLLGAGPVDSGVTQTPKHLITATGQRVTLRC | SEQ ID NO: 160 |
| | TRBC1 | SPRSGDLSVYWYQQSLDQGLQFLIQYYNGEERAKGNILERFS | |
| | constant | AQQFPDLHSELNLSSLELGDSALYFCASSVSAGSTGELFFGE | |
| | domain | GSRLTVLEDLNKVFPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |
| | Full - with | MGFRLLCCVAFCLLGAGPVDSGVTQTPKHLITATGQRVTLRC | SEQ ID NO: 161 |
| | TRBC2 | SPRSGDLSVYWYQQSLDQGLQFLIQYYNGEERAKGNILERFS | |
| | constant | AQQFPDLHSELNLSSLELGDSALYFCASSVSAGSTGELFFGE | |
| | domain | GSRLTVLEDLKNVFPPEVAVFEPSEAEISHTQKATLVCLATG | |
| | | FYPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSESYQQGVLSATILYEILLGKATLYAV | |
| | | LVSALVLMAMVKRKDSRG | |

Accordingly, the present invention provides isolated polypeptides comprising one or more amino acid sequences selected from the group consisting of SEQ ID NOs: 1-55, 91-161, 182-191, 194-203 and 214-217, fragments, variants and homologues thereof.

In one aspect, the invention provides a TCR comprising a TCR alpha chain sequence selected from the group consisting of the HD12-HD15 alpha chain sequences of Table 1, and a TCR beta chain sequence independently selected from the group consisting of the HD12-HD15 beta chain sequences of Table 1.

In one aspect, the invention provides a TCR comprising a TCR alpha chain sequence selected from the group consisting of the Patient 1, Patient 2 or Patient 3 alpha chain sequences of Table 1, and a TCR beta chain sequence independently selected from the group consisting of the Patient 1, Patient 2 or Patient 3 beta chain sequences of Table 1.

In one aspect, the invention provides a TCR comprising a TCR alpha chain sequence selected from the group consisting of the HD12, HD13, HD14, HD15, Patient 1, Patient 2 or Patient 3 alpha chain sequences of Table 1, and a TCR beta chain sequence independently selected from the group consisting of the HD12, HD13, HD14, HD15, Patient 1, Patient 2 or Patient 3 beta chain sequences of Table 1.

In alternative embodiments, sequences of the full TCR beta chains referred to in Table 1 may be replaced with corresponding sequences below.

Donor: HD12

| | | | |
|---|---|---|---|
| Beta(β) | Full - with | MDTWLVCWAIFSLLKAGLTEPEVTQTPSHQVTQMGQEVILRC | SEQ ID NO: 222 |
| | TRBC1 | VPISNHLYFYWYRQILGQKVEFLVSFYNNEISEKSEIFDDQF | |
| | constant | SVERPDGSNFTLKIRSTKLEDSAMYFCASSEAGLSYEQYFGP | |
| | domain | GTRLTVTEDLNKVFPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |

In some embodiments, SEQ ID NO: 10 may be replaced with SEQ ID NO: 222.

Donor: HD13

| | | | |
|---|---|---|---|
| Beta(β) | Full - with | MASLLFFCGAFYLLGTGSMDADVTQTPRNRITKTGKRIMLEC | SEQ ID NO: 223 |
| | TRBC1 | SQTKGHDRMYWYRQDPGLGLRLIYYSFDVKDINKGEISDGYS | |
| | constant | VSRQAQAKFSLSLESAIPNQTALYFCATGQATQETQYFGPGT | |
| | domain | RLLVLEDLNKVFPPEVAVFEPSEAEISHTQKATLVCLATGFF | |
| | | PDHVELSWWVNGKEVHSGVSTDPQPLKEQPALNDSRYCLSSR | |
| | | LRVSATFWQNPRNHFRCQVQFYGLSENDEWTQDRAKPVTQIV | |
| | | SAEAWGRADCGFTSVSYQQGVLSATILYEILLGKATLYAVLV | |
| | | SALVLMAMVKRKDF | |

In some embodiments, SEQ ID NO: 21 may be replaced with SEQ ID NO: 223.

Donor: HD14

| | | | |
|---|---|---|---|
| Beta(β) | Full-with | MGFRLLCCVAFCLLGAGPVDSGVTQTPKHLITATGQRVTLRC | SEQ ID |
| | TRBC1 | SPRSGDLSVYWYQQSLDQGLQFLIQYYNGEERAKGNILERFS | NO: 224 |
| | constant | AQQFPDLHSELNLSSLELGDSALYFCASGRGDTEAFFGQGTR | |
| | domain | LTVVEDLNKVFPPEVAVFEPSEAEISHTQKATLVCLATGFFP | |
| | | DHVELSWWVNGKEVHSGVSTDPQPLKEQPALNDSRYCLSSRL | |
| | | RVSATFWQNPRNHERCQVQFYGLSENDEWTQDRAKPVTQIVS | |
| | | AEAWGRADCGFTSVSYQQGVLSATILYEILLGKATLYAVLVS | |
| | | ALVLMAMVKRKDF | |

In some embodiments, SEQ ID NO: 32 may be replaced with SEQ ID NO: 224.

Donor: HD15

| | | | |
|---|---|---|---|
| Beta(β) | Full-with | MGCRLLCCAVLCLLGAGELVPMETGVTQTPRHLVMGMTNKKS | SEQ ID |
| S4 | TRBC1 | LKCEQHLGHNAMYWYKQSAKKPLELMFVYSLEERVENNSVPS | NO: 225 |
| population | constant | RFSPECPNSSHLFLHLHTLQPEDSALYLCASSQEEGAVYGYT | |
| | domain | FGSGTRLTVVEDLNKVEPPEVAVFEPSEAEISHTQKATLVCL | |
| | | ATGFFPDHVELSWWVNGKEVHSGVSTDPQPLKEQPALNDSRY | |
| | | CLSSRLRVSATFWQNPRNHERCQVQFYGLSENDEWTQDRAKP | |
| | | VTQIVSAEAWGRADCGFTSVSYQQGVLSATILYEILLGKATL | |
| | | YAVLVSALVLMAMVKRKDF | |
| Beta(β) | Full-with | MGPGLLHWMALCLLGTGHGDAMVIQNPRYQVTQFGKPVTLSC | SEQ ID |
| S1 IFNg | TRBC1 | SQTLNHNVMYWYQQKSSQAPKLLEHYYDKDENNEADTPDNFQ | NO: 226 |
| enriched | constant | SRRPNTSFCFLDIRSPGLGDAAMYLCATSREGLAADTQYFGP | |
| population | domain | GTRLTVLEDLNKVEPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHERCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |

In some embodiments, SEQ ID NO: 43 may be replaced with SEQ ID NO: 225.

In some embodiments, SEQ ID NO: 54 may be replaced with SEQ ID NO: 226.

Patient 1

| | | | |
|---|---|---|---|
| Beta (β) | Full-with | MGTSLLCWMALCLLGADHADTGVSQNPRHKITKRGQNVTFRC | SEQ ID |
| 1 direct | TRBC1 | DPISEHNRLYWYRQTLGQGPEFLTYFQNEAQLEKSRLLSDRF | NO: 227 |
| sequencing | constant | SAERPKGSFSTLEIQRTEQGDSAMYLCASSSGLAFYEQYFGP | |
| upon | domain | GTRLTVTEDLNKVEPPEVAVFEPSEAEISHTQKATLVCLATG | |
| sorting | | FFPDHVELSWWVNGKEVHSGVSTDPQPLKEQPALNDSRYCLS | |
| | | SRLRVSATFWQNPRNHERCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |
| Beta (β) 2 | Full-with | MVSRLLSLVSLCLLGAKHIEAGVTQFPSHSVIEKGQTVTLRC | SEQ ID |
| direct | TRBC1 | DPISGHDNLYWYRRVMGKEIKELLHEVKESKQDESGMPNNRF | NO: 228 |
| sequencing | constant | LAERTGGTYSTLKVQPAELEDSGVYFCASSQLSGRDSYEQYF | |
| upon | domain | GPGTRLTVTEDLNKVEPPEVAVFEPSEAEISHTQKATLVCLA | |
| sorting | | TGFFPDHVELSWWVNGKEVHSGVSTDPQPLKEQPALNDSRYC | |
| | | LSSRLRVSATFWQNPRNHERCQVQFYGLSENDEWTQDRAKPV | |
| | | TQIVSAEAWGRADCGFTSVSYQQGVLSATILYEILLGKATLY | |
| | | AVLVSALVLMAMVKRKDF | |
| Alpha (α) | Full-with | MGPQLLGYVVLCLLGAGPLEAQVTQNPRYLITVTGKKLTVTC | SEQ ID |
| 2 | TRBC1 | SQNMNHEYMSWYRQDPGLGLRQIYYSMNVEVTDKGDVPEGYK | NO: 229 |
| growing | constant | VSRKEKRNFPLILESPSPNQTSLYFCASSTLGGELFFGEGSR | |
| colony | domain | LTVLEDLNKVFPPEVAVFEPSEAEISHTQKATLVCLATGFFP | |
| | | DHVELSWWVNGKEVHSGVSTDPQPLKEQPALNDSRYCLSSRL | |
| | | RVSATFWQNPRNHERCQVQFYGLSENDEWTQDRAKPVTQIVS | |
| | | AEAWGRADCGFTSVSYQQGVLSATILYEILLGKATLYAVLVS | |
| | | ALVLMAMVKRKDF | |

In some embodiments, SEQ ID NO: 100 may be replaced with SEQ ID NO: 227.

In some embodiments, SEQ ID NO: 106 may be replaced with SEQ ID NO: 228.

In some embodiments, SEQ ID NO: 122 may be replaced with SEQ ID NO: 229.

Patient 2

| | | | |
|---|---|---|---|
| Beta (β) 1 | Full-with | MGTSLLCWMALCLLGADHADTGVSQNPRHKITKRGQNVTFRC | SEQ ID |
| | TRBC1 | DPISEHNRLYWYRQTLGQGPEFLTYFQNEAQLEKSRLLSDRF | NO: 230 |
| | constant | SAERPKGSFSTLEIQRTEQGDSAMYLCASSLEGRAMPRDSHQ | |
| | domain | ETQYFGPGTRLLVLEDLNKVFPPEVAVFEPSEAEISHTQKAT | |
| | | LVCLATGFFPDHVELSWWVNGKEVHSGVSTDPQPLKEQPALN | |
| | | DSRYCLSSRLRVSATFWQNPRNHERCQVQFYGLSENDEWTQD | |
| | | RAKPVTQIVSAEAWGRADCGFTSVSYQQGVLSATILYEILLG | |
| | | KATLYAVLVSALVLMAMVKRKDF | |
| Beta (β) 2 | Full-with | MGPGLLHWMALCLLGTGHGDAMVIQNPRYQVTQFGKPVTLSC | SEQ ID |
| | TRBC1 | SQTLNHNVMYWYQQKSSQAPKLLFHYYDKDFNNEADTPDNFQ | |
| | constant | SRRPNTSFCFLDIRSPGLGDAAMYLCATSWGLNEQYFGPGTR | NO: 231 |
| | domain | LTVTEDLNKVFPPEVAVFEPSEAEISHTQKATLVCLATGFFP | |
| | | DHVELSWWVNGKEVHSGVSTDPQPLKEQPALNDSRYCLSSRL | |
| | | RVSATFWQNPRNHERCQVQFYGLSENDEWTQDRAKPVTQIVS | |
| | | AEAWGRADCGFTSVSYQQGVLSATILYEILLGKATLYAVLVS | |
| | | ALVLMAMVKRKDF | |

In some embodiments, SEQ ID NO: 138 may be replaced with SEQ ID NO: 230.

In some embodiments, SEQ ID NO: 144 may be replaced with SEQ ID NO: 231.

Patient 3

| | | | |
|---|---|---|---|
| Beta(β) | Full-with | MGFRLLCCVAFCLLGAGPVDSGVTQTPKHLITATGQRVTLRC | SEQ ID |
| | TRBC1 | SPRSGDLSVYWYQQSLDQGLQFLIQYYNGEERAKGNILERFS | NO: 232 |
| | constant | AQQFPDLHSELNLSSLELGDSALYFCASSVSAGSTGELFFGE | |
| | domain | GSRLTVLEDLNKVFPPEVAVFEPSEAEISHTQKATLVCLATG | |
| | | FFPDHVELSWWVNGKEVHSGVSTDPQPLKEQPALNDSRYCLS | |
| | | SRLRVSATFWQNPRNHFRCQVQFYGLSENDEWTQDRAKPVTQ | |
| | | IVSAEAWGRADCGFTSVSYQQGVLSATILYEILLGKATLYAV | |
| | | LVSALVLMAMVKRKDF | |

In some embodiments, SEQ ID NO: 160 may be replaced with SEQ ID NO: 232.

Reduced Mispairing and Improved TCR Expression

The TCR of the invention may be expressed in a T-cell to alter the antigen specificity of the T-cell. TCR-transduced T-cells may express at least two TCR alpha and two TCR beta chains. While the endogenous TCR alpha/beta chains form a receptor that is self-tolerant, the introduced TCR alpha/beta chains form a receptor with defined specificity for the given target antigen.

However, TCR gene therapy requires sufficient expression of transferred TCRs. Transferred TCR might be diluted by the presence of the endogeneous TCR, resulting in suboptimal expression of the tumor specific TCR. Furthermore, mispairing between endogenous and introduced chains may occur to form novel receptors, which might display unexpected specificities for self-antigens and cause autoimmune damage when transferred into patients.

Hence, several strategies have been explored to reduce the risk of mispairing between endogenous and introduced TCR chains. Mutations of the TCR alpha/beta interface is one strategy currently employed to reduce unwanted mispairing. For example, the introduction of a cysteine in the constant domains of the alpha and beta chain allows the formation of a disulfide bond and enhances the pairing of the introduced chains while reducing mispairing with wild type chains.

Accordingly, the TCRs of the invention may comprise one or more mutations at the α chain/β chain interface, such that when the α chain and the β chain are expressed in a T-cell, the frequency of mispairing between said chains and endogenous TCR α and β chains is reduced. In one embodiment, the one or more mutations introduce a cysteine residue into the constant region domain of each of the α chain and the β chain, wherein the cysteine residues are capable of forming a disulphide bond between the α chain and the β chain.

Such modification of TCRs is described in for example Boulter, J. M et al. (2003) Protein Engineering 16: 707-711 and Kuball, L. et al. (2007) Blood 109: 2331-8.

In one embodiment, the one or more mutations are at amino acid positions selected from those disclosed in Table 1 of Boulter, J. M et al. (2003) Protein Engineering 16: 707-711. In one embodiment, the one or more mutations are a substitution of one or more of the following amino acids with cysteine:

| TRAC residue | TRBC residue |
|---|---|
| Threonine 48 | Serine 57 |
| Threonine 45 | Serine 77 |
| Serine 61 | Serine 57 |
| Leucine 50 | Serine 57 |
| Tyrosine 10 | Serine 17 |
| Serine 15 | Valine 13 |
| Serine 15 | Glutamate 15 |
| Threonine 45 | Aspartate 59 |
| Leucine 12 | Serine 17 |
| Serine 61 | Arginine 79 |
| Leucine 12 | Phenylalanine 14 |
| Valine 22 | Phenylalanine 14 |
| Tyrosine 43 | Leucine 63 |

In a one embodiment, the TCR comprises one or more of the following groups of mutations:
(a) a substitution of threonine at position 48 of the TCR alpha constant gene with cysteine; and/or a substitution of serine at position 57 of the TR beta constant gene with cysteine;
(b) a substitution of threonine at position 45 of the TCR alpha constant gene with cysteine; and/or a substitution of serine at position 77 of the TCR beta constant gene with cysteine;
(c) a substitution of serine at position 61 of the TCR alpha constant gene with cysteine; and/or a substitution of serine at position 57 of the TCR beta constant gene with cysteine;
(d) a substitution of leucine at position 50 of the TCR alpha constant gene with cysteine; and/or a substitution of serine at position 57 of the TCR beta constant gene with cysteine;
(e) a substitution of tyrosine at position 10 of the TCR alpha constant gene with cysteine; and/or a substitution of serine at position 17 of the TCR beta constant gene with cysteine;
(f) a substitution of serine at position 15 of the TCR alpha constant gene with cysteine; and/or a substitution of valine at position 13 of the TCR beta constant gene with cysteine;
(g) a substitution of serine at position 15 of the TCR alpha constant gene with cysteine; and/or a substitution of glutamate at position 15 of the TCR beta constant gene with cysteine;
(h) a substitution of threonine at position 45 of the TCR alpha constant gene with cysteine; and/or a substitution of aspartate at position 59 of the TCR beta constant gene with cysteine;
(i) a substitution of leucine 12 at position 48 of the TCR alpha constant gene with cysteine; and/or a substitution of serine at position 17 of the TCR beta constant gene with cysteine;
(j) a substitution of serine at position 61 of the TCR alpha constant gene with cysteine; and/or a substitution of arginine at position 79 of the TCR beta constant gene with cysteine;
(k) a substitution of leucine at position 12 of the TCR alpha constant gene with cysteine; and/or a substitution of phenylalanine at position 14 of the TCR beta constant gene with cysteine;
(l) a substitution of valine at position 22 of the TCR alpha constant gene with cysteine; and/or a substitution of phenylalanine at position 14 of the TCR beta constant gene with cysteine; and/or
(m) a substitution of tyrosine at position 43 of the TCR alpha constant gene with cysteine; and/or a substitution of leucine at position 63 of the TCR beta constant gene with cysteine.

In a preferred embodiment, the TCR comprises a substitution of threonine at position 48 of the TCR alpha constant gene with cysteine; and/or a substitution of serine at position 57 of the TCR beta constant gene with cysteine.

Another strategy to reduce mispairing relies on the introduction of polynucleotide sequences encoding siRNA, added to the genes encoding for the tumor specific TCR α and or β chains, and designed to limit the expression of the endogenous TCR genes (Okamoto S. Cancer research 69, 9003-9011, 2009).

Accordingly, the vector or polynucleotide encoding the TCRs of the invention may comprise one or more siRNA or other agents aimed at limiting or abrogating the expression of the endogenous TCR genes.

It is also possible to combine artificial nucleases, such as zinc finger nucleases (ZFN), transcription activator-like effector nucleases (TALEN) or CRISPR/Cas systems, designed to target the constant regions of the endogenous genes, e.g. TCR genes (TRAC and, or TRBC), to obtain the permanent disruption of the endogenous TCR alpha and/or beta chain genes, thus allowing full expression of the tumor specific TCR and thus reducing or abrogating the risk of TCR mispairing. This process, known as the TCR gene editing proved superior to TCR gene transfer in vitro and in vivo (Provasi E., Genovese P., Nature Medicine May; 18(5):807-15; 2012; Mastaglio S. et al. (2017) Blood 130: 606-618).

Accordingly, the TCRs of the invention may be used to edit T cell specificity by TCR disruption and genetic addition of the tumor specific TCR.

In addition, the genome editing technology allows targeted integration of a expression cassette, comprising a polynucleotide encoding a TCR of the invention, and optionally one or more promoter regions and/or other expression control sequences, into an endogenous gene disrupted by the artificial nucleases (Lombardo A., Nature biotechnology 25, 1298-1306; 2007).

Accordingly, the TCRs of the invention may be used to edit T-cell specificity by targeted integration of a polynucleotide encoding a TCR of the invention at a genomic region. The integration may be targeted by an artificial nuclease.

A cell, such as a T cell, may therefore be genetically engineered to comprise a TCR of the invention. In addition, a cell, such as a T cell, may be genetically edited by gene disruption, for example TRAC and/or TRBC disruption obtained by, for example, CRISPR/Cas9, or by targeted integration, for example of an expression cassette into an endogenous gene (such as an endogenous gene involved in antigen specificity, persistence, expansion, activity, resistance to exhaustion/senescence/inhibitory signals, homing capacity or other T-cell functions).

Another strategy developed to increase expression of the transferred TCR and to reduce TCR mispairing consists in “murinization,” which replaces the human TCR α and TCR β constant regions (e.g. the TRAC, TRBC1 and TRBC2 regions) by their murine counterparts. Murizination of TCR constant regions is described in, for example, Sommermeyer and Uckert J Immunol; 2010 (184:6223-6231). Accordingly, the TCRs of the invention may be murinized.

Isolated Polynucleotide

The invention relates to an isolated polynucleotide encoding a TCR of the invention or a part thereof, such as the α chain and/or the β chain, a variable domain or a portion thereof.

The isolated polynucleotide may be double or single stranded, and may be RNA or DNA.

It will be understood by a skilled person that numerous different polynucleotides can encode the same polypeptide as a result of the degeneracy of the genetic code. In addition, it is to be understood that the skilled person may, using routine techniques, make nucleotide substitutions, additions or deletions that do not affect the polypeptide sequence encoded by the polynucleotides of the invention to reflect the codon usage of any particular host organism in which the polypeptides of the invention are to be expressed.

The polynucleotides described herein may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Examples of nucleotide sequences encoding TCRs according to the invention are provided in the Table 2.

TABLE 2

| Donor | Chain | Nucleotide sequence | SEQ ID NO |
|---|---|---|---|
| HD12 | α (with | ATGTCACTTTCTAGCCTGCTGAAGGTGGTCACAGCTTCACTGTGGCTAGGACCTG | SEQ ID NO: 56 |
| | TRAC) | GCATTGCCCAGAAGATAACTCAAACCCAACCAGGAATGTTCGTGCAGGAAAAGGA | |
| | | GGCTGTGACTCTGGACTGCACATATGACACCAGTGATCAAAGTTATGGTCTATTC | |
| | | TGGTACAAGCAGCCCAGCAGTGGGGAAATGATTTTTCTTATTTATCAGGGGTCTT | |
| | | ATGACGAGCAAAATGCAACAGAAGGTCGCTACTCATTGAATTTCCAGAAGGCAAG | |
| | | AAAATCCGCCAACCTTGTCATCTCCGCTTCACAACTGGGGGACTCAGCAATGTAT | |
| | | TTCTGTGCAATGAGAACGGGAGGAGGTGCTGACGGACTCACCTTTGGCAAAGGGA | |
| | | CTCATCTAATCATCCAGCCCTATATCCAGAACCCTGACCCTGCCGTGTACCAGCT | |
| | | GAGAGACTCTAAATCCAGTGACAAGTCTGTCTGCCTATTCACCGATTTTGATTCT | |
| | | CAAACAAATGTGTCACAAAGTAAGGATTCTGATGTGTATATCACAGACAAAACTG | |
| | | TGCTAGACATGAGGTCTATGGACTTCAAGAGCAACAGTGCTGTGGCCTGGAGCAA | |
| | | CAAATCTGACTTTGCATGTGCAAACGCCTTCAACAACAGCATTATTCCAGAAGAC | |
| | | ACCTTCTTCCCCAGCCCAGAAAGTTCCTGTGATGTCAAGCTGGTCGAGAAAAGCT | |
| | | TTGAAACAGATACGAACCTAAACTTTCAAAACCTGTCAGTGATTGGGTTCCGAAT | |
| | | CCTCCTCCTGAAAGTGGCCGGGTTTAATCTGCTCATGACGCTGCGGCTGTGGTCC | |
| | | AGC | |
| | α (with | ATGAGCCTGTCTAGCCTGCTGAAGGTGGTCACAGCCAGCCTGTGGCTCG | SEQ ID NO: 204 |
| | TRAC) | GACCTGGAATCGCCCAGAAGATCACCCAGACACAGCCCGGCATGTTCGT | |
| | | GCAAGAGAAAGAAGCCGTGACACTGGACTGCACCTACGACACCAGCGAT | |
| | | CAGAGCTACGGCCTGTTCTGGTACAAGCAGCCTAGCAGCGGCGAGATGA | |
| | | TCTTCCTGATCTACCAGGGCAGCTACGACGAGCAGAATGCCACCGAGGG | |
| | | CAGATACAGCCTGAACTTCCAGAAGGCCCGGAAGTCCGCCAACCTGGTC | |
| | | ATTTCTGCTTCTCAGCTGGGCGACAGCGCCATGTACTTTTGCGCCATGA | |
| | | GAACAGGCGGCGGAGCCGATGGACTGACATTTGGCAAGGGCACCCACCT | |
| | | GATCATCCAGCCTTACATTCAGAACCCCGATCCTGCCGTGTACCAGCTG | |
| | | AGGGATAGCAAGAGCAGCGACAAGAGCGTGTGCCTGTTCACCGACTTCG | |
| | | ACAGCCAGACCAACGTGTCCCAGAGCAAGGACAGCGACGTGTACATCAC | |
| | | CGATAAGTGCGTGCTGGACATGCGGAGCATGGACTTCAAGAGCAACAGC | |
| | | GCCGTGGCCTGGTCCAACAAGAGCGATTTCGCCTGCGCCAACGCCTTCA | |
| | | ACAACAGCATTATCCCCGAGGACACATTCTTCCCAAGTCCTGAGAGCAG | |
| | | CTGCGACGTGAAGCTGGTGGAAAAGAGCTTCGAGACAGACACCAACCTC | |
| | | AATTTCCAGAACCTGAGCGTGATCGGCTTCCGGATCCTGCTGCTGAAAG | |
| | | TGGCCGGCTTCAACCTGCTGATGACCCTGAGACTGTGGTCCAGC | |
| | β (with | ATGGATACCTGGCTCGTATGCTGGGCAATTTTTAGTCTCTTGAAAGCAGGACTCA | SEQ ID NO: 57 |
| | TRBC1) | CAGAACCTGAAGTCACCCAGACTCCCAGCCATCAGGTCACACAGATGGGACAGGA | |
| | | AGTGATCTTGCGCTGTGTCCCCATCTCTAATCACTTATACTTCTATTGGTACAGA | |
| | | CAAATCTTGGGGCAGAAAGTCGAGTTTCTGGTTTCCTTTTATAATAATGAAATCT | |
| | | CAGAGAAGTCTGAAATATTCGATGATCAATTCTCAGTTGAAAGGCCTGATGGATC | |
| | | AAATTTCACTCTGAAGATCCGGTCCACAAAGCTGGAGGACTCAGCCATGTACTTC | |
| | | TGTGCCAGCAGTGAAGCGGGACTTTCCTACGAGCAGTACTTCGGGCCGGGCACCA | |
| | | GGCTCACGGTCACAGAGGACCTGAACAAGGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTTCCCCGACCACGTGGAGCTGAGCTGGTGGGTGAATGGGAAGG | |
| | | AGGTGCACAGTGGGGTCAGCACGGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCCGTCACCCAGATCGTCAGCGCCGA | |
| | | GGCCTGGGGTAGAGCAGACTGTGGCTTTACCTCGGTGTCCTACCAGCAAGGGGTC | |
| | | CTGTCTGCCACCATCCTCTATGAGATCCTGCTAGGGAAGGCCACCCTGTATGCTG | |
| | | TGCTGGTCAGCGCCCTTGTGTTGATGGCCATGGTCAAGAGAAAGGATTTC | |
| | β (with | ATGGATACCTGGCTCGTATGCTGGGCAATTTTTAGTCTCTTGAAAGCAGGACTCA | SEQ ID NO: 58 |
| | TRBC2) | CAGAACCTGAAGTCACCCAGACTCCCAGCCATCAGGTCACACAGATGGGACAGGA | |
| | | AGTGATCTTGCGCTGTGTCCCCATCTCTAATCACTTATACTTCTATTGGTACAGA | |
| | | CAAATCTTGGGGCAGAAAGTCGAGTTTCTGGTTTCCTTTTATAATAATGAAATCT | |
| | | CAGAGAAGTCTGAAATATTCGATGATCAATTCTCAGTTGAAAGGCCTGATGGATC | |
| | | AAATTTCACTCTGAAGATCCGGTCCACAAAGCTGGAGGACTCAGCCATGTACTTC | |
| | | TGTGCCAGCAGTGAAGCGGGACTTTCCTACGAGCAGTACTTCGGGCCGGGCACCA | |
| | | GGCTCACGGTCACAGAGGACCTGAAAAACGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTACCCCGACCACGTGGAGCTGAGCTGGTGGGTGAATGGGAAGG | |
| | | AGGTGCACAGTGGGGTCAGCACAGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCTGTCACCCAGATCGTCAGCGCCGA | |
| | | GGCCTGGGGTAGAGCAGACTGTGGCTTCACCTCCGAGTCTTACCAGCAAGGGGTC | |
| | | CTGTCTGCCACCATCCTCTATGAGATCTTGCTAGGGAAGGCCACCTTGTATGCCG | |
| | | TGCTGGTCAGTGCCCTCGTGCTGATGGCCATGGTCAAGAGAAAGGATTCCAGAGG | |
| | | C | |
| | β (with | ATGGATACTTGGCTTGTGTGCTGGGCCATCTTCAGCCTGCTGAAGGCCG | SEQ ID NO: 205 |
| | TRBC2) | GACTGACAGAGCCCGAAGTGACACAGACACCCAGCCACCAAGTGACCCA | |
| | | GATGGGCCAAGAAGTGATCCTGCGCTGCGTGCCCATCAGCAACCACCTG | |
| | | TACTTCTACTGGTACAGACAGATCCTGGGCCAGAAAGTGGAATTCCTGG | |
| | | TGTCCTTCTACAACAACGAGATCAGCGAGAAGTCCGAGATCTTCGACGA | |
| | | CCAGTTCAGCGTGGAAAGACCCGACGGCAGCAACTTCACCCTGAAGATC | |
| | | AGAAGCACCAAGCTCGAGGACAGCGCCATGTACTTTTGCGCCTCTTCTG | |
| | | AAGCCGGCCTGAGCTACGAGCAGTACTTTGGCCCTGGCACCAGACTGAC | |
| | | CGTGACCGAGGATCTGAAGAACGTGTTCCCACCTGAGGTGGCCGTGTTC | |
| | | GAACCTTCTGAGGCCGAGATCTCTCACACCCAGAAAGCCACACTCGTGT | |
| | | GTCTGGCCACCGGCTTCTATCCCGATCACGTGGAACTGTCTTGGTGGGT | |
| | | CAACGGCAAAGAGGTGCACAGCGGCGTCTGTACCGATCCTCAGCCTCTG | |
| | | AAAGAGCAGCCCGCTCTGAACGACAGCAGATACTGCCTGAGCAGCAGAC | |
| | | TGAGAGTGTCCGCCACCTTCTGGCAGAACCCCAGAAACCACTTCAGATG | |
| | | CCAGGTGCAGTTCTACGGCCTGTCCGAGAACGATGAGTGGACCCAGGAT | |
| | | AGAGCCAAGCCTGTGACTCAGATCGTGTCTGCCGAAGCCTGGGGCAGAG | |
| | | CCGATTGTGGCTTTACCAGCGAGAGCTACCAGCAGGGCGTGCTGTCTGC | |
| | | CACAATCCTGTACGAGATCCTGCTGGGCAAAGCCACTCTGTACGCCGTG | |
| | | CTGGTGTCTGCCCTGGTGCTGATGGCCATGGTCAAGCGGAAGGATAGCA | |
| | | GGGGC | |
| HD13 | α (with | ATGAAGTTGGTGACAAGCATTACTGTACTCCTATCTTTGGGTATTATGGGTGATG | SEQ ID NO: 59 |
| | TRAC) | CTAAGACCACACAGCCAAATTCAATGGAGAGTAACGAAGAAGAGCCTGTTCACTT | |
| | | GCCTTGTAACCACTCCACAATCAGTGGAACTGATTACATACATTGGTATCGACAG | |
| | | CTTCCCTCCCAGGGTCCAGAGTACGTGATTCATGGTCTTACAAGCAATGTGAACA | |
| | | ACAGAATGGCCTCTCTGGCAATCGCTGAAGACAGAAAGTCCAGTACCTTGATCCT | |
| | | GCACCGTGCTACCTTGAGAGATGCTGCTGTGTACTACTGCATCCTGAGTACCCGG | |
| | | GTCTGGGCTGGGAGTTACCAACTCACTTTCGGGAAGGGGACCAAACTCTCGGTCA | |
| | | TACCAAATATCCAGAACCCTGACCCTGCCGTGTACCAGCTGAGAGACTCTAAATC | |
| | | CAGTGACAAGTCTGTCTGCCTATTCACCGATTTTGATTCTCAAACAAATGTGTCA | |
| | | CAAAGTAAGGATTCTGATGTGTATATCACAGACAAAACTGTGCTAGACATGAGGT | |
| | | CTATGGACTTCAAGAGCAACAGTGCTGTGGCCTGGAGCAACAAATCTGACTTTGC | |
| | | ATGTGCAAACGCCTTCAACAACAGCATTATTCCAGAAGACACCTTCTTCCCCAGC | |
| | | CCAGAAAGTTCCTGTGATGTCAAGCTGGTCGAGAAAAGCTTTGAAACAGATACGA | |
| | | ACCTAAACTTTCAAAACCTGTCAGTGATTGGGTTCCGAATCCTCCTCCTGAAAGT | |
| | | GGCCGGGTTTAATCTGCTCATGACGCTGCGGCTGTGGTCCAGC | |
| | α (with | ATGAAGCTGGTCACCAGCATCACCGTGCTGCTGAGCCTGGGCATTATGG | SEQ ID NO: 206 |
| | TRAC) | GCGACGCCAAGACCACACAGCCCAACAGCATGGAAAGCAACGAAGAGGA | |
| | | ACCCGTGCATCTGCCCTGCAACCACAGCACAATCAGCGGCACCGACTAC | |
| | | ATCCACTGGTACAGACAGCTGCCCAGCCAGGGACCTGAGTATGTGATCC | |
| | | ACGGCCTGACCAGCAACGTGAACAACAGAATGGCCAGCCTGGCTATCGC | |
| | | CGAGGACAGAAAGAGCAGCACCCTGATCCTGCACAGAGCCACACTGAGA | |
| | | GATGCCGCCGTGTACTACTGCATCCTGAGCACAAGAGTGTGGGCCGGCA | |
| | | GCTACCAGCTGACATTTGGCAAGGGCACCAAGCTGAGCGTGATCCCCAA | |
| | | CATTCAGAACCCCGATCCTGCCGTGTACCAGCTGCGGGATAGCAAGAGC | |
| | | AGCGACAAGAGCGTGTGCCTGTTCACCGACTTCGACAGCCAGACCAACG | |
| | | TGTCCCAGAGCAAGGACAGCGACGTGTACATCACCGATAAGTGCGTGCT | |
| | | GGACATGCGGAGCATGGACTTCAAGAGCAACAGCGCCGTGGCCTGGTCC | |
| | | AACAAGAGCGATTTCGCCTGCGCCAACGCCTTCAACAACAGCATTATCC | |
| | | CTGAGGACACATTCTTCCCAAGTCCTGAGAGCAGCTGCGACGTGAAACT | |
| | | GGTGGAAAAGAGCTTCGAGACAGACACCAACCTGAACTTCCAGAACCTG | |
| | | TCCGTGATCGGCTTCCGGATCCTGCTGCTGAAAGTGGCCGGCTTCAACC | |
| | | TGCTGATGACCCTGAGACTGTGGTCCAGC | |
| | β (with | ATGGCCTCCCTGCTCTTCTTCTGTGGGGCCTTTTATCTCCTGGGAACAGGGTCCA | SEQ ID NO: 60 |
| | TRBC1) | TGGATGCTGATGTTACCCAGACCCCAAGGAATAGGATCACAAAGACAGGAAAGAG | |
| | | GATTATGCTGGAATGTTCTCAGACTAAGGGTCATGATAGAATGTACTGGTATCGA | |
| | | CAAGACCCAGGACTGGGCCTACGGTTGATCTATTACTCCTTTGATGTCAAAGATA | |
| | | TAAACAAAGGAGAGATCTCTGATGGATACAGTGTCTCTCGACAGGCACAGGCTAA | |
| | | ATTCTCCCTGTCCCTAGAGTCTGCCATCCCCAACCAGACAGCTCTTTACTTCTGT | |
| | | GCCACCGGCCAGGCGACCCAAGAGACCCAGTACTTCGGGCCAGGCACGCGGCTCC | |
| | | TGGTGCTCGAGGACCTGAACAAGGTGTTCCCACCCGAGGTCGCTGTGTTTGAGCC | |
| | | ATCAGAAGCAGAGATCTCCCACACCCAAAAGGCCACACTGGTGTGCCTGGCCACA | |
| | | GGCTTCTTCCCCGACCACGTGGAGCTGAGCTGGTGGGTGAATGGGAAGGAGGTGC | |
| | | ACAGTGGGGTCAGCACGGACCCGCAGCCCCTCAAGGAGCAGCCCGCCCTCAATGA | |
| | | CTCCAGATACTGCCTGAGCAGCCGCCTGAGGGTCTCGGCCACCTTCTGGCAGAAC | |
| | | CCCCGCAACCACTTCCGCTGTCAAGTCCAGTTCTACGGGCTCTCGGAGAATGACG | |
| | | AGTGGACCCAGGATAGGGCCAAACCCGTCACCCAGATCGTCAGCGCCGAGGCCTG | |
| | | GGGTAGAGCAGACTGTGGCTTTACCTCGGTGTCCTACCAGCAAGGGGTCCTGTCT | |
| | | GCCACCATCCTCTATGAGATCCTGCTAGGGAAGGCCACCCTGTATGCTGTGCTGG | |
| | | TCAGCGCCCTTGTGTTGATGGCCATGGTCAAGAGAAAGGATTTC | |
| | β (with | ATGGCCTCCCTGCTCTTCTTCTGTGGGGCCTTTTATCTCCTGGGAACAGGGTCCA | SEQ ID NO: 61 |
| | TRBC2) | TGGATGCTGATGTTACCCAGACCCCAAGGAATAGGATCACAAAGACAGGAAAGAG | |
| | | GATTATGCTGGAATGTTCTCAGACTAAGGGTCATGATAGAATGTACTGGTATCGA | |
| | | CAAGACCCAGGACTGGGCCTACGGTTGATCTATTACTCCTTTGATGTCAAAGATA | |
| | | TAAACAAAGGAGAGATCTCTGATGGATACAGTGTCTCTCGACAGGCACAGGCTAA | |
| | | ATTCTCCCTGTCCCTAGAGTCTGCCATCCCCAACCAGACAGCTCTTTACTTCTGT | |
| | | GCCACCGGCCAGGCGACCCAAGAGACCCAGTACTTCGGGCCAGGCACGCGGCTCC | |
| | | TGGTGCTCGAGGACCTGAAAAACGTGTTCCCACCCGAGGTCGCTGTGTTTGAGCC | |
| | | ATCAGAAGCAGAGATCTCCCACACCCAAAAGGCCACACTGGTGTGCCTGGCCACA | |
| | | GGCTTCTACCCCGACCACGTGGAGCTGAGCTGGTGGGTGAATGGGAAGGAGGTGC | |
| | | ACAGTGGGGTCAGCACAGACCCGCAGCCCCTCAAGGAGCAGCCCGCCCTCAATGA | |
| | | CTCCAGATACTGCCTGAGCAGCCGCCTGAGGGTCTCGGCCACCTTCTGGCAGAAC | |
| | | CCCCGCAACCACTTCCGCTGTCAAGTCCAGTTCTACGGGCTCTCGGAGAATGACG | |
| | | AGTGGACCCAGGATAGGGCCAAACCTGTCACCCAGATCGTCAGCGCCGAGGCCTG | |
| | | GGGTAGAGCAGACTGTGGCTTCACCTCCGAGTCTTACCAGCAAGGGGTCCTGTCT | |
| | | GCCACCATCCTCTATGAGATCTTGCTAGGGAAGGCCACCTTGTATGCCGTGCTGG | |
| | | TCAGTGCCCTCGTGCTGATGGCCATGGTCAAGAGAAAGGATTCCAGAGGC | |
| | β (with | ATGGCTTCTCTTCTGTTTTTCTGCGGCGCCTTCTACCTGCTCGGCACCG | SEQ ID NO: 207 |
| | TRBC2) | GATCTATGGACGCCGACGTTACCCAGACACCACGGAACAGAATCACCAA | |
| | | GACCGGCAAGCGGATCATGCTGGAATGCAGCCAGACCAAGGGCCACGAC | |
| | | CGGATGTACTGGTACAGACAGGATCCAGGACTGGGCCTGAGACTGATCT | |
| | | ACTACAGCTTCGACGTGAAGGACATCAACAAGGGCGAGATCAGCGACGG | |
| | | CTACAGCGTGTCAAGACAGGCCCAGGCCAAGTTCAGCCTGAGCCTGGAA | |
| | | AGCGCTATCCCCAACCAGACAGCCCTGTACTTTTGTGCCACCGGCCAGG | |
| | | CCACACAAGAGACACAGTATTTCGGCCCTGGCACCAGACTGCTGGTGCT | |
| | | GGAAGATCTGAAGAACGTGTTCCCACCTGAGGTGGCCGTGTTCGAGCCT | |
| | | TCTGAGGCCGAGATCTCTCACACCCAGAAAGCCACACTCGTGTGTCTGG | |
| | | CCACCGGCTTCTATCCCGATCACGTGGAACTGTCTTGGTGGGTCAACGG | |
| | | CAAAGAGGTGCACAGCGGCGTCTGTACCGATCCTCAGCCTCTGAAAGAG | |
| | | CAGCCCGCTCTGAACGACAGCAGATACTGCCTGAGCAGCAGACTGAGAG | |
| | | TGTCCGCCACCTTCTGGCAGAACCCCAGAAACCACTTCAGATGCCAGGT | |
| | | GCAGTTCTACGGCCTGAGCGAGAACGATGAGTGGACCCAGGATAGAGCC | |
| | | AAGCCTGTGACACAGATCGTGTCTGCCGAAGCCTGGGGCAGAGCCGATT | |
| | | GTGGCTTTACCAGCGAGAGCTACCAGCAGGGCGTGCTGTCTGCCACAAT | |
| | | CCTGTACGAGATCCTGCTGGGCAAAGCCACTCTGTACGCCGTGCTGGTG | |
| | | TCTGCCCTGGTGCTGATGGCCATGGTCAAGCGGAAGGATAGCAGGGGC | |
| HD14 | α | ATGATGAAATCCTTGAGAGTTTTACTGGTGATCCTGTGGCTTCAGTTAAGCTGGG | SEQ ID NO: 62 |
| | (TRAV12- | TTTGGAGCCAACAGAAGGAGGTGGAGCAGGATCCTGGACCACTCAGTGTTCCAGA | |
| | 3*01) | GGGAGCCATTGTTTCTCTCAACTGCACTTACAGCAACAGTGCTTTTCAATACTTC | |
| | (with | ATGTGGTACAGACAGTATTCCAGAAAAGGCCCTGAGTTGCTGATGTACACATACT | |
| | TRAC) | CCAGTGGTAACAAAGAAGATGGAAGGTTTACAGCACAGGTCGATAAATCCAGCAA | |
| | | GTATATCTCCTTGTTCATCAGAGACTCACAGCCCAGTGATTCAGCCACCTACCTC | |
| | | TGTGCCTCAGGAGGAGGTGCTGACGGACTCACCTTTGGCAAAGGGACTCATCTAA | |
| | | TCATCCAGCCCTATATCCAGAACCCTGACCCTGCCGTGTACCAGCTGAGAGACTC | |
| | | TAAATCCAGTGACAAGTCTGTCTGCCTATTCACCGATTTTGATTCTCAAACAAAT | |
| | | GTGTCACAAAGTAAGGATTCTGATGTGTATATCACAGACAAAACTGTGCTAGACA | |
| | | TGAGGTCTATGGACTTCAAGAGCAACAGTGCTGTGGCCTGGAGCAACAAATCTGA | |
| | | CTTTGCATGTGCAAACGCCTTCAACAACAGCATTATTCCAGAAGACACCTTCTTC | |
| | | CCCAGCCCAGAAAGTTCCTGTGATGTCAAGCTGGTCGAGAAAAGCTTTGAAACAG | |
| | | ATACGAACCTAAACTTTCAAAACCTGTCAGTGATTGGGTTCCGAATCCTCCTCCT | |
| | | GAAAGTGGCCGGGTTTAATCTGCTCATGACGCTGCGGCTGTGGTCCAGC | |
| | α | ATGAAATCCTTGAGAGTTTTACTAGTGATCCTGTGGCTTCAGTTGAGCTGGGTTT | SEQ ID NO: 192 |
| | (TRAV12- | GGAGCCAACAGAAGGAGGTGGAGCAGAATTCTGGACCCCTCAGTGTTCCAGAGGG | |
| | 2*01) | AGCCATTGCCTCTCTCAACTGCACTTACAGTGACCGAGGTTCCCAGTCCTTCTTC | |
| | (with | TGGTACAGACAATATTCTGGGAAAAGCCCTGAGTTGATAATGTTCATATACTCCA | |
| | TRAC) | ATGGTGACAAAGAAGATGGAAGGTTTACAGCACAGCTCAATAAAGCCAGCCAGTA | |
| | | TGTTTCTCTGCTCATCAGAGACTCCCAGCCCAGTGATTCAGCCACCTACCTCTGT | |
| | | GCCTCAGGAGGAGGTGCTGACGGACTCACCTTTGGCAAAGGGACTCATCTAATCA | |
| | | TCCAGCCCTATATCCAGAACCCTGACCCTGCCGTGTACCAGCTGAGAGACTCTAA | |
| | | ATCCAGTGACAAGTCTGTCTGCCTATTCACCGATTTTGATTCTCAAACAAATGTG | |
| | | TCACAAAGTAAGGATTCTGATGTGTATATCACAGACAAAACTGTGCTAGACATGA | |
| | | GGTCTATGGACTTCAAGAGCAACAGTGCTGTGGCCTGGAGCAACAAATCTGACTT | |
| | | TGCATGTGCAAACGCCTTCAACAACAGCATTATTCCAGAAGACACCTTCTTCCCC | |
| | | AGCCCAGAAAGTTCCTGTGATGTCAAGCTGGTCGAGAAAAGCTTTGAAACAGATA | |
| | | CGAACCTAAACTTTCAAAACCTGTCAGTGATTGGGTTCCGAATCCTCCTCCTGAA | |
| | | AGTGGCCGGGTTTAATCTGCTCATGACGCTGCGGCTGTGGTCCAGC | |
| | α | ATGATGAAATCCTTGAGAGTTTTACTAGTGATCCTGTGGCTTCAGTTGAGCTGGG | SEQ ID NO: 193 |
| | (TRAV12- | TTTGGAGCCAACAGAAGGAGGTGGAGCAGAATTCTGGACCCCTCAGTGTTCCAGA | |
| | 2*02) | GGGAGCCATTGCCTCTCTCAACTGCACTTACAGTGACCGAGGTTCCCAGTCCTTC | |
| | (with | TTCTGGTACAGACAATATTCTGGGAAAAGCCCTGAGTTGATAATGTCCATATACT | |
| | TRAC) | CCAATGGTGACAAAGAAGATGGAAGGTTTACAGCACAGCTCAATAAAGCCAGCCA | |
| | | GTATGTTTCTCTGCTCATCAGAGACTCCCAGCCCAGTGATTCAGCCACCTACCTC | |
| | | TGTGCCTCAGGAGGAGGTGCTGACGGACTCACCTTTGGCAAAGGGACTCATCTAA | |
| | | TCATCCAGCCCTATATCCAGAACCCTGACCCTGCCGTGTACCAGCTGAGAGACTC | |
| | | TAAATCCAGTGACAAGTCTGTCTGCCTATTCACCGATTTTGATTCTCAAACAAAT | |
| | | GTGTCACAAAGTAAGGATTCTGATGTGTATATCACAGACAAAACTGTGCTAGACA | |
| | | TGAGGTCTATGGACTTCAAGAGCAACAGTGCTGTGGCCTGGAGCAACAAATCTGA | |
| | | CTTTGCATGTGCAAACGCCTTCAACAACAGCATTATTCCAGAAGACACCTTCTTC | |
| | | CCCAGCCCAGAAAGTTCCTGTGATGTCAAGCTGGTCGAGAAAAGCTTTGAAACAG | |
| | | ATACGAACCTAAACTTTCAAAACCTGTCAGTGATTGGGTTCCGAATCCTCCTCCT | |
| | | GAAAGTGGCCGGGTTTAATCTGCTCATGACGCTGCGGCTGTGGTCCAGC | |
| | α | ATGATGAAGTCCCTGCGGGTGCTGCTGGTCATCCTGTGGCTGCAACTGA | SEQ ID NO: 208 |
| | TRAV12- | GCTGGGTCTGGTCCCAGCAGAAAGAGGTGGAACAGGACCCTGGACCTCT | |
| | 3*01 WT | GTCTGTTCCTGAGGGCGCCATCGTGTCCCTGAACTGCACCTACAGCAAC | |
| | (with | AGCGCCTTCCAGTACTTCATGTGGTACAGACAGTACAGCCGGAAGGGCC | |
| | TRAC) | CCGAGCTGCTGATGTACACATACAGCAGCGGCAACAAAGAGGACGGCCG | |
| | | GTTTACAGCCCAGGTGGACAAGAGCAGCAAGTACATCTCCCTGTTCATC | |
| | | CGGGACAGCCAGCCTAGCGATAGCGCCACATATCTGTGTGCATCTGGCG | |
| | | GCGGAGCCGATGGCCTGACATTTGGAAAGGGCACCCACCTGATCATCCA | |
| | | GCCTTACATTCAGAACCCCGATCCTGCCGTGTACCAGCTGAGAGACAGC | |
| | | AAGTCCAGCGACAAGAGCGTGTGCCTGTTCACCGACTTCGACAGCCAGA | |
| | | CCAACGTGTCCCAGAGCAAGGACAGCGACGTGTACATTACCGATAAGTG | |
| | | CGTGCTGGACATGCGGAGCATGGACTTCAAGAGCAACTCCGCCGTGGCC | |
| | | TGGTCCAACAAGAGCGATTTCGCCTGCGCCAACGCCTTCAACAACAGCA | |
| | | TTATCCCCGAGGACACATTCTTCCCAAGTCCTGAGAGCAGCTGCGACGT | |
| | | GAAGCTGGTGGAAAAGAGCTTCGAGACAGACACCAACCTGAACTTCCAG | |
| | | AACCTGAGCGTGATCGGCTTCCGGATCCTGCTGCTGAAAGTGGCCGGCT | |
| | | TCAACCTGCTGATGACCCTGAGACTGTGGTCCAGC | |
| | α | ATGAAGTCCCTGAGAGTGCTGCTGGTCATCCTGTGGCTGCAGCTGTCTT | SEQ ID NO: 209 |
| | TRAV12- | GGGTCTGGTCCCAGCAGAAAGAGGTGGAACAGAACAGCGGCCCTCTGTC | |
| | 2*01 WT | TGTTCCTGAAGGCGCTATCGCCAGCCTGAACTGCACCTACAGCGATAGA | |
| | (with | GGCAGCCAGAGCTTCTTCTGGTACAGACAGTACAGCGGCAAGAGCCCCG | |
| | TRAC) | AGCTGATCATGTTCATCTACAGCAACGGCGACAAAGAGGACGGCCGGTT | |
| | | TACAGCCCAGCTGAACAAGGCCAGCCAGTACGTGTCCCTGCTGATCAGA | |
| | | GATAGCCAGCCTAGCGACAGCGCCACCTACCTTTGTGCATCTGGTGGCG | |
| | | GAGCCGATGGCCTGACATTTGGCAAGGGAACCCACCTGATCATCCAGCC | |
| | | TTACATTCAGAACCCCGATCCTGCCGTGTACCAGCTGAGAGACAGCAAG | |
| | | AGCAGCGACAAGAGCGTGTGCCTGTTCACCGACTTCGACAGCCAGACCA | |
| | | ACGTGTCCCAGAGCAAGGACAGCGACGTGTACATCACCGATAAGTGCGT | |
| | | GCTGGACATGCGGAGCATGGACTTCAAGAGCAACAGCGCCGTGGCCTGG | |
| | | TCCAACAAGAGCGATTTCGCCTGCGCCAACGCCTTCAACAACAGCATTA | |
| | | TCCCCGAGGACACATTCTTCCCAAGTCCTGAGAGCAGCTGCGACGTGAA | |
| | | GCTGGTGGAAAAGAGCTTCGAGACAGACACCAACCTGAACTTCCAGAAC | |
| | | CTGAGCGTGATCGGCTTCCGGATCCTGCTGCTGAAAGTGGCCGGCTTCA | |
| | | ACCTGCTGATGACCCTGAGACTGTGGTCCAGC | |
| | α | ATGAAGTCCCTGAGAGTGCTGCTGGTCATCCTGTGGCTGCAGCTGTCTT | SEQ ID NO: 210 |
| | TRAV12- | GGGTCTGGTCCCAGCAGAAAGAGGTGGAACAGAACAGCGGCCCTCTGTC | |
| | 2*01 mut | TGTTCCTGAAGGCGCTATCGCCAGCCTGAACTGCACCTACAGCGATAGA | |
| | (with | GGCAGCCAGAGCTTCTTCTGGTACAGACAGTACAGCGGCAAGAGCCCCG | |
| | TRAC) | AGCTGATCATGTTCATCTACAGCAACGGCGACAAAGAGGACGGCCGGTT | |
| | | TACAGCCCAGCTGAACAAGGCCAGCCAGTACGTGTCCCTGCTGATCAGA | |
| | | GATAGCCAGCCTAGCGACAGCGCCACCTACCTTTGTGCATCTGGTGGCG | |
| | | GAGCCGATGGCCTGACATTTGGCAAGGGAACCCACCTGATCATCCAGCC | |
| | | TTACATTCAGAACCCCGATCCTGCCGTGTACCAGCTGAGAGACAGCAAG | |
| | | AGCAGCGACAAGAGCGTGTGCCTGTTCACCGACTTCGACAGCCAGACAC | |
| | | AGGTGTCCCAGAGCAAGGACAGCGACGTGTACATCACCGATAAGTGCGT | |
| | | GCTGGACATGCGGAGCATGGACTTCAAGAGCAACAGCGCCGTGGCCTGG | |
| | | TCCAACAAGAGCGATTTCGCCTGCGCCAACGCCTTCAACAACAGCATTA | |
| | | TCCCCGAGGACACATTCTTCCCAAGTCCTGAGAGCAGCTGCGACGTGAA | |
| | | GCTGGTGGAAAAGAGCTTCGAGACAGACACCAACCTGAACTTCCAGAAC | |
| | | CTGAGCGTGATCGGCTTCCGGATCCTGCTGCTGAAAGTGGCCGGCTTCA | |
| | | ACCTGCTGATGACCCTGAGACTGTGGTCCAGC | |
| | α | ATGATGAAGTCCCTGCGGGTGCTGCTGGTCATCCTGTGGCTGCAACTGA | SEQ ID NO: 211 |
| | TRAV12- | GCTGGGTCTGGTCCCAGCAGAAAGAGGTGGAACAGAACAGCGGCCCTCT | |
| | 2*02 WT | GTCTGTTCCTGAAGGCGCTATCGCCAGCCTGAACTGCACCTACAGCGAT | |
| | (with | AGAGGCAGCCAGAGCTTCTTCTGGTACAGACAGTACAGCGGCAAGAGCC | |
| | TRAC) | CCGAGCTGATCATGAGCATCTACAGCAACGGCGACAAAGAGGACGGCCG | |
| | | GTTTACAGCCCAGCTGAACAAGGCCAGCCAGTACGTGTCCCTGCTGATC | |
| | | AGAGATAGCCAGCCTAGCGACAGCGCCACCTACCTTTGTGCATCTGGTG | |
| | | GCGGAGCCGATGGCCTGACATTTGGCAAGGGAACCCACCTGATCATCCA | |
| | | GCCTTACATTCAGAACCCCGATCCTGCCGTGTACCAGCTGAGAGACAGC | |
| | | AAGAGCAGCGACAAGAGCGTGTGCCTGTTCACCGACTTCGACAGCCAGA | |
| | | CCAACGTGTCCCAGAGCAAGGACAGCGACGTGTACATCACCGATAAGTG | |
| | | CGTGCTGGACATGCGGAGCATGGACTTCAAGAGCAACAGCGCCGTGGCC | |
| | | TGGTCCAACAAGAGCGATTTCGCCTGCGCCAACGCCTTCAACAACAGCA | |
| | | TTATCCCCGAGGACACATTCTTCCCAAGTCCTGAGAGCAGCTGCGACGT | |
| | | GAAGCTGGTGGAAAAGAGCTTCGAGACAGACACCAACCTGAACTTCCAG | |
| | | AACCTGAGCGTGATCGGCTTCCGGATCCTGCTGCTGAAAGTGGCCGGCT | |
| | | TCAACCTGCTGATGACCCTGAGACTGTGGTCCAGC | |
| | α | ATGATGAAGTCCCTGCGGGTGCTGCTGGTCATCCTGTGGCTGCAACTGA | SEQ ID NO: 212 |
| | TRAV12- | GCTGGGTCTGGTCCCAGCAGAAAGAGGTGGAACAGAACAGCGGCCCTCT | |
| | 2*02 mut | GTCTGTTCCTGAAGGCGCTATCGCCAGCCTGAACTGCACCTACAGCGAT | |
| | (with | AGAGGCAGCCAGAGCTTCTTCTGGTACAGACAGTACAGCGGCAAGAGCC | |
| | TRAC) | CCGAGCTGATCATGAGCATCTACAGCAACGGCGACAAAGAGGACGGCCG | |
| | | GTTTACAGCCCAGCTGAACAAGGCCAGCCAGTACGTGTCCCTGCTGATC | |
| | | AGAGATAGCCAGCCTAGCGACAGCGCCACCTACCTTTGTGCATCTGGTG | |
| | | GCGGAGCCGATGGCCTGACATTTGGCAAGGGAACCCACCTGATCATCCA | |
| | | GCCTTACATTCAGAACCCCGATCCTGCCGTGTACCAGCTGAGAGACAGC | |
| | | AAGAGCAGCGACAAGAGCGTGTGCCTGTTCACCGACTTCGACAGCCAGA | |
| | | CACAGGTGTCCCAGAGCAAGGACAGCGACGTGTACATCACCGATAAGTG | |
| | | CGTGCTGGACATGCGGAGCATGGACTTCAAGAGCAACAGCGCCGTGGCC | |
| | | TGGTCCAACAAGAGCGATTTCGCCTGCGCCAACGCCTTCAACAACAGCA | |
| | | TTATCCCCGAGGACACATTCTTCCCAAGTCCTGAGAGCAGCTGCGACGT | |
| | | GAAGCTGGTGGAAAAGAGCTTCGAGACAGACACCAACCTGAACTTCCAG | |
| | | AACCTGAGCGTGATCGGCTTCCGGATCCTGCTGCTGAAAGTGGCCGGCT | |
| | | TCAACCTGCTGATGACCCTGAGACTGTGGTCCAGC | |
| | β (with | ATGGGCTTCAGGCTCCTCTGCTGTGTGGCCTTTTGTCTCCTGGGAGCAGGCCCAG | SEQ ID NO: 63 |
| | TRBC1) | TGGATTCTGGAGTCACACAAACCCCAAAGCACCTGATCACAGCAACTGGACAGCG | |
| | | AGTGACGCTGAGATGCTCCCCTAGGTCTGGAGACCTCTCTGTGTACTGGTACCAA | |
| | | CAGAGCCTGGACCAGGGCCTCCAGTTCCTCATTCAGTATTATAATGGAGAAGAGA | |
| | | GAGCAAAAGGAAACATTCTTGAACGATTCTCCGCACAACAGTTCCCTGACTTGCA | |
| | | CTCTGAACTAAACCTGAGCTCTCTGGAGCTGGGGGACTCAGCTTTGTATTTCTGT | |
| | | GCCAGCGGGAGGGGGGACACTGAAGCTTTCTTTGGACAAGGCACCAGACTCACAG | |
| | | TTGTAGAGGACCTGAACAAGGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTTCCCCGACCACGTGGAGCTGAGCTGGTGGGTGAATGGGAAGGAGGTGCACA | |
| | | GTGGGGTCAGCACGGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCCGTCACCCAGATCGTCAGCGCCGAGGCCTGGGG | |
| | | TAGAGCAGACTGTGGCTTTACCTCGGTGTCCTACCAGCAAGGGGTCCTGTCTGCC | |
| | | ACCATCCTCTATGAGATCCTGCTAGGGAAGGCCACCCTGTATGCTGTGCTGGTCA | |
| | | GCGCCCTTGTGTTGATGGCCATGGTCAAGAGAAAGGATTTC | |
| | β (with | ATGGGCTTCAGGCTCCTCTGCTGTGTGGCCTTTTGTCTCCTGGGAGCAGGCCCAG | SEQ ID NO: 64 |
| | TRBC2) | TGGATTCTGGAGTCACACAAACCCCAAAGCACCTGATCACAGCAACTGGACAGCG | |
| | | AGTGACGCTGAGATGCTCCCCTAGGTCTGGAGACCTCTCTGTGTACTGGTACCAA | |
| | | CAGAGCCTGGACCAGGGCCTCCAGTTCCTCATTCAGTATTATAATGGAGAAGAGA | |
| | | GAGCAAAAGGAAACATTCTTGAACGATTCTCCGCACAACAGTTCCCTGACTTGCA | |
| | | CTCTGAACTAAACCTGAGCTCTCTGGAGCTGGGGGACTCAGCTTTGTATTTCTGT | |
| | | GCCAGCGGGAGGGGGGACACTGAAGCTTTCTTTGGACAAGGCACCAGACTCACAG | |
| | | TTGTAGAGGACCTGAAAAACGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTACCCCGACCACGTGGAGCTGAGCTGGTGGGTGAATGGGAAGGAGGTGCACA | |
| | | GTGGGGTCAGCACAGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCTGTCACCCAGATCGTCAGCGCCGAGGCCTGGGG | |
| | | TAGAGCAGACTGTGGCTTCACCTCCGAGTCTTACCAGCAAGGGGTCCTGTCTGCC | |
| | | ACCATCCTCTATGAGATCTTGCTAGGGAAGGCCACCTTGTATGCCGTGCTGGTCA | |
| | | GTGCCCTCGTGCTGATGGCCATGGTCAAGAGAAAGGATTCCAGAGGC | |
| | β | ATGGGTTTTAGACTGCTGTGCTGCGTGGCCTTCTGTCTGCTTGGAGCTG | SEQ ID NO: 213 |
| | TRAV12- | GCCCTGTGGATAGCGGCGTTACCCAGACACCTAAGCACCTGATCACAGC | |
| | 3*01 WT | CACAGGCCAGCGCGTGACCCTGAGATGTTCTCCTAGAAGCGGCGACCTG | |
| | (with | AGCGTGTACTGGTATCAGCAGTCTCTGGACCAGGGCCTGCAGTTCCTGA | |
| | TRBC2) | TCCAGTACTACAACGGCGAGGAAAGAGCCAAGGGCAACATCCTGGAACG | |
| | | GTTCAGCGCCCAGCAGTTCCCAGATCTGCACAGCGAGCTGAACCTGAGC | |
| | | AGCCTGGAACTGGGAGATAGCGCCCTGTACTTCTGTGCCAGCGGCAGAG | |
| | | GCGATACCGAGGCCTTTTTTGGCCAAGGCACCAGACTGACCGTGGTGGA | |
| | | AGATCTGAAGAACGTGTTCCCACCTGAGGTGGCCGTGTTCGAGCCTTCT | |
| | | GAGGCCGAGATCAGCCACACACAGAAAGCCACACTCGTGTGTCTGGCCA | |
| | | CCGGCTTCTATCCCGATCACGTGGAACTGTCTTGGTGGGTCAACGGCAA | |
| | | AGAGGTGCACAGCGGCGTCTGTACCGATCCTCAGCCTCTGAAAGAGCAG | |
| | | CCCGCTCTGAACGACAGCAGATACTGCCTGTCCAGCAGACTGAGAGTGT | |
| | | CCGCCACCTTCTGGCAGAACCCCAGAAACCACTTCAGATGCCAGGTGCA | |
| | | GTTCTACGGCCTGAGCGAGAACGATGAGTGGACCCAGGACAGAGCTAAG | |
| | | CCCGTGACACAGATCGTGTCTGCCGAAGCTTGGGGCAGAGCCGATTGTG | |
| | | GCTTTACCAGCGAGAGCTACCAGCAGGGCGTGCTGTCTGCCACAATCCT | |
| | | GTACGAGATCCTGCTGGGCAAAGCCACTCTGTACGCCGTGCTGGTGTCT | |
| | | GCCCTGGTGCTGATGGCCATGGTCAAGCGGAAGGATAGCAGGGGC | |
| HD15 | α (with | ATGAAATCCTTGAGAGTTTTACTAGTGATCCTGTGGCTTCAGTTGAGCTGGGTTT | SEQ ID NO: 65 |
| S4 | TRAC) | GGAGCCAACAGAAGGAGGTGGAGCAGAATTCTGGACCCCTCAGTGTTCCAGAGGG | |
| population | | AGCCATTGCCTCTCTCAACTGCACTTACAGTGACCGAGGTTCCCAGTCCTTCTTC | |
| | | TGGTACAGACAATATTCTGGGAAAAGCCCTGAGTTGATAATGTTCATATACTCCA | |
| | | ATGGTGACAAAGAAGATGGAAGGTTTACAGCACAGCTCAATAAAGCCAGCCAGTA | |
| | | TGTTTCTCTGCTCATCAGAGACTCCCAGCCCAGTGATTCAGCCACCTACCTCTGT | |
| | | GCCGTGATAGGGGGAACTGACAGCTGGGGGAAATTGCAGTTTGGAGCAGGGACCC | |
| | | AGGTTGTGGTCACCCCAGATATCCAGAACCCTGACCCTGCCGTGTACCAGCTGAG | |
| | | AGACTCTAAATCCAGTGACAAGTCTGTCTGCCTATTCACCGATTTTGATTCTCAA | |
| | | ACAAATGTGTCACAAAGTAAGGATTCTGATGTGTATATCACAGACAAAACTGTGC | |
| | | TAGACATGAGGTCTATGGACTTCAAGAGCAACAGTGCTGTGGCCTGGAGCAACAA | |
| | | ATCTGACTTTGCATGTGCAAACGCCTTCAACAACAGCATTATTCCAGAAGACACC | |
| | | TTCTTCCCCAGCCCAGAAAGTTCCTGTGATGTCAAGCTGGTCGAGAAAAGCTTTG | |
| | | AAACAGATACGAACCTAAACTTTCAAAACCTGTCAGTGATTGGGTTCCGAATCCT | |
| | | CCTCCTGAAAGTGGCCGGGTTTAATCTGCTCATGACGCTGCGGCTGTGGTCCAGC | |
| | α (with | ATGAAGTCCCTGAGAGTGCTGCTGGTCATCCTGTGGCTGCAGCTGTCTT | SEQ ID NO: 218 |
| | TRAC) | GGGTCTGGTCCCAGCAGAAAGAGGTGGAACAGAACAGCGGCCCTCTGTC | |
| | | TGTTCCTGAAGGCGCTATCGCCAGCCTGAACTGCACCTACAGCGATAGA | |
| | | GGCAGCCAGAGCTTCTTCTGGTACAGACAGTACAGCGGCAAGAGCCCCG | |
| | | AGCTGATCATGTTCATCTACAGCAACGGCGACAAAGAGGACGGCCGGTT | |
| | | TACAGCCCAGCTGAACAAGGCCAGCCAGTACGTGTCCCTGCTGATCAGA | |
| | | GATAGCCAGCCTAGCGACAGCGCCACCTATCTGTGTGCCGTGATCGGCG | |
| | | GCACAGATAGCTGGGGCAAACTCCAGTTTGGCGCTGGCACACAGGTGGT | |
| | | GGTCACCCCTGACATTCAGAACCCTGATCCTGCCGTGTACCAGCTGAGA | |
| | | GACAGCAAGAGCAGCGACAAGAGCGTGTGCCTGTTCACCGACTTCGACA | |
| | | GCCAGACCAACGTGTCCCAGAGCAAGGACAGCGACGTGTACATCACCGA | |
| | | TAAGTGCGTGCTGGACATGCGGAGCATGGACTTCAAGAGCAACAGCGCC | |
| | | GTGGCCTGGTCCAACAAGAGCGATTTCGCCTGCGCCAACGCCTTCAACA | |
| | | ACAGCATTATCCCCGAGGACACATTCTTCCCAAGTCCTGAGAGCAGCTG | |
| | | CGACGTGAAGCTGGTGGAAAAGAGCTTCGAGACAGACACCAACCTGAAC | |
| | | TTCCAGAACCTGTCTGTGATCGGCTTCCGGATCCTGCTGCTGAAGGTGG | |
| | | CCGGCTTCAATCTGCTGATGACCCTGAGACTGTGGTCCAGC | |
| | β (with | ATGGGCTGCAGGCTGCTCTGCTGTGCGGTTCTCTGTCTCCTGGGAGCGGGTGAGT | SEQ ID NO: 66 |
| | TRBC1) | TGGTCCCCATGGAAACGGGAGTTACGCAGACACCAAGACACCTGGTCATGGGAAT | |
| | | GACAAATAAGAAGTCTTTGAAATGTGAACAACATCTGGGTCATAACGCTATGTAT | |
| | | TGGTACAAGCAAAGTGCTAAGAAGCCACTGGAGCTCATGTTTGTCTACAGTCTTG | |
| | | AAGAACGGGTTGAAAACAACAGTGTGCCAAGTCGCTTCTCACCTGAATGCCCCAA | |
| | | CAGCTCTCACTTATTCCTTCACCTACACACCCTGCAGCCAGAAGACTCGGCCCTG | |
| | | TATCTCTGCGCCAGCAGCCAAGAAGAGGGGGCTGTCTATGGCTACACCTTCGGTT | |
| | | CGGGGACCAGGTTAACCGTTGTAGAGGACCTGAACAAGGTGTTCCCACCCGAGGT | |
| | | CGCTGTGTTTGAGCCATCAGAAGCAGAGATCTCCCACACCCAAAAGGCCACACTG | |
| | | GTGTGCCTGGCCACAGGCTTCTTCCCCGACCACGTGGAGCTGAGCTGGTGGGTGA | |
| | | ATGGGAAGGAGGTGCACAGTGGGGTCAGCACGGACCCGCAGCCCCTCAAGGAGCA | |
| | | GCCCGCCCTCAATGACTCCAGATACTGCCTGAGCAGCCGCCTGAGGGTCTCGGCC | |
| | | ACCTTCTGGCAGAACCCCCGCAACCACTTCCGCTGTCAAGTCCAGTTCTACGGGC | |
| | | TCTCGGAGAATGACGAGTGGACCCAGGATAGGGCCAAACCCGTCACCCAGATCGT | |
| | | CAGCGCCGAGGCCTGGGGTAGAGCAGACTGTGGCTTTACCTCGGTGTCCTACCAG | |
| | | CAAGGGGTCCTGTCTGCCACCATCCTCTATGAGATCCTGCTAGGGAAGGCCACCC | |
| | | TGTATGCTGTGCTGGTCAGCGCCCTTGTGTTGATGGCCATGGTCAAGAGAAAGGA | |
| | | TTTC | |
| | β (with | ATGGGCTGCAGGCTGCTCTGCTGTGCGGTTCTCTGTCTCCTGGGAGCGGGTGAGT | SEQ ID NO: 67 |
| | TRBC2) | TGGTCCCCATGGAAACGGGAGTTACGCAGACACCAAGACACCTGGTCATGGGAAT | |
| | | GACAAATAAGAAGTCTTTGAAATGTGAACAACATCTGGGTCATAACGCTATGTAT | |
| | | TGGTACAAGCAAAGTGCTAAGAAGCCACTGGAGCTCATGTTTGTCTACAGTCTTG | |
| | | AAGAACGGGTTGAAAACAACAGTGTGCCAAGTCGCTTCTCACCTGAATGCCCCAA | |
| | | CAGCTCTCACTTATTCCTTCACCTACACACCCTGCAGCCAGAAGACTCGGCCCTG | |
| | | TATCTCTGCGCCAGCAGCCAAGAAGAGGGGGCTGTCTATGGCTACACCTTCGGTT | |
| | | CGGGGACCAGGTTAACCGTTGTAGAGGACCTGAAAAACGTGTTCCCACCCGAGGT | |
| | | CGCTGTGTTTGAGCCATCAGAAGCAGAGATCTCCCACACCCAAAAGGCCACACTG | |
| | | GTGTGCCTGGCCACAGGCTTCTACCCCGACCACGTGGAGCTGAGCTGGTGGGTGA | |
| | | ATGGGAAGGAGGTGCACAGTGGGGTCAGCACAGACCCGCAGCCCCTCAAGGAGCA | |
| | | GCCCGCCCTCAATGACTCCAGATACTGCCTGAGCAGCCGCCTGAGGGTCTCGGCC | |
| | | ACCTTCTGGCAGAACCCCCGCAACCACTTCCGCTGTCAAGTCCAGTTCTACGGGC | |
| | | TCTCGGAGAATGACGAGTGGACCCAGGATAGGGCCAAACCTGTCACCCAGATCGT | |
| | | CAGCGCCGAGGCCTGGGGTAGAGCAGACTGTGGCTTCACCTCCGAGTCTTACCAG | |
| | | CAAGGGGTCCTGTCTGCCACCATCCTCTATGAGATCTTGCTAGGGAAGGCCACCT | |
| | | TGTATGCCGTGCTGGTCAGTGCCCTCGTGCTGATGGCCATGGTCAAGAGAAAGGA | |
| | | TTCCAGAGGC | |
| | β (with | ATGGGATGTAGACTTCTGTGTTGCGCCGTGCTGTGTCTGCTTGGAGCTG | SEQ ID NO: 219 |
| | TRBC2) | GCGAACTGGTGCCTATGGAAACCGGCGTGACCCAGACACCTAGACACCT | |
| | | GGTCATGGGCATGACAAACAAGAAAAGCCTGAAGTGCGAGCAGCACCTG | |
| | | GGCCACAATGCCATGTACTGGTACAAGCAGAGCGCCAAGAAACCCCTGG | |
| | | AACTGATGTTCGTGTACAGCCTGGAAGAGAGGGTCGAGAACAACAGCGT | |
| | | GCCCAGCAGATTCAGCCCTGAGTGCCCTAATAGCAGCCACCTGTTTCTG | |
| | | CATCTGCACACCCTGCAGCCTGAGGACTCTGCCCTGTATCTGTGTGCCA | |
| | | GCAGCCAAGAGGAAGGCGCCGTTTACGGCTACACATTTGGCAGCGGCAC | |
| | | CAGACTGACCGTGGTGGAAGATCTGAAGAACGTGTTCCCACCTGAGGTG | |
| | | GCCGTGTTCGAGCCTTCTGAGGCCGAGATCAGCCACACACAGAAAGCCA | |
| | | CACTCGTGTGTCTGGCCACCGGCTTCTATCCCGATCACGTGGAACTGTC | |
| | | TTGGTGGGTCAACGGCAAAGAGGTGCACAGCGGCGTCTGTACCGATCCT | |
| | | CAGCCTCTGAAAGAGCAGCCCGCTCTGAACGACAGCAGATACTGCCTGA | |
| | | GCAGCAGACTGAGAGTGTCCGCCACCTTCTGGCAGAACCCCAGAAACCA | |
| | | CTTCAGATGCCAGGTGCAGTTCTACGGCCTGAGCGAGAACGATGAGTGG | |
| | | ACCCAGGATAGAGCCAAGCCTGTGACACAGATCGTGTCTGCCGAAGCCT | |
| | | GGGGCAGAGCCGATTGTGGCTTTACCAGCGAGAGCTACCAGCAGGGCGT | |
| | | GCTGTCTGCCACAATCCTGTACGAGATCCTGCTGGGAAAAGCCACTCTG | |
| | | TACGCTGTGCTGGTGTCCGCTCTGGTGCTGATGGCCATGGTCAAGCGGA | |
| | | AGGATAGCAGGGGC | |
| HD15 | α (with | ATGATATCCTTGAGAGTTTTACTGGTGATCCTGTGGCTTCAGTTAAGCTGGGTTT | SEQ ID NO: 68 |
| S1 IFNg | TRAC) | GGAGCCAACGGAAGGAGGTGGAGCAGGATCCTGGACCCTTCAATGTTCCAGAGGG | |
| enriched | | AGCCACTGTCGCTTTCAACTGTACTTACAGCAACAGTGCTTCTCAGTCTTTCTTC | |
| population | | TGGTACAGACAGGATTGCAGGAAAGAACCTAAGTTGCTGATGTCCGTATACTCCA | |
| | | GTGGTAATGAAGATGGAAGGTTTACAGCACAGCTCAATAGAGCCAGCCAGTATAT | |
| | | TTCCCTGCTCATCAGAGACTCCAAGCTCAGTGATTCAGCCACCTACCTCTGTGTG | |
| | | GTGCCCCGGGGGCTTTCAACTGACAGCTGGGGGAAATTGCAGTTTGGAGCAGGGA | |
| | | CCCAGGTTGTGGTCACCCCAGATATCCAGAACCCTGACCCTGCCGTGTACCAGCT | |
| | | GAGAGACTCTAAATCCAGTGACAAGTCTGTCTGCCTATTCACCGATTTTGATTCT | |
| | | CAAACAAATGTGTCACAAAGTAAGGATTCTGATGTGTATATCACAGACAAAACTG | |
| | | TGCTAGACATGAGGTCTATGGACTTCAAGAGCAACAGTGCTGTGGCCTGGAGCAA | |
| | | CAAATCTGACTTTGCATGTGCAAACGCCTTCAACAACAGCATTATTCCAGAAGAC | |
| | | ACCTTCTTCCCCAGCCCAGAAAGTTCCTGTGATGTCAAGCTGGTCGAGAAAAGCT | |
| | | TTGAAACAGATACGAACCTAAACTTTCAAAACCTGTCAGTGATTGGGTTCCGAAT | |
| | | CCTCCTCCTGAAAGTGGCCGGGTTTAATCTGCTCATGACGCTGCGGCTGTGGTCC | |
| | | AGC | |
| | α (with | ATGATCAGCCTGAGAGTGCTGCTGGTCATCCTGTGGCTGCAGCTGTCTT | SEQ ID NO: 220 |
| | TRAC) | GGGTCTGGTCCCAGCGGAAAGAGGTGGAACAGGACCCCGGACCTTTCAA | |
| | | TGTGCCTGAAGGCGCCACCGTGGCCTTCAACTGCACCTACAGCAATAGC | |
| | | GCCAGCCAGAGCTTCTTCTGGTACAGACAGGACTGCCGGAAAGAACCCA | |
| | | AGCTGCTGATGAGCGTGTACAGCAGCGGCAACGAGGACGGCAGATTCAC | |
| | | AGCCCAGCTGAACAGAGCCAGCCAGTACATCAGCCTGCTGATCCGGGAT | |
| | | AGCAAGCTGAGCGATAGCGCCACCTACCTGTGCGTGGTGCCTAGAGGCC | |
| | | TGAGCACAGATTCTTGGGGCAAGCTGCAGTTCGGAGCCGGAACACAGGT | |
| | | GGTGGTCACCCCTGACATTCAGAACCCTGATCCTGCCGTGTACCAGCTG | |
| | | AGAGACAGCAAGAGCAGCGACAAGAGCGTGTGCCTGTTCACCGACTTCG | |
| | | ACAGCCAGACCAACGTGTCCCAGAGCAAGGACAGCGACGTGTACATCAC | |
| | | CGATAAGTGCGTGCTGGACATGCGGAGCATGGACTTCAAGAGCAACAGC | |
| | | GCCGTGGCCTGGTCCAACAAGAGCGATTTCGCCTGCGCCAACGCCTTCA | |
| | | ACAACAGCATTATCCCCGAGGACACATTCTTCCCAAGTCCTGAGAGCAG | |
| | | CTGCGACGTGAAGCTGGTGGAAAAGAGCTTCGAGACAGACACCAACCTG | |
| | | AACTTCCAGAACCTGAGCGTGATCGGCTTCCGGATCCTGCTGCTGAAAG | |
| | | TGGCCGGCTTCAACCTGCTCATGACCCTGAGACTGTGGTCCAGC | |
| | β (with | ATGGGTCCTGGGCTTCTCCACTGGATGGCCCTTTGTCTCCTTGGAACAGGTCATG | SEQ ID NO: 69 |
| | TRBC1) | GGGATGCCATGGTCATCCAGAACCCAAGATACCAGGTTACCCAGTTTGGAAAGCC | |
| | | AGTGACCCTGAGTTGTTCTCAGACTTTGAACCATAACGTCATGTACTGGTACCAG | |
| | | CAGAAGTCAAGTCAGGCCCCAAAGCTGCTGTTCCACTACTATGACAAAGATTTTA | |
| | | ACAATGAAGCAGACACCCCTGATAACTTCCAATCCAGGAGGCCGAACACTTCTTT | |
| | | CTGCTTTCTTGACATCCGCTCACCAGGCCTGGGGGACGCAGCCATGTACCTGTGT | |
| | | GCCACCAGCAGGGAGGGGCTAGCGGCAGATACGCAGTATTTTGGCCCAGGCACCC | |
| | | GGCTGACAGTGCTCGAGGACCTGAACAAGGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTTCCCCGACCACGTGGAGCTGAGCTGGTGGGTGAATGGGAAGG | |
| | | AGGTGCACAGTGGGGTCAGCACGGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCCGTCACCCAGATCGTCAGCGCCGA | |
| | | GGCCTGGGGTAGAGCAGACTGTGGCTTTACCTCGGTGTCCTACCAGCAAGGGGTC | |
| | | CTGTCTGCCACCATCCTCTATGAGATCCTGCTAGGGAAGGCCACCCTGTATGCTG | |
| | | TGCTGGTCAGCGCCCTTGTGTTGATGGCCATGGTCAAGAGAAAGGATTTC | |
| | β (with | ATGGGTCCTGGGCTTCTCCACTGGATGGCCCTTTGTCTCCTTGGAACAGGTCATG | SEQ ID NO: 70 |
| | TRBC2) | GGGATGCCATGGTCATCCAGAACCCAAGATACCAGGTTACCCAGTTTGGAAAGCC | |
| | | AGTGACCCTGAGTTGTTCTCAGACTTTGAACCATAACGTCATGTACTGGTACCAG | |
| | | CAGAAGTCAAGTCAGGCCCCAAAGCTGCTGTTCCACTACTATGACAAAGATTTTA | |
| | | ACAATGAAGCAGACACCCCTGATAACTTCCAATCCAGGAGGCCGAACACTTCTTT | |
| | | CTGCTTTCTTGACATCCGCTCACCAGGCCTGGGGGACGCAGCCATGTACCTGTGT | |
| | | GCCACCAGCAGGGAGGGGCTAGCGGCAGATACGCAGTATTTTGGCCCAGGCACCC | |
| | | GGCTGACAGTGCTCGAGGACCTGAAAAACGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTACCCCGACCACGTGGAGCTGAGCTGGTGGGTGAATGGGAAGG | |
| | | AGGTGCACAGTGGGGTCAGCACAGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCTGTCACCCAGATCGTCAGCGCCGA | |
| | | GGCCTGGGGTAGAGCAGACTGTGGCTTCACCTCCGAGTCTTACCAGCAAGGGGTC | |
| | | CTGTCTGCCACCATCCTCTATGAGATCTTGCTAGGGAAGGCCACCTTGTATGCCG | |
| | | TGCTGGTCAGTGCCCTCGTGCTGATGGCCATGGTCAAGAGAAAGGATTCCAGAGG | |
| | | C | |
| | β (with | ATGGGACCTGGACTTCTTCATTGGATGGCCCTGTGTCTGCTCGGCACAG | SEQ ID NO: 221 |
| | TRBC2) | GACATGGCGACGCTATGGTCATTCAGAACCCCAGATACCAAGTGACCCA | |
| | | GTTCGGCAAGCCCGTGACACTGAGCTGTAGCCAGACACTGAACCACAAC | |
| | | GTGATGTACTGGTATCAGCAGAAGTCCTCTCAGGCCCCTAAGCTGCTGT | |
| | | TCCACTACTACGACAAGGACTTCAACAACGAGGCCGACACACCCGACAA | |
| | | CTTCCAGAGCAGAAGGCCCAATACCAGCTTCTGCTTCCTGGACATCAGA | |
| | | AGCCCTGGCCTGGGAGATGCCGCCATGTATCTGTGTGCCACCAGCAGAG | |
| | | AAGGCCTGGCCGCCGATACACAGTATTTCGGCCCTGGCACCAGACTGAC | |
| | | CGTGCTCGAGGATCTGAAGAACGTGTTCCCACCTGAGGTGGCCGTGTTC | |
| | | GAGCCTTCTGAGGCCGAGATCAGCCACACACAGAAAGCCACACTCGTGT | |
| | | GTCTGGCCACCGGCTTCTATCCCGATCACGTGGAACTGTCTTGGTGGGT | |
| | | CAACGGCAAAGAGGTGCACAGCGGCGTCTGTACCGATCCTCAGCCTCTG | |
| | | AAAGAGCAGCCCGCTCTGAACGACAGCAGATACTGCCTGAGCAGCAGAC | |
| | | TGAGAGTGTCCGCCACCTTCTGGCAGAACCCTCGGAACCACTTCAGATG | |
| | | CCAGGTGCAGTTCTACGGCCTGAGCGAGAACGATGAGTGGACCCAGGAT | |
| | | AGAGCCAAGCCTGTGACTCAGATCGTGTCTGCCGAAGCCTGGGGCAGAG | |
| | | CCGATTGTGGCTTTACCAGCGAGAGCTACCAGCAGGGCGTGCTGTCTGC | |
| | | CACAATCCTGTACGAGATCCTGCTGGGCAAAGCCACTCTGTACGCCGTG | |
| | | CTGGTGTCTGCCCTGGTGCTGATGGCCATGGTCAAGCGGAAGGATAGCA | |
| | | GGGGC | |
| Patient 1 | α (with | ATGGCTTTGCAGAGCACTCTGGGGGCGGTGTGGCTAGGGCTTCTCCTCAACTCTC | SEQ ID NO: 162 |
| direct | TRAC) | TCTGGAAGGTTGCAGAAAGCAAGGACCAAGTGTTTCAGCCTTCCACAGTGGCATC | |
| sequencing | | TTCAGAGGGAGCTGTGGTGGAAATCTTCTGTAATCACTCTGTGTCCAATGCTTAC | |
| upon | | AACTTCTTCTGGTACCTTCACTTCCCGGGATGTGCACCAAGACTCCTTGTTAAAG | |
| sorting | | GCTCAAAGCCTTCTCAGCAGGGACGATACAACATGACCTATGAACGGTTCTCTTC | |
| | | ATCGCTGCTCATCCTCCAGGTGCGGGAGGCAGATGCTGCTGTTTACTACTGTGCT | |
| | | GCCCCTAACGACTACAAGCTCAGCTTTGGAGCCGGAACCACAGTAACTGTAAGAG | |
| | | CAAATATCCAGAACCCTGACCCTGCCGTGTACCAGCTGAGAGACTCTAAATCCAG | |
| | | TGACAAGTCTGTCTGCCTATTCACCGATTTTGATTCTCAAACAAATGTGTCACAA | |
| | | AGTAAGGATTCTGATGTGTATATCACAGACAAAACTGTGCTAGACATGAGGTCTA | |
| | | TGGACTTCAAGAGCAACAGTGCTGTGGCCTGGAGCAACAAATCTGACTTTGCATG | |
| | | TGCAAACGCCTTCAACAACAGCATTATTCCAGAAGACACCTTCTTCCCCAGCCCA | |
| | | GAAAGTTCCTGTGATGTCAAGCTGGTCGAGAAAAGCTTTGAAACAGATACGAACC | |
| | | TAAACTTTCAAAACCTGTCAGTGATTGGGTTCCGAATCCTCCTCCTGAAAGTGGC | |
| | | CGGGTTTAATCTGCTCATGACGCTGCGGCTGTGGTCCAGC | |
| | β1 (with | ATGGGCACCAGCCTCCTCTGCTGGATGGCCCTGTGTCTCCTGGGGGCAGATCACG | SEQ ID NO: 163 |
| | TRBC1) | CAGATACTGGAGTCTCCCAGAACCCCAGACACAAGATCACAAAGAGGGGACAGAA | |
| | | TGTAACTTTCAGGTGTGATCCAATTTCTGAACACAACCGCCTTTATTGGTACCGA | |
| | | CAGACCCTGGGGCAGGGCCCAGAGTTTCTGACTTACTTCCAGAATGAAGCTCAAC | |
| | | TAGAAAAATCAAGGCTGCTCAGTGATCGGTTCTCTGCAGAGAGGCCTAAGGGATC | |
| | | TTTCTCCACCTTGGAGATCCAGCGCACAGAGCAGGGGGACTCGGCCATGTATCTC | |
| | | TGTGCCAGCAGCAGCGGACTAGCGTTCTACGAGCAGTACTTCGGGCCGGGCACCA | |
| | | GGCTCACGGTCACAGAGGACCTGAACAAGGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTTCCCCGACCACGTGGAGCTGAGCTGGTGGGTGAATGGGAAGG | |
| | | AGGTGCACAGTGGGGTCAGCACGGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCCGTCACCCAGATCGTCAGCGCCGA | |
| | | GGCCTGGGGTAGAGCAGACTGTGGCTTTACCTCGGTGTCCTACCAGCAAGGGGTC | |
| | | CTGTCTGCCACCATCCTCTATGAGATCCTGCTAGGGAAGGCCACCCTGTATGCTG | |
| | | TGCTGGTCAGCGCCCTTGTGTTGATGGCCATGGTCAAGAGAAAGGATTTC | |
| | β1 (with | ATGGGCACCAGCCTCCTCTGCTGGATGGCCCTGTGTCTCCTGGGGGCAGATCACG | SEQ ID NO: 164 |
| | TRBC2) | CAGATACTGGAGTCTCCCAGAACCCCAGACACAAGATCACAAAGAGGGGACAGAA | |
| | | TGTAACTTTCAGGTGTGATCCAATTTCTGAACACAACCGCCTTTATTGGTACCGA | |
| | | CAGACCCTGGGGCAGGGCCCAGAGTTTCTGACTTACTTCCAGAATGAAGCTCAAC | |
| | | TAGAAAAATCAAGGCTGCTCAGTGATCGGTTCTCTGCAGAGAGGCCTAAGGGATC | |
| | | TTTCTCCACCTTGGAGATCCAGCGCACAGAGCAGGGGGACTCGGCCATGTATCTC | |
| | | TGTGCCAGCAGCAGCGGACTAGCGTTCTACGAGCAGTACTTCGGGCCGGGCACCA | |
| | | GGCTCACGGTCACAGAGGACCTGAAAAACGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTACCCCGACCACGTGGAGCTGAGCTGGTGGGTGAATGGGAAGG | |
| | | AGGTGCACAGTGGGGTCAGCACAGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCTGTCACCCAGATCGTCAGCGCCGA | |
| | | GGCCTGGGGTAGAGCAGACTGTGGCTTCACCTCCGAGTCTTACCAGCAAGGGGTC | |
| | | CTGTCTGCCACCATCCTCTATGAGATCTTGCTAGGGAAGGCCACCTTGTATGCCG | |
| | | TGCTGGTCAGTGCCCTCGTGCTGATGGCCATGGTCAAGAGAAAGGATTCCAGAGG | |
| | | C | |
| | β2 (with | ATGGTTTCCAGGCTTCTCAGTTTAGTGTCCCTTTGTCTCCTGGGAGCAAAGCACA | SEQ ID NO: 165 |
| | TRBC1) | TAGAAGCTGGAGTTACTCAGTTCCCCAGCCACAGCGTAATAGAGAAGGGCCAGAC | |
| | | TGTGACTCTGAGATGTGACCCAATTTCTGGACATGATAATCTTTATTGGTATCGA | |
| | | CGTGTTATGGGAAAAGAAATAAAATTTCTGTTACATTTTGTGAAAGAGTCTAAAC | |
| | | AGGATGAGTCCGGTATGCCCAACAATCGATTCTTAGCTGAAAGGACTGGAGGGAC | |
| | | GTATTCTACTCTGAAGGTGCAGCCTGCAGAACTGGAGGATTCTGGAGTTTATTTC | |
| | | TGTGCCAGCAGCCAATTGTCAGGGCGCGACTCCTACGAGCAGTACTTCGGGCCGG | |
| | | GCACCAGGCTCACGGTCACAGAGGACCTGAACAAGGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTTCCCCGACCACGTGGAGCTGAGCTGGTGGGTGAATG | |
| | | GGAAGGAGGTGCACAGTGGGGTCAGCACGGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCCGTCACCCAGATCGTCAG | |
| | | CGCCGAGGCCTGGGGTAGAGCAGACTGTGGCTTTACCTCGGTGTCCTACCAGCAA | |
| | | GGGGTCCTGTCTGCCACCATCCTCTATGAGATCCTGCTAGGGAAGGCCACCCTGT | |
| | | ATGCTGTGCTGGTCAGCGCCCTTGTGTTGATGGCCATGGTCAAGAGAAAGGATTT | |
| | | C | |
| | β2 (with | ATGGTTTCCAGGCTTCTCAGTTTAGTGTCCCTTTGTCTCCTGGGAGCAAAGCACA | SEQ ID NO: 166 |
| | TRBC2) | TAGAAGCTGGAGTTACTCAGTTCCCCAGCCACAGCGTAATAGAGAAGGGCCAGAC | |
| | | TGTGACTCTGAGATGTGACCCAATTTCTGGACATGATAATCTTTATTGGTATCGA | |
| | | CGTGTTATGGGAAAAGAAATAAAATTTCTGTTACATTTTGTGAAAGAGTCTAAAC | |
| | | AGGATGAGTCCGGTATGCCCAACAATCGATTCTTAGCTGAAAGGACTGGAGGGAC | |
| | | GTATTCTACTCTGAAGGTGCAGCCTGCAGAACTGGAGGATTCTGGAGTTTATTTC | |
| | | TGTGCCAGCAGCCAATTGTCAGGGCGCGACTCCTACGAGCAGTACTTCGGGCCGG | |
| | | GCACCAGGCTCACGGTCACAGAGGACCTGAAAAACGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTACCCCGACCACGTGGAGCTGAGCTGGTGGGTGAATG | |
| | | GGAAGGAGGTGCACAGTGGGGTCAGCACAGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCTGTCACCCAGATCGTCAG | |
| | | CGCCGAGGCCTGGGGTAGAGCAGACTGTGGCTTCACCTCCGAGTCTTACCAGCAA | |
| | | GGGGTCCTGTCTGCCACCATCCTCTATGAGATCTTGCTAGGGAAGGCCACCTTGT | |
| | | ATGCCGTGCTGGTCAGTGCCCTCGTGCTGATGGCCATGGTCAAGAGAAAGGATTC | |
| | | CAGAGGC | |
| Patient 1 | α1 (with | ATGGCCTCTGCACCCATCTCGATGCTTGCGATGCTCTTCACATTGAGTGGGCTGA | SEQ ID NO: 167 |
| growing | TRAC) | GAGCTCAGTCAGTGGCTCAGCCGGAAGATCAGGTCAACGTTGCTGAAGGGAATCC | |
| colony | | TCTGACTGTGAAATGCACCTATTCAGTCTCTGGAAACCCTTATCTTTTTTGGTAT | |
| | | GTTCAATACCCCAACCGAGGCCTCCAGTTCCTTCTGAAATACATCACAGGGGATA | |
| | | ACCTGGTTAAAGGCAGCTATGGCTTTGAAGCTGAATTTAACAAGAGCCAAACCTC | |
| | | CTTCCACCTGAAGAAACCATCTGCCCTTGTGAGCGACTCCGCTTTGTACTTCTGT | |
| | | GCTGTGAGAGACGGTGGTGCTACAAACAAGCTCATCTTTGGAACTGGCACTCTGC | |
| | | TTGCTGTCCAGCCAAATATCCAGAACCCTGACCCTGCCGTGTACCAGCTGAGAGA | |
| | | CTCTAAATCCAGTGACAAGTCTGTCTGCCTATTCACCGATTTTGATTCTCAAACA | |
| | | AATGTGTCACAAAGTAAGGATTCTGATGTGTATATCACAGACAAAACTGTGCTAG | |
| | | ACATGAGGTCTATGGACTTCAAGAGCAACAGTGCTGTGGCCTGGAGCAACAAATC | |
| | | TGACTTTGCATGTGCAAACGCCTTCAACAACAGCATTATTCCAGAAGACACCTTC | |
| | | TTCCCCAGCCCAGAAAGTTCCTGTGATGTCAAGCTGGTCGAGAAAAGCTTTGAAA | |
| | | CAGATACGAACCTAAACTTTCAAAACCTGTCAGTGATTGGGTTCCGAATCCTCCT | |
| | | CCTGAAAGTGGCCGGGTTTAATCTGCTCATGACGCTGCGGCTGTGGTCCAGC | |
| | α2 (with | ATGAGGCAAGTGGCGAGAGTGATCGTGTTCCTGACCCTGAGTACTTTGAGCCTTG | SEQ ID NO: 168 |
| | TRAC) | CTAAGACCACCCAGCCCATCTCCATGGACTCATATGAAGGACAAGAAGTGAACAT | |
| | | AACCTGTAGCCACAACAACATTGCTACAAATGATTATATCACGTGGTACCAACAG | |
| | | TTTCCCAGCCAAGGACCACGATTTATTATTCAAGGATACAAGACAAAAGTTACAA | |
| | | ACGAAGTGGCCTCCCTGTTTATCCCTGCCGACAGAAAGTCCAGCACTCTGAGCCT | |
| | | GCCCCGGGTTTCCCTGAGCGACACTGCTGTGTACTACTGCCTCGTGGGTGGTTAT | |
| | | ACTGGAGGCTTCAAAACTATCTTTGGAGCAGGAACAAGACTATTTGTTAAAGCAA | |
| | | ATATCCAGAACCCTGACCCTGCCGTGTACCAGCTGAGAGACTCTAAATCCAGTGA | |
| | | CAAGTCTGTCTGCCTATTCACCGATTTTGATTCTCAAACAAATGTGTCACAAAGT | |
| | | AAGGATTCTGATGTGTATATCACAGACAAAACTGTGCTAGACATGAGGTCTATGG | |
| | | ACTTCAAGAGCAACAGTGCTGTGGCCTGGAGCAACAAATCTGACTTTGCATGTGC | |
| | | AAACGCCTTCAACAACAGCATTATTCCAGAAGACACCTTCTTCCCCAGCCCAGAA | |
| | | AGTTCCTGTGATGTCAAGCTGGTCGAGAAAAGCTTTGAAACAGATACGAACCTAA | |
| | | ACTTTCAAAACCTGTCAGTGATTGGGTTCCGAATCCTCCTCCTGAAAGTGGCCGG | |
| | | GTTTAATCTGCTCATGACGCTGCGGCTGTGGTCCAGC | |
| | β (with | ATGGGCCCCCAGCTCCTTGGCTATGTGGTCCTTTGCCTTCTAGGAGCAGGCCCCC | SEQ ID NO: 169 |
| | TRBC1) | TGGAAGCCCAAGTGACCCAGAACCCAAGATACCTCATCACAGTGACTGGAAAGAA | |
| | | GTTAACAGTGACTTGTTCTCAGAATATGAACCATGAGTATATGTCCTGGTATCGA | |
| | | CAAGACCCAGGGCTGGGCTTAAGGCAGATCTACTATTCAATGAATGTTGAGGTGA | |
| | | CTGATAAGGGAGATGTTCCTGAAGGGTACAAAGTCTCTCGAAAAGAGAAGAGGAA | |
| | | TTTCCCCCTGATCCTGGAGTCGCCCAGCCCCAACCAGACCTCTCTGTACTTCTGT | |
| | | GCCAGCAGTACGCTTGGGGGGGAGCTGTTTTTTGGAGAAGGCTCTAGGCTGACCG | |
| | | TACTGGAGGACCTGAACAAGGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTTCCCCGACCACGTGGAGCTGAGCTGGTGGGTGAATGGGAAGGAGGTGCACA | |
| | | GTGGGGTCAGCACGGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCCGTCACCCAGATCGTCAGCGCCGAGGCCTGGGG | |
| | | TAGAGCAGACTGTGGCTTTACCTCGGTGTCCTACCAGCAAGGGGTCCTGTCTGCC | |
| | | ACCATCCTCTATGAGATCCTGCTAGGGAAGGCCACCCTGTATGCTGTGCTGGTCA | |
| | | GCGCCCTTGTGTTGATGGCCATGGTCAAGAGAAAGGATTTC | |
| | β (with | ATGGGCCCCCAGCTCCTTGGCTATGTGGTCCTTTGCCTTCTAGGAGCAGGCCCCC | SEQ ID NO: 170 |
| | TRBC2) | TGGAAGCCCAAGTGACCCAGAACCCAAGATACCTCATCACAGTGACTGGAAAGAA | |
| | | GTTAACAGTGACTTGTTCTCAGAATATGAACCATGAGTATATGTCCTGGTATCGA | |
| | | CAAGACCCAGGGCTGGGCTTAAGGCAGATCTACTATTCAATGAATGTTGAGGTGA | |
| | | CTGATAAGGGAGATGTTCCTGAAGGGTACAAAGTCTCTCGAAAAGAGAAGAGGAA | |
| | | TTTCCCCCTGATCCTGGAGTCGCCCAGCCCCAACCAGACCTCTCTGTACTTCTGT | |
| | | GCCAGCAGTACGCTTGGGGGGGAGCTGTTTTTTGGAGAAGGCTCTAGGCTGACCG | |
| | | TACTGGAGGACCTGAAAAACGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTACCCCGACCACGTGGAGCTGAGCTGGTGGGTGAATGGGAAGGAGGTGCACA | |
| | | GTGGGGTCAGCACAGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCTGTCACCCAGATCGTCAGCGCCGAGGCCTGGGG | |
| | | TAGAGCAGACTGTGGCTTCACCTCCGAGTCTTACCAGCAAGGGGTCCTGTCTGCC | |
| | | ACCATCCTCTATGAGATCTTGCTAGGGAAGGCCACCTTGTATGCCGTGCTGGTCA | |
| | | GTGCCCTCGTGCTGATGGCCATGGTCAAGAGAAAGGATTCCAGAGGC | |
| Patient 2 | α 1 (with | ATGCTCCTGCTGCTCGTCCCAGCGTTCCAGGTGATTTTTACCCTGGGAGGAACCA | SEQ ID NO: 171 |
| | TRAC) | GAGCCCAGTCTGTGACCCAGCTTGACAGCCAAGTCCCTGTCTTTGAAGAAGCCCC | |
| | | TGTGGAGCTGAGGTGCAACTACTCATCGTCTGTTTCAGTGTATCTCTTCTGGTAT | |
| | | GTGCAATACCCCAACCAAGGACTCCAGCTTCTCCTGAAGTATTTATCAGGATCCA | |
| | | CCCTGGTTGAAAGCATCAACGGTTTTGAGGCTGAATTTAACAAGAGTCAAACTTC | |
| | | CTTCCACTTGAGGAAACCCTCAGTCCATATAAGCGACACGGCTGAGTACTTCTGT | |
| | | GCTGTGACCCTGCTTTCGATTGAGCCTTCGGCTGGGGGTTACCAGAAAGTTACCT | |
| | | TTGGAATTGGAACAAAGCTCCAAGTCATCCCAAATATCCAGAACCCTGACCCTGC | |
| | | CGTGTACCAGCTGAGAGACTCTAAATCCAGTGACAAGTCTGTCTGCCTATTCACC | |
| | | GATTTTGATTCTCAAACAAATGTGTCACAAAGTAAGGATTCTGATGTGTATATCA | |
| | | CAGACAAAACTGTGCTAGACATGAGGTCTATGGACTTCAAGAGCAACAGTGCTGT | |
| | | GGCCTGGAGCAACAAATCTGACTTTGCATGTGCAAACGCCTTCAACAACAGCATT | |
| | | ATTCCAGAAGACACCTTCTTCCCCAGCCCAGAAAGTTCCTGTGATGTCAAGCTGG | |
| | | TCGAGAAAAGCTTTGAAACAGATACGAACCTAAACTTTCAAAACCTGTCAGTGAT | |
| | | TGGGTTCCGAATCCTCCTCCTGAAAGTGGCCGGGTTTAATCTGCTCATGACGCTG | |
| | | CGGCTGTGGTCCAGC | |
| | α 2 (with | ATGACATCCATTCGAGCTGTATTTATATTCCTGTGGCTGCAGCTGGACTTGGTGA | SEQ ID NO: 172 |
| | TRAC) | ATGGAGAGAATGTGGAGCAGCATCCTTCAACCCTGAGTGTCCAGGAGGGAGACAG | |
| | | CGCTGTTATCAAGTGTACTTATTCAGACAGTGCCTCAAACTACTTCCCTTGGTAT | |
| | | AAGCAAGAACTTGGAAAAAGACCTCAGCTTATTATAGACATTCGTTCAAATGTGG | |
| | | GCGAAAAGAAAGACCAACGAATTGCTGTTACATTGAACAAGACAGCCAAACATTT | |
| | | CTCCCTGCACATCACAGAGACCCAACCTGAAGACTCGGCTGTCTACTTCTGTGCA | |
| | | GCAACCTCCCGCGATGACATGCGCTTTGGAGCAGGGACCAGACTGACAGTAAAAC | |
| | | CAAATATCCAGAACCCTGACCCTGCCGTGTACCAGCTGAGAGACTCTAAATCCAG | |
| | | TGACAAGTCTGTCTGCCTATTCACCGATTTTGATTCTCAAACAAATGTGTCACAA | |
| | | AGTAAGGATTCTGATGTGTATATCACAGACAAAACTGTGCTAGACATGAGGTCTA | |
| | | TGGACTTCAAGAGCAACAGTGCTGTGGCCTGGAGCAACAAATCTGACTTTGCATG | |
| | | TGCAAACGCCTTCAACAACAGCATTATTCCAGAAGACACCTTCTTCCCCAGCCCA | |
| | | GAAAGTTCCTGTGATGTCAAGCTGGTCGAGAAAAGCTTTGAAACAGATACGAACC | |
| | | TAAACTTTCAAAACCTGTCAGTGATTGGGTTCCGAATCCTCCTCCTGAAAGTGGC | |
| | | CGGGTTTAATCTGCTCATGACGCTGCGGCTGTGGTCCAGC | |
| | β 1 (with | ATGGGCACCAGCCTCCTCTGCTGGATGGCCCTGTGTCTCCTGGGGGCAGATCACG | SEQ ID NO: 173 |
| | TRBC1) | CAGATACTGGAGTCTCCCAGAACCCCAGACACAAGATCACAAAGAGGGGACAGAA | |
| | | TGTAACTTTCAGGTGTGATCCAATTTCTGAACACAACCGCCTTTATTGGTACCGA | |
| | | CAGACCCTGGGGCAGGGCCCAGAGTTTCTGACTTACTTCCAGAATGAAGCTCAAC | |
| | | TAGAAAAATCAAGGCTGCTCAGTGATCGGTTCTCTGCAGAGAGGCCTAAGGGATC | |
| | | TTTCTCCACCTTGGAGATCCAGCGCACAGAGCAGGGGGACTCGGCCATGTATCTC | |
| | | TGTGCCAGCAGCTTAGAAGGAAGGGCCATGCCCAGGGACAGCCACCAAGAGACCC | |
| | | AGTACTTCGGGCCAGGCACGCGGCTCCTGGTGCTCGAGGACCTGAACAAGGTGTT | |
| | | CCCACCCGAGGTCGCTGTGTTTGAGCCATCAGAAGCAGAGATCTCCCACACCCAA | |
| | | AAGGCCACACTGGTGTGCCTGGCCACAGGCTTCTTCCCCGACCACGTGGAGCTGA | |
| | | GCTGGTGGGTGAATGGGAAGGAGGTGCACAGTGGGGTCAGCACGGACCCGCAGCC | |
| | | CCTCAAGGAGCAGCCCGCCCTCAATGACTCCAGATACTGCCTGAGCAGCCGCCTG | |
| | | AGGGTCTCGGCCACCTTCTGGCAGAACCCCCGCAACCACTTCCGCTGTCAAGTCC | |
| | | AGTTCTACGGGCTCTCGGAGAATGACGAGTGGACCCAGGATAGGGCCAAACCCGT | |
| | | CACCCAGATCGTCAGCGCCGAGGCCTGGGGTAGAGCAGACTGTGGCTTTACCTCG | |
| | | GTGTCCTACCAGCAAGGGGTCCTGTCTGCCACCATCCTCTATGAGATCCTGCTAG | |
| | | GGAAGGCCACCCTGTATGCTGTGCTGGTCAGCGCCCTTGTGTTGATGGCCATGGT | |
| | | CAAGAGAAAGGATTTC | |
| | β 1 (with | ATGGGCACCAGCCTCCTCTGCTGGATGGCCCTGTGTCTCCTGGGGGCAGATCACG | SEQ ID NO: 174 |
| | TRBC2) | CAGATACTGGAGTCTCCCAGAACCCCAGACACAAGATCACAAAGAGGGGACAGAA | |
| | | TGTAACTTTCAGGTGTGATCCAATTTCTGAACACAACCGCCTTTATTGGTACCGA | |
| | | CAGACCCTGGGGCAGGGCCCAGAGTTTCTGACTTACTTCCAGAATGAAGCTCAAC | |
| | | TAGAAAAATCAAGGCTGCTCAGTGATCGGTTCTCTGCAGAGAGGCCTAAGGGATC | |
| | | TTTCTCCACCTTGGAGATCCAGCGCACAGAGCAGGGGGACTCGGCCATGTATCTC | |
| | | TGTGCCAGCAGCTTAGAAGGAAGGGCCATGCCCAGGGACAGCCACCAAGAGACCC | |
| | | AGTACTTCGGGCCAGGCACGCGGCTCCTGGTGCTCGAGGACCTGAAAAACGTGTT | |
| | | CCCACCCGAGGTCGCTGTGTTTGAGCCATCAGAAGCAGAGATCTCCCACACCCAA | |
| | | AAGGCCACACTGGTGTGCCTGGCCACAGGCTTCTACCCCGACCACGTGGAGCTGA | |
| | | GCTGGTGGGTGAATGGGAAGGAGGTGCACAGTGGGGTCAGCACAGACCCGCAGCC | |
| | | CCTCAAGGAGCAGCCCGCCCTCAATGACTCCAGATACTGCCTGAGCAGCCGCCTG | |
| | | AGGGTCTCGGCCACCTTCTGGCAGAACCCCCGCAACCACTTCCGCTGTCAAGTCC | |
| | | AGTTCTACGGGCTCTCGGAGAATGACGAGTGGACCCAGGATAGGGCCAAACCTGT | |
| | | CACCCAGATCGTCAGCGCCGAGGCCTGGGGTAGAGCAGACTGTGGCTTCACCTCC | |
| | | GAGTCTTACCAGCAAGGGGTCCTGTCTGCCACCATCCTCTATGAGATCTTGCTAG | |
| | | GGAAGGCCACCTTGTATGCCGTGCTGGTCAGTGCCCTCGTGCTGATGGCCATGGT | |
| | | CAAGAGAAAGGATTCCAGAGGC | |
| | β 2 (with | ATGGGTCCTGGGCTTCTCCACTGGATGGCCCTTTGTCTCCTTGGAACAGGTCATG | SEQ ID NO: 175 |
| | TRBC1) | GGGATGCCATGGTCATCCAGAACCCAAGATACCAGGTTACCCAGTTTGGAAAGCC | |
| | | AGTGACCCTGAGTTGTTCTCAGACTTTGAACCATAACGTCATGTACTGGTACCAG | |
| | | CAGAAGTCAAGTCAGGCCCCAAAGCTGCTGTTCCACTACTATGACAAAGATTTTA | |
| | | ACAATGAAGCAGACACCCCTGATAACTTCCAATCCAGGAGGCCGAACACTTCTTT | |
| | | CTGCTTTCTTGACATCCGCTCACCAGGCCTGGGGGACGCAGCCATGTACCTGTGT | |
| | | GCCACCAGCTGGGGGCTAAACGAGCAGTACTTCGGGCCGGGCACCAGGCTCACGG | |
| | | TCACAGAGGACCTGAACAAGGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTTCCCCGACCACGTGGAGCTGAGCTGGTGGGTGAATGGGAAGGAGGTGCACA | |
| | | GTGGGGTCAGCACGGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCCGTCACCCAGATCGTCAGCGCCGAGGCCTGGGG | |
| | | TAGAGCAGACTGTGGCTTTACCTCGGTGTCCTACCAGCAAGGGGTCCTGTCTGCC | |
| | | ACCATCCTCTATGAGATCCTGCTAGGGAAGGCCACCCTGTATGCTGTGCTGGTCA | |
| | | GCGCCCTTGTGTTGATGGCCATGGTCAAGAGAAAGGATTTC | |
| | β 2 (with | ATGGGTCCTGGGCTTCTCCACTGGATGGCCCTTTGTCTCCTTGGAACAGGTCATG | SEQ ID NO: 176 |
| | TRBC2) | GGGATGCCATGGTCATCCAGAACCCAAGATACCAGGTTACCCAGTTTGGAAAGCC | |
| | | AGTGACCCTGAGTTGTTCTCAGACTTTGAACCATAACGTCATGTACTGGTACCAG | |
| | | CAGAAGTCAAGTCAGGCCCCAAAGCTGCTGTTCCACTACTATGACAAAGATTTTA | |
| | | ACAATGAAGCAGACACCCCTGATAACTTCCAATCCAGGAGGCCGAACACTTCTTT | |
| | | CTGCTTTCTTGACATCCGCTCACCAGGCCTGGGGGACGCAGCCATGTACCTGTGT | |
| | | GCCACCAGCTGGGGGCTAAACGAGCAGTACTTCGGGCCGGGCACCAGGCTCACGG | |
| | | TCACAGAGGACCTGAAAAACGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTACCCCGACCACGTGGAGCTGAGCTGGTGGGTGAATGGGAAGGAGGTGCACA | |
| | | GTGGGGTCAGCACAGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCTGTCACCCAGATCGTCAGCGCCGAGGCCTGGGG | |
| | | TAGAGCAGACTGTGGCTTCACCTCCGAGTCTTACCAGCAAGGGGTCCTGTCTGCC | |
| | | ACCATCCTCTATGAGATCTTGCTAGGGAAGGCCACCTTGTATGCCGTGCTGGTCA | |
| | | GTGCCCTCGTGCTGATGGCCATGGTCAAGAGAAAGGATTCCAGAGGC | |
| Patient 3 | α 1 (with | ATGCTGACTGCCAGCCTGTTGAGGGCAGTCATAGCCTCCATCTGTGTTGTATCCA | SEQ ID NO: 177 |
| | TRAC) | GCATGGCTCAGAAGGTAACTCAAGCGCAGACTGAAATTTCTGTGGTGGAGAAGGA | |
| | | GGATGTGACCTTGGACTGTGTGTATGAAACCCGTGATACTACTTATTACTTATTC | |
| | | TGGTACAAGCAACCACCAAGTGGAGAATTGGTTTTCCTTATTCGTCGGAACTCTT | |
| | | TTGATGAGCAAAATGAAATAAGTGGTCGGTATTCTTGGAACTTCCAGAAATCCAC | |
| | | CAGTTCCTTCAACTTCACCATCACAGCCTCACAAGTCGTGGACTCAGCAGTATAC | |
| | | TTCTGTGCTCTGCCCGACAAGGTGATATTTGGGCCAGGGACAAGCTTATCAGTCA | |
| | | TTCCAAATATCCAGAACCCTGACCCTGCCGTGTACCAGCTGAGAGACTCTAAATC | |
| | | CAGTGACAAGTCTGTCTGCCTATTCACCGATTTTGATTCTCAAACAAATGTGTCA | |
| | | CAAAGTAAGGATTCTGATGTGTATATCACAGACAAAACTGTGCTAGACATGAGGT | |
| | | CTATGGACTTCAAGAGCAACAGTGCTGTGGCCTGGAGCAACAAATCTGACTTTGC | |
| | | ATGTGCAAACGCCTTCAACAACAGCATTATTCCAGAAGACACCTTCTTCCCCAGC | |
| | | CCAGAAAGTTCCTGTGATGTCAAGCTGGTCGAGAAAAGCTTTGAAACAGATACGA | |
| | | ACCTAAACTTTCAAAACCTGTCAGTGATTGGGTTCCGAATCCTCCTCCTGAAAGT | |
| | | GGCCGGGTTTAATCTGCTCATGACGCTGCGGCTGTGGTCCAGC | |
| | α 2 (with | ATGCTCCTTGAACATTTATTAATAATCTTGTGGATGcAGcTGAcATGGGTcAGTG | SEQ ID NO: 178 |
| | TRAC) | GTCAACAGCTGAATCAGAGTCCTCAATCTATGTTTATCCAGGAAGGAGAAGATGT | |
| | | CTCCATGAACTGCACTTCTTCAAGCATATTTAACACCTGGCTATGGTACAAGCAG | |
| | | GAACCTGGGGAAGGTCCTGTCCTCTTGATAGCCTTATATAAGGCTGGTGAATTGA | |
| | | CCTCAAATGGAAGACTGACTGCTCAGTTTGGTATAACCAGAAAGGACAGCTTCCT | |
| | | GAATATCTCAGCATCCATACCTAGTGATGTAGGCATCTACTTCTGTGCTGGGCTA | |
| | | TATGCTACAAACAAGCTCATCTTTGGAACTGGCACTCTGCTTGCTGTCCAGCCAA | |
| | | ATATCCAGAACCCTGACCCTGCCGTGTACCAGCTGAGAGACTCTAAATCCAGTGA | |
| | | CAAGTCTGTCTGCCTATTCACCGATTTTGATTCTCAAACAAATGTGTCACAAAGT | |
| | | AAGGATTCTGATGTGTATATCACAGACAAAACTGTGCTAGACATGAGGTCTATGG | |
| | | ACTTCAAGAGCAACAGTGCTGTGGCCTGGAGCAACAAATCTGACTTTGCATGTGC | |
| | | AAACGCCTTCAACAACAGCATTATTCCAGAAGACACCTTCTTCCCCAGCCCAGAA | |
| | | AGTTCCTGTGATGTCAAGCTGGTCGAGAAAAGCTTTGAAACAGATACGAACCTAA | |
| | | ACTTTCAAAACCTGTCAGTGATTGGGTTCCGAATCCTCCTCCTGAAAGTGGCCGG | |
| | | GTTTAATCTGCTCATGACGCTGCGGCTGTGGTCCAGC | |
| | β (with | ATGGGCTTCAGGCTCCTCTGCTGTGTGGCCTTTTGTCTCCTGGGAGCAGGCCCAG | SEQ ID NO: 179 |
| | TRBC1) | TGGATTCTGGAGTCACACAAACCCCAAAGCACCTGATCACAGCAACTGGACAGCG | |
| | | AGTGACGCTGAGATGCTCCCCTAGGTCTGGAGACCTCTCTGTGTACTGGTACCAA | |
| | | CAGAGCCTGGACCAGGGCCTCCAGTTCCTCATTCAGTATTATAATGGAGAAGAGA | |
| | | GAGCAAAAGGAAACATTCTTGAACGATTCTCCGCACAACAGTTCCCTGACTTGCA | |
| | | CTCTGAACTAAACCTGAGCTCTCTGGAGCTGGGGGACTCAGCTTTGTATTTCTGT | |
| | | GCCAGCAGCGTATCGGCAGGGAGCACCGGGGAGCTGTTTTTTGGAGAAGGCTCTA | |
| | | GGCTGACCGTACTGGAGGACCTGAACAAGGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTTCCCCGACCACGTGGAGCTGAGCTGGTGGGTGAATGGGAAGG | |
| | | AGGTGCACAGTGGGGTCAGCACGGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCCGTCACCCAGATCGTCAGCGCCGA | |
| | | GGCCTGGGGTAGAGCAGACTGTGGCTTTACCTCGGTGTCCTACCAGCAAGGGGTC | |
| | | CTGTCTGCCACCATCCTCTATGAGATCCTGCTAGGGAAGGCCACCCTGTATGCTG | |
| | | TGCTGGTCAGCGCCCTTGTGTTGATGGCCATGGTCAAGAGAAAGGATTTC | |
| | β (with | ATGGGCTTCAGGCTCCTCTGCTGTGTGGCCTTTTGTCTCCTGGGAGCAGGCCCAG | SEQ ID NO: 180 |
| | TRBC2) | TGGATTCTGGAGTCACACAAACCCCAAAGCACCTGATCACAGCAACTGGACAGCG | |
| | | AGTGACGCTGAGATGCTCCCCTAGGTCTGGAGACCTCTCTGTGTACTGGTACCAA | |
| | | CAGAGCCTGGACCAGGGCCTCCAGTTCCTCATTCAGTATTATAATGGAGAAGAGA | |
| | | GAGCAAAAGGAAACATTCTTGAACGATTCTCCGCACAACAGTTCCCTGACTTGCA | |
| | | CTCTGAACTAAACCTGAGCTCTCTGGAGCTGGGGGACTCAGCTTTGTATTTCTGT | |
| | | GCCAGCAGCGTATCGGCAGGGAGCACCGGGGAGCTGTTTTTTGGAGAAGGCTCTA | |
| | | GGCTGACCGTACTGGAGGACCTGAAAAACGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTACCCCGACCACGTGGAGCTGAGCTGGTGGGTGAATGGGAAGG | |
| | | AGGTGCACAGTGGGGTCAGCACAGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCTGTCACCCAGATCGTCAGCGCCGA | |
| | | GGCCTGGGGTAGAGCAGACTGTGGCTTCACCTCCGAGTCTTACCAGCAAGGGGTC | |
| | | CTGTCTGCCACCATCCTCTATGAGATCTTGCTAGGGAAGGCCACCTTGTATGCCG | |
| | | TGCTGGTCAGTGCCCTCGTGCTGATGGCCATGGTCAAGAGAAAGGATTCCAGAGG | |
| | | C | |

Accordingly, the invention provides an isolated polynucleotide comprising one or more nucleotide sequences selected from the group consisting of SEQ ID NOs: 56-70, 162-180, 192, 193, 204-213 and 218-221, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

The invention also provides a TCR comprising an α chain encoded by a nucleotide sequence selected from the group consisting of SEQ ID NOs: 56, 59, 62, 65, 68, 162, 167, 168, 171, 172, 177, 178, 192, 193, 204, 206, 208-212, 218 and 220, and variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

The invention also provides a TCR comprising a R chain encoded by a nucleotide sequence selected from the group consisting of SEQ ID Nos: 57, 58, 60, 61, 63, 64, 66, 67, 69, 70, 163, 164, 165, 166, 169, 170, 173, 174, 175, 176, 179, 180, 205, 207, 213, 219 and 221, and variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

Further provided by the invention are isolated polynucleotide sequences derived from the sequences present in Table 2. For example, the present invention provides an isolated polynucleotide encoding a variable region of a TCR according to the invention, wherein the isolated polynucleotide comprises a stretch of nucleotides of any one of SEQ ID Nos: 56-70, 162-180, 192, 193, 204-213 and 218-221.

The variant sequences may have additions, deletions or substitutions, of one or more bases. If the variation involves addition(s) or deletion(s) they may either occur in threes or be balanced (i.e. an addition for each deletion) so that the variation does not cause a frame-shift for translation of the remainder of the sequence.

Some or all of the variations may be “silent” in the sense that they do not affect the sequence of the encoded protein due to the degeneracy of the genetic code.

Some or all of the variations may produce conservative amino acid substitutions, additions or deletions as explained above. The variation may be concentrated in one or more regions, such as the regions encoding the constant regions, the linker, or the framework regions of the α or β chains, or they may be spread throughout the molecule.

The variant sequence should retain the capacity to encode all or part of a TCR amino acid sequence which binds to a WT1 peptide.

Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation may also involve the removal of mRNA instability motifs and cryptic splice sites.

Vector

The invention provides a vector comprising a polynucleotide described herein.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous Cdna segment), to be transferred into a target cell. The vector may serve the purpose of maintaining the heterologous nucleic acid (DNA or RNA) within the cell, facilitating the replication of the vector comprising a segment of nucleic acid, or facilitating the expression of the protein encoded by a segment of nucleic acid. Vectors may be non-viral or viral. Examples of vectors used in recombinant nucleic acid techniques include, but are not limited to, plasmids, chromosomes, artificial chromosomes and viruses. The vector may be single stranded or double stranded. It may be linear and optionally the vector comprises one or more homology arms. The vector may also be, for example, a naked nucleic acid (e.g. DNA). In its simplest form, the vector may itself be a nucleotide of interest.

The vectors used in the invention may be, for example, plasmid or virus vectors and may include a promoter for the expression of a polynucleotide and optionally a regulator of the promoter.

Vectors comprising polynucleotides used in the invention may be introduced into cells using a variety of techniques known in the art, such as transformation, transfection and transduction. Several techniques are known in the art, for example transduction with recombinant viral vectors, such as retroviral, lentiviral, adenoviral, adeno-associated viral, baculoviral and herpes simplex viral vectors, Sleeping Beauty vectors; direct injection of nucleic acids and biolistic transformation.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nature Biotechnology 1996 14; 556) and combinations thereof.

The term “transfection” is to be understood as encompassing the delivery of polynucleotides to cells by both viral and non-viral delivery.

In addition, the invention may employ gene targeting protocols, for example the delivery of DNA-modifying agents.

The term “vector” includes an expression vector i.e. a construct capable of in vivo or in vitro/ex vivo expression. Expression may be controlled by a vector sequence, or, for example in the case of insertion at a target site, expression may be controlled by a target sequence. A vector may be integrated or tethered to the cell's DNA.

Viral delivery systems include but are not limited to adenovirus vector, an adeno-associated viral (AAV) vector, a herpes viral vector, a retroviral vector, a lentiviral vector, and a baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al (“Retroviruses” 1997 Cold Spring Harbour Laboratory Press Eds: J M Coffin, S M Hughes, H E Varmus pp 758-763).

Lentiviruses also belong to the retrovirus family, but they can infect both dividing and non-dividing cells (Lewis et al (1992) EMBO J. 3053-3058).

The vector may be capable of transferring a nucleotide sequence encoding a WT1-specific TCR described herein to a cell, such as a T-cell, such that the cell expresses the WT1-specific TCR. Preferably the vector will be capable of sustained high-level expression in T-cells, so that the introduced TCR may compete successfully with the endogenous TCR for a limited pool of CD3 molecules.

Increasing the supply of CD3 molecules may increase TCR expression, for example, in a cell that has been modified to express the TCRs of the invention. Accordingly, the vector of the invention may further comprise one or more genes encoding CD3-gamma, CD3-delta, CD3-epsilon and/or CD3-zeta. In one embodiment, the vector of the invention comprises a gene encoding CD3-zeta. The vector may comprise a gene encoding CD8. The vector may encode a selectable marker or a suicide gene, to increase the safety profile of the genetically engineered cell, e.g. a cell of the invention, or a cell that has been modified to express the TCRs of the invention (Bonini, Science 1997, Ciceri, Bonini Lancet Oncol. 2009, Oliveira et al., STM 2015). The genes comprised in the vector of the invention may be linked by self-cleaving sequences, such as the 2A self-cleaving sequence.

Alternatively one or more separate vectors encoding a CD3 gene may be provided for co-transfer to a cell simultaneously, sequentially or separately with one or more vectors of the invention, e.g. one or more vectors encoding TCRs of the invention.

Cell

The invention relates to a cell comprising a polynucleotide or a vector according to the invention.

The cell may be a T-cell, a lymphocyte, or a stem cell. The T-cell, the lymphocyte, or the stem cell may be selected from the group consisting of CD4 cells, CD8 cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CK) cells, hematopoietic stem cells and pluripotent stem cells.

The type of cell may be selected in order to provide desirable and advantageous in vivo persistence and to provide desirable and advantageous functions and characteristics to the cells of invention.

The cell may have been isolated from a subject.

The cell of the invention may be provided for use in adoptive cell transfer. As used herein the term “adoptive cell transfer” refers to the administration of a cell population to a patient. Typically, the cells are T-cells isolated from a subject and then genetically modified and cultured in vitro in order to express a TCR of the invention before being administered to the patient.

Adoptive cell transfer may be allogenic or autologous.

By “autologous cell transfer” it is to be understood that the starting population of cells (which are then transduced according to a method of the invention, or are transduced with a vector according to the invention) is obtained from the same subject as that to which the transduced T-cell population is administered. Autologous transfer is advantageous as it avoids problems associated with immunological incompatibility and is available to subjects irrespective of the availability of a genetically matched donor.

By “allogeneic cell transfer” it is to be understood that the starting population of cells (which are then transduced according to a method of the invention, or are transduced with a vector according to the invention) is obtained from a different subject as that to which the transduced cell population is administered. Preferably, the donor will be genetically matched to the subject to which the cells are administered to minimise the risk of immunological incompatibility. Alternatively, the donor may be mismatched and unrelated to the patient.

Suitable doses of transduced cell populations are such as to be therapeutically and/or prophylactically effective. The dose to be administered may depend on the subject and condition to be treated, and may be readily determined by a skilled person.

The cell may be derived from a T-cell isolated from a subject. The T-cell may be part of a mixed cell population isolated from the subject, such as a population of peripheral blood lymphocytes (PBL). T-cells within the PBL population may be activated by methods known in the art, such as using anti-CD3 and/or anti-CD28 antibodies or cell sized beads conjugated with anti-CD3 and/or anti-CD28 antibodies.

The T-cell may be a CD4⁺ helper T cell or a CD8⁺ cytotoxic T cell. The cell may be in a mixed population of CD4⁺ helper T cell/CD8⁺ cytotoxic T-cells. Polyclonal activation, for example using anti-CD3 antibodies optionally in combination with anti-CD28 antibodies will trigger the proliferation of CD4⁺ and CD8⁺ T-cells.

The cell may be isolated from the subject to which the genetically modified cell is to be adoptively transferred. In this respect, the cell may be made by isolating a T-cell from a subject, optionally activating the T-cell, transferring the TCR gene to the cell ex vivo. Subsequent immunotherapy of the subject may then be carried out by adoptive transfer of the TCR-transduced cells. As used herein this process refers to autologous T-cell transfer—i.e. the TCR-transduced cells are administered to the same subject from which the T-cells were originally derived.

Alternatively the T-cell may be isolated from a different subject, such that it is allogeneic. The T-cell may be isolated from a donor subject. For example, if the subject is undergoing allogeneic haematopoietic stem cell transplantation (Allo-HSCT) or solid organ transplantation or cell transplantation or stem cell therapy, the cell may be derived from the donor, from which the organs, tissues or cells are derived. The donor and the subject undergoing treatment may be siblings.

Alternatively the cell may be, or may be derived from, a stem cell, such as a haematopoietic stem cell (HSC). Gene transfer into HSCs does not lead to TCR expression at the cell surface as stem cells do not express CD3 molecules. However, when stem cells differentiate into lymphoid precursors that migrate to the thymus, the initiation of CD3 expression leads to the surface expression of the introduced TCR in thymocytes.

An advantage of this approach is that the mature T-cells, once produced, express only the introduced TCR and little or no endogenous TCR chains, because the expression of the introduced TCR chains suppresses rearrangement of endogenous TCR gene segments to form functional TCR alpha and beta genes. A further benefit is that the gene-modified stem cells are a continuous source of mature T-cells with the desired antigen specificity. The cell may therefore be a gene-modified stem cell, preferably a gene-modified hematopoeitic stem cell, which, upon differentiation, produces a T-cell expressing a TCR of the invention.

Other approaches known in the art may be used to reduce, limit, prevent, silence, or abrogate expression of endogenous genes in the cells of the invention or cells prepared by the methods of the invention.

As used herein the term “disrupting” refers to reducing, limiting, preventing, silencing, or abrogating expression of a gene. The person skilled in the art is able to use any method known in the art to disrupt an endogenous gene, e.g., any suitable method for genome editing, gene silencing, gene knock-down or gene knock-out.

For example, an endogenous gene may be disrupted with an artificial nuclease. An artificial nuclease is, e.g., an artificial restriction enzyme engineered to selectively target a specific polynucleotide sequence (e.g. encoding a gene of interest) and induce a double strand break in said polynucleotide sequence. Typically, the double strand break (DSB) will be repaired by error-prone non-homologous end joining (NHEJ) thereby resulting in the formation of a non-functional polynucleotide sequence, which may be unable to express an endogenous gene.

In some embodiments, the artificial nuclease is selected from the group consisting of zinc finger nucleases (ZFN), transcription activator-like effector nucleases (TALEN) and CRISPR/Cas (e.g. CRISPR/Cas9).

The methods of preparing a cell (e.g. a T-cell) of the invention may comprise the step of targeted integration of an expression cassette into an endogenous gene (e.g. an endogenous TCR α chain gene and/or an endogenous TCR β chain gene). As used herein the term expression cassette refers to a polynucleotide sequence (e.g. a DNA polynucleotide sequence) comprising one or more polynucleotide sequences encoding one or more genes of interest such that said genes of interest are capable of expression. Endogenous sequences may facilitate expression from the expression cassette, and/or transcription control sequences within the expression cassette may facilitate expression. For example, the expression cassette may comprise a polynucleotide sequence of the invention, or a polynucleotide sequence encoding a TCR of the invention, operably linked to an expression control sequence, e.g. a promoter or an enhancer sequence. The one or more genes of interest may be located between one or more sets of restriction sites. Suitably, the restriction sites may facilitate the integration of the expression cassette into, e.g., a vector, a plasmid, or genomic DNA (e.g. host cell genomic DNA).

For example, an expression cassette of the invention may be transferred from a first polynucleotide sequence, e.g. on a vector, to another by ‘cutting’, e.g. excising, the expression cassette using one or more suitable restriction enzymes and ‘pasting’, e.g. integrating, the expression cassette into a second polynucleotide sequence.

The expression cassette may comprise a polynucleotide of the invention. The expression cassette may comprise a polynucleotide encoding one or more TCRs of the invention. The expression cassette may further comprise an antibiotic resistance gene or other selectable marker gene that allows cells that have successfully integrated the expression cassette into their DNA to be identified. The polynucleotide sequences comprised in the expression cassette may be operably linked to expression control sequences, e.g. a suitable promoter or enhancer sequence. The person skilled in the art will be able to select suitable expression control sequences.

The invention also contemplates a cell expressing a TCR of the invention, which has been engineered to disrupt one or more endogenous MHC genes. Disruption of an endogenous MHC gene can reduce or prevent expression of MHC on the engineered cell surface. Accordingly, such an engineered cell with reduced or no MHC expression will have limited or no capacity to present antigens on its cell surface. Such a cell is particularly advantageous for adoptive cell transfer since the cell will be non-alloreactive, e.g., the cell will not present antigens which could be recognized by the immune system of a subject receiving the adoptively transferred cell. As a result, the transferred cell will not be recognized as ‘non-self’ and an adverse immune reaction to the cell can be avoided. Such a cell is termed a ‘universal cell’ since it is suitable for adoptive transfer to a variety of different hosts regardless of HLA type.

Accordingly, the invention provides a method of preparing a non-alloreactive universal T-cell, which expresses a TCR of the invention. Further provided by the invention is a non-alloreactive universal T-cell, which expresses a TCR of the invention.

The invention further contemplates cells which have been engineered to disrupt one more endogenous genes to modify the cell to enhance advantageous properties, characteristics or functions of the cell and/or reduce undesirable properties, characteristics or functions. For example, by disrupting an endogenous cell the persistence, expansion, activity, resistance to exhaustion/senescence/inhibitory signals, homing capacity, or other cell functions may be modified. As used in this context, the term ‘modify’ refers to a change in one or more characteristics relative to an equivalent unmodified cell, e.g. a cell in which an endogenous gene has not been disrupted. For example, the change may be an increase, an enhancement or an introduction of a characteristic or function of the cell relative to an equivalent unmodified cell. Alternatively, the change may be a decrease, suppression or abrogation of a characteristic or function of the cell relative to an equivalent unmodified cell.

The polynucleotides and vectors of the invention may be transferred into specific T-cell subsets, including CD4 and or CD8, naive, memory stem T cells, central memory, effector memory or effector cells, or in other cellular subsets such as to promote different in vivo length of persistence and function in the cells of the invention.

The polynucleotides and vectors of the invention may also be transferred into T-cell subsets such as naïve, memory stem T cells, central memory cells, effector memory cells, effectors.

The polynucleotides and vectors of the invention may also be transferred into T-cell subsets with different polarizations, such as Th0/Tc0, Th1/Tc1, Th2/Tc2, Th17, Th22 or others, depending on the cytokine background most appropriate to target a particular tumor type.

Furthermore, the polynucleotides and vectors of the invention encoding the antigen-specific regions of the TCRs of the present invention may be transferred in other cellular subsets, including gamma/delta T-cells, NK cells, NKT cells, cytokine-induced killer (CIK) cells, hematopoietic stem cells or other cells, in order to obtain the therapeutic effect.

Further provided by the invention is a method of preparing a cell, which comprises the step of transducing a cell in vitro or ex vivo with a vector of the invention. Various methods for transduction of a cell with a vector are known in the art (see e.g. Sambrook et al).

The invention also provides a method of producing a T-cell expressing a TCR of the invention by inducing the differentiation of a stem cell which comprises a polynucleotide or a vector of the invention.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (e.g. CD3, CD4, CD8, CD25, CD127, CD152, CXCR3, or CCR4) may be purified from a starting population of cells. Alternatively, or in addition, a population of cells that does not express another marker may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD3, CD4, CD8, CD25, CD127, CD152, CXCR3, or CCR4) may be achieved by using an agent that binds to that marker, preferably substantially specifically to that marker. An agent that binds to a cellular marker may be an antibody, for example antibody which binds to CD3, CD4, CD8, CD25, CD127, CD152, CXCR3, or CCR4.

The term “antibody” refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSCs.

Chimeric Molecules

In another aspect, the invention provides a chimeric molecule comprising a TCR of the invention, a TCR encoded by a polynucleotide of the invention, or a portion thereof, conjugated to a non-cellular substrate. The conjugation may be covalent or non-covalent.

The non-cellular substrate may be a nanoparticle, an exosome, or any non-cellular substrate known in the art.

The chimeric molecule of the invention may be soluble.

In another aspect the invention provides a chimeric molecule comprising a TCR of the invention, a TCR encoded by a polynucleotide of the invention, or a portion thereof, conjugated to a toxin or an antibody.

The toxin or antibody may be cytotoxic. The toxin may be a cytotoxic molecule or compound, e.g. a radioactive molecule or compound. The TCR portion of the chimeric molecule may confer the ability to recognize cells expressing WT1 protein or peptides. Thus, the chimeric molecule may specifically recognize and/or bind to WT1-expressing tumor cells. Accordingly, the chimeric molecules of the invention may provide WT1-targeted delivery of cytotoxic toxins, antibodies and/or compounds.

WT1-Related Diseases

WT1 is widely expressed on a variety of hematological and solid tumors, while showing limited expression on various healthy tissues (e.g. gonads, uterus, kidney, mesothelium, progenitor cells in different tissues). The inventors have identified and determined the amino acid sequences of TCRs that recognise WT1 peptides. Furthermore, they have demonstrated that T-cells expressing TCRs according to the invention target and kill cells which present WT1 peptide or overexpress WT1 protein.

Accordingly, the invention provides a method for treating and/or preventing a disease associated with expression of WT1, which comprises the step of administering a TCR, an isolated polynucleotide, a vector, or a cell of the invention to a subject in need thereof. The invention also provides a method for treating and/or preventing a disease associated with expression of WT1, comprises the step of administering a cell prepared by the method of the invention to a subject in need thereof.

Further provided by the invention is a TCR of the invention, an isolated polynucleotide of the invention, a vector of the invention, a cell of the invention, or a cell prepared by the method of the invention for use in treating and/or preventing a disease associated with expression of WT1.

The term ‘preventing’ is intended to refer to averting, delaying, impeding or hindering the contraction of the disease. The treatment may, for example, prevent or reduce the likelihood of developing or contracting a disease associated with expression of WT1.

‘Treating’ as used herein refers to caring for a diseased subject, in order to ameliorate, cure or reduce the symptoms of the disease, or in order to reduce, halt or delay the progression of the disease.

The subject may be a human subject. The human subject may be a child. For example, the child may be less than 10 years in age, less than 9 years in age, less than 8 years in age, less than 7 years in age, less than 6 years in age, less than 5 years in age, less than 4 years in age, less than 3 years in age, or less than 2 years in age. The human subject may be an infant.

The subject may have been previously determined to be in need of a TCR, an isolated polynucleotide, a vector, or a cell of the invention, or a cell prepared by the method of the invention on the basis of expression of WT1. For example, the subject may have a cell population that exhibits increased expression of WT1 relative to a healthy control cell population. A variety of techniques known in the art may be used to determine WT1 expression—e.g. quantitative RT-PCR can be used to determine the amount of WT1 RNA transcript, which is indicative of WT1 protein expression. The person skilled in the art will also appreciate that WT1 protein expression may be determined by performing western blots using commercially available antibodies specific for WT1.

The subject may also have been previously identified as having an alteration (e.g. mutation or deletion) in a WT1 gene. Such an alteration may be hereditary. Thus, the disease associated with expression of WT1 may be a hereditary disease. Examples of hereditary diseases associated with expression of WT1 include but are not limited to WAGR (Wilms tumor-Aniridia-Genitourinary malformation-Retardation) syndrome, Denys-Drash syndrome (DDS), Frasier syndrome (FS), genitourinary anomalies (abnormalities of the reproductive and urinary systems) syndrome.

Subjects with hereditary diseases associated with expression of WT1 may be at higher risk of developing a proliferative disorder (e.g. a cancer).

The disease associated with expression of WT1 may be a proliferative disorder.

The proliferative disorder may be a hematological malignancy or a solid tumor. The hematological malignancy may be selected from the group consisting of acute myeloid leukemia (AML), chronic myeloid leukemia (CML), lymphoblastic leukemia, myelodisplastic syndromes, lymphoma, multiple myeloma, non Hodgkin lymphoma, and Hodgkin lymphoma.

The solid tumor may be selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

The disease associated with expression of WT1 may be selected from a group consisting of acute myeloid leukemia (AML), chronic myeloid leukemia (CML), lymphoblastic leukemia, myelodisplastic syndromes, lymphoma, multiple myeloma, non Hodgkin lymphoma, and Hodgkin lymphoma, lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

Pharmaceutical Composition

The TCRs of the invention, the polynucleotides of the invention, the vectors of the invention, the cells of the invention, the cells prepared by the methods of the invention, the chimeric molecules of the invention, and the mixed cell population of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy products is preferably performed in compliance with FACT-JACIE International Standards for cellular therapy.

Method of Treatment

In another aspect, the invention provides a method for treating and/or preventing a disease associated with expression of WT1, which comprises the step of administering a TCR of the invention, an isolated polynucleotide of the invention, a vector of the invention, a cell of the invention, a cell prepared by a method of the invention, a chimeric molecule of the invention, or a mixed cell population of the invention to a subject in need thereof.

The subject may be a human subject. The subject may be a non-human animal subject.

The subject may have a disease associated with expression of WT1. The subject may be at risk of developing a diseases associated with expression of WT1. The subject may have been previously determined to be at risk of developing a disease associated with expression of WT1. The subject may have an increased risk of developing a disease associated with WT1.

The increased risk may have been determined by genetic screening and/or by reviewing the subject's family history. The subject may express genetic markers indicative of increased risk of developing a disease associated with expression of WT1.

Suitably, a person skilled in the art will be aware of genetic risk factors (e.g. genetic markers) associated with increased risk of developing a disease associated with WT1. The skilled person may be able to use any suitable method or technique known in the art to determine whether the subject has an increased risk of developing a disease associated with expression of WT1.

The subject may have previously received treatment for a disease associated with expression of WT1. The subject may be in remission. The subject may be resistant to chemotherapy.

The subject may be resistant to an anti-WT1 therapy.

In one embodiment, the method for treating and/or preventing a disease associated with expression of WT1 comprises the step of administering a chemotherapy to the subject. The chemotherapy may be administered to the subject simultaneously, sequentially or separately with the TCR of the invention, the isolated polynucleotide of the invention, the vector of the invention, the cell according of the invention, the cell prepared by the method of the invention, or the chimeric molecule of the invention.

In another aspect, the invention provides a method of treating and/or preventing a disease associated with expression of WT1, which comprises the step of administering a mixed cell population, wherein the mixed cell population comprises a plurality of cell populations each expressing a different TCR of the invention.

In another aspect, the invention provides a mixed cell population comprising a plurality of cell populations each expressing a different TCR of the invention.

In another aspect, the invention provides a method for preparing a mixed cell population comprising a plurality of cell populations each expressing a different TCR of the invention, wherein the method comprises the step of transducing a cell in vitro or ex vivo with a vector of the invention.

In another aspect, the invention provides a mixed cell population for use in treating and/or preventing a disease associated with expression of WT1, wherein the mixed cell population comprises a plurality of cell populations each expressing a different TCR of the invention.

For example, the mixed cell population may comprise a first cell population expressing a first TCR of the invention and a second cell population expressing a second TCR of the invention.

For example, the mixed cell population may comprise a first cell population expressing a first TCR of the invention, a second cell population expressing a second TCR of the invention, and a third cell population expressing a third TCR of the invention, and so on.

Each cell population of the mixed cell population may, for example, express a single TCR of the invention only. The endogenous TCR genes of the cell populations in the mixed cell population may be disrupted or deleted. Expression of endogenous TCR genes of the cells in the mixed cell population may be disrupted, e.g. by gene editing with an artificial nuclease.

In another aspect, the invention provides use of TCR of the invention, an isolated polynucleotide of the invention, a vector of the invention, a cell of the invention, a cell prepared by a method of the invention, a chimeric molecule of the invention, or a mixed cell population of the invention, for the manufacture of a medicament for the treatment of a disease associated with expression of WT1.

Both human and veterinary treatments are within the scope of the invention.

The practice of the invention will employ, unless otherwise indicated, conventional techniques of cell biology, molecular biology, histology, immunology, oncology, which are within the capabilities of a person of ordinary skill in the art. Such techniques are explained in the literature.

See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

Various preferred features and embodiments of the invention will now be described by way of non-limiting examples.

### EXAMPLES

### Example 1

Materials and Methods

Peptides

The WT1 protein sequence previously published by Gessler et al. (Doubrovina, E. et al. Blood 120: 1633-1646 (2012)) was adopted to design the peptides used for the stimulation and isolation of WT1-specific T cells. This sequence contains 575 amino acids and includes the first 126 amino acids in the N-terminus missing in the (exon 5+, KTS+) isoform of WT1. It is composed of 141 pentadecapeptides spanning the whole sequence of the WT1 protein, each overlapping the next one by 11 amino acids. Starting from the original pool described in Doubrovina et al., in order to increase the probability to enrich for WT1-specific T cells restricted to peptides processed and presented by different HLA alleles (and in particular by the HLA-A*02:01 restriction element), we used 3 different protocols.
1. Stimulation with WT1 pool-137:
For Healthy Donor 12 (HD12), peripheral blood mononuclear cells (PBMCs) were stimulated with a WT1 pool of 137 pentadecapeptides (indicated as WT1 pool-137) obtained by excluding peptides 40, 41, 63, 64 in order to avoid the isolation of T cells specific for the WT1 37-45 epitope (VLDFAPPGA (SEQ ID NO: 72), an immunodominant peptide restricted to the HLA-A*02:01 allele) and the WT1 126-134 epitope (RMFPNAPYL (SEQ ID NO: 71), an immunogenic peptide which has been described to be processed by the immunoproteasome (Jaigirdar, A. et al. J Immunother. 39(3):105-16 (2016) and presented by the HLA-A*02:01 allele).
2. Stimulation with WT1-HLA-A*02:01 pool:
For HD13, HD14, HD15, PBMCs were stimulated with a pool composed of defined peptides already known to be possibly restricted to the HLA-A*02:01 allele (Doubrovina, E. et al. Blood 120: 1633-1646 (2012)). Selected peptides indicated in Table 3 were pooled at a concentration of 13.6 μg/ml per peptide. These peptides are labelled according to the nomenclature already used for the WT1 pool (141 peptides) described above (indicated as WT1-HLA-A*02:01 pool). We did not include in the new pool peptides VLDFAPPGA (SEQ ID NO: 72) (P40-41) and RMFPNAPYL (SEQ ID NO: 71) (P63-64).
3. Stimulation with a single peptide:
PBMCs of HD15 were also stimulated with a single peptide (P91) chosen for its HLA-restriction (possibly HLA-A*02:01), its natural processing and its expression on primary leukemic blasts (as reported in Doubrovina et al.).

Peptides were synthesised by PRIMM to specifications of validated sequence, 70% purity, sterility and absence of endotoxin. These peptides were mixed in equal amounts in the WT1 pool composed of 137 peptides (WT1 pool-137) at a concentration of 1 μg/ml per peptide. Additionally, 24 subpools were generated, each containing up to 12 peptides (4.17 μg/ml per peptide) according to a specific mapping matrix in order to have each peptide included in only two overlapping subpools as shown in Table 4.

TABLE 3
Peptides included in the WT1 HLA-A*02:01 pool.

| Peptide number | Peptide sequence | SEQ ID NO |
|---|---|---|
| P4 | PTACPLPHFPPSLPP | SEQ ID NO: 80 |
| P7 | LPPTHSPTHPPRAGT | SEQ ID NO: 81 |
| P13 | LLAAILDFLLLQDPA | SEQ ID NO: 82 |
| P20 | RSGPGCLQQPEQQGV | SEQ ID NO: 83 |
| P25 | IWAKLGAAEASAERL | SEQ ID NO: 84 |
| P33 | SDVRDLNALLPAVPS | SEQ ID NO: 85 |
| P37 | GGGGGCALPVSGAAQ | SEQ ID NO: 86 |
| P91 | CMTWNQMNLGATLKG | SEQ ID NO: 87 |
| P92 | NQMNLGATLKGVAAG | SEQ ID NO: 88 |
| P129 | TCQRKFSRSDHLKTH | SEQ ID NO: 89 |
| P131 | SDHLKTHTRTHTGKT | SEQ ID NO: 90 |

TABLE 4
Mapping grid strategy.

| | SP1 | SP2 | SP3 | SP4 | SP5 | SP6 | SP7 | SP8 | SP9 | SP10 | SP11 | SP12 |
|---|---|---|---|---|---|---|---|---|---|---|---|---|
| SP13 | 1 | 2 | 3 | 4 | 5 | 6 | 7 | 8 | 9 | 10 | 11 | 12 |
| SP14 | 13 | 14 | 15 | 16 | 17 | 18 | 19 | 20 | 21 | 22 | 23 | 24 |
| SP15 | 25 | 26 | 27 | 28 | 29 | 30 | 31 | 32 | 33 | 34 | 35 | 36 |
| SP16 | 37 | 38 | 39 | 40 | 41 | 42 | 43 | 44 | 45 | 46 | 47 | 48 |
| SP17 | 49 | 50 | 51 | 52 | 53 | 54 | 55 | 56 | 57 | 58 | 59 | 60 |
| SP18 | 61 | 62 | 63 | 64 | 65 | 66 | 67 | 68 | 69 | 70 | 71 | 72 |
| SP19 | 73 | 74 | 75 | 76 | 77 | 78 | 79 | 80 | 81 | 82 | 83 | 84 |
| SP20 | 85 | 86 | 87 | 88 | 89 | 90 | 91 | 92 | 93 | 94 | 95 | 96 |
| SP21 | 97 | 98 | 99 | 100 | 101 | 102 | 103 | 104 | 105 | 106 | 107 | 108 |
| SP22 | 109 | 110 | 111 | 112 | 113 | 114 | 115 | 116 | 117 | 118 | 119 | 120 |
| SP23 | 121 | 122 | 123 | 124 | 125 | 126 | 127 | 128 | 129 | 130 | 131 | 132 |
| SP24 | 133 | 134 | 135 | 136 | 137 | 138 | 139 | 140 | 141 | | | |

Isolation of Peripheral Blood Mononuclear Cells

Peripheral blood was obtained from 4 healthy donors (HDs) at San Raffaele Hospital (OSR) upon informed consent. Peripheral blood mononuclear cells (PBMCs) were isolated using Ficoll-Hypaque density gradient centrifugation.

Immortalised B Cells

Autologous B cells were isolated from PBMCs of healthy donors using the 0019 Microbeads (Miltenyi Biotec). Cells were transduced with a lentiviral vector harboring the BCL-6/BCL-XL transgene (Kwakkenbos, M. J. et al. Nat. Med. January; 16(1):123-(2010)) and the H/F pseudotype (Lévy, C. et al. Molecular Therapy 20 9, 1699-1712, (2012) and cultured in Iscove's Modified Dulbecco's Medium (IMDM) (Euroclone/Lonza) supplemented with 10% fetal bovine serum (FBS; Carlo Erba), 1% penicillin-streptomycin (Euroclone/Lonza), 2 mM glutamine and 50 ng/ml of IL21 (Miltenyi Biotec). B-cells were re-stimulated every 5 days by co-culture with irradiated (80 Gy) mouse L-cell fibroblasts expressing CD40L (3T3-CD40L) at a B-cell:3T3-CD40L ratio of 10:1.

Cell Lines

T2 and EBV-BLCLs cell lines were cultured in IMDM (Euroclone/Lonza) both supplemented with 1% penicillin-streptomycin, 2 mM glutamine and 10% FBS.

Leukemic Cells

Primary AML cells were obtained from the OSR Leukemia biobank and selected according to the expression of WT1 (determined by quantitative PCR) and of the HLA typing. In co-culture experiments, leukemic blasts were kept in X-VIVO 15 (Euroclone/Lonza) medium supplemented with 5% HS, 1% penicillin-streptomycin, 2 mM glutamine, IL3 and G-CSF (Peprotech; both 20 ng/ml).

HLA Typing

Healthy donor samples, Epstein-Barr virus (EBV)-B lymphoblastoid cell lines (BLCLs) and primary leukemic cells were typed for HLA-A, HLA-B, HLA-C alleles at high resolution at the HLA laboratory of OSR.

Flow Cytometry

FITC-, PE-, PerCP-, APC-, PE-Cy7, APC Cy7-, Pacific Blue and Brillant Violet-conjugated antibodies directed to human CD3, CD4, CD8, CD107a, interferon (IFN)γ, Tumor necrosis factor (TNF)α, CD33, CD117, CD34, CD14, anti-active Caspase 3, and HLA-A2 were used.

Cells were incubated with antibodies for 15 minutes at 4° C. and washed with phosphate-buffered saline (PBS) containing 1% FBS. For Caspase 3 staining, cells were incubated for one hour at 4° C. Zombie Aqua Fixable Viability kit (Biolegend) was used to stain dead cells according to the manufacturer's instructions. Flow cytometry data were acquired using one of the following cell analysers: BD Canto II flow cytometer, BD LSRFortessa, Cytoflex S (Beckman Coulter). Data were analysed by Flow Jo software (Tree star Inc). For intracellular evaluation of cytokine secretion and expression of degranulation markers, the Fix/Perm buffer set (Biolegend) was used according to the manufacturer's instructions.

Stimulation, Isolation and Expansion of WT1-Specific T-Cells

Freshly isolated PBMCs were resuspended in X-VIVO 15 (Euroclone/Lonza) supplemented with 5% human AB serum, 1% penicillin-streptomycin, 2 mM glutamine and 1 μg/ml CD28 monoclonal antibody (BD Biosciences), seeded at a density of 10⁷ cells/ml and stimulated with: 1) WT1 pool-137 for HD12, 2) WT1-HLA-A*02:01 pool for HD13-HD14-HD15, 3) single peptide (P91) for HD15.

For experiments performed with 1) and 2), antigen-specific T-cells were isolated after 26-30 hours by CD137 expression. More specifically, cells were stained with the PE-conjugated CD137 antibody and sorted using anti-PE microbeads (Miltenyi Biotec). The CD137− fraction was depleted of the CD3 cells using CD3-Microbeads (Miltenyi Biotec), irradiated 30 Gy and used as peptide-loaded antigen presenting cells (APCs) in a co-culture with the CD137+ fraction at a ratio of 100:1 when possible or at least 20:1 and a final density of 5×10⁶ cells/ml. X-VIVO 15 supplemented with 5% human AB serum, 1% penicillin-streptomycin, 2 mM glutamine, 5 ng/ml IL7, 5 ng/ml IL15 and 10 ng/ml IL21 was used as medium. Media, including cytokines, was replaced every 2-3 days.

For the experiment performed with 3), antigen-specific T cells were stimulated with P91 epitope in RPMI (Euroclone/Lonza) supplemented with 5% human AB serum. After 6 hours, cells were harvested, washed with PBS, labelled with the IFNγ-catch reagent and incubated for 45 minutes at 37° C. Afterwards, cells were stained with a PE-labelled antibody to IFNγ, enriched by using anti-PE microbeads and separated using the MACS system (Miltenyi Biotec). IFNγ-enriched T cells were co-cultured with IFNγ-CD3− fraction irradiated with 30 Gy at a ratio of 100:1 and seeded at a density of 5×10⁶ cells/ml. X-VIVO 15 supplemented with 5% human AB serum, 1% penicillin-streptomycin, 2 mM glutamine, 5 ng/ml IL7, 5 ng/ml IL15 and 10 ng/ml IL21 was used as medium. Media, including cytokines, was replaced every 2-3 days.

After ˜20 days T cells were pelleted and used for TCR sequencing analysis.

Re-stimulation of expanded antigen-specific T-cells

Cells originally stimulated using either protocol 1) or 2) (as described above) were re-stimulated every 7-14 days with WT1-pulsed autologous APCs (PBMC CD3-depleted cells). In the initial re-stimulations, cells were washed 2 days before and plated in cytokine-free medium. APCs were irradiated with 30 Gy, pulsed with the peptide pool overnight in X-VIVO 15 supplemented with 5% AB serum or at least 3 hours on a rotator in IMDM without serum. Pulsed APCs were co-cultured with effector cells in X-VIVO 15 supplemented with 5% human AB serum, 1% penicillin-streptomycin, 2 mM glutamine, 1 μg/ml CD28 monoclonal antibody and IL7 (5 ng/ml), IL15 (5 ng/ml), IL21 (10 ng/ml).

Assessment of T Cell Response

The percentage of T-cells responding to the WT1 pool-137 or to the WT1-HLA*A02:01 pool was measured by performing a 6 hour co-culture of the effector cells with autologous APCs (ratio of at least 1:1) pulsed with the desired antigen (WT1 pool-137 or WT1-HLA*02:01 pool, WT1 subpools or unrelated peptide pool as control). Co-cultures were seeded in X-VIVO 15 supplemented with 5% human AB serum, 1% penicillin-streptomycin, 2 mM glutamine and supplemented with the CD28 monoclonal antibody (1 μg/ml), Golgi Stop Protein transport inhibitor (BD Biosciences; 1 μg/ml) and CD107a-FITC antibody (BD Biosciences; 4 μl/well) for assessment of degranulation. Cells were then fixed, permeabilised and stained intracellularly to determine the percentage of CD3+CD8+ or CD3+CD4+ cells secreting IFNγ and expressing CD107a.

Mapping of Immunogenic Peptides

WT1-specific T-cells of HD12 enriched using the WT1 pool-137 were seeded in different wells and co-cultured with autologous APCs loaded with one of each of the WT1 subpools. WT1-specific T-cells of HD13 and HD14, enriched using the WT1 HLA-A*02:01 pool were seeded in different wells and co-cultured with autologous APCs loaded with the individual peptides included in the WT1-HLA*A02:01 pool. For HD15, mapping of immunogenic peptides was not performed due to a reduced cellularity.

Each co-culture was seeded at an effector to target ratio of at least 1:1. T-cell responses to each subpool or peptide were measured as previously described by FACS analysis. For HD12, deconvolution of the mapping grid was essential to determine which shared peptide was eliciting a T cell response. Once determined the immunogenic epitopes, T-cells of HD12, HD13, HD14 were further stimulated with APCs loaded with the individual peptides.

Evaluation of T Cell Ability to Recognise WT1-Expressing Cells

WT1-specificity and HLA-restricted ability of T-cells to recognise target cells was measured with different experimental procedures. For HD13 and HD14, the percentage of living target cells expressing the Caspase 3 were determined. Primary leukemic blasts and T cells were incubated at an effector to target (E:T) ratio of 10:1, 4:1, 1:1, 1:4 and 1:10 for 6 hours. As a negative control, target cells were cultured with unrelated T lymphocytes. Cells were fixed, permeabilised using the Fix/Perm buffer set (Biolegend) and stained with anti-active Caspase-3-antibody conjugated to Pacific Blue (Biolegend). Dead cells were visualised upon staining with Zombie Aqua Fixable Viability kit (Biolegend).

For the remaining donors, due to a reduced fitness of the expanded WT1-specific T cells, it was not possible to perform these functional assays.

Enrichment of IFNγ-Secreting Cells

In order to enrich T cells specific for WT1 expanded from HD13, we performed the IFNγ capture assay (Miltenyi Biotec). Briefly, T cells were stimulated with the immunogenic recognised epitope for 6 h. Cells were harvested, washed with PBS and labelled with the IFNγ-catch reagent. After 45 minutes incubation at 37° C., cells were stained with a PE-labelled antibody to the IFNγ. IFNγ-secreting cells were afterwards enriched by using anti-PE microbeads and separated using the MACS system (Miltenyi Biotec). IFNγ-enriched T cells were expanded using the protocol described in the following paragraph.

Expansion of WT1-Specific T Cells

Upon several restimulations with autologous APCs, to further expand WT1-specific T cells from HD12, HD13, HD14, different protocols were used.

For HD12 and HD13, a rapid expansion protocol (REP) was used as previously described (Riddell, S. R. et al. Science 80 (1992); ME, D., L T, N., Westwood, J., J R, W. & S A, R. Cancer J. (2000)).

For HD14, WT1-expanded T cells were stimulated with allogeneic irradiated (30 Gy) feeder cells derived from 3 different donors (2 of them harbouring the HLA-A*0201 allele) as well as T2 irradiated (100 Gy) cells both pulsed with the P13 peptide (effector:T2:feeder ratio=1:5:1).

Assessment of T Cell Clonality

In order to determine the clonality of the expanded WT1-specific T cells, the 10 Test Beta Mark TCR V beta repertoire kit (Beckman Coulter) was used according to the manufacturer's recommendations.

TCR Repertoire Sequencing

WT1-specific T cells were collected at different time points over the co-culture time frame and RNA was extracted by using the Arcturus Pico Pure RNA extraction kit (Life Technology).

Complementarity determining region (CDR) 3 sequences of the WT1-specific T cells were amplified by using a modified RACE approach (Ruggiero, E. et al. Nat. Commun. 6,8081 (2015)). Samples were sequenced by using an Illumina MiSeq sequencer and CDR3 clonotypes identified using the MiXCR software (Bolotin, D A et al. Nature Methods 12, 380-381 (2015)).

Lentiviral Vectors

TCR α and β chain genes isolated from HD12, HD13, HD14 and HD15 were codon-optimised, cysteine-modified (Kuball, J. et al. (2007) Blood 109: 2331-8) and cloned in a lentiviral vector (LV) under a bidirectional promoter (European Patent No. 1616012). For WT1-specific T cells originating from HD14 the MiXCR analysis revealed the occurrence of 3 possible TRAV genes in the generation of the same CDR3 region recognising peptide 13. Thus, we ordered 3 different TCR constructs harbouring one of the following genes: TRAV12-3*01, TRAV12-2*01, TRAV12-2*02. For TCRs harbouring TRAV12-2*01 and TRAV12-2*02 genes, we tested: 1) codon optimised, cysteine-modified forms and 2) codon optimised, cysteine-modified forms further mutagenised in order to remove one N-glycosylation site in the TCR alpha constant domain (Kuball, J et al. (2009) J Exp Med 206: 463-75). In particular, we substituted the amino acid N at position 36 in a N-X-S/T motif with the amino acid Q.

HD14-derived TCRs were named as follows:
TRAV 12-3*01—cysteine modified, codon optimized
TRAV12-2*01 WT—cysteine modified, codon optimized
TRAV12-2*01 mut—cysteine modified, codon optimized, mutagenized to remove a N glycosylation site
TRAV12-2*02 WT—cysteine modified, codon optimized
TRAV12-2*02 mut—cysteine modified, codon optimized, mutagenized to remove a N glycosylation site

For each TCR, the alpha chain was cloned in antisense orientation under the minimal human CMV promoter and the beta chain in sense orientation under the PGK promoter. LVs were packaged by an integrase-competent third-generation construct and pseudotyped by the vescicular stomatitis virus (VSV) envelope.

Vector Transductions

For transduction with HD13- and HD14-TCR lentiviral vectors, T lymphocytes isolated from healthy individuals were activated and sorted using magnetic beads conjugated to antibodies to CD3 and CD28 (ClinExVivo CD3/CD28; Invitrogen), following the manufacturer's instructions. Cells were seeded at a concentration of 1-2×10⁶ cells/ml and cultured in IMDM supplemented with 1% penicillin, 1% streptomycin, 10% FBS and 5 ng/ml of each IL-7 and IL-15. For transduction, T lymphocytes were plated at 2.5×10⁶ cells/ml and infected with the LV for 24 h. Afterwards, T cells were cultured at 10⁶ cells/ml and expanded. Transduction efficiency was determined by measuring the percentage of the CD3⁺ T cells expressing the specific Vβ (HD13: no antibody available for the Vs; HD14: Vβ 12).

TCR Editing of T Lymphocytes

PBMCs from HDs were activated and sorted using magnetic beads conjugated to antibodies to CD3 and CD28 (ClinExVivo CD3/CD28; Invitrogen) and seeded at a concentration of 1-2×10⁶ cells/ml in X-VIVO 15 supplemented with 1% penicillin, 1% streptomycin, 5% FBS and 5 ng/ml of each IL-7 and IL-15. After 2 days, T cells were electroporated with RNP complexes (originated from the combination of TRAC or TRBC guides and Cas9 protein) simultaneously. Edited T lymphocytes were transduced at day 3 with a LV encoding for the HD12-, HD13- and HD14-derived TCRs. After 6 days, beads were detached and cells were seeded at a concentration of 1×10⁶ cells/ml. After 14 days, transduction efficiency was determined by measuring the percentage of CD3+ T cells expressing the specific Vβ (HD12:V322; HD13: no antibody available for the Vs; HD14: Vβ 312). HD12-edited T cells were stained with MIX G (containing anti-VP22 antibody conjugated to FITC fluorochrome-IO Test® Beta Mark kit, Beckman Coulter) and sorted using anti-FITC Microbeads (Miltenyi Biotec) following the manufacturer's instructions.

Functional Assays with Engineered T Lymphocytes

The ability of HD12, HD13 and HD14-engineered T cells (either by TCR gene transfer or TCR gene editing) to recognise target cells was measured upon co-culture with: (a) for HD13 and HD14 TCRs, T2 cells either pulsed with a peptide pool (WT1 pool or an unrelated one) or with subpools (1 and 14, both containing peptide 13, or with an unrelated one) at an (E) effector:target (T) ratio of 1:1; (b) for HD12 TCR, an EBV cell line harbouring the HLA-C*07:02 allele pulsed with peptide 103 or with an unrelated peptide as control; (c) for HD14 TCR, primary AML blasts selected according to the expression of the HLA-A*0201 allele and of the WT1 antigen (at different E:T ratios, i.e. 50:1; 5:1). After 6 hour co-culture, the percentage of responding cells was determined by evaluating CD107a expression and/or IFNγ secretion on CD8+T lymphocytes by cytofluorimetric analysis for assays involving T2 cells or EBV cell lines and active Cas3 expression on living target cells for assays involving primary AML blasts.

Results

Generation of Functional WT1-CTLs from Healthy Donors.

We stimulated PBMCs from HD12 using a pool of 137 pentadecapeptides (WT1 pool-137), which differs from the original pool described by Doubrovina et al. as we excluded peptides 40, 41, 63, 64. These peptides were excluded in order to avoid the isolation of T cells specific for the WT1 37-45 epitope (VLDFAPPGA, SEQ ID NO: 72) and the WT1 126-134 epitope (RMFPNAPYL, SEQ ID NO: 71).

Furthermore, we stimulated PBMCs from additional 3 donors (HD13-HD15) with the WT1-HLA-A*02:01 pool. After 26-30 hours, CD137+ T cells were sorted and co-cultured with the CD137− population, further depleted of the CD3 fraction, and irradiated at 30 Gy.

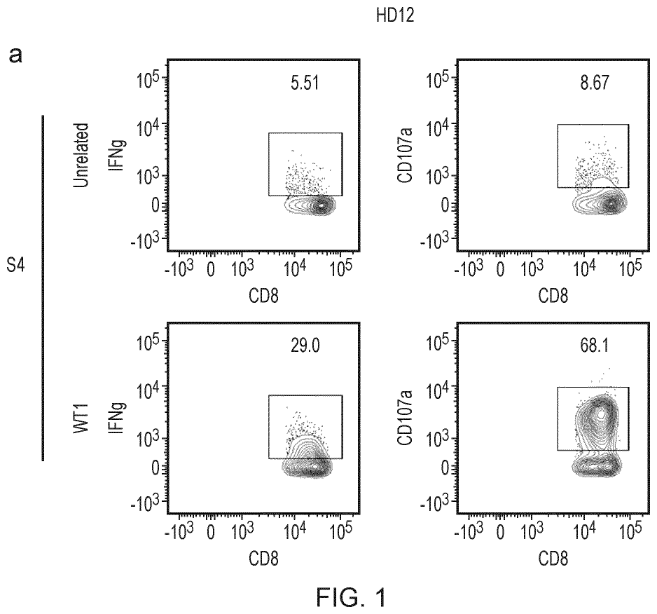

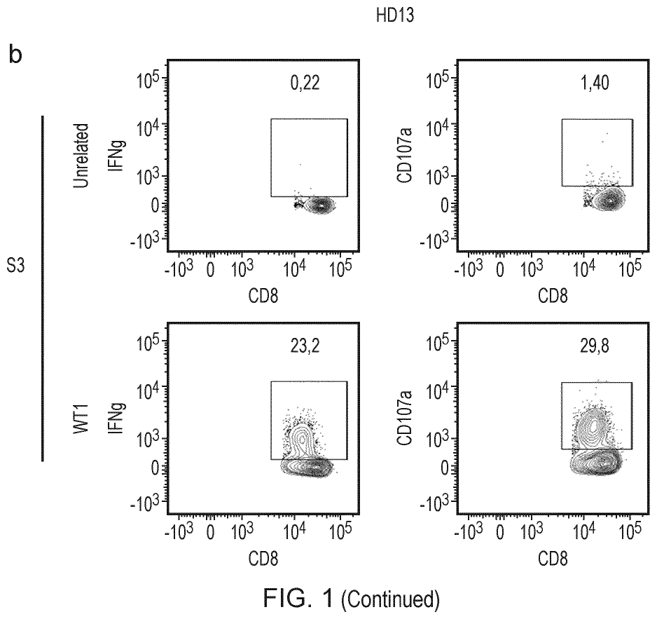

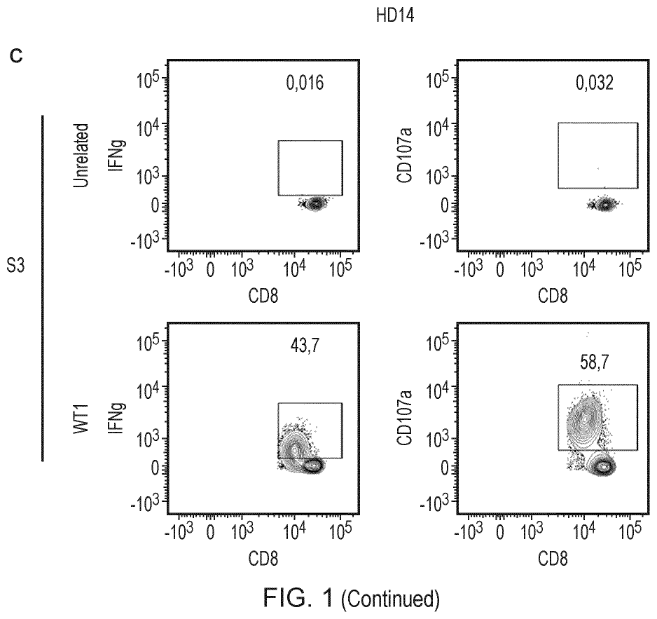

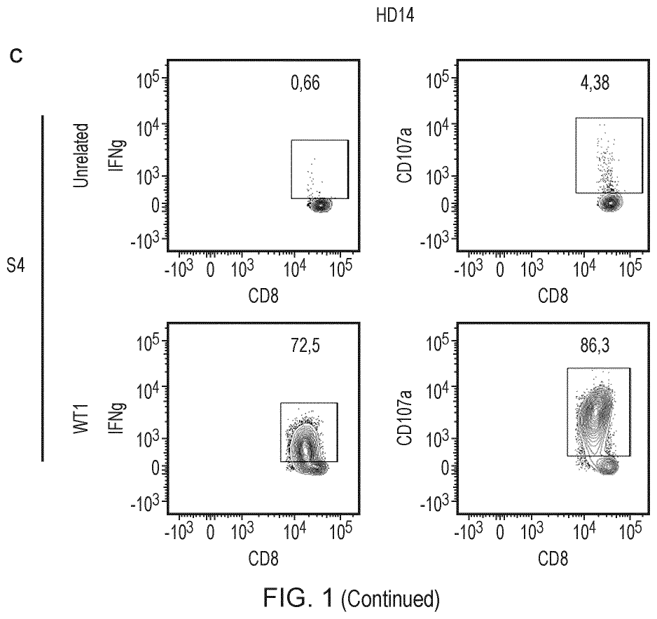

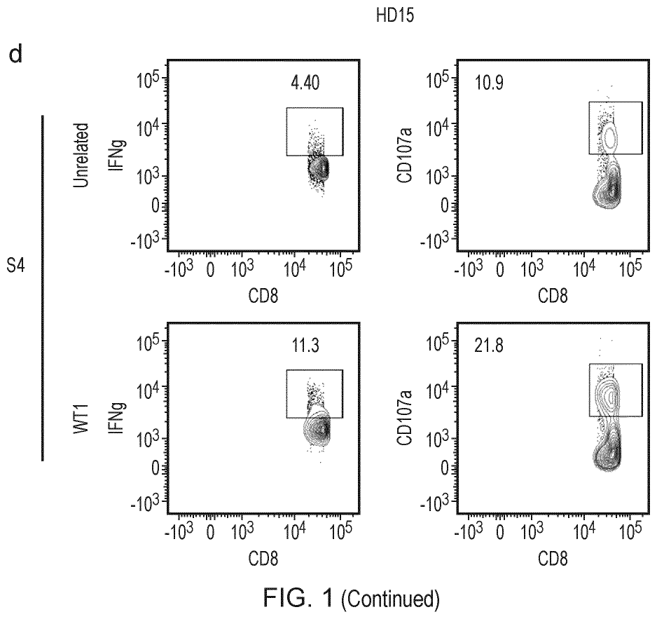

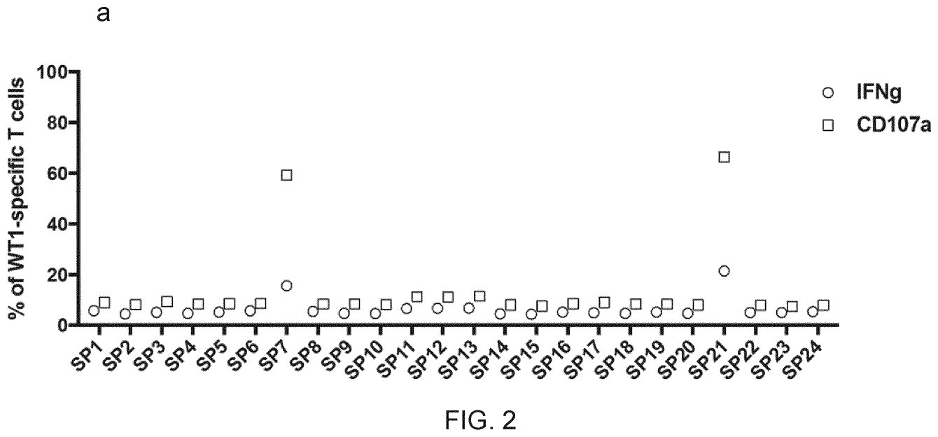

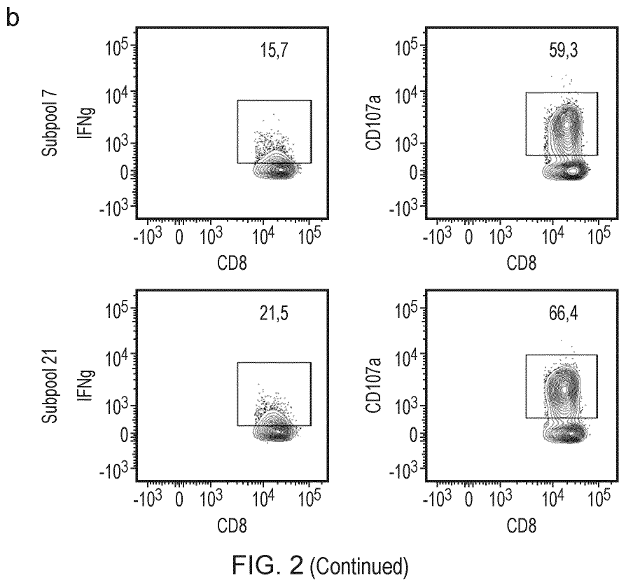

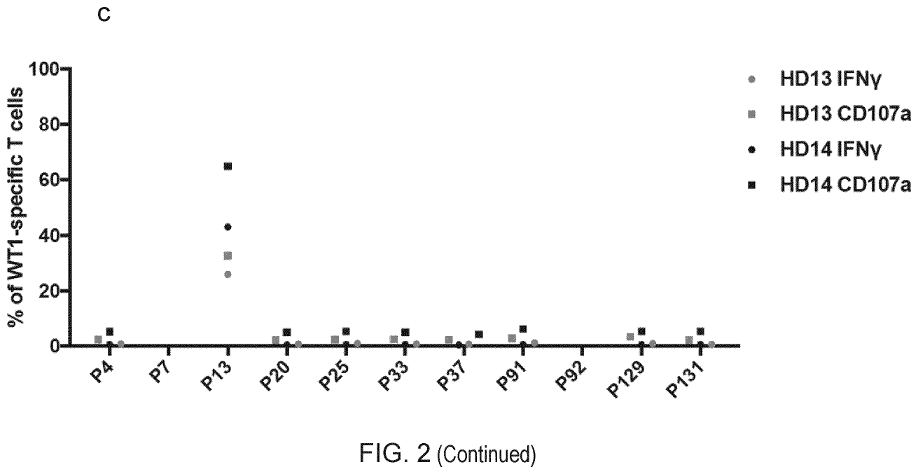

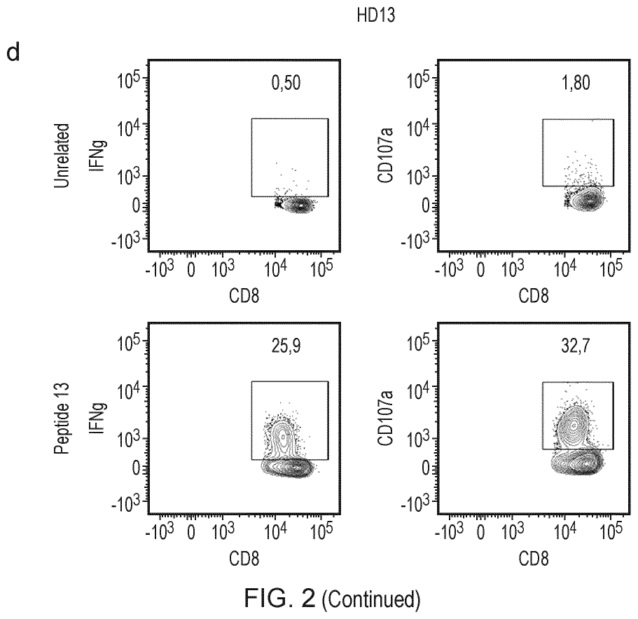

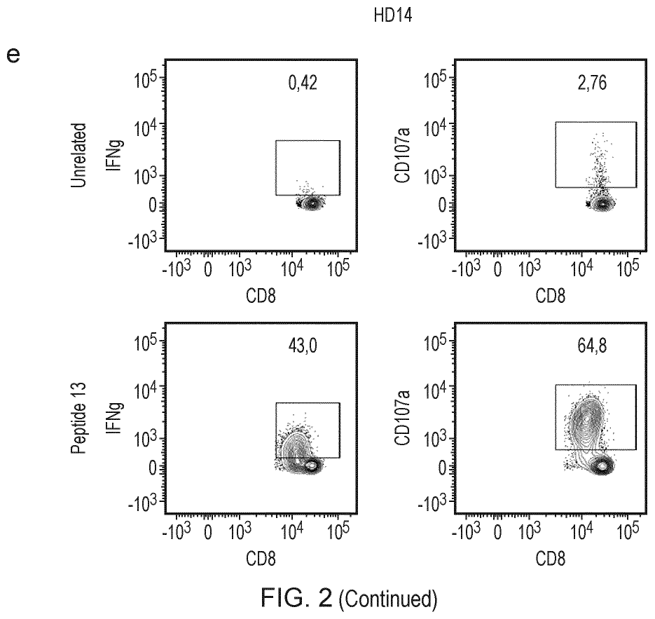

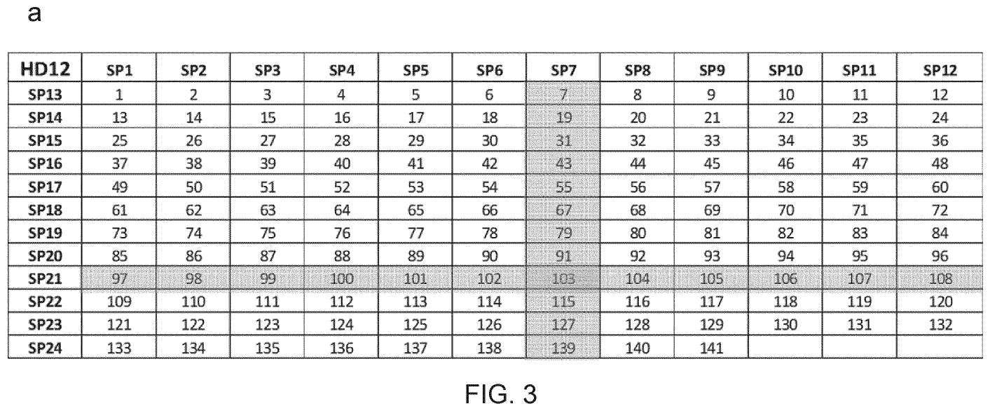

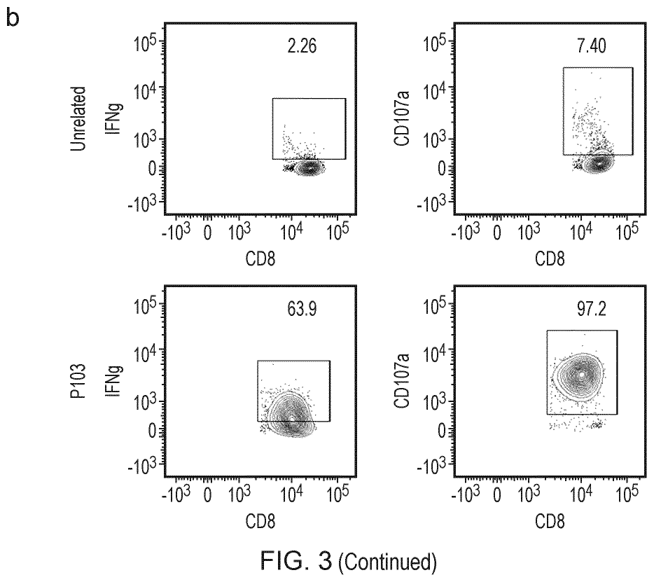

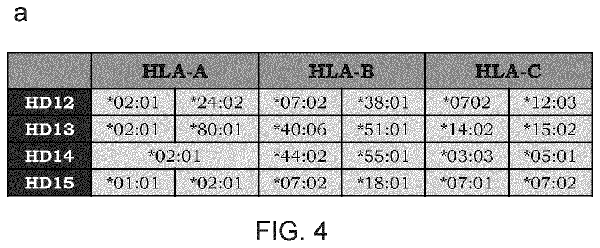

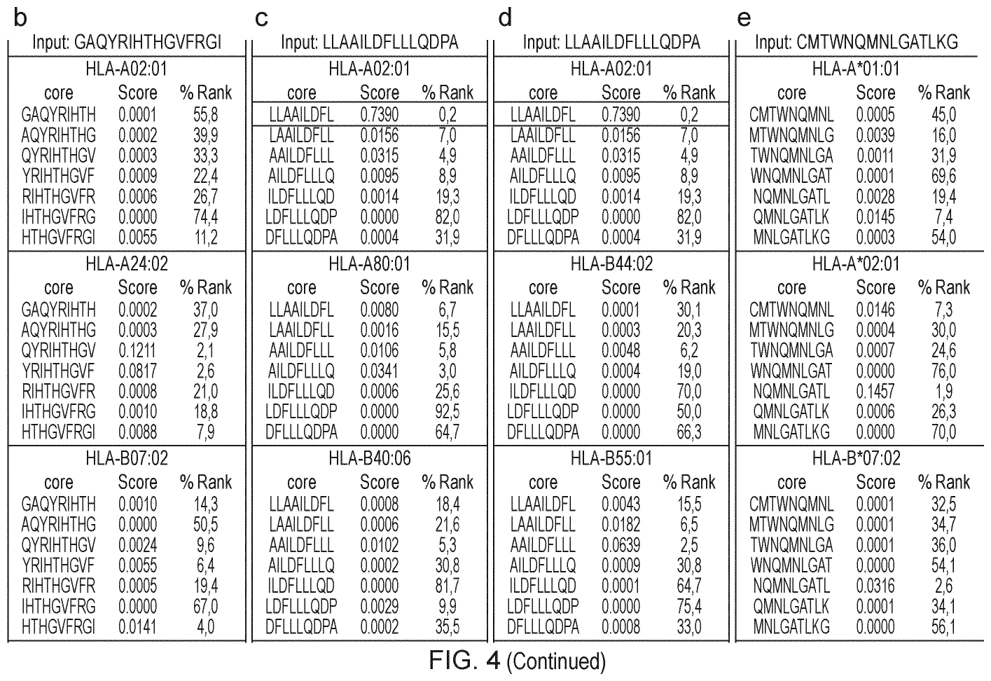

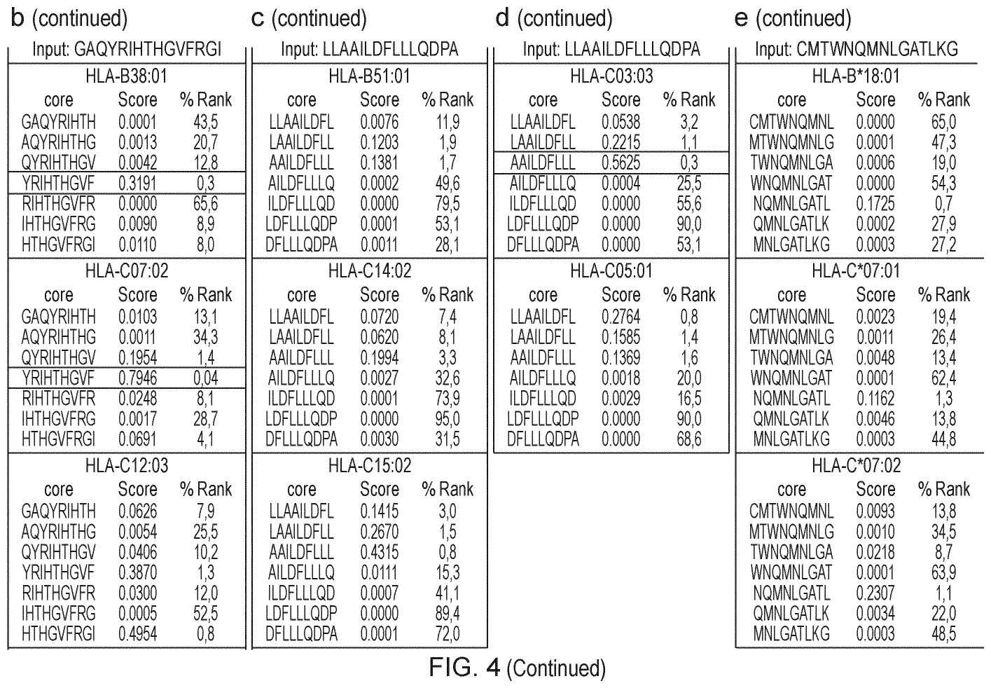

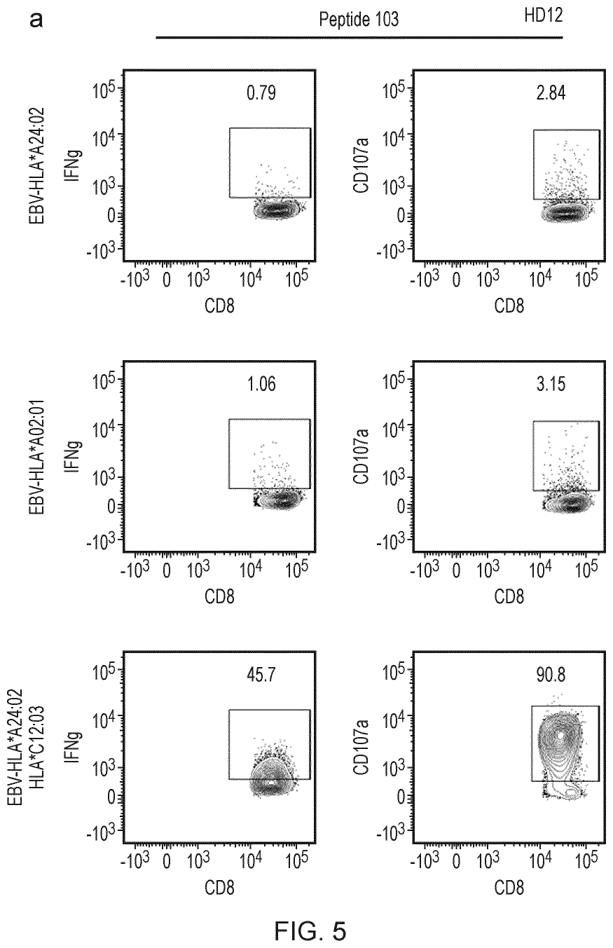

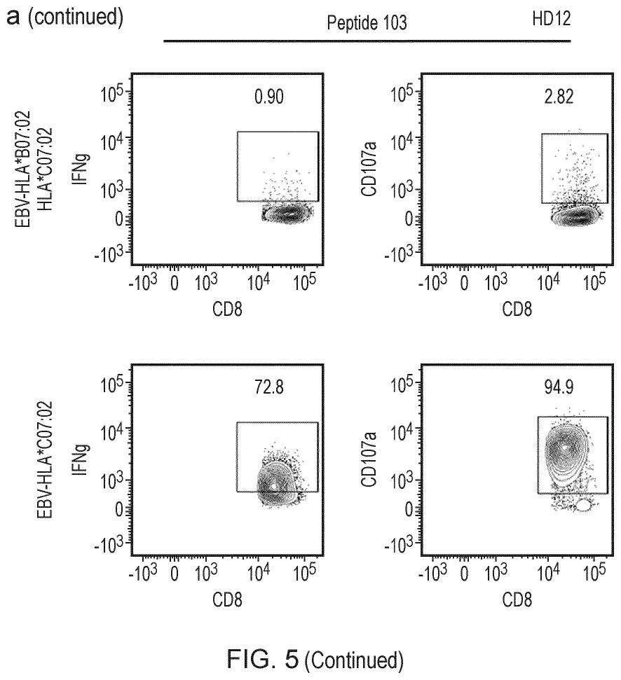

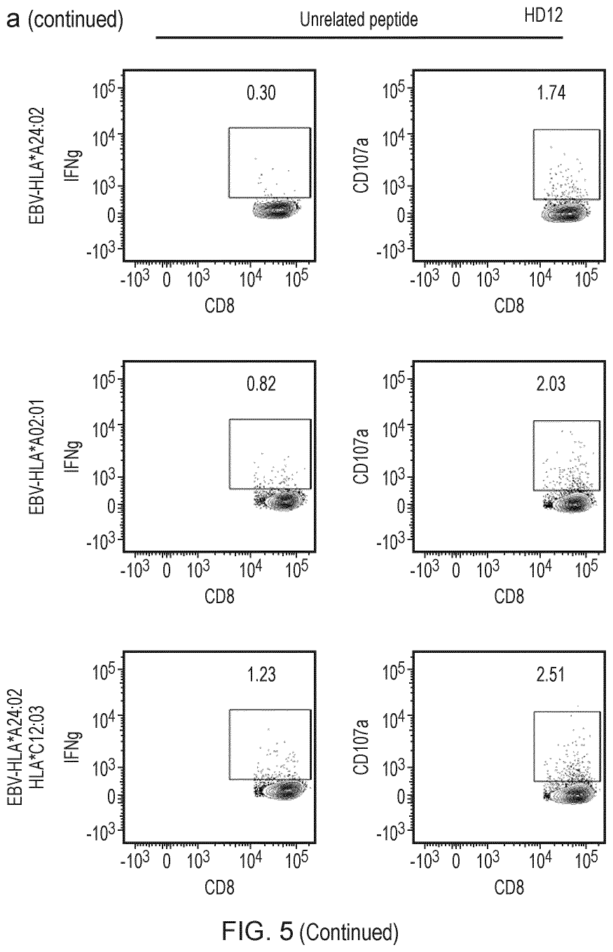

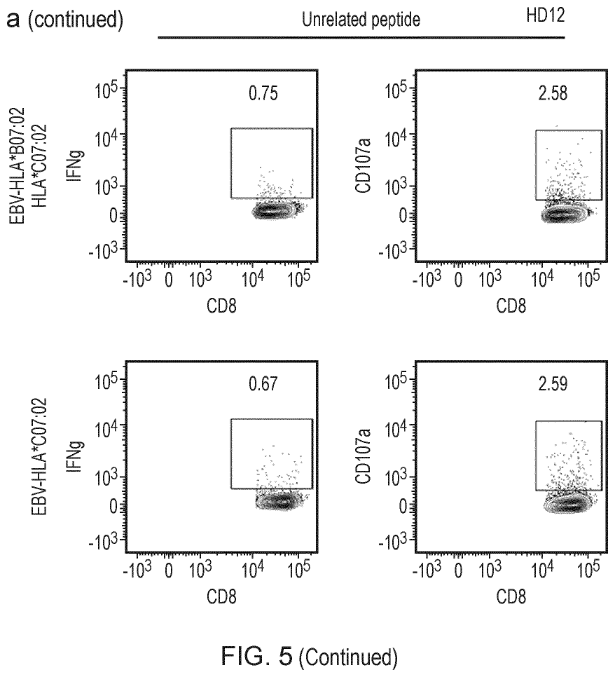

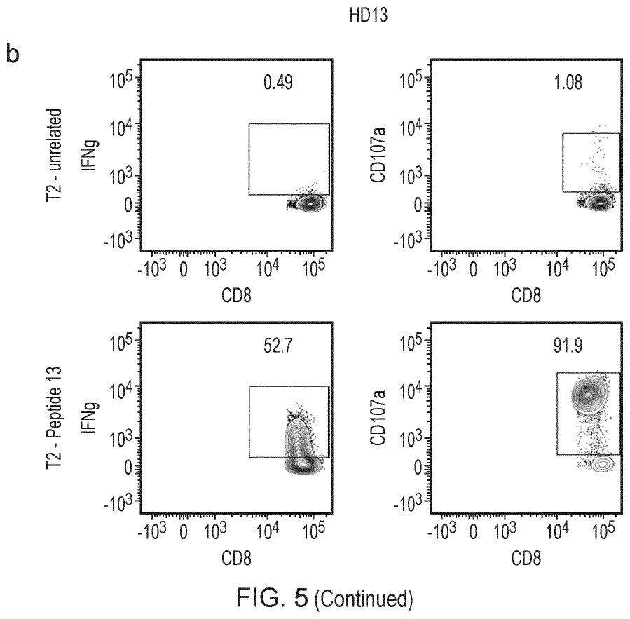

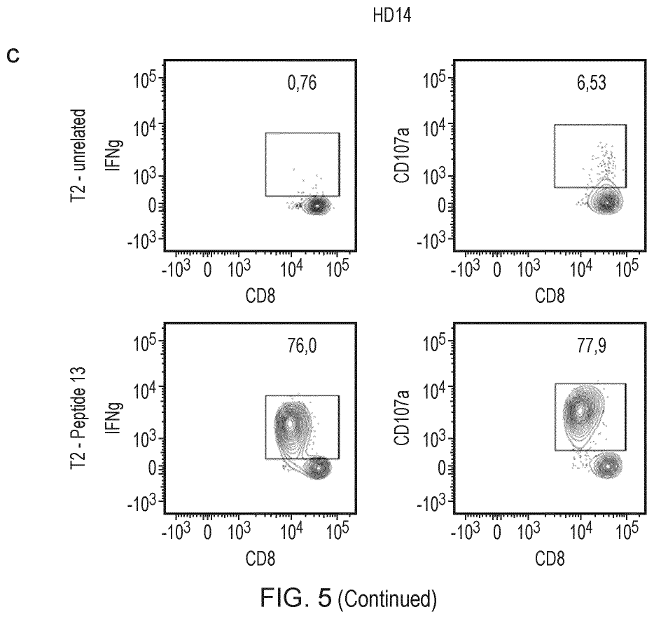

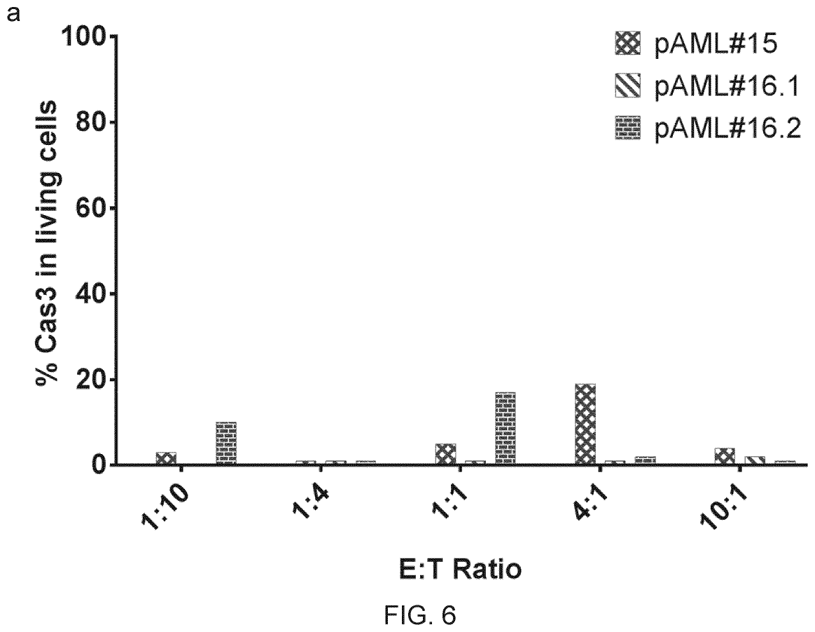

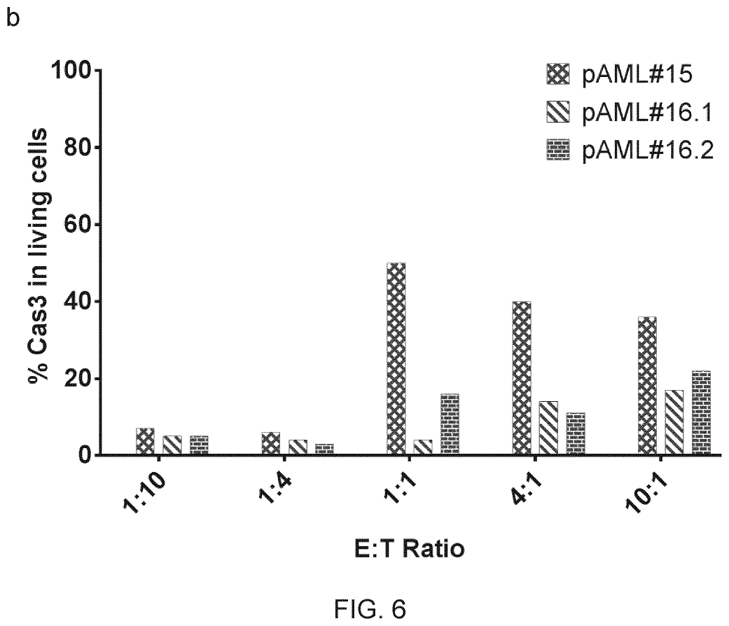

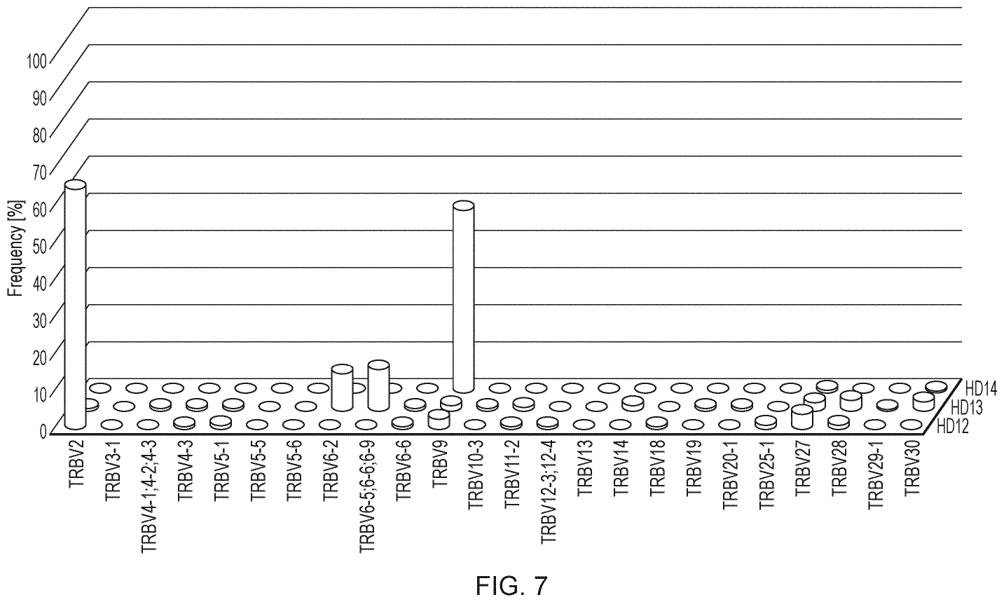

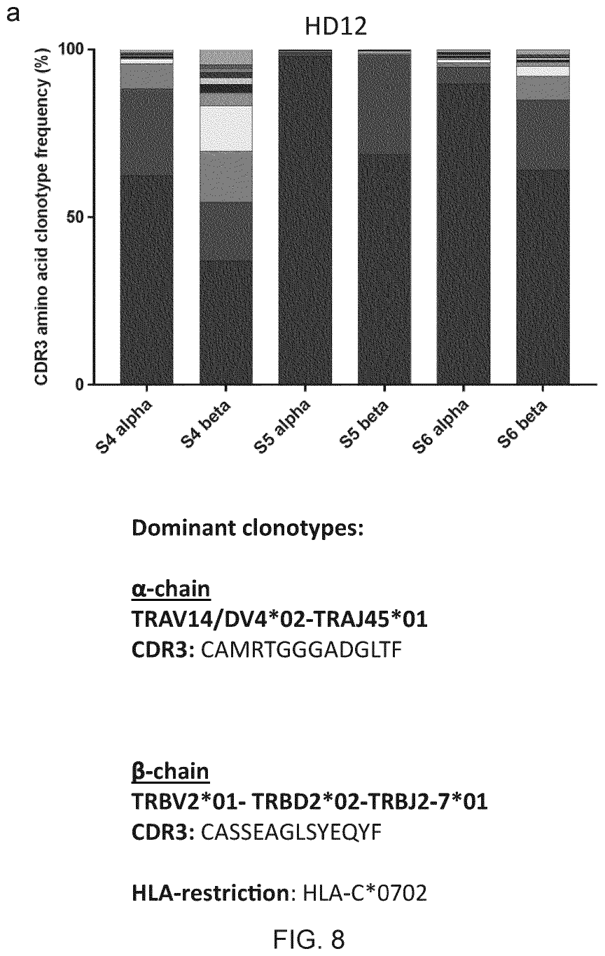

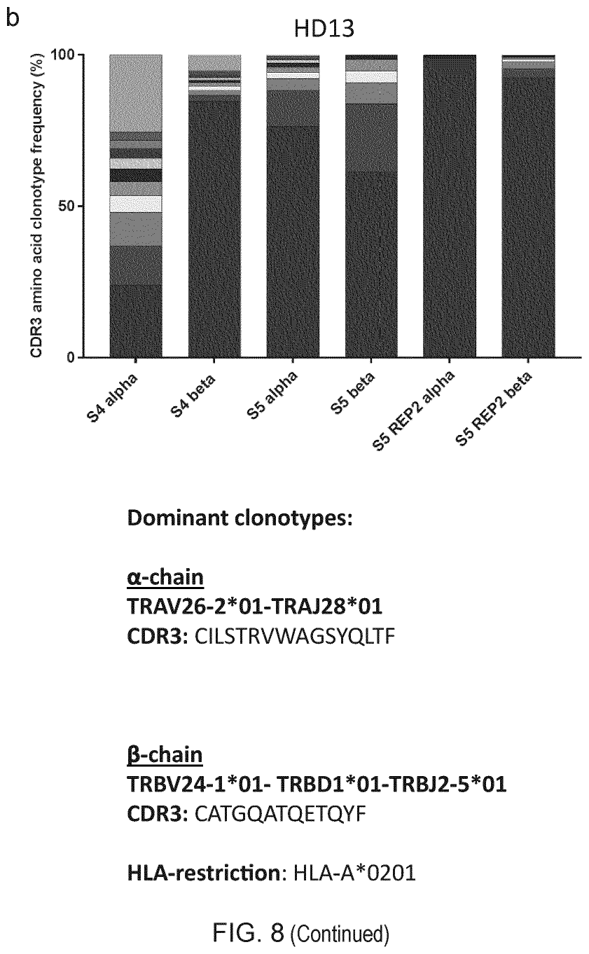

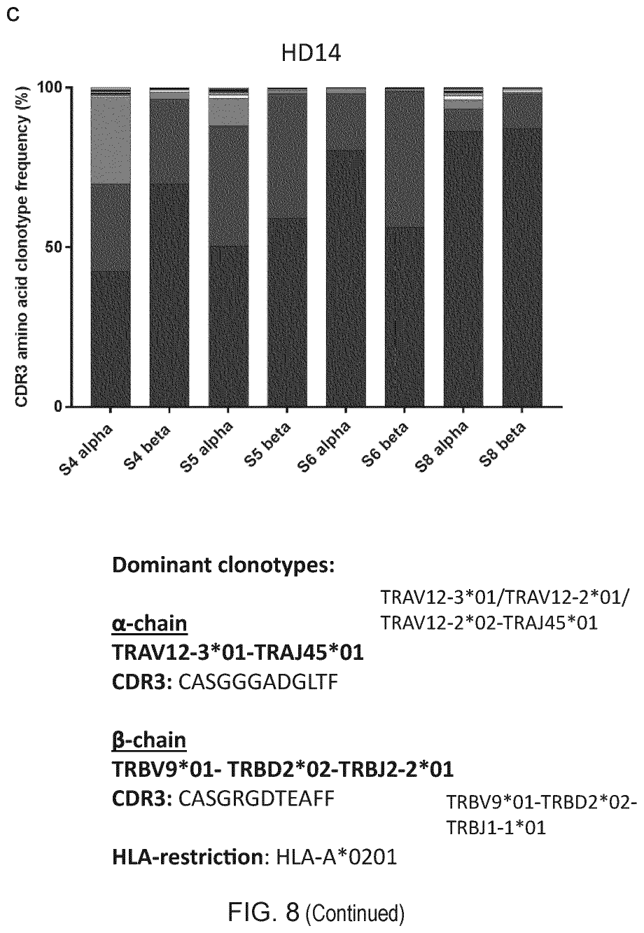

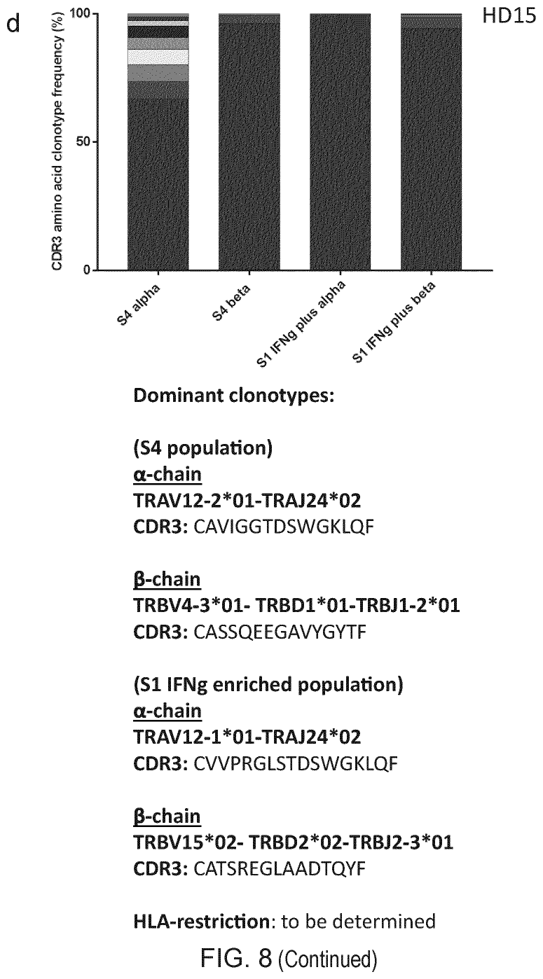

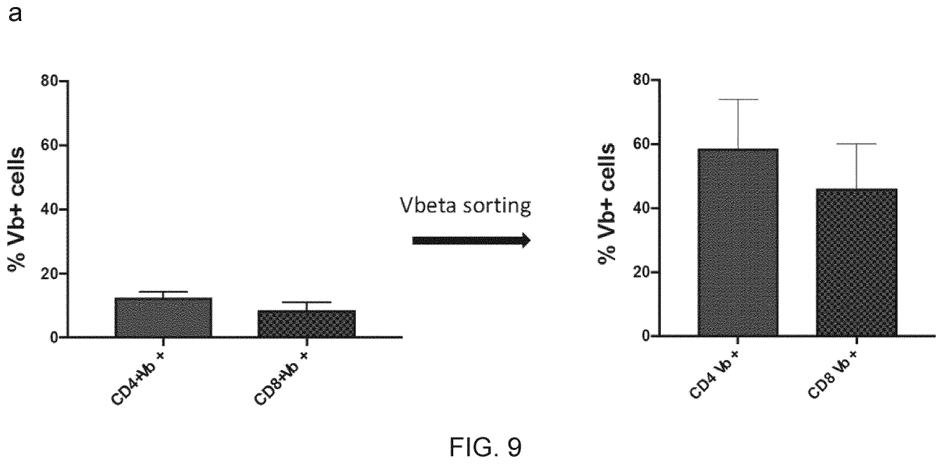

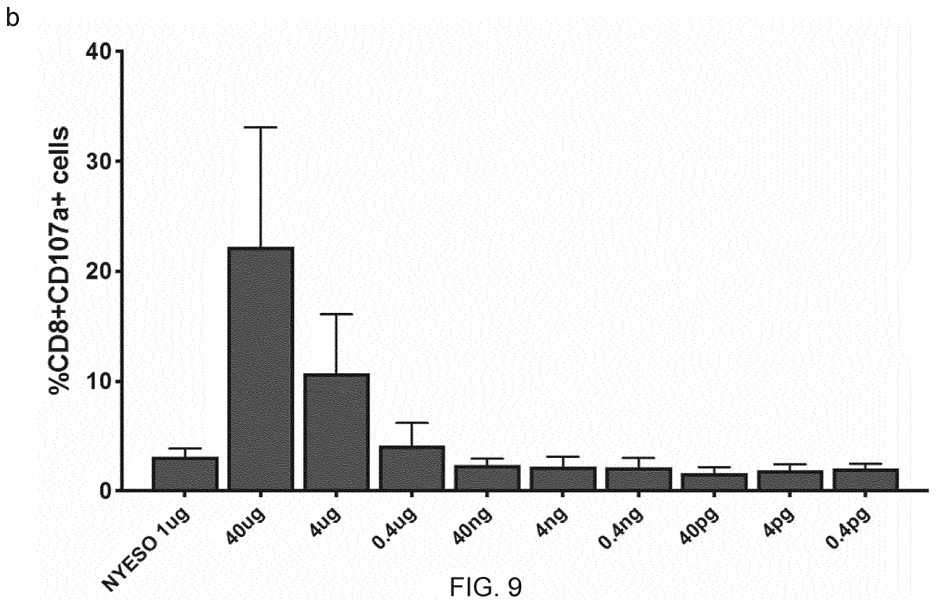

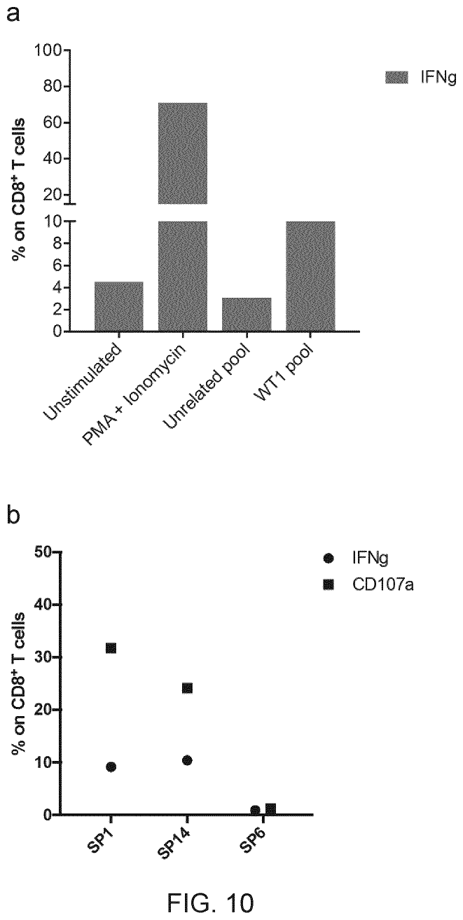

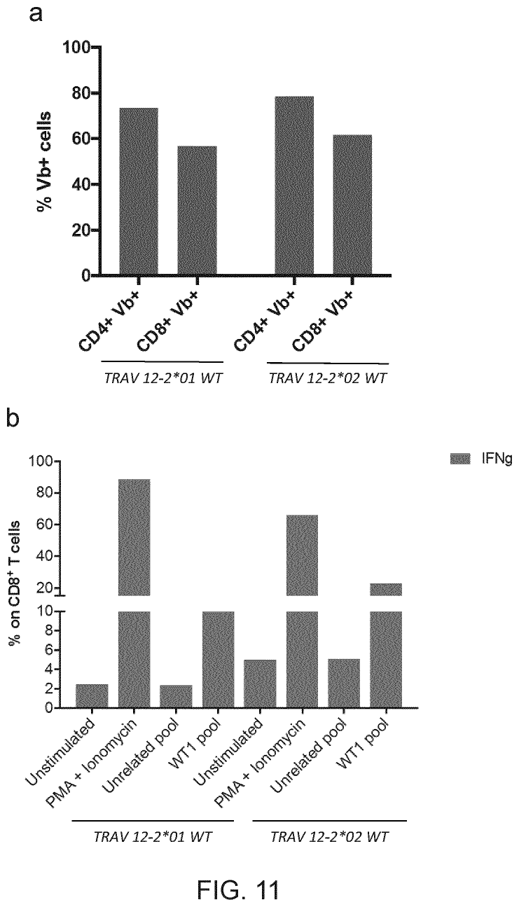

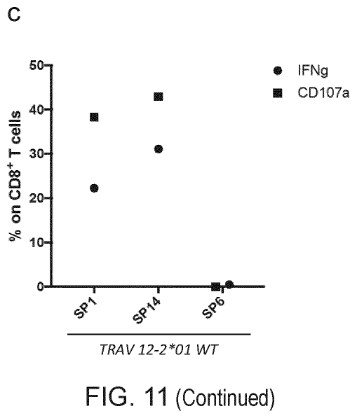

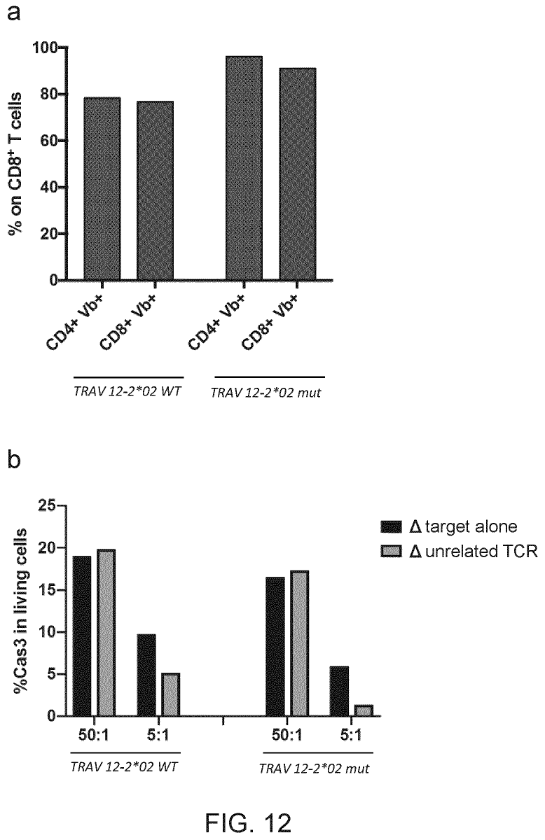

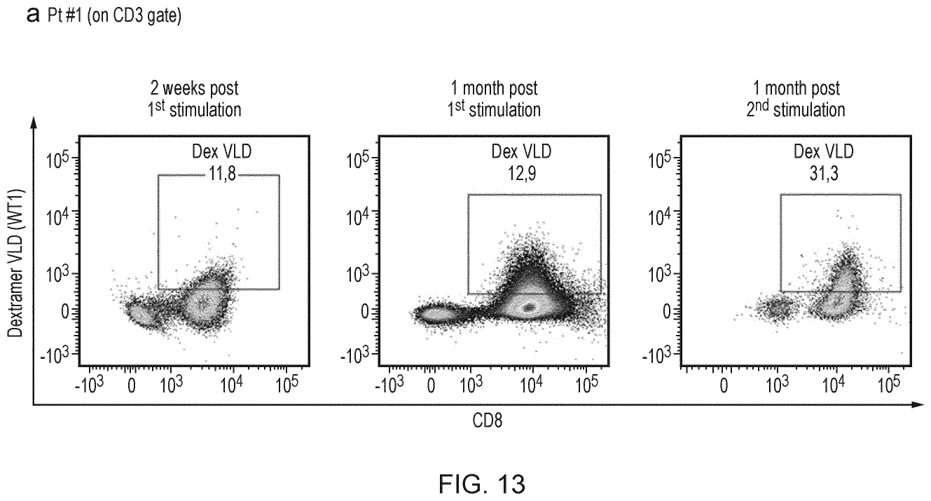

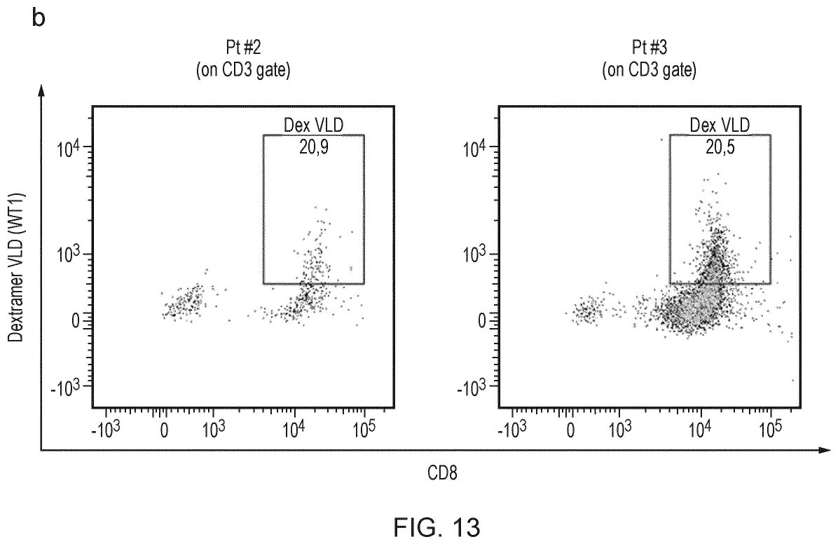

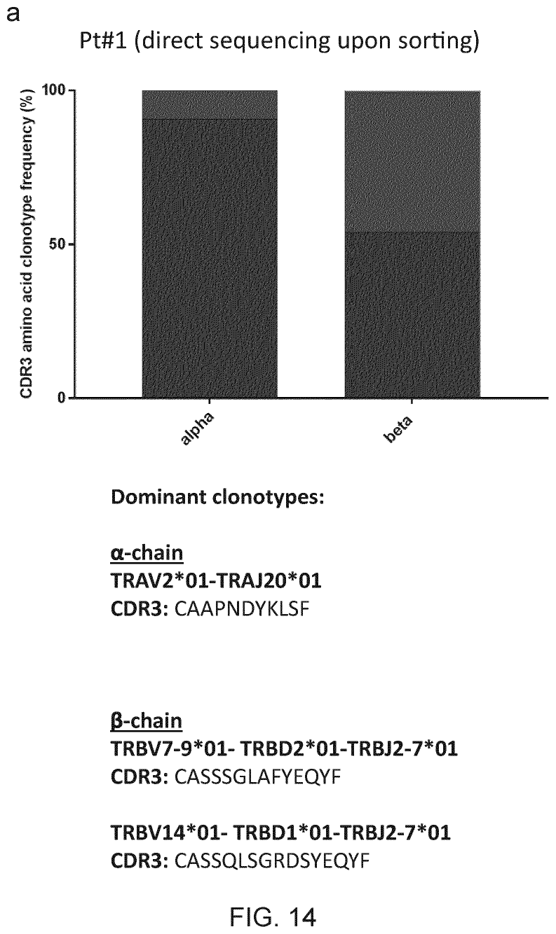

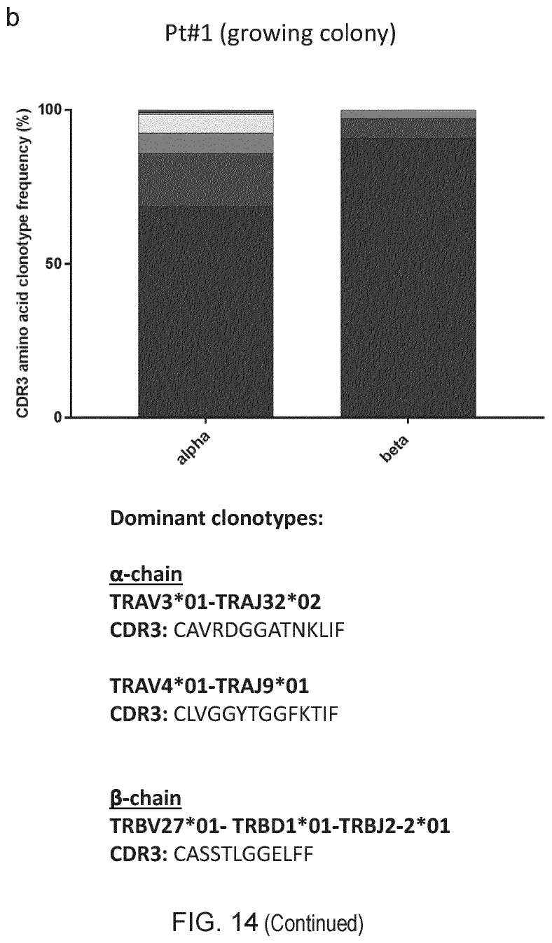

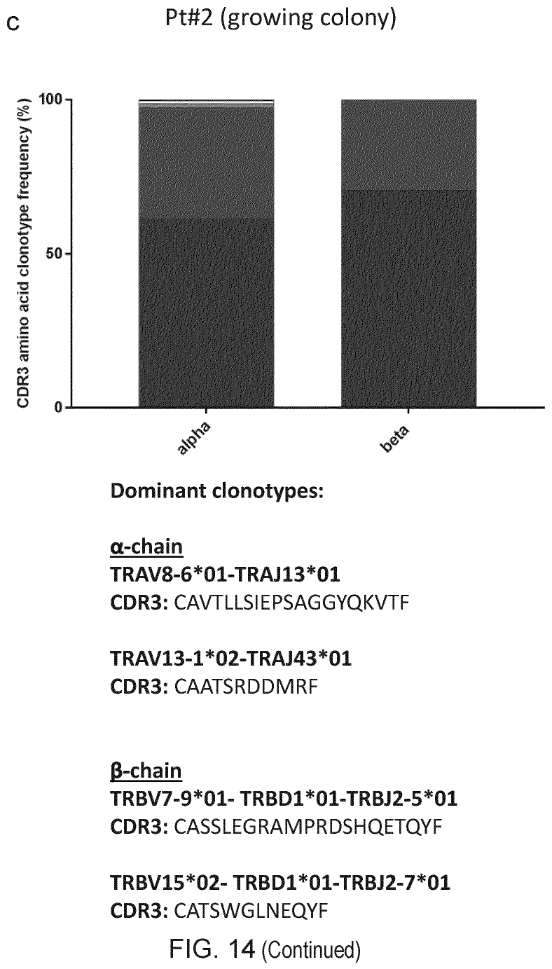

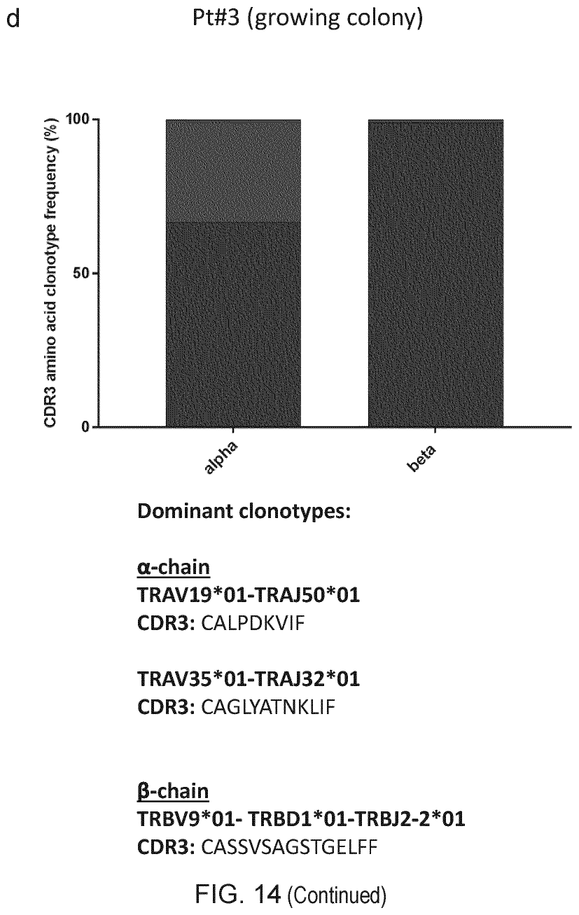

Cells were repetitively stimulated with APCs represented by CD3− cells loaded with the peptide pool. Expansion of WT1-specific T cells was evaluated over time by cytofluorimetric analysis to assess cytokine release (IFNγ, IL-2, TNF-α) and expression of degranulation marker (CD107a). As a negative control, cells were stimulated with a peptide pool originated from an unrelated antigen. Overall, we observed expansion of tumor-specific T lymphocytes in the CD8 fraction upon at least three stimulations with WT1 pool (FIG. 1, a,b,c,d).

For HD15, in a separate experiment, we stimulated PBMCs with a single peptide (P91) and enriched WT1-specific T cells by using the IFNγ catch assay. Upon ˜20 days of culture, T cells were used for TCR sequencing analysis.

Mapping of WT1 Epitopes Eliciting a T Cell Response.

In order to identify which pentadecapeptide of the WT1 pool-137 elicited an immune response in HD12, we used a mapping grid strategy as previously described by Doubrovina et al. Briefly, WT1 overlapping pentadecapeptides were subdivided into 24 subpools (SPs) containing up to 12 peptides in which each peptide of the 141 peptides described in Doubrovina et al. was uniquely contained within two intersecting SPs. Enriched WT1-specific T cells were co-cultured for 6 hours with irradiated APCs (autologous immortalised B cells) pulsed with the 24 SPs and we measured the percentage of IFNγ secretion and CD107a expression by flow cytometry. This strategy enables the detection of the immunogenic peptide by the deconvolution of the mapping grid. For HD13 and HD14, we stimulated autologous APCs with each of the individual peptides included in the WT1 HLA-A*02:01 pool and used them as target cells in a 6-hour co-culture experiment with WT1-specific T cells. For HD15, WT1-enriched T cells originated upon stimulation of the PBMCs with the WT1 HLA-A*02:01 pool, we did not perform the mapping of the immunogenic peptides due to the reduced cellularity.

We observed a substantial secretion of IFNγ and expression of CD107a after stimulation of T cells with subpools SP7 and SP21 (FIG. 2a, b). For HD13 and HD14 (FIG. 2c), stimulated with the WT1 pool HLA-A*02:01, there was a robust immune response towards autologous APCs pulsed with the P13 peptide (FIG. 2d, e for HD13 and HD14, respectively).

Once identified for HD12 the SP recognised by WT1-specific T cells, T lymphocytes were re-stimulated using CD3-depleted PBMCs pulsed with the specific peptide identified upon deconvolution of the mapping grid (FIG. 3a; highlighted is the immunogenic peptide identified).

In a stepwise approach, to validate the immunogenic pentadecapeptide, we tested T cells in a 6 hour co-culture with autologous irradiated immortalised B cells loaded with the 15mer eliciting the immune response and with at least one unrelated 15mer. Increased expression of CD107a and IFNγ secretion was observed for peptide 103 (FIG. 3b). The identified immunogenic pentadecapeptide was further used to re-stimulate T cells in order to provide an enrichment of the epitope-specific population. For HD13 and HD14, T cells were restimulated with autologous APCs stimulated with the recognised peptide (P13).

In silico prediction of peptide-MHC binding In order to predict for each HD characterised by the expansion of CD8-specific T cells the exact binding nonamers and their HLA-restrictions, we used the NetMHCpan 4.0 server (Jurtz V. et al. (2017) The Journal of Immunology). Binding prediction was performed only for peptides presented by HLA class I molecules, which have a strong preference for peptides of 9 amino acids. A defined peptide will be identified as a strong binder if the % Rank is below the 0.5% and as weak binder if the % Rank is between 0.5% and 2%.

In order to determine HLA-alleles harboured from HD12-HD15, DNA of each individual was HLA-typed (for HLA-A, HLA-B, HLA-C alleles) at high resolution in the HLA and Chimerism Laboratory of Ospedale San Raffaele (FIG. 4a).

For HD12, 2 strong binders were identified: peptide YRIHTHGVF (SEQ ID NO: 73) either on the HLA-B*38:01 allele or on the HLA-C*07:02 allele (stronger binding) (FIG. 4b).

For HD13 and HD14, peptide LLAAILDFL (SEQ ID NO: 74) was identified as a strong binder in combination with the HLA-A*02:01 allele (FIG. 4c, d); in addition for HD14, peptide AAILDFLLL (SEQ ID NO: 75) was evidenced as a strong binder when presented by HLA-C*03:03 allele (FIG. 4d).

For HD15, no strong binders were predicted.

Identified WT1 Peptides Represent Immunogenic Peptides Presented by Different HLA Alleles

In order to determine the HLA restriction of the antigen-specific T cells identified for each HD analysed, T lymphocytes were co-cultured with target cells expressing (or not) specific HLA class I alleles harboured by the HD.

For HD12, we used as a target a panel of EBV-BLCLs harbouring a single HLA allele in common with the HD, pulsed either with the relevant peptide or with an unrelated one. Results showed an increase in the number of cells expressing CD107a and secreting IFNγ upon co-culture with each EBV-BLCLs harboring the HLA-C*07:02 allele and pulsed with the WT1 P103 peptide (FIG. 5a).

For HD13 and HD14, stimulated with a pool of peptides previously reported to be able to elicit an immune response when presented by the HLA-A*02:01 allele, we directly performed a functional validation by co-culturing with T2 cells pulsed either with the specific immunogenic epitope (P13) or with an unrelated one. Flow cytometry results showed a great increase in the percentage of cells expressing the CD107a marker and secreting IFNγ upon co-culture of T lymphocytes with T2 cells pulsed with P13 (FIG. 5, b, c).

Assessment of Peptide Processing by FACS.

To determine the ability of WT1-expanded T cells isolated from HD13 (FIG. 6a) and HD14 (FIG. 6b) to recognise a naturally processed peptide and kill target cells, we evaluated the expression of active Caspase 3 in living primary blasts upon 6 hour co-culture with T lymphocytes. As target cells we used primary blasts from 3 AML patients selected according to the high expression of the WT1 antigen and to the HLA typing (HLA-A*02:01). As a control, we included co-cultures of unrelated effector cells with the same leukemic blasts used for HD13 and HD14. Results showed the ability of both HD13 as well as HD14 T cells to recognise primary leukemic blasts, with HD14 showing a greater elimination of AML blasts at the different effector to target ratios used.

For HD12, due to the low cellularity of the cell population we did not perform any test to validate the natural processing of the recognised peptide.

Overall, the ability of the WT1-specific T cells originated from HD13 and HD14 to recognise WT1-expressing target cells (leukemic blasts) indicated not only the natural processing of the recognised peptide but also its immunogenicity.

Immunoprofiling of WT1-Specific T Cells.

To characterise the newly identify WT1-specific TCRs, we performed both flow cytometry of the TCR Vβ families and TCR sequencing. FACS results indicated the prevalence of a specific Vβ for HD12 and 14; for HD13 an exhaustive determination of the predominant Vβ was not possible, as the IO Test Beta Mark TCR V beta repertoire guarantees a coverage of 75% of the complete repertoire of V beta (FIG. 7). For HD15 WT1-specific T cells, the flow cytometry assessment of the expressed Vβ families was not performed due to the low cellularity and the reduced cell fitness. TCRαβ sequencing of WT1-specific T cells highlighted the increasing predominance of one CDR3 clonotype over time for both TCR chains in HD12, HD13, HD14 (FIG. 8a-c). For HD15, we observed a clear expansion of specific TCR chains both upon stimulations with the WT1 HLA-A*02:01 pool and upon stimulation with the individual peptide (P91) followed by IFNγ enrichment (FIG. 8d).

Functional Validation of the Newly Cloned TCRs

TCR α and β sequences isolated from HD12, HD13, HD14 and HD15 and recognising WT1 epitopes restricted to HLA class I alleles were further modified in order to increase their surface expression and reduce mispairing with endogenous TCR chains. For HD14 TCRs we further mutagenised the receptors in order to increase their functional avidity as described in Kuball, J et al. (2009) J Exp Med 206: 463-75. TCR genes obtained from HD12, HD13 and HD14 (all different forms generated as described in Materials and Methods of Example 1) were cloned into bidirectional lentiviral vectors to promote robust and coordinate expression of both TCR chains in transduced lymphocytes. Viral production for lentiviral vectors encoding cloned TCRs was performed.

T cells from healthy individuals were transduced with lentiviral vectors previously generated from HD12, HD13 and HD14.

For HD12 TCR, activated T cells originating from 3 different healthy donors were edited as described in the Materials and Methods section (Example 1). T cells were transduced with the LV encoding the specific TCR α and β chain genes upon disruption of the endogenous TCR repertoire. After 14 days, transduction efficiency was evaluated measuring the percentage of cells expressing the VP22. Transduced T cells were sorted according to the V3 expression on the cell surface (FIG. 9a).

The functional avidity of HD12-transduced edited T cells was assessed by co-culture with the EBV− cell line harbouring the HLA-C*07:02 allele pulsed either with the NYESO-1 peptide as a negative control or with decreasing concentrations (from 40 μg to 0.4 μg) of the peptide 103 (E:T ratio=1:1). Ability of TCR-transduced T lymphocytes to recognise target cells was evaluated by cytofluorimetric analysis determining the expression of CD107a on CD8 T cells. Results showed that HD12-transduced edited T cells specifically recognise target cells expressing the HLA allele of interest even at a peptide concentration of 0.4 μg (FIG. 9b).

For testing HD13 and HD14-derived TCRs, which recognise an HLA-A*02:01-restricted epitope (peptide 13), activated T lymphocytes isolated from one healthy individual were transduced with the newly produced lentiviral vectors. Transduction efficiency for HD13 could not be measured due to the absence of an antibody recognising its specific V3. Transduction efficiency of HD14-transduced T cells (either with TCR TRAV12-2*01 WT or with TCR TRAV12-2*02 WT) was measured by evaluating the percentage of Vβ expression on CD4 and CD8 T cells (FIG. 11a).

T cells transduced with HD13 and HD14-derived TCRs were tested for their functional avidity in two distinct co-culture experiments. In the first experiment, HD13 TCR transfer T cells and HD14 TCR transfer T cells (either the one harbouring the TRAV12-2*01 WT gene or the TRAV12-2*02 WT gene) were co-cultured with T2 cells pulsed with WT1 pool or, as a control, an unrelated pool. In the second experiment, HD13 TCR transfer T cells and HD14 TCR transfer T cells (harbouring the TRAV12-2*01 WT gene) were co-cultured with T2 cells pulsed with subpools 1 and 14 (both containing the peptide 13) and subpool 6 (as a negative control). As a readout we measured the expression of CD107a and/or IFNγ secretion on CD8 T cells by cytofluorimetric analysis. We observed a specific recognition of the target cells pulsed with the WT1 pool both in T cells transduced with HD13 and HD14-derived TCRs (FIG. 10a and FIG. 11b) and the specific recognition of subpools 1 and 14 (FIG. 10b and FIG. 11c). Furthermore, HD14-derived TCRs (either the one harbouring the TRAV12-2*02 WT gene or the TRAV12-2*02 mut gene) were used in the TCR editing approach in T cells from one healthy donor. After 14 days, transduction efficiency was evaluated measuring the percentage of cells expressing the VP12 (FIG. 12a).

To determine the ability of WT1-edited T cells expressing HD14-derived TCRs (TRAV12-2*02 WT gene or the TRAV12-2*02 mut gene) to kill primary leukemic blasts, we evaluated the expression of active Caspase 3 in target cells upon 6 hour co-culture with T lymphocytes. As control, we included co-cultures of unrelated effector cells with the leukemic blasts and target cells cultured without effectors. Results showed the ability of HD14-edited T cells to recognise primary leukemic blasts at the different effector to target ratios used (FIG. 12b).

Discussion

The possibility to redirect T cell specificity against antigens expressed by tumor cells, through genetic manipulation, has opened up a new therapeutic window for cancer immunotherapy. In particular, the recent string of impressive clinical results obtained with CAR-redirected T cells (June et al. (2015) Science Translational Medicine 280-287) have significantly raised expectations among the scientific community, patient associations, pharma and general public. The full exploitation of this strategy largely relies on the identification of receptors specific for relevant tumor antigens. Ideally, tumor antigens must be molecules differentially expressed by tumor cells and healthy tissues, highly immunogenic, and possibly involved in cancer development and/or progression. WT1 is a very attractive target for cancer immunotherapy, and was ranked first in a list of 75 cancer antigens within a National Cancer Institute prioritisation project (Cheever (2009) Clin. Cancer Res. 15: 5323-5337). WT1 is overexpressed by cancer cells 10 to 1000 fold more than by healthy tissues (Inoue (1997) Blood 89: 1405-1412), and it is overexpressed in many different hematological malignancies, including acute myeloid and lymphoblastic leukemias and myelodisplastic syndromes, and by several solid tumors, such as lung cancer, breast cancer, esophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma (Haruo Sugiyama (2010) Jpn. J. Clin. Oncol. 40: 377-387). Vaccination against WT1 has resulted in objective antitumor responses in some cancer patients (Van Driessche et al. (2012) Oncologist 17: 250-259). More recently, clinical trials con WT1-specific T cells, isolated, expanded and adoptively transferred in patients with acute leukemia proved safe and mediated antileukemic activity (Chapuis et al. (2013) Sci Transl Med 5: 174ra27).

However, the low frequency of high-avidity T cells naturally reactive against WT1 has limited up to now the full exploitation of this antigen in adoptive T cell therapy.

The identification of WT1-reactive T cells, and of the genetic sequences of WT1 specific TCRs, opens up several novel therapeutic opportunities.

The TCR genetic sequences can be used in their natural forms, or modified, for example by murinisation of the constant TCR regions, or by cystein modification of the human TCR constant regions, to facilitate proper pairing of the TCR chains, or by codon optimisation of the genes, to modify their level of expression.

Natural or modified TCR genes might be transferred in specific T cell subsets, including CD4 and or CD8, naive, memory stem T cells, central memory, effector memory or effector cells, or in other cellular subsets such as to promote a different length of persistence and different functions in the engineered cells in vivo. The TCR genes could be also transferred in T cell subsets with different polarisation, such as Th0/Tc0, Th1/Tc1, Th2/Tc2, Th17, Th22 or others, depending on the cytokine milieu most proper to target each possible tumor type. Furthermore these genes, or chimeric genes designed to include the antigen-specific regions of the TCR, can be transferred in other cellular subsets, including gamma/delta T cells, NK cells, NKT cells, hematopoietic stem cells or other cells, to obtain the therapeutic effect. It is furthermore envisaged that natural or modified molecules designed to include the antigen-specific regions of the TCR, could be engineered or coupled to non cellular substrates such as nanoparticles, exosomes, or others, or might be used as soluble molecules, alone or coupled to other molecules such as toxins or antibodies, thus exploiting their ability to recognise tumor cells, thus conferring tumor specificity to cytotoxic compounds.

Genetic transfer of a novel TCR, such as the TCRs described herein, into T lymphocytes, suffers some limitations intrinsic to TCR biology. Specifically, the tumor-specific alpha and beta TCR chains are expressed in lymphocytes that already bear an endogenous TCR on the cell surface. Gene-modified cells thus express at least two different TCRs that compete for binding to the CD3 complex, leading to mutual TCR dilution and reduced T cell avidity and anti-tumor efficacy (Heemskerk, M. H. (2007) Blood 109: 235-243). Furthermore, since TCRs are heterodimers, the alpha and beta chains of the endogenous TCR might mispair with the respective alpha and beta chains of the transgenic TCR to produce new hybrid receptors, with unpredictable and potentially harmful specificities (Bendle, G. M. (2010) Nature Medicine 16: 565-570; van Loenen, M. M (2010) Proceedings of the National Academy of Sciences of the United States of America 107: 10972-10977). These limitations, that represent major concerns in TCR gene transfer-based adoptive immunotherapy, both in the autologous and in the allogeneic settings, can be addressed by several strategies, specifically designed with the aim of increasing TCR expression and fostering the correct pairing between tumor specific TCR chains. These strategies include murinisation of the constant regions (Cohen C. J. (2006) Cancer Research 66: 8878-8886) the cystein modification of the constant regions of the tumor specific TCR genes (Kuball J. (2007) Blood 109: 2331-2338), or the addition of siRNA designed to limit the expression of the endogenous TCR genes (Okamoto S (2009) Cancer Research 69: 9003-9011). Our group demonstrated that combining artificial nucleases, such as zinc finger nucleases (ZFNs), TALENs or CRISPR/Cas, designed to target the constant regions of the endogenous TCR genes (TRAC and TRBC), it is possible to obtain the permanent disruption of the endogenous TCR alpha and/or beta chain genes, thus allowing full expression of the tumor specific TCR. This process, known as the TCR gene editing proved superior to TCR gene transfer in vitro and in vivo (Provasi E., Genovese P. (2012) Nature Medicine 18: 807-15; Mastaglio S. et al. (2017) Blood 130: 606-618). In addition, the genome editing technology allows fostering of the targeted integration of a genetic cassette, inclusive of the tumor-specific TCR genes and promoter regions, into the endogenous gene disrupted by the artificial nucleases (Lombardo A. (2007) Nature Biotechnology 25: 1298-1306).

Finally, the genome editing technology allows the genetic disruption of multiple genes in a single cell: it can thus be envisaged that TCR gene editing could be coupled with the nuclease-based disruption of additional genes in the target cell, with the aim of modifying the persistence, expansion, activity, resistance to exhaustion/senescence/inhibitory signals, homing capacity, or other functions of the WT1-specific cellular product. Thus, based on a single antigenic specificity, we might envisage a wide array of therapeutic approaches, each one tailored to the specific tumor type and tumor environment.

### Example 2

Materials and Methods

Isolation of WT1-Specific T Cells from Patient Samples

Bone Marrow Aspirate Samples of 3 patients (Pt) diagnosed with Acute Myeloid Leukemia who underwent Allogeneic Hematopoietic Transplantation, harvested and cryopreserved at the institutional BioBank facility according to the Declaration of Helsinki, were thawed in X-VIVO 15 (Euroclone/Lonza) supplemented with Human Serum 5%, Penicillin/Streptamycin 1% and Glutamine 1%. A few hours after thawing, samples were washed in Phosphate Buffered Saline (w/o Ca and Mg) supplemented with EDTA and Fetal Bovine Serum 10% and subsequently incubated in a total volume of 50 μl with Dasatinib 50 nM for 30 minutes. After incubation, and without washing, the samples were stained with HLA*0201-restricted APC-conjugated dextramers loaded with the VLDFAPPGA (SEQ ID NO: 72) (WT1) epitope (ImmuDex) and incubated for 1.5 h on ice.

2 different approaches were tested to isolate WT1-specific T cells:
1. 100 Cells from Patient 1 were directly sorted using a BD FACS Aria cell sorter in reverse transcription buffer (SmartScribe; Takara Clontech) in a 1.5 ml Eppendorf tube. Afterwards, the sample was heated at 65° C. for 2 min followed by 5 min on ice and TCRαβ gene-specific cDNA synthesis was performed (Ruggiero E. et al. (2015) Nat. Commun. 6: 8081).
2. 500000-2000000 cells from Patients 1, 2, and 3 were stained with anti-APC magnetic microbeads (Miltenyi Biotec) according to the manufacturer's instructions and positively selected using the MACS system (Miltenyi Biotec). The positive fraction, enriched in WT1 VLDFAPPGA (SEQ ID NO: 72) specificities, was then cultured in U-bottom wells pre-coated with anti-CD3 and anti-CD28 monoclonal antibodies (1:2 ratio) in X-VIVO 15 (Euroclone/Lonza) supplemented with Human Serum 5%, Penicillin/Streptamycin 1%, Glutamine 1%, IL-2 60 IU/ml, IL-7 5 ng/ml and IL-15 5 ng/ml. Medium was changed every 3-4 days and cells split if confluence was reached.

TCR Repertoire Sequencing

RNA was extracted from WT1-enriched T cells of Patients 1, 2 and 3 by using the Arcturus Pico Pure RNA extraction kit (Life Technology). Complementarity determining region (CDR) 3 sequences of the WT1-specific T cells were amplified by using a modified RACE approach (Ruggiero E. et al. (2015) Nat. Commun. 6: 8081). Samples were sequenced by using an Illumina MiSeq sequencer and CDR3 clonotypes identified using the MiXCR software (Bolotin, D A et al. (2015) Nature Methods 12: 380-381).

Results

The enrichment in WT1 specificities was detected in each individual patient analysed. For Patient 1, anti-VLDFAPPGA (SEQ ID NO: 72) (WT1) enrichment occurred at three different stages of stimulation (2 weeks and 1 month after first stimulation, 1 month after second stimulation), detected by Dextramer staining at flow cytometry (FIG. 13a). For Patient 2 and Patient 3, 2 growing colonies specific for WT1 were detected, one for each patient, as assessed by APC-conjugated Dextramer at flow cytometry (FIG. 13b).

TCRαβ sequencing of WT1-specific T cells highlighted the increasing predominance of defined CDR3 clonotypes for both TCR chains in each patient analysed (FIG. 14a-d).

All publications mentioned in the above specification are herein incorporated by reference. Various modifications and variations of the described invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cell biology, immunology, immunotherapy, molecular biology, oncology or related fields are intended to be within the scope of the following claims.

1. A T-cell receptor (TCR), which binds to a Wilms tumour 1 protein (WT1) peptide when presented by a major histocompatibility complex (MHC), wherein the TCR:
(i) comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions;
(ii) comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions;
(iii) comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions;
(iv) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(v) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vi) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vii) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(viii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(ix) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(x) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xiii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xiv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xvi) comprises a CDR3α comprising the amino acid sequence of CALPDKVIF (SEQ ID NO: 148) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xvii) comprises a CDR3α comprising the amino acid sequence of CAGLYATNKLIF (SEQ ID NO: 153) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xviii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xix) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(xx) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(xxi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions; or
(xxii) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions.

2. The TCR of claim 1 comprising the following CDR sequences:
(SEQ ID NO: 23)
(i) CDR1α-NSAFQY,
(SEQ ID NO: 24)
CDR2α-TYSSGN,
(SEQ ID NO: 25)
CDR3α-CASGGGADGLTF,
(SEQ ID NO: 28)
CDR1β-SGDLS,
(SEQ ID NO: 29)
CDR2β-YYNGEE,
and
(SEQ ID NO: 30)
CDR3β-CASGRGDTEAFF,
or variants thereof each having up to three amino acid substitutions, additions or deletions;
(SEQ ID NO: 1)
(ii) CDR1α-TSDQSYG,
(SEQ ID NO: 2)
CDR2α-QGSYDEQN,
(SEQ ID NO: 3)
CDR3α-CAMRTGGGADGLTF,
(SEQ ID NO: 6)
CDR1β-SNHLY,
(SEQ ID NO: 7)
CDR2β-FYNNEI,
and
(SEQ ID NO: 8)
CDR3β-CASSEAGLSYEQYF,
or variants thereof each having up to three amino acid substitutions, additions or deletions;
(SEQ ID NO: 12)
(iii) CDR1α-TISGTDY,
(SEQ ID NO: 13)
CDR2α-GLTSN,
(SEQ ID NO: 14)
CDR3α-CILSTRVWAGSYQLTF,
(SEQ ID NO: 17)
CDR1β-KGHDR,
(SEQ ID NO: 18)
CDR2β-SFDVKD,
and
(SEQ ID NO: 19)
CDR3β-CATGQATQETQYF,
or variants thereof each having up to three amino acid substitutions, additions or deletions;
(SEQ ID NO: 34)
(iv) CDR1α-DRGSQS,
(SEQ ID NO: 35)
CDR2α-IYSNGD,
(SEQ ID NO: 36)
CDR3α-CAVIGGTDSWGKLQF,
(SEQ ID NO: 39)
CDR1β-LGHNA,
(SEQ ID NO: 40)
CDR2β-YSLEER,
and
(SEQ ID NO: 41)
CDR3β-CASSQEEGAVYGYTF,
or variants thereof each having up to three amino acid substitutions, additions or deletions;
(SEQ ID NO: 34)
(v) CDR1α-DRGSQS,
(SEQ ID NO: 35)
CDR2α-IYSNGD,
(SEQ ID NO: 36)
CDR3α-CAVIGGTDSWGKLQF,
(SEQ ID NO: 50)
CDR1β-LNHNV,
(SEQ ID NO: 51)
CDR2β-YYDKDF,
and
(SEQ ID NO: 52)
CDR3β-CATSREGLAADTQYF,
or variants thereof each having up to three amino acid substitutions, additions or deletions;
(SEQ ID NO: 45)
(vi) CDR1α-NSASQS,
(SEQ ID NO: 46)
CDR2α-VYSSGN,
(SEQ ID NO: 47)
CDR3α-CVVPRGLSTDSWGKLQF,
(SEQ ID NO: 50)
CDR1β-LNHNV,
(SEQ ID NO: 51)
CDR2β-YYDKDF,
and
(SEQ ID NO: 52)
CDR3β-CATSREGLAADTQYF,
or variants thereof each having up to three amino acid substitutions, additions or deletions;
(SEQ ID NO: 45)
(vii) CDR1α-NSASQS,
(SEQ ID NO: 46)
CDR2α-VYSSGN,
(SEQ ID NO: 47)
CDR3α-CVVPRGLSTDSWGKLQF,
(SEQ ID NO: 39)
CDR1β-LGHNA,
(SEQ ID NO: 40)
CDR2β-YSLEER,
and
(SEQ ID NO: 41)
CDR3β-CASSQEEGAVYGYTF,
or variants thereof each having up to three amino acid substitutions, additions or deletions;
(SEQ ID NO: 91)
(viii) CDR1α-VSNAYN,
(SEQ ID NO: 92)
CDR2α-GSKP,
(SEQ ID NO: 93)
CDR3α-CAAPNDYKLSF,
(SEQ ID NO: 96)
CDR1β-SEHNR,
(SEQ ID NO: 97)
CDR2β-FQNEAQ,
and
(SEQ ID NO: 98)
CDR3β-CASSSGLAFYEQYF,
or variants thereof each having up to three amino acid substitutions, additions or deletions;
(ix)
(SEQ ID NO: 91)
CDR1α-VSNAYN,
(SEQ ID NO: 92)
CDR2α-GSKP,
(SEQ ID NO: 93)
CDR3α-CAAPNDYKLSF,
(SEQ ID NO: 102)
CDR1β-SGHDN,
(SEQ ID NO: 103)
CDR2β-FVKESK,
and
(SEQ ID NO: 104)
CDR3β-CASSQLSGRDSYEQYF,
or variants thereof each having up to three amino acid substitutions, additions or deletions;
(x)
(SEQ ID NO: 108)
CDR1α-VSGNPY,
(SEQ ID NO: 109)
CDR2α-YITGDNLV,
(SEQ ID NO: 110)
CDR3α-CAVRDGGATNKLIF,
(SEQ ID NO: 118)
CDR1β-MNHEY,
(SEQ ID NO: 119)
CDR2β-SMNVEV,
and
(SEQ ID NO: 120)
CDR3β-CASSTLGGELFF,
or variants thereof each having up to three amino acid substitutions, additions or deletions;
(xi)
(SEQ ID NO: 113)
CDR1α-NIATNDY,
(SEQ ID NO: 114)
CDR2α-GYKTK,
(SEQ ID NO: 115)
CDR3α-CLVGGYTGGFKTIF,
(SEQ ID NO: 118)
CDR1β-MNHEY,
(SEQ ID NO: 119)
CDR2β-SMNVEV,
and
(SEQ ID NO: 120)
CDR3β-CASSTLGGELFF,
or variants thereof each having up to three amino acid substitutions, additions or deletions;
(xii)
(SEQ ID NO: 124)
CDR1α-SSVSVY,
(SEQ ID NO: 125)
CDR2α-YLSGSTLV,
(SEQ ID NO: 126)
CDR3α-CAVTLLSIEPSAGGYQKVTF,
(SEQ ID NO: 134)
CDR1β-SEHNR,
(SEQ ID NO: 135)
CDR2β-FQNEAQ,
and
(SEQ ID NO: 136)
CDR3β-CASSLEGRAMPRDSHQETQYF,
or variants thereof each having up to three amino acid substitutions, additions or deletions;
(xiii)
(SEQ ID NO: 124)
CDR1α-SSVSVY,
(SEQ ID NO: 125)
CDR2α-YLSGSTLV,
(SEQ ID NO: 126)
CDR3α-CAVTLLSIEPSAGGYQKVTF,
(SEQ ID NO: 140)
CDR1β-LNHNV,
(SEQ ID NO: 141)
CDR2β-YYDKDF,
and
(SEQ ID NO: 142)
CDR3β-CATSWGLNEQYF,
or variants thereof each having up to three amino acid substitutions, additions or deletions;
(xiv)
(SEQ ID NO: 129)
CDR1α-DSASNY,
(SEQ ID NO: 130)
CDR2α-IRSNVGE,
(SEQ ID NO: 131)
CDR3α-CAATSRDDMRF,
(SEQ ID NO: 134)
CDR1β-SEHNR,
(SEQ ID NO: 135)
CDR2β-FQNEAQ,
and
(SEQ ID NO: 136)
CDR3β-CASSLEGRAMPRDSHQETQYF,
or variants thereof each having up to three amino acid substitutions, additions or deletions;
(xv)
(SEQ ID NO: 129)
CDR1α-DSASNY,
(SEQ ID NO: 130)
CDR2α-IRSNVGE,
(SEQ ID NO: 131)
CDR3α-CAATSRDDMRF,
(SEQ ID NO: 140)
CDR1β-LNHNV,
(SEQ ID NO: 141)
CDR2β-YYDKDF,
and
(SEQ ID NO: 142)
CDR3β-CATSWGLNEQYF,
or variants thereof each having up to three amino acid substitutions, additions or deletions;
(xvi)
(SEQ ID NO: 146)
CDR1α-TRDTTYY,
(SEQ ID NO: 147)
CDR2α-RNSFDEQN,
(SEQ ID NO: 148)
CDR3α-CALPDKVIF,
(SEQ ID NO: 156)
CDR1β-SGDLS,
(SEQ ID NO: 157)
CDR2β-YYNGEE,
and
(SEQ ID NO: 158)
CDR3β-CASSVSAGSTGELFF,
or variants thereof each having up to three amino acid substitutions, additions or deletions;
(xvii)
(SEQ ID NO: 151)
CDR1α-SIFNT,
(SEQ ID NO: 152)
CDR2α-LYKAGEL,
(SEQ ID NO: 153)
CDR3α-CAGLYATNKLIF,
(SEQ ID NO: 156)
CDR1β-SGDLS,
(SEQ ID NO: 157)
CDR2β-YYNGEE,
and
(SEQ ID NO: 158)
CDR3β-CASSVSAGSTGELFF,
or variants thereof each having up to three amino acid substitutions, additions or deletions;
(xviii)
(SEQ ID NO: 91)
CDR1α-VSNAYN,
(SEQ ID NO: 92)
CDR2α-GSKP,
(SEQ ID NO: 93)
CDR3α-CAAPNDYKLSF,
(SEQ ID NO: 118)
CDR1β-MNHEY,
(SEQ ID NO: 119)
CDR2β-SMNVEV,
and
(SEQ ID NO: 120)
CDR3β-CASSTLGGELFF,
or variants thereof each having up to three amino acid substitutions, additions or deletions;
(xix)
(SEQ ID NO: 108)
CDR1α-VSGNPY,
(SEQ ID NO: 109)
CDR2α-YITGDNLV,
(SEQ ID NO: 110)
CDR3α-CAVRDGGATNKLIF,
(SEQ ID NO: 96)
CDR1β-SEHNR,
(SEQ ID NO: 97)
CDR2β-FQNEAQ,
and
(SEQ ID NO: 98)
CDR3β-CASSSGLAFYEQYF,
or variants thereof each having up to three amino acid substitutions, additions or deletions;
(xx)
(SEQ ID NO: 108)
CDR1α-VSGNPY,
(SEQ ID NO: 109)
CDR2α-YITGDNLV,
(SEQ ID NO: 110)
CDR3α-CAVRDGGATNKLIF,
(SEQ ID NO: 102)
CDR1β-SGHDN,
(SEQ ID NO: 103)
CDR2β-FVKESK,
and
(SEQ ID NO: 104)
CDR3β-CASSQLSGRDSYEQYF,
or variants thereof each having up to three amino acid substitutions, additions or deletions;
(xxi)
(SEQ ID NO: 113)
CDR1α-NIATNDY,
(SEQ ID NO: 114)
CDR2α-GYKTK,
(SEQ ID NO: 115)
CDR3α-CLVGGYTGGFKTIF,
(SEQ ID NO: 96)
CDR1β-SEHNR,
(SEQ ID NO: 97)
CDR2β-FQNEAQ,
and
(SEQ ID NO: 98)
CDR3β-CASSSGLAFYEQYF,
or variants thereof each having up to three amino acid substitutions, additions or deletions;
(xxii)
(SEQ ID NO: 113)
CDR1α-NIATNDY,
(SEQ ID NO: 114)
CDR2α-GYKTK,
(SEQ ID NO: 115)
CDR3α-CLVGGYTGGFKTIF,
(SEQ ID NO: 102)
CDR1β-SGHDN,
(SEQ ID NO: 103)
CDR2β-FVKESK,
and
(SEQ ID NO: 104)
CDR3β-CASSQLSGRDSYEQYF,
or variants thereof each having up to three amino acid substitutions, additions or deletions; or
(xxiii)
(SEQ ID NO: 182)
CDR1α-DRGSQS,
(SEQ ID NO: 183)
CDR2α-IYSNGD,
(SEQ ID NO: 25)
CDR3α-CASGGGADGLTF,
(SEQ ID NO: 28)
CDR1β-SGDLS,
(SEQ ID NO: 29)
CDR2β-YYNGEE,
and
(SEQ ID NO: 30)
CDR3β-CASGRGDTEAFF,
or variants thereof each having up to three amino acid substitutions, additions or deletions.

3. The TCR of claim 1 comprising:
(i) an α chain variable domain comprising the amino acid sequence of SEQ ID NO: 26 or a variant thereof having at least 75% sequence identity thereto; and a β chain variable domain comprising the amino acid sequence of SEQ ID NO: 31 or a variant thereof having at least 75% sequence identity thereto;
(ii) an α chain variable domain comprising the amino acid sequence of SEQ ID NO: 4 or a variant thereof having at least 75% sequence identity thereto; and a β chain variable domain comprising the amino acid sequence of SEQ ID NO: 9 or a variant thereof having at least 75% sequence identity thereto;
(iii) an α chain variable domain comprising the amino acid sequence of SEQ ID NO: 15 or a variant thereof having at least 75% sequence identity thereto; and a β chain variable domain comprising the amino acid sequence of SEQ ID NO: 20 or a variant thereof having at least 75% sequence identity thereto;
(iv) an α chain variable domain comprising the amino acid sequence of SEQ ID NO: 37 or a variant thereof having at least 75% sequence identity thereto; and a β chain variable domain comprising the amino acid sequence of SEQ ID NO: 42 or a variant thereof having at least 75% sequence identity thereto;
(v) an α chain variable domain comprising the amino acid sequence of SEQ ID NO: 37 or a variant thereof having at least 75% sequence identity thereto; and a β chain variable domain comprising the amino acid sequence of SEQ ID NO: 53 or a variant thereof having at least 75% sequence identity thereto;
(vi) an α chain variable domain comprising the amino acid sequence of SEQ ID NO: 48 or a variant thereof having at least 75% sequence identity thereto; and a β chain variable domain comprising the amino acid sequence of SEQ ID NO: 53 or a variant thereof having at least 75% sequence identity thereto;
(vii) an α chain variable domain comprising the amino acid sequence of SEQ ID NO: 48 or a variant thereof having at least 75% sequence identity thereto; and a β chain variable domain comprising the amino acid sequence of SEQ ID NO: 42 or a variant thereof having at least 75% sequence identity thereto;
(viii) an α chain variable domain comprising the amino acid sequence of SEQ ID NO: 94 or a variant thereof having at least 75% sequence identity thereto; and a β chain variable domain comprising the amino acid sequence of SEQ ID NO: 99 or a variant thereof having at least 75% sequence identity thereto;
(ix) an α chain variable domain comprising the amino acid sequence of SEQ ID NO: 94 or a variant thereof having at least 75% sequence identity thereto; and a β chain variable domain comprising the amino acid sequence of SEQ ID NO: 105 or a variant thereof having at least 75% sequence identity thereto;
(x) an α chain variable domain comprising the amino acid sequence of SEQ ID NO: 111 or a variant thereof having at least 75% sequence identity thereto; and a β chain variable domain comprising the amino acid sequence of SEQ ID NO: 121 or a variant thereof having at least 75% sequence identity thereto;
(xi) an α chain variable domain comprising the amino acid sequence of SEQ ID NO: 116 or a variant thereof having at least 75% sequence identity thereto; and a β chain variable domain comprising the amino acid sequence of SEQ ID NO: 121 or a variant thereof having at least 75% sequence identity thereto;
(xii) an α chain variable domain comprising the amino acid sequence of SEQ ID NO: 127 or a variant thereof having at least 75% sequence identity thereto; and a β chain variable domain comprising the amino acid sequence of SEQ ID NO: 137 or a variant thereof having at least 75% sequence identity thereto;
(xiii) an α chain variable domain comprising the amino acid sequence of SEQ ID NO: 127 or a variant thereof having at least 75% sequence identity thereto; and a β chain variable domain comprising the amino acid sequence of SEQ ID NO: 143 or a variant thereof having at least 75% sequence identity thereto;
(xiv) an α chain variable domain comprising the amino acid sequence of SEQ ID NO: 132 or a variant thereof having at least 75% sequence identity thereto; and a β chain variable domain comprising the amino acid sequence of SEQ ID NO: 137 or a variant thereof having at least 75% sequence identity thereto;
(xv) an α chain variable domain comprising the amino acid sequence of SEQ ID NO: 132 or a variant thereof having at least 75% sequence identity thereto; and a β chain variable domain comprising the amino acid sequence of SEQ ID NO: 143 or a variant thereof having at least 75% sequence identity thereto;
(xvi) an α chain variable domain comprising the amino acid sequence of SEQ ID NO: 149 or a variant thereof having at least 75% sequence identity thereto; and a β chain variable domain comprising the amino acid sequence of SEQ ID NO: 159 or a variant thereof having at least 75% sequence identity thereto;
(xvii) an α chain variable domain comprising the amino acid sequence of SEQ ID NO: 154 or a variant thereof having at least 75% sequence identity thereto; and a β chain variable domain comprising the amino acid sequence of SEQ ID NO: 159 or a variant thereof having at least 75% sequence identity thereto;
(xviii) an α chain variable domain comprising the amino acid sequence of SEQ ID NO: 94 or a variant thereof having at least 75% sequence identity thereto; and a β chain variable domain comprising the amino acid sequence of SEQ ID NO: 121 or a variant thereof having at least 75% sequence identity thereto;
(xix) an α chain variable domain comprising the amino acid sequence of SEQ ID NO: 111 or a variant thereof having at least 75% sequence identity thereto; and a β chain variable domain comprising the amino acid sequence of SEQ ID NO: 99 or a variant thereof having at least 75% sequence identity thereto;
(xx) an α chain variable domain comprising the amino acid sequence of SEQ ID NO: 111 or a variant thereof having at least 75% sequence identity thereto; and a β chain variable domain comprising the amino acid sequence of SEQ ID NO: 105 or a variant thereof having at least 75% sequence identity thereto;
(xxi) an α chain variable domain comprising the amino acid sequence of SEQ ID NO: 116 or a variant thereof having at least 75% sequence identity thereto; and a β chain variable domain comprising the amino acid sequence of SEQ ID NO: 99 or a variant thereof having at least 75% sequence identity thereto;
(xxii) an α chain variable domain comprising the amino acid sequence of SEQ ID NO: 116 or a variant thereof having at least 75% sequence identity thereto; and a β chain variable domain comprising the amino acid sequence of SEQ ID NO: 105 or a variant thereof having at least 75% sequence identity thereto;
(xxiii) an α chain variable domain comprising the amino acid sequence selected from the group consisting of SEQ ID NO: 185, 190 or a variant thereof having at least 75% sequence identity thereto; and a β chain variable domain comprising the amino acid sequence of SEQ ID NO: 31 or a variant thereof having at least 75% sequence identity thereto.

4. The TCR of claim 1 comprising:
(i) an α chain comprising the amino acid sequence of SEQ ID NO: 27 or a variant thereof having at least 75% sequence identity thereto; and a β chain comprising an amino acid sequence selected from the group consisting of SEQ ID NO: 32, SEQ ID NO: 33, SEQ ID NO: 203 and variants of SEQ ID NOs: 32, 33 and 203 having at least 75% sequence identity thereto;
(ii) an α chain comprising the amino acid sequence of SEQ ID NO: 5 or a variant thereof having at least 75% sequence identity thereto; and a β chain comprising an amino acid sequence selected from the group consisting of SEQ ID NO: 10, SEQ ID NO: 11, SEQ ID NO: 195 and variants of SEQ ID NOs: 10, 11 and 195 having at least 75% sequence identity thereto;
(iii) an α chain comprising the amino acid sequence of SEQ ID NO: 16 or a variant thereof having at least 75% sequence identity thereto; and a β chain comprising an amino acid sequence selected from the group consisting of SEQ ID NO: 21, SEQ ID NO: 22, SEQ ID NO: 197 and variants of SEQ ID NOs: 21, 22 and 197 having at least 75% sequence identity thereto;
(iv) an α chain comprising the amino acid sequence of SEQ ID NO: 38 or a variant thereof having at least 75% sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75% sequence identity thereto;
(v) an α chain comprising the amino acid sequence of SEQ ID NO: 38 or a variant thereof having at least 75% sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75% sequence identity thereto;
(vi) an α chain comprising the amino acid sequence of SEQ ID NO: 49 or a variant thereof having at least 75% sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75% sequence identity thereto;
(vii) an α chain comprising the amino acid sequence of SEQ ID NO: 49 or a variant thereof having at least 75% sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75% sequence identity thereto;
(viii) an α chain comprising the amino acid sequence of SEQ ID NO: 95 or a variant thereof having at least 75% sequence identity thereto; and a β chain comprising an amino acid sequence selected from the group consisting of SEQ ID NO: 100, SEQ ID NO: 101 and variants of SEQ ID NOs: 100 and 101 having at least 75% sequence identity thereto;
(ix) an α chain comprising the amino acid sequence of SEQ ID NO: 95 or a variant thereof having at least 75% sequence identity thereto; and a β chain comprising an amino acid sequence selected from the group consisting of SEQ ID NO: 106, SEQ ID NO: 107 and variants of SEQ ID NOs: 106 and 107 having at least 75% sequence identity thereto;
(x) an α chain comprising the amino acid sequence of SEQ ID NO: 112 or a variant thereof having at least 75% sequence identity thereto; and a β chain comprising an amino acid sequence selected from the group consisting of SEQ ID NO: 122, SEQ ID NO: 123 and variants of SEQ ID NOs: 122 and 123 having at least 75% sequence identity thereto;
(xi) an α chain comprising the amino acid sequence of SEQ ID NO: 117 or a variant thereof having at least 75% sequence identity thereto; and a β chain comprising an amino acid sequence selected from the group consisting of SEQ ID NO: 122, SEQ ID NO: 123 and variants of SEQ ID NOs: 122 and 123 having at least 75% sequence identity thereto;
(xii) an α chain comprising the amino acid sequence of SEQ ID NO: 128 or a variant thereof having at least 75% sequence identity thereto; and a β chain comprising an amino acid sequence selected from the group consisting of SEQ ID NO: 138, SEQ ID NO: 139 and variants of SEQ ID NOs: 138 and 139 having at least 75% sequence identity thereto;
(xiii) an α chain comprising the amino acid sequence of SEQ ID NO: 128 or a variant thereof having at least 75% sequence identity thereto; and a β chain comprising an amino acid sequence selected from the group consisting of SEQ ID NO: 144, SEQ ID NO: 145 and variants of SEQ ID NOs: 144 and 145 having at least 75% sequence identity thereto;
(xiv) an α chain comprising the amino acid sequence of SEQ ID NO: 133 or a variant thereof having at least 75% sequence identity thereto; and a β chain comprising an amino acid sequence selected from the group consisting of SEQ ID NO: 138, SEQ ID NO: 139 and variants of SEQ ID NOs: 138 and 139 having at least 75% sequence identity thereto;
(xv) an α chain comprising the amino acid sequence of SEQ ID NO: 133 or a variant thereof having at least 75% sequence identity thereto; and a β chain comprising an amino acid sequence selected from the group consisting of SEQ ID NO: 144, SEQ ID NO: 145 and variants of SEQ ID NOs: 144 and 145 having at least 75% sequence identity thereto;
(xvi) an α chain comprising the amino acid sequence of SEQ ID NO: 150 or a variant thereof having at least 75% sequence identity thereto; and a β chain comprising an amino acid sequence selected from the group consisting of SEQ ID NO: 160, SEQ ID NO: 161 and variants of SEQ ID NOs: 160 and 161 having at least 75% sequence identity thereto;
(xvii) an α chain comprising the amino acid sequence of SEQ ID NO: 155 or a variant thereof having at least 75% sequence identity thereto; and a β chain comprising an amino acid sequence selected from the group consisting of SEQ ID NO: 160, SEQ ID NO: 161 and variants of SEQ ID NOs: 160 and 161 having at least 75% sequence identity thereto;
(xviii) an α chain comprising the amino acid sequence of SEQ ID NO: 95 or a variant thereof having at least 75% sequence identity thereto; and a β chain comprising an amino acid sequence selected from the group consisting of SEQ ID NO: 122, SEQ ID NO: 123 and variants of SEQ ID NOs: 122 and 123 having at least 75% sequence identity thereto;
(xix) an α chain comprising the amino acid sequence of SEQ ID NO: 112 or a variant thereof having at least 75% sequence identity thereto; and a β chain comprising an amino acid sequence selected from the group consisting of SEQ ID NO: 100, SEQ ID NO: 101 and variants of SEQ ID NOs: 100 and 101 having at least 75% sequence identity thereto;
(xx) an α chain comprising the amino acid sequence of SEQ ID NO: 112 or a variant thereof having at least 75% sequence identity thereto; and a β chain comprising an amino acid sequence selected from the group consisting of SEQ ID NO: 106, SEQ ID NO: 107 and variants of SEQ ID NOs: 106 and 107 having at least 75% sequence identity thereto;
(xxi) an α chain comprising the amino acid sequence of SEQ ID NO: 117 or a variant thereof having at least 75% sequence identity thereto; and a β chain comprising an amino acid sequence selected from the group consisting of SEQ ID NO: 100, SEQ ID NO: 101 and variants of SEQ ID NOs: 100 and 101 having at least 75% sequence identity thereto;
(xxii) an α chain comprising the amino acid sequence of SEQ ID NO: 117 or a variant thereof having at least 75% sequence identity thereto; and a β chain comprising an amino acid sequence selected from the group consisting of SEQ ID NO: 106, SEQ ID NO: 107 and variants of SEQ ID NOs: 106 and 107 having at least 75% sequence identity thereto;
(xxiii) (a) an α chain comprising an amino acid sequence selected from the group consisting of SEQ ID NOs: 186, 191, 198, 199, 200, 201, 202 and variants of SEQ ID NOs: 186, 191, 198, 199, 200, 201 and 202 having at least 75% sequence identity thereto; and a β chain comprising the amino acid sequence of SEQ ID NO: 32 or a variant thereof having at least 75% sequence identity thereto;
(b) an α chain comprising an amino acid sequence selected from the group consisting of SEQ ID NOs: 186, 191, 198, 199, 200, 201, 202 and variants of SEQ ID NOs: 186, 191, 198, 199, 200, 201 and 202 having at least 75% sequence identity thereto; and a β chain comprising the amino acid sequence of SEQ ID NO: 33 or a variant thereof having at least 75% sequence identity thereto; or
(c) an α chain comprising an amino acid sequence selected from the group consisting of SEQ ID NOs: 186, 191, 198, 199, 200, 201, 202 and variants of SEQ ID NOs: 186, 191, 198, 199, 200, 201 and 202 having at least 75% sequence identity thereto; and a β chain comprising the amino acid sequence of SEQ ID NO: 203 or a variant thereof having at least 75% sequence identity thereto.
(xxiv) an α chain comprising the amino acid sequence of SEQ ID NO: 194 or a variant thereof having at least 75% sequence identity thereto; and a β chain comprising an amino acid sequence selected from the group consisting of SEQ ID NO: 10, SEQ ID NO: 11, SEQ ID NO: 195 and variants of SEQ ID NOs: 10, 11 and 195 having at least 75% sequence identity thereto;
(xxv) an α chain comprising the amino acid sequence of SEQ ID NO: 196 or a variant thereof having at least 75% sequence identity thereto; and a β chain comprising an amino acid sequence selected from the group consisting of SEQ ID NO: 21, SEQ ID NO: 22, SEQ ID NO: 197 and variants of SEQ ID NOs: 21, 22 and 197 having at least 75% sequence identity thereto;
(xxvi) an α chain comprising the amino acid sequence of SEQ ID NO: 214 or a variant thereof having at least 75% sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75% sequence identity thereto;
(xxvii) an α chain comprising the amino acid sequence of SEQ ID NO: 214 or a variant thereof having at least 75% sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75% sequence identity thereto;
(xxviii) an α chain comprising the amino acid sequence of SEQ ID NO: 216 or a variant thereof having at least 75% sequence identity thereto; and a β chain comprising an amino acid sequence selected from the group consisting of SEQ ID NO: 54, SEQ ID NO: 55, SEQ ID NO: 217 and variants of SEQ ID NOs: 54, 55 and 217 having at least 75% sequence identity thereto; or
(xxix) an α chain comprising the amino acid sequence of SEQ ID NO: 216 or a variant thereof having at least 75% sequence identity thereto; and a β chain comprising an amino acid sequence selected from the group consisting of SEQ ID NO: 43, SEQ ID NO: 44, SEQ ID NO: 215 and variants of SEQ ID NOs: 43, 44 and 215 having at least 75% sequence identity thereto.

5. (canceled)

6. The TCR of claim 1 which binds to an MHC I and/or MHC II peptide complex.

7. The TCR of claim 1, which is restricted to a human leukocyte antigen (HLA) allele.

8. A TCR according to:
a. part (ii) or (xxiv) of claim 1, which is restricted to HLA-B*38:01 or HLA-C*07:02;
b. part (iii), (viii)-(xxii) or (xxv) of claim 1, which is restricted to HLA-A*02:01; or
c. part (i) or (xxiii) of claim 1, which is restricted to HLA-A*02:01 or HLA-C*03:03.

9. The TCR of claim 1 comprising one or more mutations at the α chain/β chain interface, such that when the α chain and the β chain are expressed in a T-cell, the frequency of mispairing between said chains and endogenous TCR α and β chains is reduced.

10. The TCR of claim 9, wherein the one or more mutations introduce a cysteine residue into the constant region domain of each of the α chain and the β chain, wherein the cysteine residues are capable of forming a disulphide bond between the α chain and the β chain.

11. The TCR of claim 1, which comprises a murinised constant region.

12. The TCR of claim 1, wherein the TCR is a soluble TCR.

13. An isolated polynucleotide encoding an α chain of a T-cell receptor (TCR), and/or a β chain of a TCR, wherein the TCR binds to a Wilms tumour 1 protein (WT1) peptide when presented by a major histocompatibility complex (MHC), and wherein the TCR:
(i) comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions;
(ii) comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions;
(iii) comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions;
(iv) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(v) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vi) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vii) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(viii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(ix) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(x) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xiii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xiv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xvi) comprises a CDR3α comprising the amino acid sequence of CALPDKVIF (SEQ ID NO: 148) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xvii) comprises a CDR3α comprising the amino acid sequence of CAGLYATNKLIF (SEQ ID NO: 153) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xviii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xix) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(xx) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(xxi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions; or
(xxii) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions.

14. The isolated polynucleotide of claim 13, wherein the polynucleotide encodes the α chain linked to the β chain.

15. The isolated polynucleotide of claim 13, which further encodes one or more short interfering RNA (siRNA) or other agents capable of reducing or preventing expression of one or more endogenous TCR genes.

16. A vector comprising a polynucleotide according to claim 13.

17. The vector of claim 16 comprising a polynucleotide, which encodes one or more CD3 chains, CD8, a suicide gene and/or a selectable marker.

18. A cell comprising a TCR according to claim 1 or a polynucleotide encoding the α chain and/or the β chain thereof,
optionally wherein the cell further comprises a vector which encodes one or more CD3 chains, CD8, a suicide gene and/or a selectable marker.

19. The cell of claim 18, wherein the cell is a T-cell, a lymphocyte, or a stem cell, optionally wherein the T-cell, the lymphocyte, or the stem cell is selected from the group consisting of CD4 cells, CD8 cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CK) cells, hematopoietic stem cells and pluripotent stem cells.

20. The cell of claim 19, wherein the cell is a T-cell which has been isolated from a subject.

21. The cell of claim 18, wherein an endogenous gene encoding a TCR α chain and/or an endogenous gene encoding a TCR β chain is disrupted.

22. A method of preparing a cell, which comprises the step of introducing a vector according to claim 16 into a cell in vitro, ex vivo or in vivo, for example by transfection or transduction.

23. The method of claim 22, which comprises the step of T-cell editing, which comprises disrupting an endogenous gene encoding a TCR α chain and/or an endogenous gene encoding a TCR β chain with an artificial nuclease.

24. The method of claim 23, which comprises the step of targeted integration of an expression cassette into the endogenous gene encoding the TCR α chain and/or the endogenous gene encoding the TCR β chain disrupted by the artificial nuclease, wherein the expression cassette comprises a polynucleotide sequence encoding a T-cell receptor (TCR), which binds to a Wilms tumour 1 protein (WT1) peptide when presented by a major histocompatibility complex (MHC), wherein the TCR:
(i) comprises a CDR3α comprising the amino acid sequence of CASGGGADGLTF (SEQ ID NO: 25) or a variant thereof having up to three acid substitutions, additions or deletions, and a CDR3β comprising the amino acid sequence of CASGRGDTEAFF (SEQ ID NO: 30) or a variant thereof having up to three amino acid substitutions, additions or deletions;
(ii) comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions;
(iii) comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions;
(iv) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(v) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vi) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vii) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(viii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(ix) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(x) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xiii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xiv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xvi) comprises a CDR3α comprising the amino acid sequence of CALPDKVIF (SEQ ID NO: 148) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xvii) comprises a CDR3α comprising the amino acid sequence of CAGLYATNKLIF (SEQ ID NO: 153) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xviii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xix) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(xx) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(xxi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions; or
(xxii) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions.

25. The method of claim 22, which comprises the step of disrupting one or more endogenous genes encoding an MHC.

26. The method of claim 22, which comprises the step of disrupting one or more endogenous genes to modify the persistence, expansion, activity, resistance to exhaustion/senescence/inhibitory signals, homing capacity, or other T-cell functions.

27. (canceled)

28. A chimeric molecule comprising the TCR of claim 1, or a portion thereof, conjugated to a non-cellular substrate, a toxin and/or an antibody, optionally wherein the non-cellular substrate is selected from the group consisting of nanoparticles, exosomes and other non-cellular substrates.

29-30. (canceled)

31. A method for treating and/or preventing a disease associated with expression of WT1, which comprises the step of administering the TCR of claim 1, at least one polynucleotide encoding said TCR, or a cell comprising said TCR to a subject in need thereof.

32. The method of claim 31, wherein the disease associated with expression of WT1 is a proliferative disorder.

33. (canceled)

34. A method of adoptive cell transfer comprising administering a cell according to claim 18 to a subject in need thereof.

35. The method of claim 32, wherein the proliferative disorder is a hematological malignancy or a solid tumor.

36. The method of claim 35, wherein the hematological malignancy is acute myeloid leukemia.

37. The method of claim 35, wherein the proliferative disorder is a solid tumor.

38. The cell of claim 21, wherein the endogenous gene encoding a TCR α chain and/or the endogenous gene encoding a TCR β chain is disrupted by insertion of an expression cassette comprising a polynucleotide sequence encoding a T-cell receptor (TCR), which binds to a Wilms tumour 1 protein (WT1) peptide when presented by a major histocompatibility complex (MHC), wherein the TCR:
(i) comprises a CDR3α comprising the amino acid sequence of CASGGGADGLTF (SEQ ID NO: 25) or a variant thereof having up to three amino acid substitutions, additions or deletions, and a CDR3β comprising the amino acid sequence of CASGRGDTEAFF (SEQ ID NO: 30) or a variant thereof having up to three amino acid substitutions, additions or deletions;
(ii) comprises a CDR3α comprising the amino acid sequence of CAMRTGGGADGLTF (SEQ ID NO: 3) or a variant thereof having up to three amino acid substitutions, additions or deletions, and a CDR3β comprising the amino acid sequence of CASSEAGLSYEQYF (SEQ ID NO: 8) or a variant thereof having up to three amino acid substitutions, additions or deletions;
(iii) comprises a CDR3α comprising the amino acid sequence of CILSTRVWAGSYQLTF (SEQ ID NO: 14) or a variant thereof having up to three amino acid substitutions, additions or deletions, and a CDR3β comprising the amino acid sequence of CATGQATQETQYF (SEQ ID NO: 19) or a variant thereof having up to three amino acid substitutions, additions or deletions;
(iv) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(v) comprises a CDR3α comprising the amino acid sequence of CAVIGGTDSWGKLQF (SEQ ID NO: 36) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vi) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CATSREGLAADTQYF (SEQ ID NO: 52) or a variant thereof having up to three amino acid substitutions, additions or deletions;
(vii) comprises a CDR3α comprising the amino acid sequence of CVVPRGLSTDSWGKLQF (SEQ ID NO: 47) or a variant thereof having up to three amino acid substitutions, additions or deletions, and a CDR3β comprising the amino acid sequence of CASSQEEGAVYGYTF (SEQ ID NO: 41) or a variant thereof having up to three amino acid substitutions, additions or deletions;
(viii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(ix) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(x) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xiii) comprises a CDR3α comprising the amino acid sequence of CAVTLLSIEPSAGGYQKVTF (SEQ ID NO: 126) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xiv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CASSLEGRAMPRDSHQETQYF (SEQ ID NO: 136) or a variant thereof having up to three amino acid substitutions, additions or deletions;
(xv) comprises a CDR3α comprising the amino acid sequence of CAATSRDDMRF (SEQ ID NO: 131) or a variant thereof having up to three amino acid substitutions, additions or deletions, and a CDR3β comprising the amino acid sequence of CATSWGLNEQYF (SEQ ID NO: 142) or a variant thereof having up to three amino acid substitutions, additions or deletions;
(xvi) comprises a CDR3α comprising the amino acid sequence of CALPDKVIF (SEQ ID NO: 148) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xvii) comprises a CDR3α comprising the amino acid sequence of CAGLYATNKLIF (SEQ ID NO: 153) or a variant thereof having up to three amino acid substitutions, additions or deletions, and a CDR3β comprising the amino acid sequence of CASSVSAGSTGELFF (SEQ ID NO: 158) or a variant thereof having up to three amino acid substitutions, additions or deletions;
(xviii) comprises a CDR3α comprising the amino acid sequence of CAAPNDYKLSF (SEQ ID NO: 93) or a variant thereof having up to three amino acid substitutions, additions or deletions, and a CDR3β comprising the amino acid sequence of CASSTLGGELFF (SEQ ID NO: 120) or a variant thereof having up to three amino acid substitutions, additions or deletions;
(xix) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions;
(xx) comprises a CDR3α comprising the amino acid sequence of CAVRDGGATNKLIF (SEQ ID NO: 110) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions;
(xxi) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSSGLAFYEQYF (SEQ ID NO: 98) or a variant thereof having up to three amino acid substitutions, additions or deletions; or
(xxii) comprises a CDR3α comprising the amino acid sequence of CLVGGYTGGFKTIF (SEQ ID NO: 115) or a variant thereof having up to three amino acid substitutions, additions or deletions, and a CDR3β comprising the amino acid sequence of CASSQLSGRDSYEQYF (SEQ ID NO: 104) or a variant thereof having up to three amino acid substitutions, additions or deletions.

39. The cell of claim 21, wherein one or more endogenous genes encoding an MHC is disrupted.

40. The cell of claim 21, wherein an endogenous gene involved in persistence, expansion, activity, resistance to exhaustion/senescence/inhibitory signals, homing capacity, or other T-cell functions is disrupted.


SEQUENCE LISTING (250 sequences)

SEQ ID NO: 1 - protein, 7 aa, Artificial Sequence
  Example T-cell receptor (TCR) alpha chain, CDR1alpha
TSDQSYG

SEQ ID NO: 2 - protein, 8 aa, Artificial Sequence
  Example TCR alpha chain, CDR2alpha
QGSYDEQN

SEQ ID NO: 3 - protein, 14 aa, Artificial Sequence
  Example TCR alpha chain, CDR3alpha
CAMRTGGGADGLTF

SEQ ID NO: 4 - protein, 136 aa, Artificial Sequence
  Example TCR alpha chain, variable region
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMRTGGGADGLTFGKGTHLIIQPY

SEQ ID NO: 5 - protein, 276 aa, Artificial Sequence
  Example TCR alpha chain, full - with TRAC constant domain
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMRTGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 5 aa, Artificial Sequence
  Example TCR beta chain, CDR1beta
SNHLY

SEQ ID NO: 7 - protein, 6 aa, Artificial Sequence
  Example TCR beta chain, CDR2beta
FYNNEI

SEQ ID NO: 8 - protein, 14 aa, Artificial Sequence
  Example TCR beta chain, CDR3beta
CASSEAGLSYEQYF

SEQ ID NO: 9 - protein, 134 aa, Artificial Sequence
  Example TCR beta chain, variable region
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEAGLSYEQYFGPGTRLTVTE

SEQ ID NO: 10 - protein, 310 aa, Artificial Sequence
  Example TCR beta chain, full - with TRBC1 constant domain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEAGLSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 11 - protein, 312 aa, Artificial Sequence
  Example TCR beta chain, full - with TRBC2 constant domain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEAGLSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - protein, 7 aa, Artificial Sequence
  Example TCR alpha chain, CDR1alpha
TISGTDY

SEQ ID NO: 13 - protein, 5 aa, Artificial Sequence
  Example TCR alpha chain, CDR2alpha
GLTSN

SEQ ID NO: 14 - protein, 16 aa, Artificial Sequence
  Example TCR alpha chain, CDR3alpha
CILSTRVWAGSYQLTF

SEQ ID NO: 15 - protein, 131 aa, Artificial Sequence
  Example TCR alpha chain, variable region
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILSTRVWAGSYQLTFGKGTKLSVIPN

SEQ ID NO: 16 - protein, 271 aa, Artificial Sequence
  Example TCR alpha chain, full - with TRAC constant domain
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILSTRVWAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 5 aa, Artificial Sequence
  Example TCR beta chain, CDR1beta
KGHDR

SEQ ID NO: 18 - protein, 6 aa, Artificial Sequence
  Example TCR beta chain, CDR2beta
SFDVKD

SEQ ID NO: 19 - protein, 13 aa, Artificial Sequence
  Example TCR beta chain, CDR3beta
CATGQATQETQYF

SEQ ID NO: 20 - protein, 132 aa, Artificial Sequence
  Example TCR beta chain, variable region
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATGQATQETQYFGPGTRLLVLE

SEQ ID NO: 21 - protein, 308 aa, Artificial Sequence
  Example TCR beta chain, with TRBC1 constant domain
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATGQATQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 22 - protein, 310 aa, Artificial Sequence
  Example TCR beta chain, with TRBC2 constant domain
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATGQATQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 6 aa, Artificial Sequence
  Example TCR alpha chain (with TRAV12-3*01), CDR1alpha
NSAFQY

SEQ ID NO: 24 - protein, 6 aa, Artificial Sequence
  Example TCR alpha chain (with TRAV12-3*01), CDR2alpha
TYSSGN

SEQ ID NO: 25 - protein, 12 aa, Artificial Sequence
  Example TCR alpha chain (with TRAV12-3*01 or TRAV12-2*01), CDR3alpha
CASGGGADGLTF

SEQ ID NO: 26 - protein, 133 aa, Artificial Sequence
  Example TCR alpha chain (with TRAV12-3*01), variable region
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCASGGGADGLTFGKGTHLIIQPY

SEQ ID NO: 27 - protein, 273 aa, Artificial Sequence
  Example TCR alpha chain (with TRAV12-3*01), full - with TRAC constant domain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCASGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 5 aa, Artificial Sequence
  Example TCR beta chain, CDR1beta
SGDLS

SEQ ID NO: 29 - protein, 6 aa, Artificial Sequence
  Example TCR beta chain, CDR2beta
YYNGEE

SEQ ID NO: 30 - protein, 12 aa, Artificial Sequence
  Example TCR beta chain, CDR3beta
CASGRGDTEAFF

SEQ ID NO: 31 - protein, 131 aa, Artificial Sequence
  Example TCR beta chain, variable region
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGRGDTEAFFGQGTRLTVVE

SEQ ID NO: 32 - protein, 307 aa, Artificial Sequence
  Example TCR beta chain, full - with TRBC1 constant domain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGRGDTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 309 aa, Artificial Sequence
  Example TCR beta chain, full - with TRBC2 constant domain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGRGDTEAFFGQGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 34 - protein, 6 aa, Artificial Sequence
  Example TCR alpha chain S4 population, CDR1alpha
DRGSQS

SEQ ID NO: 35 - protein, 6 aa, Artificial Sequence
  Example TCR alpha chain S4 population, CDR2alpha
IYSNGD

SEQ ID NO: 36 - protein, 15 aa, Artificial Sequence
  Example TCR alpha chain S4 population, CDR3alpha
CAVIGGTDSWGKLQF

SEQ ID NO: 37 - protein, 135 aa, Artificial Sequence
  Example TCR alpha chain S4 population, variable region
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVIGGTDSWGKLQFGAGTQVVVTPD

SEQ ID NO: 38 - protein, 275 aa, Artificial Sequence
  Example TCR alpha chain S4 population, full - with TRAC constant domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVIGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 5 aa, Artificial Sequence
  Example TCR beta chain S4 population, CDR1beta
LGHNA

SEQ ID NO: 40 - protein, 6 aa, Artificial Sequence
  Example TCR beta chain S4 population, CDR2beta
YSLEER

SEQ ID NO: 41 - protein, 15 aa, Artificial Sequence
  Example TCR beta chain S4 population, CDR3beta
CASSQEEGAVYGYTF

SEQ ID NO: 42 - protein, 137 aa, Artificial Sequence
  Example TCR beta chain S4 population, variable region
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEEGAVYGYTFGSGTRLTVVE

SEQ ID NO: 43 - protein, 313 aa, Artificial Sequence
  Example TCR beta chain S4 population, full - with TRBC1 constant domain
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEEGAVYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 44 - protein, 315 aa, Artificial Sequence
  Example TCR beta chain S4 population, full - with TRBC2 constant domain
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEEGAVYGYTFGSGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 6 aa, Artificial Sequence
  Example TCR alpha chain S1 IFNg enriched population, CDR1alpha
NSASQS

SEQ ID NO: 46 - protein, 6 aa, Artificial Sequence
  Example TCR alpha chain S1 IFNg enriched population, CDR2alpha
VYSSGN

SEQ ID NO: 47 - protein, 17 aa, Artificial Sequence
  Example TCR alpha chain S1 IFNg enriched population, CDR3alpha
CVVPRGLSTDSWGKLQF

SEQ ID NO: 48 - protein, 136 aa, Artificial Sequence
  Example TCR alpha chain S1 IFNg enriched population, variable region
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVPRGLSTDSWGKLQFGAGTQVVVTPD

SEQ ID NO: 49 - protein, 276 aa, Artificial Sequence
  Example TCR alpha chain S1 IFNg enriched population, full - with TRAC constant domain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVPRGLSTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 5 aa, Artificial Sequence
  Example TCR beta chain S1 IFNg enriched population, CDR1beta
LNHNV

SEQ ID NO: 51 - protein, 6 aa, Artificial Sequence
  Example TCR beta chain S1 IFNg enriched population, CDR2beta
YYDKDF

SEQ ID NO: 52 - protein, 15 aa, Artificial Sequence
  Example TCR beta chain S1 IFNg enriched population, CDR3beta
CATSREGLAADTQYF

SEQ ID NO: 53 - protein, 134 aa, Artificial Sequence
  Example TCR beta chain S1 IFNg enriched population, variable region
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSREGLAADTQYFGPGTRLTVLE

SEQ ID NO: 54 - protein, 310 aa, Artificial Sequence
  Example TCR beta chain S1 IFNg enriched population, full - with TRBC1 constant domain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSREGLAADTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 55 - protein, 312 aa, Artificial Sequence
  Example TCR beta chain S1 IFNg enriched population, full - with TRBC2 constant domain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSREGLAADTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 56 - DNA, 828 bp, Artificial Sequence
  nucleotide sequence encoding TCR, donor HD12 alpha chain (with TRAC)
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagaacgggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 57 - DNA, 930 bp, Artificial Sequence
  nucleotide sequence encoding TCR, donor HD12 beta chain (with TRBC1)
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagcgggactttcctacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 58 - DNA, 936 bp, Artificial Sequence
  nucleotide sequence encoding TCR, donor HD12 beta chain (with TRBC2)
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagcgggactttcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 59 - DNA, 813 bp, Artificial Sequence
  nucleotide sequence encoding TCR, donor HD13 alpha chain (with TRAC)
atgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagtacccgggtctgggctgggagttaccaactcactttcgggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 60 - DNA, 924 bp, Artificial Sequence
  nucleotide sequence encoding TCR, donor HD13 beta chain (with TRBC1)
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccggccaggcgacccaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 61 - DNA, 930 bp, Artificial Sequence
  nucleotide sequence encoding TCR, donor HD13 beta chain (with TRBC2)
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccggccaggcgacccaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 62 - DNA, 819 bp, Artificial Sequence
  nucleotide sequence encoding TCR, donor HD14 alpha chain (TRAV12-3*01) (with TRAC)
atgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcctcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 63 - DNA, 921 bp, Artificial Sequence
  nucleotide sequence encoding TCR, donor HD14 beta chain (with TRBC1)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcgggaggggggacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 64 - DNA, 927 bp, Artificial Sequence
  nucleotide sequence encoding TCR, donor HD14 beta chain (with TRBC2)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcgggaggggggacactgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 65 - DNA, 825 bp, Artificial Sequence
  nucleotide sequence encoding TCR, donor HD15 S4 population alpha chain (with TRAC)
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgatagggggaactgacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 66 - DNA, 939 bp, Artificial Sequence
  nucleotide sequence encoding TCR, donor HD15 S4 population beta chain (with TRBC1)
atgggctgcaggctgctctgctgtgcggttctctgtctcctgggagcgggtgagttggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagaagagggggctgtctatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 67 - DNA, 945 bp, Artificial Sequence
  nucleotide sequence encoding TCR, donor HD15 S4 population beta chain (with TRBC2)
atgggctgcaggctgctctgctgtgcggttctctgtctcctgggagcgggtgagttggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagaagagggggctgtctatggctacaccttcggttcggggaccaggttaaccgttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 68 - DNA, 828 bp, Artificial Sequence
  nucleotide sequence encoding TCR, donor HD15 S1 IFNg enriched population alpha chain (with TRAC)
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgccccgggggctttcaactgacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 69 - DNA, 930 bp, Artificial Sequence
  nucleotide sequence encoding TCR, donor HD15 S1 IFNg enriched population beta chain (with TRBC1)
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagggaggggctagcggcagatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 70 - DNA, 936 bp, Artificial Sequence
  nucleotide sequence encoding TCR, donor HD15 S1 IFNg enriched population beta chain (with TRBC2)
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagggaggggctagcggcagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 71 - protein, 9 aa, Artificial Sequence
  Wilms tumour 1 protein (WT1) 126-134 epitope
RMFPNAPYL

SEQ ID NO: 72 - protein, 9 aa, Artificial Sequence
  WT1 37-45 epitope
VLDFAPPGA

SEQ ID NO: 73 - protein, 9 aa, Artificial Sequence
  HD12 strong binder peptide
YRIHTHGVF

SEQ ID NO: 74 - protein, 9 aa, Artificial Sequence
  HD13 and HD14 strong binder peptide
LLAAILDFL

SEQ ID NO: 75 - protein, 9 aa, Artificial Sequence
  HD14 strong binder peptide
AAILDFLLL

SEQ ID NO: 76 - protein, 140 aa, Artificial Sequence
  amino acid sequence of an alpha chain constant domain encoded by a TRAC gene
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 77 - protein, 176 aa, Artificial Sequence
  amino acid sequence of a beta chain constant domain encoded by a TRBC1 gene
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 78 - protein, 178 aa, Artificial Sequence
  amino acid sequence of a beta chain constant domain encoded by a TRBC2 gene
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 79 - protein, 575 aa, Homo sapiens
SRQRPHPGALRNPTACPLPHFPPSLPPTHSPTHPPRAGTAAQAPGPRRLLAAILDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 80 - protein, 15 aa, Artificial Sequence
  peptide P4
PTACPLPHFPPSLPP

SEQ ID NO: 81 - protein, 15 aa, Artificial Sequence
  peptide P7
LPPTHSPTHPPRAGT

SEQ ID NO: 82 - protein, 15 aa, Artificial Sequence
  peptide P13
LLAAILDFLLLQDPA

SEQ ID NO: 83 - protein, 15 aa, Artificial Sequence
  peptide P20
RSGPGCLQQPEQQGV

SEQ ID NO: 84 - protein, 15 aa, Artificial Sequence
  peptide P25
IWAKLGAAEASAERL

SEQ ID NO: 85 - protein, 15 aa, Artificial Sequence
  peptide P33
SDVRDLNALLPAVPS

SEQ ID NO: 86 - protein, 15 aa, Artificial Sequence
  peptide P37
GGGGGCALPVSGAAQ

SEQ ID NO: 87 - protein, 15 aa, Artificial Sequence
  peptide P91
CMTWNQMNLGATLKG

SEQ ID NO: 88 - protein, 15 aa, Artificial Sequence
  peptide P92
NQMNLGATLKGVAAG

SEQ ID NO: 89 - protein, 15 aa, Artificial Sequence
  peptide P129
TCQRKFSRSDHLKTH

SEQ ID NO: 90 - protein, 15 aa, Artificial Sequence
  peptide P131
SDHLKTHTRTHTGKT

SEQ ID NO: 91 - protein, 6 aa, Artificial Sequence
  Example TCR alpha chain direct sequencing upon sorting, CDR1alpha
VSNAYN

SEQ ID NO: 92 - protein, 4 aa, Artificial Sequence
  Example TCR alpha chain direct sequencing upon sorting, CDR2alpha
GSKP

SEQ ID NO: 93 - protein, 11 aa, Artificial Sequence
  Example TCR alpha chain direct sequencing upon sorting, CDR3alpha
CAAPNDYKLSF

SEQ ID NO: 94 - protein, 130 aa, Artificial Sequence
  Example TCR alpha chain direct sequencing upon sorting, variable region
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAAPNDYKLSFGAGTTVTVRAN

SEQ ID NO: 95 - protein, 270 aa, Artificial Sequence
  Example TCR alpha chain direct sequencing upon sorting, full - with TRAC constant domain
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAAPNDYKLSFGAGTTVTVRANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - protein, 5 aa, Artificial Sequence
  Example TCR beta 1 chain direct sequencing upon sorting, CDR1beta
SEHNR

SEQ ID NO: 97 - protein, 6 aa, Artificial Sequence
  Example TCR beta 1 chain direct sequencing upon sorting, CDR2beta
FQNEAQ

SEQ ID NO: 98 - protein, 14 aa, Artificial Sequence
  Example TCR beta 1 chain direct sequencing upon sorting, CDR3beta
CASSSGLAFYEQYF

SEQ ID NO: 99 - protein, 134 aa, Artificial Sequence
  Example TCR beta 1 chain direct sequencing upon sorting, variable region
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSGLAFYEQYFGPGTRLTVTE

SEQ ID NO: 100 - protein, 310 aa, Artificial Sequence
  Example TCR beta 1 chain direct sequencing upon sorting, full - with TRBC1 constant domain
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSGLAFYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 101 - protein, 312 aa, Artificial Sequence
  Example TCR beta 1 chain direct sequencing upon sorting, full - with TRBC2 constant domain
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSGLAFYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 102 - protein, 5 aa, Artificial Sequence
  Example TCR beta 2 chain direct sequencing upon sorting, CDR1beta
SGHDN

SEQ ID NO: 103 - protein, 6 aa, Artificial Sequence
  Example TCR beta 2 chain direct sequencing upon sorting, CDR2beta
FVKESK

SEQ ID NO: 104 - protein, 16 aa, Artificial Sequence
  Example TCR beta 2 chain direct sequencing upon sorting, CDR3beta
CASSQLSGRDSYEQYF

SEQ ID NO: 105 - protein, 136 aa, Artificial Sequence
  Example TCR beta 2 chain direct sequencing upon sorting, variable region
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQLSGRDSYEQYFGPGTRLTVTE

SEQ ID NO: 106 - protein, 312 aa, Artificial Sequence
  Example TCR beta 2 chain direct sequencing upon sorting, full - with TRBC1 constant domain
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQLSGRDSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 107 - protein, 314 aa, Artificial Sequence
  Example TCR beta 2 chain direct sequencing upon sorting, full - with TRBC2 constant domain
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQLSGRDSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 108 - protein, 6 aa, Artificial Sequence
  Example TCR alpha 1 chain growing colony, CDR1alpha
VSGNPY

SEQ ID NO: 109 - protein, 8 aa, Artificial Sequence
  Example TCR alpha 1 chain growing colony, CDR2alpha
YITGDNLV

SEQ ID NO: 110 - protein, 14 aa, Artificial Sequence
  Example TCR alpha 1 chain growing colony, CDR3alpha
CAVRDGGATNKLIF

SEQ ID NO: 111 - protein, 134 aa, Artificial Sequence
  Example TCR alpha 1 chain growing colony, variable region
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDGGATNKLIFGTGTLLAVQPN

SEQ ID NO: 112 - protein, 274 aa, Artificial Sequence
  Example TCR alpha 1 chain growing colony, full - with TRAC constant domain
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDGGATNKLIFGTGTLLAVQPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 113 - protein, 7 aa, Artificial Sequence
  Example TCR alpha 2 chain growing colony, CDR1alpha
NIATNDY

SEQ ID NO: 114 - protein, 5 aa, Artificial Sequence
  Example TCR alpha 2 chain growing colony, CDR2alpha
GYKTK

SEQ ID NO: 115 - protein, 14 aa, Artificial Sequence
  Example TCR alpha 2 chain growing colony, CDR3alpha
CLVGGYTGGFKTIF

SEQ ID NO: 116 - protein, 129 aa, Artificial Sequence
  Example TCR alpha 2 chain growing colony, variable region
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGGYTGGFKTIFGAGTRLFVKAN

SEQ ID NO: 117 - protein, 269 aa, Artificial Sequence
  Example TCR alpha 2 chain growing colony, full - with TRAC constant domain
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 118 - protein, 5 aa, Artificial Sequence
  Example TCR beta chain growing colony, CDR1beta
MNHEY

SEQ ID NO: 119 - protein, 6 aa, Artificial Sequence
  Example TCR beta chain growing colony, CDR2beta
SMNVEV

SEQ ID NO: 120 - protein, 12 aa, Artificial Sequence
  Example TCR beta chain growing colony, CDR3beta
CASSTLGGELFF

SEQ ID NO: 121 - protein, 131 aa, Artificial Sequence
  Example TCR beta chain growing colony, variable region
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTLGGELFFGEGSRLTVLE

SEQ ID NO: 122 - protein, 307 aa, Artificial Sequence
  Example TCR beta chain growing colony, full - with TRBC1 constant domain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTLGGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 123 - protein, 309 aa, Artificial Sequence
  Example TCR beta chain growing colony, full - with TRBC2 constant domain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTLGGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 124 - protein, 6 aa, Artificial Sequence
  Example TCR alpha 1 chain, CDR1alpha
SSVSVY

SEQ ID NO: 125 - protein, 8 aa, Artificial Sequence
  Example TCR alpha 1 chain, CDR2alpha
YLSGSTLV

SEQ ID NO: 126 - protein, 20 aa, Artificial Sequence
  Example TCR alpha 1 chain, CDR3alpha
CAVTLLSIEPSAGGYQKVTF

SEQ ID NO: 127 - protein, 140 aa, Artificial Sequence
  Example TCR alpha 1 chain, variable region
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVTLLSIEPSAGGYQKVTFGIGTKLQVIPN

SEQ ID NO: 128 - protein, 280 aa, Artificial Sequence
  Example TCR alpha 1 chain, full - with TRAC constant domain
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVTLLSIEPSAGGYQKVTFGIGTKLQVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 129 - protein, 6 aa, Artificial Sequence
  Example TCR alpha 2 chain, CDR1alpha
DSASNY

SEQ ID NO: 130 - protein, 7 aa, Artificial Sequence
  Example TCR alpha 2 chain, CDR2alpha
IRSNVGE

SEQ ID NO: 131 - protein, 11 aa, Artificial Sequence
  Example TCR alpha 2 chain, CDR3alpha
CAATSRDDMRF

SEQ ID NO: 132 - protein, 130 aa, Artificial Sequence
  Example TCR alpha 2 chain, variable region
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATSRDDMRFGAGTRLTVKPN

SEQ ID NO: 133 - protein, 270 aa, Artificial Sequence
  Example TCR alpha 2 chain, full - with TRAC constant domain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATSRDDMRFGAGTRLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 134 - protein, 5 aa, Artificial Sequence
  Example TCR beta 1 chain, CDR1beta
SEHNR

SEQ ID NO: 135 - protein, 6 aa, Artificial Sequence
  Example TCR beta 1 chain, CDR2beta
FQNEAQ

SEQ ID NO: 136 - protein, 21 aa, Artificial Sequence
  Example TCR beta 1 chain, CDR3beta
CASSLEGRAMPRDSHQETQYF

SEQ ID NO: 137 - protein, 141 aa, Artificial Sequence
  Example TCR beta 1 chain, variable region
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLEGRAMPRDSHQETQYFGPGTRLLVLE

SEQ ID NO: 138 - protein, 317 aa, Artificial Sequence
  Example TCR beta 1 chain, full - with TRBC1 constant domain
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLEGRAMPRDSHQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 139 - protein, 319 aa, Artificial Sequence
  Example TCR beta 1 chain, full - with TRBC2 constant domain
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLEGRAMPRDSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 140 - protein, 5 aa, Artificial Sequence
  Example TCR beta 2 chain, CDR1beta
LNHNV

SEQ ID NO: 141 - protein, 6 aa, Artificial Sequence
  Example TCR beta 2 chain, CDR2beta
YYDKDF

SEQ ID NO: 142 - protein, 12 aa, Artificial Sequence
  Example TCR beta 2 chain, CDR3beta
CATSWGLNEQYF

SEQ ID NO: 143 - protein, 131 aa, Artificial Sequence
  Example TCR beta 2 chain, variable region
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSWGLNEQYFGPGTRLTVTE

SEQ ID NO: 144 - protein, 307 aa, Artificial Sequence
  Example TCR beta 2 chain, full - with TRBC1 constant domain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSWGLNEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 145 - protein, 309 aa, Artificial Sequence
  Example TCR beta 2 chain, full - with TRBC2 constant domain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSWGLNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 146 - protein, 7 aa, Artificial Sequence
  Example TCR alpha 1 chain, CDR1alpha
TRDTTYY

SEQ ID NO: 147 - protein, 8 aa, Artificial Sequence
  Example TCR alpha 1 chain, CDR2alpha
RNSFDEQN

SEQ ID NO: 148 - protein, 9 aa, Artificial Sequence
  Example TCR alpha 1 chain, CDR3alpha
CALPDKVIF

SEQ ID NO: 149 - protein, 131 aa, Artificial Sequence
  Example TCR alpha 1 chain, variable region
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALPDKVIFGPGTSLSVIPN

SEQ ID NO: 150 - protein, 271 aa, Artificial Sequence
  Example TCR alpha 1 chain, full - with TRAC constant domain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALPDKVIFGPGTSLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 151 - protein, 5 aa, Artificial Sequence
  Example TCR alpha 2 chain, CDR1alpha
SIFNT

SEQ ID NO: 152 - protein, 7 aa, Artificial Sequence
  Example TCR alpha 2 chain, CDR2alpha
LYKAGEL

SEQ ID NO: 153 - protein, 12 aa, Artificial Sequence
  Example TCR alpha 2 chain, CDR3alpha
CAGLYATNKLIF

SEQ ID NO: 154 - protein, 129 aa, Artificial Sequence
  Example TCR alpha 2 chain, variable region
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGLYATNKLIFGTGTLLAVQPN

SEQ ID NO: 155 - protein, 269 aa, Artificial Sequence
  Example TCR alpha 2 chain, full - with TRAC constant domain
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGLYATNKLIFGTGTLLAVQPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 156 - protein, 5 aa, Artificial Sequence
  Example TCR beta chain, CDR1beta
SGDLS

SEQ ID NO: 157 - protein, 6 aa, Artificial Sequence
  Example TCR beta chain, CDR2beta
YYNGEE

SEQ ID NO: 158 - protein, 15 aa, Artificial Sequence
  Example TCR beta chain, CDR3beta
CASSVSAGSTGELFF

SEQ ID NO: 159 - protein, 134 aa, Artificial Sequence
  Example TCR beta chain, variable region
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVSAGSTGELFFGEGSRLTVLE

SEQ ID NO: 160 - protein, 310 aa, Artificial Sequence
  Example TCR beta chain, full - with TRBC1 constant domain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVSAGSTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 161 - protein, 312 aa, Artificial Sequence
  Example TCR beta chain, full - with TRBC2 constant domain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVSAGSTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 162 - DNA, 810 bp, Artificial Sequence
  nucleotide sequence encoding TCR, donor Patient 1 direct sequencing upon sorting alpha chain (with TRAC)
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgcccctaacgactacaagctcagctttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 163 - DNA, 930 bp, Artificial Sequence
  nucleotide sequence encoding TCR, donor Patient 1 direct sequencing upon sorting beta1 chain (with TRBC1)
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcagcggactagcgttctacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 164 - DNA, 936 bp, Artificial Sequence
  nucleotide sequence encoding TCR, donor Patient 1 direct sequencing upon sorting beta1 chain (with TRBC2)
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcagcggactagcgttctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 165 - DNA, 936 bp, Artificial Sequence
  nucleotide sequence encoding TCR, donor Patient 1 direct sequencing upon sorting beta2 chain (with TRBC1)
atggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaattgtcagggcgcgactcctacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 166 - DNA, 942 bp, Artificial Sequence
  nucleotide sequence encoding TCR, donor Patient 1 direct sequencing upon sorting beta2 chain (with TRBC2)
atggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaattgtcagggcgcgactcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 167 - DNA, 822 bp, Artificial Sequence
  nucleotide sequence encoding TCR, donor Patient 1 growing colony alpha1 chain (with TRAC)
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacggtggtgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 168 - DNA, 807 bp, Artificial Sequence
  nucleotide sequence encoding TCR, donor Patient 1 growing colony alpha2 chain (with TRAC)
atgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtggttatactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 169 - DNA, 921 bp, Artificial Sequence
  nucleotide sequence encoding TCR, donor Patient 1 growing colony beta chain (with TRBC1)
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtacgcttgggggggagctgttttttggagaaggctctaggctgaccgtactggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 170 - DNA, 927 bp, Artificial Sequence
  nucleotide sequence encoding TCR, donor Patient 1 growing colony beta chain (with TRBC2)
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtacgcttgggggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 171 - DNA, 840 bp, Artificial Sequence
  nucleotide sequence encoding TCR, donor Patient 2 alpha1 chain (with TRAC)
atgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttgaaagcatcaacggttttgaggctgaatttaacaagagtcaaacttccttccacttgaggaaaccctcagtccatataagcgacacggctgagtacttctgtgctgtgaccctgctttcgattgagccttcggctgggggttaccagaaagttacctttggaattggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 172 - DNA, 810 bp, Artificial Sequence
  nucleotide sequence encoding TCR, donor Patient 2 alpha2 chain (with TRAC)
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaacctcccgcgatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 173 - DNA, 951 bp, Artificial Sequence
  nucleotide sequence encoding TCR, donor Patient 2 beta1 chain (with TRBC1)
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagaaggaagggccatgcccagggacagccaccaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 174 - DNA, 957 bp, Artificial Sequence
  nucleotide sequence encoding TCR, donor Patient 2 beta1 chain (with TRBC2)
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagaaggaagggccatgcccagggacagccaccaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 175 - DNA, 921 bp, Artificial Sequence
  nucleotide sequence encoding TCR, donor Patient 2 beta2 chain (with TRBC1)
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagctgggggctaaacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 176 - DNA, 927 bp, Artificial Sequence
  nucleotide sequence encoding TCR, donor Patient 2 beta2 chain (with TRBC2)
atgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagctgggggctaaacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 177 - DNA, 813 bp, Artificial Sequence
  nucleotide sequence encoding TCR, donor Patient 3 alpha1 chain (with TRAC)
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgcccgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 178 - DNA, 807 bp, Artificial Sequence
  nucleotide sequence encoding TCR, donor Patient 3 alpha2 chain (with TRAC)
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggctatatgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 179 - DNA, 930 bp, Artificial Sequence
  nucleotide sequence encoding TCR, donor Patient 3 beta chain (with TRBC1)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtatcggcagggagcaccggggagctgttttttggagaaggctctaggctgaccgtactggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 180 - DNA, 936 bp, Artificial Sequence
  nucleotide sequence encoding TCR, donor Patient 3 beta chain (with TRBC2)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtatcggcagggagcaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 181 - protein, 15 aa, Artificial Sequence
  Wilms tumour 1 protein (WT1) peptide
GAQYRIHTHGVFRGI

SEQ ID NO: 182 - protein, 6 aa, Artificial Sequence
  Example TCR alpha chain (with TRAV12-2*01 or TRAV12-2*02 ), CDR1alpha
DRGSQS

SEQ ID NO: 183 - protein, 6 aa, Artificial Sequence
  Example TCR alpha chain (with TRAV12-2*01 or or TRAV12-2*02 ), CDR2alpha
IYSNGD

SEQ ID NO: 184 - skipped

SEQ ID NO: 185 - protein, 132 aa, Artificial Sequence
  Example TCR alpha chain (with TRAV12-2*01), variable region
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCASGGGADGLTFGKGTHLIIQPY

SEQ ID NO: 186 - protein, 272 aa, Artificial Sequence
  Example TCR alpha chain (with TRAV12-2*01), full - with TRAC constant domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCASGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 187-189 - skipped (3 entries)

SEQ ID NO: 190 - protein, 133 aa, Artificial Sequence
  Example TCR alpha chain (with TRAV12-2*02 ), variable region
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCASGGGADGLTFGKGTHLIIQPY

SEQ ID NO: 191 - protein, 273 aa, Artificial Sequence
  Example TCR alpha chain (with TRAV12-2*02 ), full - with TRAC constant domain
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCASGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 192 - DNA, 816 bp, Artificial Sequence
  nucleotide sequence encoding TCR, donor HD14 alpha chain (TRAV12-2*01) (with TRAC)
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgcctcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 193 - DNA, 819 bp, Artificial Sequence
  nucleotide sequence encoding TCR, donor HD14 alpha chain (TRAV12-2*02) (with TRAC)
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgcctcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 194 - protein, 276 aa, Artificial Sequence
  Example TCR alpha chain, full - with TRAC constant domain
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMRTGGGADGLTFGKGTHLIIQPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 195 - protein, 312 aa, Artificial Sequence
  Example TCR beta chain, full - with TRBC2 constant domain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEAGLSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 196 - protein, 271 aa, Artificial Sequence
  Example TCR alpha chain, full - with TRAC constant domain
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILSTRVWAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 197 - protein, 310 aa, Artificial Sequence
  Example TCR beta chain, full - with TRBC2 constant domain
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATGQATQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 198 - protein, 273 aa, Artificial Sequence
  Example TCR alpha chain (TRAV12-3*01 WT), full - with TRAC constant domain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCASGGGADGLTFGKGTHLIIQPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 199 - protein, 272 aa, Artificial Sequence
  Example TCR alpha chain (TRAV12-2*01 WT), full - with TRAC constant domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCASGGGADGLTFGKGTHLIIQPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 200 - protein, 272 aa, Artificial Sequence
  Example TCR alpha chain (TRAV12-2*01 mut), full - with TRAC constant domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCASGGGADGLTFGKGTHLIIQPYIQNPDPAVYQLRDSKSSDKSVCLFTDFDSQTQVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 201 - protein, 273 aa, Artificial Sequence
  Example TCR alpha chain (TRAV12-2*02 WT), full - with TRAC constant domain
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCASGGGADGLTFGKGTHLIIQPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 202 - protein, 273 aa, Artificial Sequence
  Example TCR alpha chain (TRAV12-2*02 mut), full - with TRAC constant domain
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCASGGGADGLTFGKGTHLIIQPYIQNPDPAVYQLRDSKSSDKSVCLFTDFDSQTQVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 203 - protein, 309 aa, Artificial Sequence
  Example TCR beta chain (TRAV12-3*01 WT), full - with TRBC2 constant domain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGRGDTEAFFGQGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 204 - DNA, 828 bp, Artificial Sequence
  nucleotide sequence encoding TCR, donor HD12 alpha chain (with TRAC)
atgagcctgtctagcctgctgaaggtggtcacagccagcctgtggctcggacctggaatcgcccagaagatcacccagacacagcccggcatgttcgtgcaagagaaagaagccgtgacactggactgcacctacgacaccagcgatcagagctacggcctgttctggtacaagcagcctagcagcggcgagatgatcttcctgatctaccagggcagctacgacgagcagaatgccaccgagggcagatacagcctgaacttccagaaggcccggaagtccgccaacctggtcatttctgcttctcagctgggcgacagcgccatgtacttttgcgccatgagaacaggcggcggagccgatggactgacatttggcaagggcacccacctgatcatccagccttacattcagaaccccgatcctgccgtgtaccagctgagggatagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctcaatttccagaacctgagcgtgatcggcttccggatcctgctgctgaaagtggccggcttcaacctgctgatgaccctgagactgtggtccagc

SEQ ID NO: 205 - DNA, 936 bp, Artificial Sequence
  nucleotide sequence encoding TCR, donor HD12 beta chain (with TRBC2)
atggatacttggcttgtgtgctgggccatcttcagcctgctgaaggccggactgacagagcccgaagtgacacagacacccagccaccaagtgacccagatgggccaagaagtgatcctgcgctgcgtgcccatcagcaaccacctgtacttctactggtacagacagatcctgggccagaaagtggaattcctggtgtccttctacaacaacgagatcagcgagaagtccgagatcttcgacgaccagttcagcgtggaaagacccgacggcagcaacttcaccctgaagatcagaagcaccaagctcgaggacagcgccatgtacttttgcgcctcttctgaagccggcctgagctacgagcagtactttggccctggcaccagactgaccgtgaccgaggatctgaagaacgtgttcccacctgaggtggccgtgttcgaaccttctgaggccgagatctctcacacccagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgtccgagaacgatgagtggacccaggatagagccaagcctgtgactcagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggc

SEQ ID NO: 206 - DNA, 813 bp, Artificial Sequence
  nucleotide sequence encoding TCR, donor HD13 alpha chain (with TRAC)
atgaagctggtcaccagcatcaccgtgctgctgagcctgggcattatgggcgacgccaagaccacacagcccaacagcatggaaagcaacgaagaggaacccgtgcatctgccctgcaaccacagcacaatcagcggcaccgactacatccactggtacagacagctgcccagccagggacctgagtatgtgatccacggcctgaccagcaacgtgaacaacagaatggccagcctggctatcgccgaggacagaaagagcagcaccctgatcctgcacagagccacactgagagatgccgccgtgtactactgcatcctgagcacaagagtgtgggccggcagctaccagctgacatttggcaagggcaccaagctgagcgtgatccccaacattcagaaccccgatcctgccgtgtaccagctgcgggatagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccctgaggacacattcttcccaagtcctgagagcagctgcgacgtgaaactggtggaaaagagcttcgagacagacaccaacctgaacttccagaacctgtccgtgatcggcttccggatcctgctgctgaaagtggccggcttcaacctgctgatgaccctgagactgtggtccagc

SEQ ID NO: 207 - DNA, 930 bp, Artificial Sequence
  nucleotide sequence encoding TCR, donor HD13 beta chain (with TRBC2)
atggcttctcttctgtttttctgcggcgccttctacctgctcggcaccggatctatggacgccgacgttacccagacaccacggaacagaatcaccaagaccggcaagcggatcatgctggaatgcagccagaccaagggccacgaccggatgtactggtacagacaggatccaggactgggcctgagactgatctactacagcttcgacgtgaaggacatcaacaagggcgagatcagcgacggctacagcgtgtcaagacaggcccaggccaagttcagcctgagcctggaaagcgctatccccaaccagacagccctgtacttttgtgccaccggccaggccacacaagagacacagtatttcggccctggcaccagactgctggtgctggaagatctgaagaacgtgttcccacctgaggtggccgtgttcgagccttctgaggccgagatctctcacacccagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggc

SEQ ID NO: 208 - DNA, 819 bp, Artificial Sequence
  nucleotide sequence encoding TCR, donor HD14 alpha chain TRAV12-3*01 WT (with TRAC)
atgatgaagtccctgcgggtgctgctggtcatcctgtggctgcaactgagctgggtctggtcccagcagaaagaggtggaacaggaccctggacctctgtctgttcctgagggcgccatcgtgtccctgaactgcacctacagcaacagcgccttccagtacttcatgtggtacagacagtacagccggaagggccccgagctgctgatgtacacatacagcagcggcaacaaagaggacggccggtttacagcccaggtggacaagagcagcaagtacatctccctgttcatccgggacagccagcctagcgatagcgccacatatctgtgtgcatctggcggcggagccgatggcctgacatttggaaagggcacccacctgatcatccagccttacattcagaaccccgatcctgccgtgtaccagctgagagacagcaagtccagcgacaagagcgtgtgcctgttcaccgacttcgacagccagaccaacgtgtcccagagcaaggacagcgacgtgtacattaccgataagtgcgtgctggacatgcggagcatggacttcaagagcaactccgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtccagc

SEQ ID NO: 209 - DNA, 816 bp, Artificial Sequence
  nucleotide sequence encoding TCR, donor HD14 alpha chain TRAV12-2*01 WT (with TRAC)
atgaagtccctgagagtgctgctggtcatcctgtggctgcagctgtcttgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgttcatctacagcaacggcgacaaagaggacggccggtttacagcccagctgaacaaggccagccagtacgtgtccctgctgatcagagatagccagcctagcgacagcgccacctacctttgtgcatctggtggcggagccgatggcctgacatttggcaagggaacccacctgatcatccagccttacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtccagc

SEQ ID NO: 210 - DNA, 816 bp, Artificial Sequence
  nucleotide sequence encoding TCR, donor HD14 alpha chain TRAV12-2*01 mut (with TRAC)
atgaagtccctgagagtgctgctggtcatcctgtggctgcagctgtcttgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgttcatctacagcaacggcgacaaagaggacggccggtttacagcccagctgaacaaggccagccagtacgtgtccctgctgatcagagatagccagcctagcgacagcgccacctacctttgtgcatctggtggcggagccgatggcctgacatttggcaagggaacccacctgatcatccagccttacattcagaaccccgatcctgccgtgtaccagctgagagacagcaagagcagcgacaagagcgtgtgcctgttcaccgacttcgacagccagacacaggtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtccagc

SEQ ID NO: 211 - DNA, 819 bp, Artificial Sequence
  nucleotide sequence encoding TCR, donor HD14 alpha chain TRAV12-2*02 WT (with TRAC)
atgatgaagtccctgcgggtgctgctggtcatcctgtggctgcaactgagctgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctacctttgtgcatctggtggcggagccgatggcctgacatttggcaagggaacccacctgatcatccagccttacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtccagc

SEQ ID NO: 212 - DNA, 819 bp, Artificial Sequence
  nucleotide sequence encoding TCR, donor HD14 alpha chain TRAV12-2*02 mut (with TRAC)
atgatgaagtccctgcgggtgctgctggtcatcctgtggctgcaactgagctgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctacctttgtgcatctggtggcggagccgatggcctgacatttggcaagggaacccacctgatcatccagccttacattcagaaccccgatcctgccgtgtaccagctgagagacagcaagagcagcgacaagagcgtgtgcctgttcaccgacttcgacagccagacacaggtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtccagc

SEQ ID NO: 213 - DNA, 927 bp, Artificial Sequence
  nucleotide sequence encoding TCR, donor HD14 beta chain TRAV12-3*01 WT (with TRBC2)
atgggttttagactgctgtgctgcgtggccttctgtctgcttggagctggccctgtggatagcggcgttacccagacacctaagcacctgatcacagccacaggccagcgcgtgaccctgagatgttctcctagaagcggcgacctgagcgtgtactggtatcagcagtctctggaccagggcctgcagttcctgatccagtactacaacggcgaggaaagagccaagggcaacatcctggaacggttcagcgcccagcagttcccagatctgcacagcgagctgaacctgagcagcctggaactgggagatagcgccctgtacttctgtgccagcggcagaggcgataccgaggccttttttggccaaggcaccagactgaccgtggtggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgtccagcagactgagagtgtccgccaccttctggcagaaccccagaaaccacttcagatgccaggtgcagttctacggcctgagcgagaacgatgagtggacccaggacagagctaagcccgtgacacagatcgtgtctgccgaagcttggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggc

SEQ ID NO: 214 - protein, 275 aa, Artificial Sequence
  Example TCR alpha chain S4 population, full - with TRAC constant domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVIGGTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 215 - protein, 315 aa, Artificial Sequence
  Example TCR beta chain S4 population, full - with TRBC2 constant domain
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEEGAVYGYTFGSGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 216 - protein, 276 aa, Artificial Sequence
  Example TCR alpha chain S1 IFNg enriched population, full - with TRAC constant domain
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVPRGLSTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 217 - protein, 312 aa, Artificial Sequence
  Example TCR beta chain S1 IFNg enriched population, full - with TRBC2 constant domain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSREGLAADTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 218 - DNA, 825 bp, Artificial Sequence
  nucleotide sequence encoding TCR, donor HD15 S4 population alpha chain (with TRAC)
atgaagtccctgagagtgctgctggtcatcctgtggctgcagctgtcttgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgttcatctacagcaacggcgacaaagaggacggccggtttacagcccagctgaacaaggccagccagtacgtgtccctgctgatcagagatagccagcctagcgacagcgccacctatctgtgtgccgtgatcggcggcacagatagctggggcaaactccagtttggcgctggcacacaggtggtggtcacccctgacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtctgtgatcggcttccggatcctgctgctgaaggtggccggcttcaatctgctgatgaccctgagactgtggtccagc

SEQ ID NO: 219 - DNA, 945 bp, Artificial Sequence
  nucleotide sequence encoding TCR, donor HD15 S4 population beta chain (with TRBC2)
atgggatgtagacttctgtgttgcgccgtgctgtgtctgcttggagctggcgaactggtgcctatggaaaccggcgtgacccagacacctagacacctggtcatgggcatgacaaacaagaaaagcctgaagtgcgagcagcacctgggccacaatgccatgtactggtacaagcagagcgccaagaaacccctggaactgatgttcgtgtacagcctggaagagagggtcgagaacaacagcgtgcccagcagattcagccctgagtgccctaatagcagccacctgtttctgcatctgcacaccctgcagcctgaggactctgccctgtatctgtgtgccagcagccaagaggaaggcgccgtttacggctacacatttggcagcggcaccagactgaccgtggtggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgctgtgctggtgtccgctctggtgctgatggccatggtcaagcggaaggatagcaggggc

SEQ ID NO: 220 - DNA, 828 bp, Artificial Sequence
  nucleotide sequence encoding TCR, donor HD15 S1 IFNg enriched population alpha chain (with TRAC)
atgatcagcctgagagtgctgctggtcatcctgtggctgcagctgtcttgggtctggtcccagcggaaagaggtggaacaggaccccggacctttcaatgtgcctgaaggcgccaccgtggccttcaactgcacctacagcaatagcgccagccagagcttcttctggtacagacaggactgccggaaagaacccaagctgctgatgagcgtgtacagcagcggcaacgaggacggcagattcacagcccagctgaacagagccagccagtacatcagcctgctgatccgggatagcaagctgagcgatagcgccacctacctgtgcgtggtgcctagaggcctgagcacagattcttggggcaagctgcagttcggagccggaacacaggtggtggtcacccctgacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctcatgaccctgagactgtggtccagc

SEQ ID NO: 221 - DNA, 936 bp, Artificial Sequence
  nucleotide sequence encoding TCR, donor HD15 S1 IFNg enriched population beta chain (with TRBC2)
atgggacctggacttcttcattggatggccctgtgtctgctcggcacaggacatggcgacgctatggtcattcagaaccccagataccaagtgacccagttcggcaagcccgtgacactgagctgtagccagacactgaaccacaacgtgatgtactggtatcagcagaagtcctctcaggcccctaagctgctgttccactactacgacaaggacttcaacaacgaggccgacacacccgacaacttccagagcagaaggcccaataccagcttctgcttcctggacatcagaagccctggcctgggagatgccgccatgtatctgtgtgccaccagcagagaaggcctggccgccgatacacagtatttcggccctggcaccagactgaccgtgctcgaggatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccctcggaaccacttcagatgccaggtgcagttctacggcctgagcgagaacgatgagtggacccaggatagagccaagcctgtgactcagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggc

SEQ ID NO: 222 - protein, 310 aa, Artificial Sequence; identical to SEQ ID NO: 10
  Donor HD12 TCR beta chain, full - with TRBC1 constant domain

SEQ ID NO: 223 - protein, 308 aa, Artificial Sequence; identical to SEQ ID NO: 21
  Donor HD13 TCR beta chain, full - with TRBC1 constant domain

SEQ ID NO: 224 - protein, 307 aa, Artificial Sequence; identical to SEQ ID NO: 32
  Donor HD14 TCR beta chain, full - with TRBC1 constant domain

SEQ ID NO: 225 - protein, 313 aa, Artificial Sequence; identical to SEQ ID NO: 43
  Donor HD15 TCR beta chain S4 population, full - with TRBC1 constant domain

SEQ ID NO: 226 - protein, 310 aa, Artificial Sequence; identical to SEQ ID NO: 54
  Donor HD15 TCR beta chain S1 IFNg enriched population, full - with TRBC1 constant domain

SEQ ID NO: 227 - protein, 310 aa, Artificial Sequence; identical to SEQ ID NO: 100
  Patient 1 beta 1 chain direct sequencing upon sorting, full - with TRBC1 constant domain

SEQ ID NO: 228 - protein, 312 aa, Artificial Sequence; identical to SEQ ID NO: 106
  Patient 1 beta 2 chain direct sequencing upon sorting, full - with TRBC1 constant domain

SEQ ID NO: 229 - protein, 307 aa, Artificial Sequence; identical to SEQ ID NO: 122
  Patient 1 alpha 2 chain growing colony, full - with TRBC1 constant domain

SEQ ID NO: 230 - protein, 317 aa, Artificial Sequence; identical to SEQ ID NO: 138
  Patient 2 TCR beta 1 chain, full - with TRBC1 constant domain

SEQ ID NO: 231 - protein, 307 aa, Artificial Sequence; identical to SEQ ID NO: 144
  Patient 2 TCR beta 2 chain, full - with TRBC1 constant domain

SEQ ID NO: 232 - protein, 310 aa, Artificial Sequence; identical to SEQ ID NO: 160
  Patient 23 TCR beta chain, full - with TRBC1 constant domain

SEQ ID NO: 233 - protein, 9 aa, Artificial Sequence
  core peptide sequence
GAQYRIHTH

SEQ ID NO: 234 - protein, 9 aa, Artificial Sequence
  core peptide sequence
AQYRIHTHG

SEQ ID NO: 235 - protein, 9 aa, Artificial Sequence
  core peptide sequence
QYRIHTHGV

SEQ ID NO: 236 - protein, 9 aa, Artificial Sequence
  core peptide sequence
RIHTHGVFR

SEQ ID NO: 237 - protein, 9 aa, Artificial Sequence
  core peptide sequence
IHTHGVFRG

SEQ ID NO: 238 - protein, 9 aa, Artificial Sequence
  core peptide sequence
HTHGVFRGI

SEQ ID NO: 239 - protein, 9 aa, Artificial Sequence
  core peptide sequence
LAAILDFLL

SEQ ID NO: 240 - protein, 9 aa, Artificial Sequence
  core peptide sequence
AILDFLLLQ

SEQ ID NO: 241 - protein, 9 aa, Artificial Sequence
  core peptide sequence
ILDFLLLQD

SEQ ID NO: 242 - protein, 9 aa, Artificial Sequence
  core peptide sequence
LDFLLLQDP

SEQ ID NO: 243 - protein, 9 aa, Artificial Sequence
  core peptide sequence
DFLLLQDPA

SEQ ID NO: 244 - protein, 9 aa, Artificial Sequence
  core peptide sequence
CMTWNQMNL

SEQ ID NO: 245 - protein, 9 aa, Artificial Sequence
  core peptide sequence
MTWNQMNLG

SEQ ID NO: 246 - protein, 9 aa, Artificial Sequence
  core peptide sequence
TWNQMNLGA

SEQ ID NO: 247 - protein, 9 aa, Artificial Sequence
  core peptide sequence
WNQMNLGAT

SEQ ID NO: 248 - protein, 9 aa, Artificial Sequence
  core peptide sequence
NQMNLGATL

SEQ ID NO: 249 - protein, 9 aa, Artificial Sequence
  core peptide sequence
QMNLGATLK

SEQ ID NO: 250 - protein, 9 aa, Artificial Sequence
  core peptide sequence
MNLGATLKG